(19) United States
(12) Patent Application Publication
(10) Pub. No.: US 2021/0392895 A1
(43) Pub. Date: Dec. 23, 2021

(54) NOVEL OXADIAZOLES

(71) Applicant: PI INDUSTRIES LIMITED, Rajasthan (IN)

(72) Inventors: Paras Raybhan BHUJADE, Maharashtra (IN); Maruti N NAIK, Karnataka (IN); Rajesh PAWAR, Betul-MP (IN); Pooja TRIVEDI, Rajasthan (IN); Rohit Arvind DENGALE, Maharashtra (IN); Shantanu Ganesh KULKARNI, Maharashtra (IN); Nitin Ramesh TEMBHARE, Maharashtra (IN); Santosh Shridhar AUTKAR, Maharashtra (IN); Ruchi GARG, Varanasi-UP (IN); Hagalavadi M VENKATESHA, Karnataka (IN); Alexander G.M. KLAUSENER, Pulheim (DE)

(73) Assignee: PI INDUSTRIES LIMITED, Rajasthan (IN)

(21) Appl. No.: 17/250,938

(22) PCT Filed: Sep. 30, 2019
(86) PCT No.: PCT/IB2019/058276

§ 371 Date: Mar. 31, 2021

(30) Foreign Application Priority Data
Oct. 1, 2018 (IN) 201811037146

(51) Int. Cl. A01N 43/82; C07D 413/12; C07D 413/14; A01P 3/00

(52) U.S. Cl. CPC A01N 43/82; A01P 3/00; C07D 413/14; C07D 413/12

(57) ABSTRACT

The present invention relates to novel compound of Formula I,

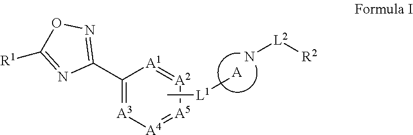

wherein, R¹, A¹, A², A³, A⁴, A⁵, L¹, A, L² and R² are as defined in the detailed description. The present invention also relates to a combination or a composition comprising the compound of Formula I.

### FIELD OF THE INVENTION

The present invention relates to novel oxadiazoles, their N-oxides, metal complexes, isomers, polymorphs and/or the agriculturally acceptable salts thereof and to a process for preparing the same. Further, the present invention relates to a combination and to compositions comprising novel oxadiazoles of the present invention. Still further, the present invention relates to the use of novel oxadiazoles for controlling or preventing phytopathogenic fungi and to a method for controlling or preventing harmful phytopathogenic fungi.

### BACKGROUND

Oxadiazoles have already been disclosed in the literature. For example in JP56065881, JP63162680, JPS6061573, JPS6296480, JPS6051188, JP2005336101, WO2005051932, EP3165093, EP3165094, EP3167716, EP3165093, JP2017190296, U.S. Pat. No. 4,488,897, WO2015185485, WO2017055469, WO2017055473, WO2017076739, WO2017076740, WO2017081311, WO2017085098, WO2017085100, WO2017093019, WO2017093348, WO2017102006, WO2017103219, WO2017103223, WO2017109044, WO2017110861, WO2017110862, WO2017110863, WO2017110864, WO2017110865, WO2017111152, WO2017118689, WO2017148797, WO2017157962, WO2017162868, WO2017169893, WO2017174158, WO2017178245, WO2017178549, WO2017198852, WO2017207757, WO2017211649, WO2017211650, WO2017211652, WO2017213252, WO2017220485, WO201772247, WO201776742, WO201776757, WO201776935, WO201781309, WO201781310, WO201781311, WO201781312, WO2018015447, WO2018015449, WO2018015458, WO2018056340, WO2018055135, WO2018080859, WO2018118781, WO2018117034, WO2018153730 and WO2018114393, various oxadiazoles have been disclosed.

The oxadiazoles reported in the above literature have disadvantages in certain aspects, such as that they exhibit a narrow spectrum of application, or they do not have satisfactory fungicidal activity, particularly at low application rates.

Therefore, it is an object of the present invention to provide compounds having an improved/enhanced activity and/or a broader activity spectrum against phytopathogenic fungi.

This objective is achieved by the use of novel oxadiazoles of the present invention for controlling or preventing phytopathogenic fungi.

### SUMMARY

The present invention relates to novel oxadiazoles of Formula I.

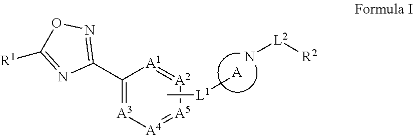

wherein, R¹, A¹, A², A³, A⁴, A⁵, L¹, A, L² and R² are as defined in the detailed description.

The compound of Formula I have now been found to have advantages over the compounds reported in the literature in either of improved fungicidal activity, broader spectrum biological activity, lower application rates, biological or environmental properties, and/or enhanced plant compatibility.

More specifically, the present invention further relates to combinations comprising novel oxadiazoles and at least one further pesticidally active substance for controlling or preventing phytopathogenic fungi which are difficult to control or prevent.

The present invention still further relates to compositions comprising novel oxadiazoles or novel oxadiazoles in combination with further pesticidally active substances.

The present invention still further relates to a method and use of novel oxadiazoles, of combinations or of compositions thereof for controlling and or preventing plant diseases, particularly phytopathogenic fungi.

### DETAILED DESCRIPTION

### Definitions

The definitions provided herein for the terminologies used in the present disclosure are for illustrative purpose only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “agronomic” refers to the production of field crops such as for food, feed and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management.

Compounds of the present disclosure may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated.

The term “alkyl”, used either alone or in compound words such as “alkylthio” or “haloalkyl” or —N(alkyl) or alkylcarbonylalkyl or alkylsuphonylamino includes straight-chain or branched C₁ to C₂₄ alkyl, preferably C₁ to C₁₅ alkyl, more preferably C₁ to C₁₀ alkyl, most preferably C₁ to C₆ alkyl. Non-limiting examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxy, halogen, carbonyl, carbonyloxy and the like, are at the end.

The term “alkenyl”, used either alone or in compound words includes straight-chain or branched C₂ to C₂₄ alkenes, preferably C₂ to C₁₅ alkenes, more preferably C₂ to C₁₀ alkenes, most preferably C₂ to C₆ alkenes. Non-limiting examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

Non-limiting examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term “alkynyl” can also include moieties comprised of multiple triple bonds such as 2,5-hexadiynyl.

The term “cycloalkyl” means alkyl closed to form a ring. Non-limiting examples include cyclopropyl, cyclopentyl and cyclohexyl. This definition also applies to cycloalkyl as a part of a composite substituent, for example cycloalkylalkyl etc., unless specifically defined elsewhere.

The term “cycloalkenyl” means alkenyl closed to form a ring including monocyclic, partially unsaturated hydrocarbyl groups. Non-limiting examples include cyclopropenyl, cyclopentenyl and cyclohexenyl. This definition also applies to cycloalkenyl as a part of a composite substituent, for example cycloalkenylalkyl etc., unless specifically defined elsewhere.

The term “cycloalkynyl” means alkynyl closed to form a ring including monocyclic, partially unsaturated groups. Non-limiting examples include cyclopropynyl, cyclopentynyl and cyclohexynyl.

This definition also applies to cycloalkynyl as a part of a composite substituent, for example cycloalkynylalkyl etc., unless specifically defined elsewhere.

The term “cycloalkoxy”, “cycloalkenyloxy” and the like are defined analogously. Non limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl”, “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Non-limiting examples of “haloalkylsulfinyl” include CF₃S(O), CCl₃S(O), CF₃CH₂S(O) and CF₃CF₂S(O). Non-limiting examples of “haloalkylsulfonyl” include CF₃S(O)₂, CCl₃S(O)₂, CF₃CH₂S(O)₂ and CF₃CF₂S(O)₂.

The term “hydroxy” means OH, Amino means —NRR, wherein R can be H or any possible substituent such as alkyl. Carbonyl means —C(═O)—, carbonyloxy means —OC(═O)—, sulfinyl means SO, sulfonyl means S(O)₂.

The term “alkoxy” used either alone or in compound words included C₁ to C₂₄ alkoxy, preferably C₁ to C₁₅ alkoxy, more preferably C₁ to C₁₀ alkoxy, most preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “alkoxyalkyl” denotes alkoxy substitution on alkyl. Non-limiting examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkoxyalkoxy” denotes alkoxy substitution on alkoxy.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Halocycloalkyl, halocycloalkenyl, alkylcycloalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

The term “alkylthioalkyl” denotes alkylthio substitution on alkyl. Non-limiting examples of “alkylthioalkyl” include —CH₂SCH₂, —CH₂SCH₂CH₂, CH₃CH₂SCH₂, CH₃CH₂CH₂CH₂SCH₂ and CH₃CH₂SCH₂CH₂. “Alkylthioalkoxy” denotes alkylthio substitution on alkoxy. The term “cycloalkylalkylamino” denotes cycloalkyl substitution on alkyl amino.

The terms “alkoxyalkoxyalkyl”, “alkylaminoalkyl”, “dialkylaminoalkyl”, “cycloalkylaminoalkyl”, “cycloalkylaminocarbonyl” and the like, are defined analogously to “alkylthioalkyl” or “cycloalkylalkylamino”.

The term “alkoxycarbonyl” is an alkoxy group bonded to a skeleton via a carbonyl group (—CO—). This definition also applies to alkoxycarbonyl as a part of a composite substituent, for example cycloalkylalkoxycarbonyl and the like, unless specifically defined elsewhere.

The term “alkoxycarbonylalkylamino” denotes alkoxy carbonyl substitution on alkyl amino. “Alkylcarbonylalkylamino” denotes alkyl carbonyl substitution on alkyl amino. The terms alkylthioalkoxycarbonyl, cycloalkylalkylaminoalkyl and the like are defined analogously.

Non-limiting examples of “alkylsulfinyl” include methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl and the different isomers. The term “arylsulfinyl” includes Ar—S(O), wherein Ar can be any carbocyle or heterocylcle. This definition also applies to alkylsulphinyl as a part of a composite substituent, for example haloalkylsulphinyl etc., unless specifically defined elsewhere.

Non-limiting examples of “alkylsulfonyl” include methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl and the different isomers. The term “arylsulfonyl” includes Ar—S(O)₂, wherein Ar can be any carbocyle or heterocylcle. This definition also applies to alkylsulphonyl as a part of a composite substituent, for example alkylsulphonylalkyl etc., unless defined elsewhere.

“Alkylamino”, “dialkylamino”, and the like, are defined analogously to the above examples.

The term “carbocycle or carbocyclic” includes “aromatic carbocyclic ring system” and “non-aromatic carbocylic ring system” or polycyclic or bicyclic (spiro, fused, bridged, nonfused) ring compounds in which ring may be aromatic or non-aromatic (where aromatic indicates that the Huckel rule is satisfied and non-aromatic indicates that the Huckel rule is not satisfied).

The term “heterocycle or heterocyclic” includes “aromatic heterocycle or heteroaryl ring system” and “non-aromatic heterocycle ring system” or polycyclic or bicyclic (spiro, fused, bridged, nonfused) ring compounds in which ring may be aromatic or non-aromatic, wherein the heterocycle ring contains at least one heteroatom selected from N, O, S(O)₀₋₂, and or C ring member of the heterocycle may be replaced by C(═O), C(═S), C(═CR*R*) and C═NR*, * indicates integers.

The term “non-aromatic heterocycle” or “non-aromatic heterocyclic” means three- to fifteen-membered, preferably three- to twelve-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; non-limiting examples oxetanyl, oxiranyl, aziridinyl, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-1-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, pyrrolinyl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, piperidinyl, 2-piperidinyl, 3-piperidinyl, 4-piperidinyl, pyrazynyl, morpholinyl, thiomorphlinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, cycloserines, 2,3,4,5-tetrahydro[1H]azepin-1- or -2- or -3- or -4- or -5- or -6- or -7-yl, 3,4,5,6-tetra-hydro[2H]azepin-2- or -3- or -4- or -5- or -6- or -7-yl, 2,3,4,7-tetrahydro[1]azepin-1- or -2- or -3- or -4- or -5- or -6- or -7-yl, 2,3,6,7-tetrahydro[1H]azepin-1- or -2- or -3- or -4- or -5- or -6- or -7-yl, hexahydroazepin-1- or -2- or -3- or -4-yl, tetra- and hexahydrooxepinyl such as 2,3,4,5-tetrahydro[1H]oxepin-2- or -3- or -4- or -5- or -6- or -7-yl, 2,3,4,7-tetrahydro[1H]oxepin-2- or -3- or -4- or -5- or -6- or -7-yl, 2,3,6,7-tetrahydro[1H]oxepin-2- or -3- or -4- or -5- or -6- or -7-yl, hexahydroazepin-1- or -2- or -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl. This definition also applies to heterocyclyl as a part of a composite substituent, for example heterocyclylalkyl etc., unless specifically defined elsewhere.

The term “heteroaryl” or “aromatic heterocyclic” means 5 or 6-membered, fully unsaturated monocyclic ring system containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom; 5-membered heteroaryl groups which, in addition to carbon atoms, may contain one to four nitrogen atoms or one to three nitrogen atoms and one sulphur or oxygen atom as ring members, non-limiting examples furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, non-limiting examples 1-pyrrolyl, 1-pyrazolyl, 1,2,4-triazol-1-yl, 1-imidazolyl, 1,2,3-triazol-1-yl and 1,3,4-triazol-1-yl.

6-membered heteroaryl which contains one to four nitrogen atoms: 6-membered heteroaryl groups which, in addition to carbon atoms, may contain, respectively, one to three and one to four nitrogen atoms as ring members, non-limiting examples 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: non-limiting examples indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl; benzofused 6-membered heteroaryl which contains one to three nitrogen atoms: non-limiting examples quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

The term “trialkylsilyl” includes 3 branched and/or straight-chain alkyl radicals attached to and linked through a silicon atom such as trimethylsilyl, triethylsilyl and t-butyl-dimethylsilyl. “Halotrialkylsilyl” denotes at least one of the three alkyl radicals is partially or fully substituted with halogen atoms which may be the same or different. The term“alkoxytrialkylsilyl” denotes at least one of the three alkyl radicals is substituted with one or more alkoxy radicals which may be the same or different. The term “trialkylsilyloxy” denotes a trialkylsilyl moiety attached through oxygen. Non-limiting examples of “alkylcarbonyl” include C(═O)CH₃, C(═O)CH₂CH₂CH₃ and C(═O)CH(CH₃)₂. Non-limiting examples of “alkoxycarbonyl” include CH₃OC(═O), CH₃CH₂OC(═O), CH₃CH₂CH₂OC(═O), (CH₃)₂CHOC(═O) and the different butoxy -or pentoxycarbonyl isomers. Non-limiting examples of “alkylaminocarbonyl” include CH₃NHC(═O), CH₃CH₂NHC(═O), CH₃CH₂CH₂NHC(═O), (CH₃)₂CHNHC(═O) and the different butylamino -or pentylaminocarbonyl isomers. Non-limiting examples of “dialkylaminocarbonyl” include (CH₃)₂NC(═O), (CH₃CH₂)₂NC(═O), CH₃CH₂(CH₃)NC(═O), CH₃CH₂CH₂(CH₃)NC(═O) and (CH₃)₂CHN(CH₃)C(═O). Non-limiting examples of “alkoxyalkylcarbonyl” include CH₃OCH₂C(═O), CH₃OCH₂CH₂C(═O), CH₃CH₂OCH₂C(═O), CH₃CH₂CH₂CH₂OCH₂C(═O) and CH₃CH₂OCH₂CH₂C(═O). Non-limiting examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O). The term haloalkylsufonylaminocarbonyl, alkylsulfonylaminocarbonyl, alkylthioalkoxycarbonyl, alkoxycarbonylalkyl amino and the like are defined analogously

Non-limiting examples of “alkylaminoalkylcarbonyl” include CH₃NHCH₂C(═O), CH₃NHCH₂CH₂C(═O), CH₃CH₂NHCH₂C(═O), CH₃CH₂CH₂CH₂NHCH₂C(═O) and CH₃CH₂NHCH₂CH₂C(═O).

The term “amide” means A-R′C═ONR″—B, wherein R′ and R″ indicates substituents and A and B indicate any group.

The term “thioamide” means A-R′C═SNR″—B, wherein R′ and R″ indicates substituents and A and B indicate any group.

The total number of carbon atoms in a substituent group is indicated by the “Cᵢ-Cⱼ” prefix where i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of Formula I is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4 then the number of substituents may be selected from the integers between 0 and 4 inclusive.

When a group contains a substituent which can be hydrogen, then, when this substituent is taken as hydrogen, it is recognized that said group is being un-substituted.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned in the description and the description/claims though might form a critical part of the present invention of the present invention, any deviation from such numerical values shall still fall within the scope of the present invention if that deviation follows the same scientific principle as that of the present invention disclosed in the present invention. The inventive compound of the present invention may, if appropriate, be present as mixtures of different possible isomeric forms, especially of stereoisomers, for example E and Z, threo and erythro, and also optical isomers, but if appropriate also of tautomers. Both the E and the Z isomers, and also the threo and erythro isomers, and the optical isomers, any desired mixtures of these isomers and the possible tautomeric forms are disclosed and claimed.

The term “pest” for the purpose of the present disclosure includes but is not limited to fungi, stramenopiles (oomycetes), bacteria, nematodes, mites, ticks, insects and rodents.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

For the purpose of the present disclosure the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include but are not limited to agricultural crops such as wheat, rye, barley, triticale, oats or rice; beet, e.g. sugar beet or fodder beet; fruits and fruit trees, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit and citrus trees, such as oranges, lemons, grapefruits or mandarins; any horticultural plants, vegetables, such as spinach, lettuce, asparagus, cabbages, carrots, onions, tomatoes, potatoes, cucurbits or paprika; lauraceous plants, such as avocados, cinnamon or camphor; cucurbitaceae; oleaginous plants; energy and raw material plants, such as cereals, corn, soybean, other leguminous plants, rape, sugar cane or oil palm; tobacco; nuts;

coffee; tea; cacao; bananas; peppers; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, the plant for the purpose of the present invention includes but is not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for use of human and animals.

The term “plant parts” is understood to mean all parts and organs of plants above and below the ground. For the purpose of the present disclosure the term plant parts includes but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxillary buds, meristems, nodes and internodes.

The term “locus thereof” includes soil, surroundings of plant or plant parts and equipment or tools used before, during or after sowing/planting a plant or a plant part.

Application of the compounds of the present disclosure or the compound of the present disclosure in a composition optionally comprising other compatible compounds to a plant or a plant material or locus thereof include application by a technique known to a person skilled in the art which include but is not limited to spraying, coating, dipping, fumigating, impregnating, injecting and dusting.

The term “applied” means adhered to a plant or plant part either physically or chemically including impregnation.

Accordingly, novel oxadiazoles of the present invention are represented by Formula I and include N-oxides, metal complexes, isomers, polymorphs or the agriculturally acceptable salts thereof.

The present invention relates to a compound of Formula I:

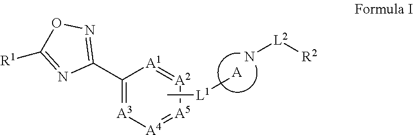

wherein, the substituents and definitions are as defined hereinafter.
R¹ is selected from the group consisting of C₁-C₂-monohaloalkyl, C₁-C₂-dihaloalkyl, C₁-C₂-trihaloalkyl, C₁-C₂-tetrahaloalkyl, and C₁-C₂-pentahaloalkyl.

In one embodiment R¹ is difluoromethyl or trifluoromethyl. In a particular embodiment, R¹ is trifluoromethyl.

A¹ is C or N. In a particular embodiment, A¹ is C.

A² is C or N.

A³ is C or N. In a particular embodiment, A³ is C.

A⁴ is C or N.

A⁵ is C or N.

In some of the embodiments, no more than two of A¹, A², A³, A⁴, and A⁵ are nitrogen.

In another embodiment, no more than one of A², A⁴, and A⁵ are nitrogen.

In yet another embodiment, no more than one of A² and A⁴ are nitrogen.

In some of the embodiments, A¹, A², A³, A⁴, and A⁵ are independently and optionally substituted with one or more R^{G} selected from the group consisting of hydrogen, halogen, cyano, nitro, amino, hydroxy, C₁-C₆-alkyl, C₃-C₆-cycloalkyl, C₁-C₆-haloalkyl, C₁-C₆-hydroxyalkyl, C₁-C₆-alkoxy, C₁-C₆-alkoxy-C₁-C₆-alkyl, and C₁-C₆-haloalkoxy.

In another embodiment, A¹, A², A³, A⁴, and A⁵ are independently and optionally substituted with R^{G} selected from the group consisting of hydrogen, halogen, cyano, C₁-C₆-alkyl, C₁-C₆-alkoxy, C₃-C₆-cycloalkyl, and C₁-C₆-haloalkoxy.

In a particular embodiment, A¹, A², A³, A⁴, and A⁵ are independently and optionally substituted with R^{G} selected from the group consisting of hydrogen, halogen, methyl, ethyl, propyl, isopropyl, methoxy, trifluoro methoxy, trifluoro methyl, difluoro methyl, and cyclopropyl.

Alternatively, two R^{G} together with the atoms to which they are attached may form a 3-, 4-, 5-, or 6-membered carbocyclic ring or ring system or 4-, 5-, or 6-membered heterocyclic ring or ring system; wherein C atom ring members of the carbocyclic or the heterocyclic ring or ring system may be replaced by C(═O) or C(═S); and heteroatom in the heterocyclic ring or ring system is selected from N, O or S(O)₀₋₂; wherein, the carbocyclic or the heterocyclic ring or ring system formed by two R^{G} may optionally further be substituted with one or more of halogen, C₁-C₆-alkyl, C₃-C₆-cycloalkyl, C₁-C₆-haloalkyl, C₁-C₆-hydroxyalkyl, C₁-C₆-alkoxy, and C₁-C₆-haloalkoxy.

In some of the embodiments, L¹ is O, S(═O)₀₋₂, NR⁶, or

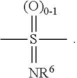

In another embodiment, L¹ is O, S(═O)₀₋₂, or NR⁶.

In a particular embodiment, L¹ is O, S, S(═O)₂, N-methyl, N-ethyl, N-propyl, N-isopropyl, and N-cyclopropyl.

L¹ may be attached to either of A², A⁴, or A⁵.

In some of the embodiments, A is a nitrogen containing 3-, 4-, 5- or 6-membered nonaromatic heterocyclic ring; wherein, ring A additionally may comprise a hetero atom selected from N, O, and S(═O)₀₋₂; wherein, ring A may be optionally substituted with one or more R^{A}.

In another embodiment, A is a nitrogen containing 4-, 5- or 6-membered nonaromatic heterocyclic ring; wherein, ring A may be optionally substituted with one or more R^{A}.

In some embodiments, the substituent R^{A} on A is independently selected from hydrogen, halogen, cyano, nitro, amino, hydroxy, oxo, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₆-alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, C₁-C₆-haloalkoxycarbonyl, C₁-C₆-alkylthio, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, C₁-C₆-dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, C₁-C₆-dialkylaminocarbonyl, C₁-C₆-alkoxycarbonyloxy, C₁-C₆-alkylaminocarbonyloxy, C₁-C₆-dialkylaminocarbonyloxy, sulfilimines, sulfoximines, sulfonamide, and sulfonamide.

In another embodiment, the substituent R^{A} on A is independently selected from halogen, cyano, C₁-C₆-alkyl, C₂-C₆-alkenyl, C₂-C₆-alkynyl, C₁-C₆-alkoxy, and C₃-C₈-cycloalkyl.

In a particular embodiment, the substituent R^{A} on A is independently selected from fluorine, bromine, chlorine, iodine, cyano, methyl, ethyl, propyl, isopropyl, methoxy, ethoxy, and cyclopropyl.

In some of the embodiments, L² is (C(═O))₁₋₂, (CR⁸R⁹)₁₋₃, S(═O)₀₋₂, NR¹⁸, or

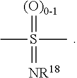

In another embodiment, L² is C(═O), (CR⁸R⁹), S(═O)₂, or NR¹⁸.

In a particular embodiment, L² is C(═O), (CH₂), (CHCH₃), or S(═O)₂.

Alternatively, R^{A} and R¹⁸; or R^{A} and R⁸ or R⁹; together with the atom to which they are attached may form a saturated or unsaturated or partially unsaturated 4-, 5-, or 6-membered heterocyclic ring or ring system comprising one or more N; wherein, the heterocyclic ring or ring system may optionally further be substituted with one or more of halogen, C₁-C₆-alkyl, C₃-C₆-cycloalkyl, C₁-C₆-haloalkyl, C₁-C₆-hydroxyalkyl, C₁-C₆-alkoxy, C₁-C₆-alkylaminocarbonyl, C₁-C₆-haloalkoxy, —(CR¹²R¹³)₀₋₁—C(═O)—NR¹⁴R¹⁵, —(CR¹²R¹³)₀₋₁—NR¹⁴—C(═O)—(C₁-C₆-alkyl), —(CR¹²R¹³)₀₋₁—NR¹⁴—S(═O)₀₋₂—(C₁-C₆-alkyl), and —(CR¹²R¹³)₀₋₁—NR¹⁴—C(═O)—NR¹⁴;

In some of the embodiments, R² is selected from hydrogen, halogen, cyano, nitro, amino, hydroxy, C₁-C₆ alkyl, C₂-C₆-alkenyl, C₂-C₆-alkynyl, C₃-C₈-cycloalkyl, C₃-C₈-cycloalkylalkyl, C₁-C₆ haloalkyl, C₁-C₆ alkoxy C₁-C₄ alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, aryloxy, heteroaryloxy, C₄-C₈-heterocyclyloxy, C₃-C₈-cycloalkyloxy, C₁-C₆-haloalkoxycarbonyl, C₁-C₆ alkylthio, C₁-C₆-haloalkylthio, C₁-C₆-haloalkylsulfonyl, C₁-C₆ alkylsulfinyl, C₁-C₆-alkylsulfonyl, C₁-C₆-alkylamino, C₄-C₈-heterocyclylamino, heteroarylamino, arylamino, C₁-C₆ dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, C₁-C₆-dialkylaminocarbonyl, C₁-C₆-alkoxycarbonyloxy, C₁-C₆-alkylaminocarbonyloxy, or C₁-C₆-dialkylaminocarbonyloxy, sulfilimines, sulfoximines, sulfonamide, and sulfinamide; R² may optionally further be substituted with one or more R⁷.

In another embodiment, R² is selected from hydrogen, C₁-C₆-alkyl, C₂-C₆-alkenyl, C₂-C₆-alkynyl, C₃-C₈-cycloalkyl, C₃-C₈-cycloalkylalkyl, C₁-C₆ haloalkyl, C₁-C₆ alkoxy C₁-C₄ alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, aryloxy, heteroaryloxy, C₄-C₈-heterocyclyloxy, C₃-C₈-cycloalkyloxy, C₁-C₆-haloalkoxycarbonyl, C₁-C₆ alkylthio, C₁-C₆-haloalkylthio, C₁-C₆ haloalkylsulfinyl, C₁-C₆-haloalkylsulfonyl, C₁-C₆ alkylsulfinyl, C₁-C₆-alkylsulfonyl, C₁-C₆ alkylamino, C₄-C₈-heterocyclylamino, heteroarylamino, arylamino; R² may optionally further be substituted with one or more R⁷.

In a particular embodiment, R² is selected from hydrogen, C₁-C₆-alkyl, C₃-C₈-cycloalkyl, C₁-C₆-haloalkyl, C₃-C₈-halocycloalkyl, C₁-C₆-alkoxy, C₁-C₆-haloalkoxy, heteroarylamino, and arylamino; R² may optionally further be substituted with one or more R⁷.

In the first alternate embodiment, R² is phenyl, benzyl, naphthyl, a 5- or 6-membered aromatic ring, an 8- to 11-membered aromatic multi-cyclic ring system, an 8- to 11-membered aromatic fused ring system, a 5- or 6-membered heteroaromatic ring, an 8- to 11-membered heteroaromatic multi-cyclic ring system or an 8- to 11-membered heteroaromatic fused ring system; wherein heteroatom of the heteroaromatic ring or ring system is selected from N, O or S, and each aromatic or heteroaromatic ring or ring system may be optionally substituted with one or more substituents selected from R³.

In another first alternate embodiment, R² is phenyl, benzyl, a 5- or 6-membered aromatic ring, a 5- or 6-membered heteroaromatic ring; wherein heteroatom of the heteroaromatic ring or ring system is N, and each aromatic or heteroaromatic ring or ring system may be optionally substituted with one or more substituents selected from R³.

In a particular first alternate embodiment, R² is phenyl, benzyl, a 5- or 6-membered heteroaromatic ring; wherein heteroatom of the heteroaromatic ring or ring system is N, and each aromatic or heteroaromatic ring or ring system may be optionally substituted with one or more substituents selected from R³.

In the second alternate embodiment, R² is a 3- to 7-membered nonaromatic carbocyclic ring, a 4-, 5-, 6- or 7-membered nonaromatic heterocyclic ring, an 8- to 15-membered nonaromatic multi-cyclic ring system, an 5- to 15 membered spirocyclic ring system, or an 8- to 15-membered nonaromatic fused ring system, wherein, the heteroatom of the nonaromatic heterocyclic ring or ring system is selected from N, O or S(O)₀₋₂, and C ring member of the nonaromatic carbocyclic or nonaromatic heterocyclic ring or ring system may be replaced with C(═O), C(═S), C(═CR²⁰R²¹) or C(═NR¹⁹) and each nonaromatic carbocyclic or nonaromatic heterocyclic ring or ring system may be optionally substituted with one or more substituents selected from R³.

In another second alternate embodiment, R² is a 3- to 7-membered nonaromatic carbocyclic ring, a 4-, 5-, 6- or 7-membered nonaromatic heterocyclic ring, the heteroatom of the nonaromatic heterocyclic ring or ring system is selected from N, O or S(O)₀₋₂, and each nonaromatic carbocyclic or nonaromatic heterocyclic ring or ring system may be optionally substituted with one or more substituents selected from R³.

In a particular second alternate embodiment, R² is a 3- to 6-membered nonaromatic carbocyclic ring, a 4-, 5-, 6- or 7-membered nonaromatic heterocyclic ring, the heteroatom of the nonaromatic heterocyclic ring or ring system is selected from N, O or S(O)₀₋₂, and each nonaromatic carbocyclic or nonaromatic heterocyclic ring or ring system may be optionally substituted with one or more substituents selected from R³.

In some of the embodiments, R³ is independently selected from halogen, cyano, nitro, hydroxy, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₈-cycloalkyl, C₃-C₈-halocycloalkyl, C₃-C₈-cycloalkyl-C₁-C₆-alkyl, C₃-C₈-cycloalkyl-C₃-C₈-cycloalkyl, C₃-C₈-cycloalkenyl, C₁-C₆-alkoxy-C₁-C₆-alkyl, C₃-C₈-cycloalkoxy-C₁-C₆-alkyl, C₁-C₆-alkylsulfinyl-C₁-C₆-alkyl, C₁-C₆-alkylsulfonyl-C₁-C₆-alkyl, C₁-C₆-alkylamino, di-C₁-C₆-alkylamino, C₁-C₆-alkylamino-C₁-C₆-alkyl, di-C₁-C₆-alkylamino-C₁-C₆-alkyl, C₁-C₆-haloalkylamino-C₁-C₆-alkyl, C₃-C₈-cycloalkylamino, C₃-C₈-cycloalkylamino-C₁-C₆-alkyl, C₁-C₆-alkylcarbonyl, C₁-C₆-haloalkoxy-C₁-C₆-alkyl, C₁-C₆-hydroxyalkyl, C₂-C₆-hydroxyalkenyl, C₂-C₆-hydroxyalkynyl, C₂-C₆-alkenyloxy, C₂-C₆-haloalkenyloxy, C₂-C₆-alkynyloxy, C₁-C₆-alkylcarbonylalkoxy, C₁-C₆-alkylthio, C₁-C₆-haloalkylthio, C₃-C₈-cycloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkylsulfonyl, C₃-C₈-cycloalkylsulfinyl, C₁-C₆-alkylsulfonylamino, C₁-C₆-haloalkylsulfonylamino, C₁-C₆-alkylsulfonyloxy, C₆-C₁₀-arylsulfonyloxy, C₆-C₁₀-arylsulfonyl, C₆-C₁₀-arylsulfinyl, C₆-C₁₀-arylthio, C₁-C₆-cyanoalkyl, C₁-C₆-haloalkylamino, C₁-C₆-alkoxyamino, C₁-C₆-haloalkoxyamino, C₁-C₆-alkoxycarbonylamino, C₁-C₆-alkylcarbonyl-C₁-C₆-alkylamino, C₂-C₆-alkenylthio, di(C₁-C₆-haloalkyl)amino-C₁-C₆-alkyl, C₁-C₆-alkylaminocarbonylamino, di(C₁-C₆-haloalkyl)amino, sulfilimines, sulfoximines, or SF₅; wherein, R³ may be optionally substituted with halogen, cyano, amino, C₁-C₆-alkyl, C₁-C₆-alkoxy, C₁-C₆-alkylthio, and C₃-C₈-cycloalkyl.

In another embodiment, R³ is independently selected from halogen, cyano, C₁-C₆-alkyl, C₁-C₆-haloalkyl, C₂-C₆-haloalkenyl, C₃-C₈-cycloalkyl, C₃-C₈-halocycloalkyl, C₁-C₆-alkoxy, and C₁-C₆-haloalkoxy.

In a particular embodiment, R³ is independently selected from halogen, cyano, C₁-C₆-alkyl, C₁-C₆-haloalkyl, C₃-C₈-cycloalkyl, and C₁-C₆-alkoxy.

In some of the embodiments, R⁷ is selected from C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆ alkoxy C₁-C₄ alkyl, aryloxy, heteroaryloxy, arylamino, heteroarylamino, arylthio, heteroarylthio, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, C₁-C₆-alkylamino-C₁-C₆-alkyl, C₁-C₆-haloalkoxycarbonyl, and amino-C₁-C₆-alkyl.

In another embodiment, R⁷ is selected from halogen, hydroxy, amino, C₁-C₆-alkyl, and C₃-C₈-cycloalkyl.

In an alternate embodiment, two R⁷ together with the atoms to which they are attached may form a 3-, 4-, 5-, or 6-membered carbocyclic ring or ring system or 4-, 5-, or 6-membered heterocyclic ring or ring system; wherein C atom ring members of the carbocyclic or the heterocyclic ring or ring system may be replaced by C(═O) or C(═S); and heteroatom in the heterocyclic ring or ring system is selected from N, O or S; or

In the first alternate embodiment, R⁷ is phenyl, benzyl, a 5-membered aromatic ring, a 5- or 6-membered heteroaromatic ring; wherein heteroatom of the heteroaromatic ring is selected from N, O or S.

In the second alternate embodiment, R⁷ is a 3- to 7-membered nonaromatic carbocyclic ring, a 4-, 5-, 6- or 7-membered nonaromatic heterocyclic ring, wherein, the heteroatom of the nonaromatic heterocyclic ring is selected from N, O or S(O)₀₋₂, and C ring member of the nonaromatic carbocyclic or nonaromatic heterocyclic ring may be replaced with C(═O), C(═S), C(═CR²²R²³), or C(═NR²⁴).

In another second embodiment, R⁷ is a 3- to 7-membered nonaromatic carbocyclic ring, a 4-, 5-, 6- or 7-membered nonaromatic heterocyclic ring, wherein, the heteroatom of the nonaromatic heterocyclic ring is selected from N.

In another particular embodiment, R⁷ is a 3- to 6-membered nonaromatic carbocyclic ring, a 4-, 5-, 6- or 7-membered nonaromatic heterocyclic ring, wherein, the heteroatom of the nonaromatic heterocyclic ring is selected from N.

The substituent R⁷ may be further substituted with one or more R¹⁶ on C atom and with one or more R¹⁷ on N atom,

In one embodiment, the substituents R⁴, R⁵, R⁸, R⁹, R¹², R¹³, R¹⁶, R²⁰, R²¹, R²², and R²³ are independently selected from hydrogen, halogen, cyano, nitro, NR¹⁰R¹¹, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, C₃-C₆-halocycloalkyl, C₁-C₄-alkoxy, C₃-C₈-cycloalkoxy, or C₁-C₄-haloalkoxy.

In another embodiment, the substituents R⁴, R⁵, R⁸, R⁹, R¹², R¹³, R¹⁶, R²⁰, R²¹, R²², and R²³ are independently selected from hydrogen, halogen, cyano, C₁-C₄-alkyl, C₁-C₄-haloalkyl, C₃-C₆-cycloalkyl, C₃-C₆-halocycloalkyl, C₁-C₄-alkoxy, C₃-C₈-cycloalkoxy, and C₁-C₄-haloalkoxy.

In one embodiment, the substituents R⁶, R¹⁰, R¹¹, R¹⁴, R¹⁵, R¹⁷, R¹⁸, R¹⁹, and R²⁴ are independently selected from the group of hydrogen, cyano, hydroxy, NR^{b}R^{c}, (C═O)—R^{d}, S(O)₀₋₂R^{e}, C₁-C₆-alkyl, C₁-C₆-haloalkyl, C₁-C₆-alkoxy, C₁-C₆-haloalkoxy, C₃-C₆-cycloalkyl, phenyl, aryl-C₁-C₆-alkyl, heteroaryl, heteroaryl-C₁-C₆-alkyl, C₁-C₆-alkylamino, di-C₁-C₆-alkylamino, tri-C₁-C₆-alkylamino, or C₃-C₈-cycloalkyl.

In one embodiment, the substituents R⁶, R¹⁰, R¹¹, R¹⁴, R¹⁵, R¹⁷, R¹⁸, R¹⁹, and R²⁴ are selected from the group of hydrogen, cyano, (C═O)—R^{d}, S(O)₀₋₂R^{e}, C₁-C₆-haloalkyl, C₁-C₆-alkoxy, C₁-C₆-haloalkoxy, and C₃-C₆-cycloalkyl.

In one embodiment, R^{b} and R^{c} represent hydrogen, hydroxyl, cyano, amino, C₁-C₄-alkyl, C₁-C₄-haloalkoxy, C₁-C₄-alkoxy, C₃-C₈-cycloalkyl, or C₃-C₈-halocycloalkyl.

In another embodiment, R^{b} and R^{c} represent hydrogen, C₁-C₄-alkyl, C₁-C₄-haloalkyl, C₁-C₄-alkoxy, or C₃-C₈-cycloalkyl.

In one embodiment, R^{d} represents hydrogen, hydroxy, halogen, NR^{b}R^{c}, C₁-C₆-haloalkyl, C₁-C₆-alkoxy, C₁-C₆-haloalkoxy, C₃-C₈-cycloalkyl or C₃-C₈-halocycloalkyl.

In another embodiment, R^{d} represents hydrogen, hydroxy, halogen, NR^{b}R^{c}, C₁-C₆-alkyl, C₁-C₆-haloalkyl, C₁-C₆-alkoxy, or C₃-C₈-cycloalkyl.

In one embodiment, R^{e} represents hydrogen, halogen, cyano, amino, C₁-C₆-alkyl, C₁-C₆-haloalkyl, C₁-C₆-alkoxy, C₁-C₆-haloalkoxy, C₃-C₈-cycloalkyl, or C₃-C₈-halocycloalkyl.

In another embodiment, R^{e} represents hydrogen, amino, C₁-C₆-alkyl, C₁-C₆-haloalkyl, C₁-C₆-alkoxy, or C₃-C₈-cycloalkyl.

Particularly, the compound of Formula I is selected from the group consisting of:
(S)-(3-methoxyphenyl)(3-(4-(5-(trifluoromethyl)-1,2,4-oxadiazol-3-yl)phenoxy)pyrrolidin-1-yl)methanone; (S)-1-(3-(4-(5-(trifluoromethyl)-1,2,4-oxadiazol-3-yl)phenoxy)pyrrolidin-1-yl)ethan-1-one; (S)-(3-bromophenyl)(3-(4-(5-(trifluoromethyl)-1,2,4-oxadiazol-3-yl)phenoxy)pyrrolidin-1-yl)methanone; (S)-4-(3-(4-(5-(trifluoromethyl)-1,2,4-oxadiazol-3-yl)phenoxy)pyrrolidine-1-carbonyl)benzonitrile; (S)-2-(3,4-dimethoxyphenyl)-1-(3-(4-(5-(trifluoromethyl)-1,2,4-oxadiazol-3-yl)phenoxy)pyrrolidin-1-yl)ethan-1-one; (S)-(2-fluorophenyl)(3-(4-(5-(trifluoromethyl)-1,2,4-oxadiazol-3-yl)phenoxy)pyrrolidin-1-yl)methanone; (S)-pyridin-2-yl(3-(4-(5-(trifluoromethyl)-1,2,4-oxadiazol-3-yl)phenoxy)pyrrolidin-1-yl)methanone; (S)-(4-(dimethylamino)phenyl)(3-(4-(5-(trifluoromethyl)-1,2,4-oxadiazol-3-yl)phenoxy)pyrrolidin-1-yl)methanone; (S)-cyclobutyl(3-(4-(5-(trifluoromethyl)-1,2,4-oxadiazol-3-yl)phenoxy)pyrrolidin-1-yl)methanone; (S)-(4-methoxyphenyl) (3-(4-(5-(trifluoromethyl)-1,2,4-oxadiazol-3-yl)phenoxy)pyrrolidin-1-yl)methanone; (S)-2-phenyl-1-(3-(4-(5-(trifluoromethyl)-1,2,4-oxadiazol-3-yl)phenoxy)pyrrolidin-1-yl)ethan-1-one; (S)-pyridin-3-yl(3-(4-(5-(trifluoromethyl)-1,2,4-oxadiazol-3-yl)phenoxy)pyrrolidin-1-yl)methanone; (S)-pyridin-4-yl(3-(4-(5-(trifluoromethyl)-1,2,4-oxadiazol-3-yl)phenoxy)pyrrolidin-1-yl)methanone; (S)-(4-fluorophenyl)(3-(4-(5-(trifluoromethyl)-1,2,4-oxadiazol-3-yl)phenoxy)pyrrolidin-1-yl)methanone; (S)-phenyl(3-(4-(5-(trifluoromethyl)-1,2,4-oxadiazol-3-yl)phenoxy)pyrrolidin-1-yl)methanone; (3-bromophenyl) (3-(4-(5-(trifluoromethyl)-1,2,4-oxadiazol-3-yl)phenoxy)azetidin-1-yl)methanone; (3-methoxyphenyl)(3-(4-(5-(trifluoromethyl)-1,2,4-oxadiazol-3-yl)phenoxy) azetidin-1-yl)methanone; (3-fluorophenyl)(3-(4-(5-(trifluoromethyl)-1,2,4-oxadiazol-3-yl)phenoxy) azetidin-1-yl)methanone; (3-(4-(5-(trifluoromethyl)-1,2,4-oxadiazol-3-yl)phenoxy)azetidin-1-yl)(4-(trifluoromethyl)phenyl)methanone; (2-fluorophenyl)(3-(4-(5-(trifluoromethyl)-1,2,4-oxadiazol-3-yl)phenoxy)azetidin-1-yl)methanone; phenyl(3-(4-(5-(trifluoromethyl)-1,2,4-oxadiazol-3-yl)phenoxy)azetidin-1-yl)methanone; 3-(4-((1-(benzylsulfonyl)azetidin-3-yl)oxy)phenyl)-5-(trifluoromethyl)-1,2,4-oxadiazole; tert-butyl (S)-3-(4-(5-(trifluoromethyl)-1,2,4-oxadiazol-3-yl)phenoxy)pyrrolidine-1-carboxylate; (S)-2-(3-methoxyphenyl)-1-(3-(4-(5-(trifluoromethyl)-1,2,4-oxadiazol-3-yl)phenoxy)pyrrolidin-1-yl)ethan-1-one; (S)-3-(4-((1-(phenylsulfonyl)pyrrolidin-3-yl)oxy)phenyl)-5-(trifluoromethyl)-1,2,4-oxadiazole; (S)-3-(4-((1-((3-fluorophenyl)sulfonyl)pyrrolidin-3-yl)oxy)phenyl)-5-(trifluoromethyl)-1,2,4-oxadiazole; tert-butyl (S)-3-((6-(5-(trifluoromethyl)-1,2,4-oxadiazol-3-yl)pyridin-3-yl)oxy)pyrrolidine-1-carboxylate; (S)-(2-fluorophenyl)(3-((6-(5-(trifluoromethyl)-1,2,4-oxadiazol-3-yl)pyridin-3-yl)oxy)pyrrolidin-1-yl)methanone; (S)-(4-methoxyphenyl)(3-((6-(5-(trifluoromethyl)-1,2,4-oxadiazol-3-yl)pyridin-3-yl)oxy)pyrrolidin-1-yl)methanone; (S)-(3-fluorophenyl)(3-((6-(5-(trifluoromethyl)-1,2,4-oxadiazol-3-yl)pyridin-3-yl)oxy)pyrrolidin-1-yl)methanone; (S)-pyridin-3-yl(3-((6-(5-(trifluoromethyl)-1,2,4-oxadiazol-3-yl)pyridin-3-yl)oxy)pyrrolidin-1-yl)methanone; (S)-pyridin-4-yl(3-((6-(5-(trifluoromethyl)-1,2,4-oxadiazol-3-yl)pyridin-3-yl)oxy)pyrrolidin-1-yl)methanone; (S)-3-(4-((1-((2-fluorophenyl)sulfonyl)pyrrolidin-3-yl)oxy)phenyl)-5-(trifluoromethyl)-1,2,4-oxadiazole; (S)-3-(4-((1-((4-methoxyphenyl)sulfonyl)pyrrolidin-3-yl)oxy)phenyl)-5-(trifluoromethyl)-1,2,4-oxadiazole; (S)-3-(4-((1-((4-fluorophenyl)sulfonyl)pyrrolidin-3-yl)oxy)phenyl)-5-(trifluoromethyl)-1,2,4-oxadiazole; (S)-(4-(trifluoromethoxy)phenyl)(3-(4-(5-(trifluoromethyl)-1,2,4-oxadiazol-3-yl)phenoxy)pyrrolidin-1-yl)methanone; (S)—N-(2-fluorophenyl)-3-(4-(5-(trifluoromethyl)-1,2,4-oxadiazol-3-yl)phenoxy)pyrrolidine-1-carboxamide; (S)—N-(4-fluorophenyl)-3-(4-(5-(trifluoromethyl)-1,2,4-oxadiazol-3-yl)phenoxy)pyrrolidine-1-carboxamide; (S)—N-(4-methoxyphenyl)-3-(4-(5-(trifluoromethyl)-1,2,4-oxadiazol-3-yl)phenoxy)pyrrolidine-1-carboxamide; (S)-3-(4-((1-(ethylsulfonyl)pyrrolidin-3-yl)oxy)phenyl)-5-(trifluoromethyl)-1,2,4-oxadiazole; (S)-3-(4-((1-(cyclopropylsulfonyl)pyrrolidin-3-yl)oxy)phenyl)-5-(trifluoromethyl)-1,2,4-oxadiazole; (S)-3-(4-((1-((2,4-difluorophenyl)sulfonyl)pyrrolidin-3-yl)oxy)phenyl)-5-(trifluoromethyl)-1,2,4-oxadiazole; (S)-cyclopropyl(3-(4-(5-(trifluoromethyl)-1,2,4-oxadiazol-3-yl)phenoxy)pyrrolidin-1-yl)methanone; (S)-(4-chlorophenyl)(3-(4-(5-(trifluoromethyl)-1,2,4-oxadiazol-3-yl)phenoxy)pyrrolidin-1-yl)methanone; (S)-(2-fluorophenyl)(3-((5-(5-(trifluoromethyl)-1,2,4-oxadiazol-3-yl)pyridin-2-yl)oxy)pyrrolidin-1-yl)methanone; (S)-(4-methoxyphenyl)(3-((5-(5-(trifluoromethyl)-1,2,4-oxadiazol-3-yl)pyridin-2-yl)oxy)pyrrolidin-1-yl)methanone; (S)-(3-fluorophenyl)(3-((5-(5-(trifluoromethyl)-1,2,4-oxadiazol-3-yl)pyridin-2-yl)oxy)pyrrolidin-1-yl)methanone; (S)-pyridin-3-yl(3-((5-(5-(trifluoromethyl)-1,2,4-oxadiazol-3-yl)pyridin-2-yl)oxy)pyrrolidin-1-yl)methanone; (S)-pyridin-4-yl(3-((5-(5-(trifluoromethyl)-1,2,4-oxadiazol-3-yl)pyridin-2-yl)oxy)pyrrolidin-1-yl)methanone; (S)-3-(6-((1-(ethylsulfonyl)pyrrolidin-3-yl)oxy)pyridin-3-yl)-5-(trifluoromethyl)-1,2,4-oxadiazole; (S)-1,1-dimethyl-3-((5-(5-(trifluoromethyl)-1,2,4-oxadiazol-3-yl)pyridin-2-yl)oxy)pyrrolidin-1-ium 2,2,2-trifluoroacetate; (S)-(3-(2-fluoro-4-(5-(trifluoromethyl)-1,2,4-oxadiazol-3-yl)phenoxy)pyrrolidin-1-yl)(2-fluorophenyl)methanone; (S)-(3-(2-fluoro-4-(5-(trifluoromethyl)-1,2,4-oxadiazol-3-yl)phenoxy)pyrrolidin-1-yl)(4-methoxyphenyl)methanone; (S)-(3-(2-fluoro-4-(5-(trifluoromethyl)-1,2,4-oxadiazol-3-yl)phenoxy)pyrrolidin-1-yl)(3-fluorophenyl)methanone; (S)-2-(pyridin-2-yl)-1-(3-(4-(5-(trifluoromethyl)-1,2,4-oxadiazol-3-yl)phenoxy)pyrrolidin-1-yl)ethan-1-one; (S)-(6-methoxypyridin-3-yl)(3-(4-(5-(trifluoromethyl)-1,2,4-oxadiazol-3-yl)phenoxy)pyrrolidin-1-yl)methanone; (S)-pyrimidin-5-yl(3-(4-(5-(trifluoromethyl)-1,2,4-oxadiazol-3-yl)phenoxy)pyrrolidin-1-yl)methanone; (S)-(3-(2-fluoro-4-(5-(trifluoromethyl)-1,2,4-oxadiazol-3-yl)phenoxy)pyrrolidin-1-yl)(pyridin-3-yl)methanone; (S)-(3-(2-fluoro-4-(5-(trifluoromethyl)-1,2,4-oxadiazol-3-yl)phenoxy)pyrrolidin-1-yl)(pyridin-4-yl)methanone; tert-butyl (S)-3-(2-fluoro-4-(5-(trifluoromethyl)-1,2,4-oxadiazol-3-yl)phenoxy)pyrrolidine-1-carboxylate; (S)-3-(4-((1-(ethylsulfonyl)pyrrolidin-3-yl)oxy)-3-fluorophenyl)-5-(trifluoromethyl)-1,2,4-oxadiazole; tert-butyl (S)-3-(3-fluoro-4-(5-(trifluoromethyl)-1,2,4-oxadiazol-3-yl)phenoxy)pyrrolidine-1-carboxylate; (S)-(3-(3-fluoro-4-(5-(trifluoromethyl)-1,2,4-oxadiazol-3-yl)phenoxy)pyrrolidin-1-yl)(2-fluorophenyl)methanone; (S)-(3-(3-fluoro-4-(5-(trifluoromethyl)-1,2,4-oxadiazol-3-yl)phenoxy)pyrrolidin-1-yl)(3-fluorophenyl)methanone; (S)-(3-(3-fluoro-4-(5-(trifluoromethyl)-1,2,4-oxadiazol-3-yl)phenoxy)pyrrolidin-1-yl)(pyridin-3-yl)methanone; (S)-(3-(3-fluoro-4-(5-(trifluoromethyl)-1,2,4-oxadiazol-3-yl)phenoxy)pyrrolidin-1-yl)(pyridin-4-yl)methanone; (S)-3-(4-((1-(ethylsulfonyl)pyrrolidin-3-yl)oxy)-2-fluorophenyl)-5-(trifluoromethyl)-1,2,4-oxadiazole; (S)-1-(3-(3-fluoro-4-(5-(trifluoromethyl)-1,2,4-oxadiazol-3-yl)phenoxy)pyrrolidin-1-yl)ethan-1-one; (S)-1-(3-(2-fluoro-4-(5-(trifluoromethyl)-1,2,4-oxadiazol-3-yl)phenoxy)pyrrolidin-1-yl)ethan-1-one; tert-butyl (R)-3-((5-(5-(trifluoromethyl)-1,2,4-oxadiazol-3-yl)pyridin-2-yl)oxy)pyrrolidine-1-carboxylate; (R)-3-(6-((1-(phenylsulfonyl)pyrrolidin-3-yl)oxy)pyridin-3-yl)-5-(trifluoromethyl)-1,2,4-oxadiazole; (R)-3-(6-((1-(ethylsulfonyl)pyrrolidin-3-yl)oxy)pyridin-3-yl)-5-(trifluoromethyl)-1,2,4-oxadiazole; (R)-3-(6-((1-((4-fluorophenyl)sulfonyl)pyrrolidin-3-yl)oxy)pyridin-3-yl)-5-(trifluoromethyl)-1,2,4-oxadiazole; (R)-(2-fluorophenyl)(3-((5-(5-(trifluoromethyl)-1,2,4-oxadiazol-3-yl)pyridin-2-yl)oxy)pyrrolidin-1-yl)methanone; (R)-(4-methoxyphenyl)(3-((5-(5-(trifluoromethyl)-1,2,4-oxadiazol-3-yl)pyridin-2-yl)oxy)pyrrolidin-1-yl)methanone; (R)-3-(6-((1-(cyclopropylsulfonyl)pyrrolidin-3-yl)oxy)pyridin-3-yl)-5-(trifluoromethyl)-1,2,4-oxadiazole; (R)-(3-fluorophenyl)(3-((5-(5-(trifluoromethyl)-1,2,4-oxadiazol-3-yl)pyridin-2-yl)oxy)pyrrolidin-1-yl)methanone; (R)-pyridin-4-yl(3-((5-(5-(trifluoromethyl)-1,2,4-oxadiazol-3-yl)pyridin-2-yl)oxy)pyrrolidin-1-yl)methanone; (R)-pyridin-3-yl(3-((5-(5-(trifluoromethyl)-1,2,4-oxadiazol-3-yl)pyridin-2-yl)oxy)pyrrolidin-1-yl)methanone; tert-butyl 3-(methyl(4-(5-(trifluoromethyl)-1,2,4-oxadiazol-3-yl)phenyl)amino)azetidine-1-carboxylate; phenyl(4-(4-(5-(trifluoromethyl)-1,2,4-oxadiazol-3-yl)phenoxy)piperidin-1-yl)methanone; tert-butyl 4-(4-(5-(trifluoromethyl)-1,2,4-oxadiazol-3-yl)phenoxy)piperidine-1-carboxylate; (3-(methyl(4-(5-(trifluoromethyl)-1,2,4-oxadiazol-3-yl)phenyl)amino)azetidin-1-yl)(phenyl)methanone; (3-chlorophenyl)(3-(methyl(4-(5-(trifluoromethyl)-1,2,4-oxadiazol-3-yl)phenyl)amino)azetidin-1-yl)methanone; 1-(3-(methyl(4-(5-(trifluoromethyl)-1,2,4-oxadiazol-3-yl)phenyl)amino)azetidin-1-yl)ethan-1-one; (2-fluorophenyl)(4-(4-(5-(trifluoromethyl)-1,2,4-oxadiazol-3-yl)phenoxy)piperidin-1-yl)methanone; tert-butyl 3-((4-(5-(trifluoromethyl)-1,2,4-oxadiazol-3-yl)phenyl)thio)pyrrolidine-1-carboxylate; 1-(4-(4-(5-(trifluoromethyl)-1,2,4-oxadiazol-3-yl)phenoxy)piperidin-1-yl)ethan-1-one; (3-fluorophenyl)(3-(methyl(4-(5-(trifluoromethyl)-1,2,4-oxadiazol-3-yl)phenyl)amino)azetidin-1-yl)methanone; (3-(methyl(4-(5-(trifluoromethyl)-1,2,4-oxadiazol-3-yl)phenyl)amino)azetidin-1-yl)(p-tolyl)methanone; (4-methoxyphenyl)(3-(methyl(4-(5-(trifluoromethyl)-1,2,4-oxadiazol-3-yl)phenyl)amino)azetidin-1-yl)methanone; 1-(3-(methyl(4-(5-(trifluoromethyl)-1,2,4-oxadiazol-3-yl)phenyl)amino)azetidin-1-yl)-2-phenylethan-1-one; 1-(3-(methyl(4-(5-(trifluoromethyl)-1,2,4-oxadiazol-3-yl)phenyl)amino)azetidin-1-yl)propan-1-one; (3-(methyl(4-(5-(trifluoromethyl)-1,2,4-oxadiazol-3-yl)phenyl)amino)azetidin-1-yl)(4-(trifluoromethyl)phenyl)methanone; (4-(4-(5-(trifluoromethyl)-1,2,4-oxadiazol-3-yl)phenoxy)piperidin-1-yl)(4-(trifluoromethyl)phenyl)methanone; p-tolyl(4-(4-(5-(trifluoromethyl)-1,2,4-oxadiazol-3-yl)phenoxy)piperidin-1-yl)methanone; (S)-3-(6-((1-(phenylsulfonyl)pyrrolidin-3-yl)oxy)pyridin-3-yl)-5-(trifluoromethyl)-1,2,4-oxadiazole; (S)-3-(6-((1-((4-methoxyphenyl)sulfonyl)pyrrolidin-3-yl)oxy)pyridin-3-yl)-5-(trifluoromethyl)-1,2,4-oxadiazole; (S)-3-(6-((1-(cyclopropylsulfonyl)pyrrolidin-3-yl)oxy)pyridin-3-yl)-5-(trifluoromethyl)-1,2,4-oxadiazole; (S)-3-(6-((1-tosylpyrrolidin-3-yl)oxy)pyridin-3-yl)-5-(trifluoromethyl)-1,2,4-oxadiazole; (S)-3-(6-((1-((3-chlorophenyl)sulfonyl)pyrrolidin-3-yl)oxy)pyridin-3-yl)-5-(trifluoromethyl)-1,2,4-oxadiazole; (S)-5-(trifluoromethyl)-3-(6-((1-((4-(trifluoromethyl)phenyl)sulfonyl)pyrrolidin-3-yl)oxy)pyridin-3-yl)-1,2,4-oxadiazole; (S)-3-(6-((1-(propylsulfonyl)pyrrolidin-3-yl)oxy)pyridin-3-yl)-5-(trifluoromethyl)-1,2,4-oxadiazole; (S)-3-(6-((1-(methylsulfonyl)pyrrolidin-3-yl)oxy)pyridin-3-yl)-5-(trifluoromethyl)-1,2,4-oxadiazole; (S)-3-(6-((1-(m-tolylsulfonyl)pyrrolidin-3-yl)oxy)pyridin-3-yl)-5-(trifluoromethyl)-1,2,4-oxadiazole; (S)-5-(trifluoromethyl)-3-(4-((1-((trifluoromethyl)sulfonyl)pyrrolidin-3-yl)oxy)phenyl)-1,2,4-oxadiazole; (S)-3-(4-((1-(propylsulfonyl)pyrrolidin-3-yl)oxy)phenyl)-5-(trifluoromethyl)-1,2,4-oxadiazole; (S)-3-(4-((1-((4-bromophenyl)sulfonyl)pyrrolidin-3-yl)oxy)phenyl)-5-(trifluoromethyl)-1,2,4-oxadiazole; (S)-3-(4-((1-(pyridin-3-ylsulfonyl)pyrrolidin-3-yl)oxy)phenyl)-5-(trifluoromethyl)-1,2,4-oxadiazole; (S)-5-(trifluoromethyl)-3-(4-((1-((4-(trifluoromethyl)phenyl)sulfonyl)pyrrolidin-3-yl)oxy)phenyl)-1,2,4-oxadiazole; (S)-3-(4-((1-tosylpyrrolidin-3-yl)oxy)phenyl)-5-(trifluoromethyl)-1,2,4-oxadiazole; (S)-3-(4-((1-((2,4-dichlorophenyl)sulfonyl)pyrrolidin-3-yl)oxy)phenyl)-5-(trifluoromethyl)-1,2,4-oxadiazole; (S)-4-((3-(4-(5-(trifluoromethyl)-1,2,4-oxadiazol-3-yl)phenoxy)pyrrolidin-1-yl)sulfonyl)benzonitrile; (S)-3-(4-((1-((3-chlorophenyl)sulfonyl)pyrrolidin-3-yl)oxy)phenyl)-5-(trifluoromethyl)-1,2,4-oxadiazole; (4-(dimethylamino)phenyl)(3-(methyl(4-(5-(trifluoromethyl)-1,2,4-oxadiazol-3-yl)phenyl)amino)azetidin-1-yl)methanone; (4-fluorophenyl)(3-(methyl(4-(5-(trifluoromethyl)-1,2,4-oxadiazol-3-yl)phenyl)amino)azetidin-1-yl)methanone; (2-fluorophenyl)(3-(methyl(4-(5-(trifluoromethyl)-1,2,4-oxadiazol-3-yl)phenyl)amino)azetidin-1-yl)methanone; (3-(methyl(4-(5-(trifluoromethyl)-1,2,4-oxadiazol-3-yl)phenyl)amino)azetidin-1-yl)(m-tolyl)methanone; tert-butyl 3-(3-(5-(trifluoromethyl)-1,2,4-oxadiazol-3-yl)phenoxy)pyrrolidine-1-carboxylate; 2,2-dimethyl-1-(3-(methyl(4-(5-(trifluoromethyl)-1,2,4-oxadiazol-3-yl)phenyl)amino)azetidin-1-yl)propan-1-one; 2,2-dimethyl-1-(3-(3-(5-(trifluoromethyl)-1,2,4-oxadiazol-3-yl)phenoxy)pyrrolidin-1-yl)propan-1-one; (3-chlorophenyl)(4-(4-(5-(trifluoromethyl)-1,2,4-oxadiazol-3-yl)phenoxy)piperidin-1-yl)methanone; (2-fluorophenyl)(3-(3-(5-(trifluoromethyl)-1,2,4-oxadiazol-3-yl)phenoxy)pyrrolidin-1-yl)methanone; (3-fluorophenyl)(3-(3-(5-(trifluoromethyl)-1,2,4-oxadiazol-3-yl)phenoxy)pyrrolidin-1-yl)methanone; (4-fluorophenyl)(3-(3-(5-(trifluoromethyl)-1,2,4-oxadiazol-3-yl)phenoxy)pyrrolidin-1-yl)methanone; p-tolyl(3-(3-(5-(trifluoromethyl)-1,2,4-oxadiazol-3-yl)phenoxy)pyrrolidin-1-yl)methanone; (S)-3-(4-((1-(isopropylsulfonyl)pyrrolidin-3-yl)oxy)phenyl)-5-(trifluoromethyl)-1,2,4-oxadiazole; (S)-3-(4-((1-benzylpyrrolidin-3-yl)oxy)phenyl)-5-(trifluoromethyl)-1,2,4-oxadiazole; 2-phenyl-1-(4-(4-(5-(trifluoromethyl)-1,2,4-oxadiazol-3-yl)phenoxy)piperidin-1-yl)ethan-1-one; 2,2-dimethyl-1-(4-(4-(5-(trifluoromethyl)-1,2,4-oxadiazol-3-yl)phenoxy)piperidin-1-yl)propan-1-one; (4-methoxyphenyl)(4-(4-(5-(trifluoromethyl)-1,2,4-oxadiazol-3-yl)phenoxy)piperidin-1-yl)methanone; m-tolyl(3-(3-(5-(trifluoromethyl)-1,2,4-oxadiazol-3-yl)phenoxy)pyrrolidin-1-yl)methanone; (4-fluorophenyl)(4-(4-(5-(trifluoromethyl)-1,2,4-oxadiazol-3-yl)phenoxy)piperidin-1-yl)methanone; (3-(3-(5-(trifluoromethyl)-1,2,4-oxadiazol-3-yl)phenoxy)pyrrolidin-1-yl)(4-(trifluoromethyl)phenyl)methanone; (3-chlorophenyl)(3-(3-(5-(trifluoromethyl)-1,2,4-oxadiazol-3-yl)phenoxy)pyrrolidin-1-yl)methanone; 2-(4-chlorophenyl)-1-(4-(4-(5-(trifluoromethyl)-1,2,4-oxadiazol-3-yl)phenoxy)piperidin-1-yl)ethan-1-one; (4-methoxyphenyl)(3-(3-(5-(trifluoromethyl)-1,2,4-oxadiazol-3-yl)phenoxy)pyrrolidin-1-yl)methanone; (S)-3-(4-((1-((3-methylthiophen-2-yl)sulfonyl)pyrrolidin-3-yl)oxy)phenyl)-5-(trifluoromethyl)-1,2,4-oxadiazole; (S)-3-(4-((1-((1-methyl-1H-imidazol-4-yl)sulfonyl)pyrrolidin-3-yl)oxy)phenyl)-5-(trifluoromethyl)-1,2,4-oxadiazole; (S)-3-(6-((1-((3-fluorophenyl)sulfonyl)pyrrolidin-3-yl)oxy)pyridin-3-yl)-5-(trifluoromethyl)-1,2,4-oxadiazole; (S)-4-((3-((5-(5-(trifluoromethyl)-1,2,4-oxadiazol-3-yl)pyridin-2-yl)oxy)pyrrolidin-1-yl)sulfonyl)benzonitrile; (S)-5-(trifluoromethyl)-3-(6-((1-((3-(trifluoromethyl)phenyl)sulfonyl)pyrrolidin-3-yl)oxy)pyridin-3-yl)-1,2,4-oxadiazole; (S)-3-(6-((1-((2-fluorophenyl)sulfonyl)pyrrolidin-3-yl)oxy)pyridin-3-yl)-5-(trifluoromethyl)-1,2,4-oxadiazole; (S)-3-(6-((1-(pyridin-3-ylsulfonyl)pyrrolidin-3-yl)oxy)pyridin-3-yl)-5-(trifluoromethyl)-1,2,4-oxadiazole; (S)-3-(6-((1-(benzylsulfonyl)pyrrolidin-3-yl)oxy)pyridin-3-yl)-5-(trifluoromethyl)-1,2,4-oxadiazole; (S)-3-(6-((1-(isopropylsulfonyl)pyrrolidin-3-yl)oxy)pyridin-3-yl)-5-(trifluoromethyl)-1,2,4-oxadiazole; tert-butyl (R)-3-((4-(5-(trifluoromethyl)-1,2,4-oxadiazol-3-yl)phenyl)thio)pyrrolidine-1-carboxylate; m-tolyl(4-(4-(5-(trifluoromethyl)-1,2,4-oxadiazol-3-yl)phenoxy)piperidin-1-yl)methanone; (S)-3-(6-((1-((2-chlorophenyl)sulfonyl)pyrrolidin-3-yl)oxy)pyridin-3-yl)-5-(trifluoromethyl)-1,2,4-oxadiazole; (S)-3-(6-((1-((4-chlorobenzyl)sulfonyl)pyrrolidin-3-yl)oxy)pyridin-3-yl)-5-(trifluoromethyl)-1,2,4-oxadiazole; (S)-3-(6-((1-((2-methoxyethyl)sulfonyl)pyrrolidin-3-yl)oxy)pyridin-3-yl)-5-(trifluoromethyl)-1,2,4-oxadiazole; (S)-3-(4-((1-(4-methylbenzyl)pyrrolidin-3-yl)oxy)phenyl)-5-(trifluoromethyl)-1,2,4-oxadiazole; N-methyl-1-(phenylsulfonyl)-N-(4-(5-(trifluoromethyl)-1,2,4-oxadiazol-3-yl)phenyl)azetidin-3-amine; (R)-1-(3-((4-(5-(trifluoromethyl)-1,2,4-oxadiazol-3-yl)phenyl)thio)pyrrolidin-1-yl)ethan-1-one; tert-butyl 3-(methyl(4-(5-(trifluoromethyl)-1,2,4-oxadiazol-3-yl)phenyl)amino)pyrrolidine-1-carboxylate; N-methyl-1-tosyl-N-(4-(5-(trifluoromethyl)-1,2,4-oxadiazol-3-yl)phenyl)azetidin-3-amine; 1-((2-fluorophenyl)sulfonyl)-N-methyl-N-(4-(5-(trifluoromethyl)-1,2,4-oxadiazol-3-yl)phenyl)azetidin-3-amine; 1-((4-methoxyphenyl)sulfonyl)-N-methyl-N-(4-(5-(trifluoromethyl)-1,2,4-oxadiazol-3-yl)phenyl)azetidin-3-amine; N-methyl-N-(4-(5-(trifluoromethyl)-1,2,4-oxadiazol-3-yl)phenyl)-1-((3-(trifluoromethyl)phenyl)sulfonyl)azetidin-3-amine; (R)-m-tolyl(3-((4-(5-(trifluoromethyl)-1,2,4-oxadiazol-3-yl)phenyl)thio)pyrrolidin-1-yl)methanone; N-methyl-N-(4-(5-(trifluoromethyl)-1,2,4-oxadiazol-3-yl)phenyl)-1-((4-(trifluoromethyl)phenyl)sulfonyl)azetidin-3-amine; 1-((3-chlorophenyl)sulfonyl)-N-methyl-N-(4-(5-(trifluoromethyl)-1,2,4-oxadiazol-3-yl)phenyl)azetidin-3-amine; (S)-3-(6-((1-(phenethylsulfonyl)pyrrolidin-3-yl)oxy)pyridin-3-yl)-5-(trifluoromethyl)-1,2,4-oxadiazole; (R)-(4-methoxyphenyl)(3-((4-(5-(trifluoromethyl)-1,2,4-oxadiazol-3-yl)phenyl)thio)pyrrolidin-1-yl)methanone; (R)-phenyl(3-((4-(5-(trifluoromethyl)-1,2,4-oxadiazol-3-yl)phenyl)thio)pyrrolidin-1-yl)methanone; (R)-2-phenyl-1-(3-((4-(5-(trifluoromethyl)-1,2,4-oxadiazol-3-yl)phenyl)thio)pyrrolidin-1-yl)ethan-1-one; pyridin-4-yl(4-(4-(5-(trifluoromethyl)-1,2,4-oxadiazol-3-yl)phenoxy)piperidin-1-yl)methanone; (S)-3-(4-((1-(4-chlorobenzyl)pyrrolidin-3-yl)oxy)phenyl)-5-(trifluoromethyl)-1,2,4-oxadiazole; (S)-3-(4-((1-isopropylpyrrolidin-3-yl)oxy)phenyl)-5-(trifluoromethyl)-1,2,4-oxadiazole; (R)-2-phenyl-1-(3-((4-(5-(trifluoromethyl)-1,2,4-oxadiazol-3-yl)phenyl)sulfonyl)pyrrolidin-1-yl)ethan-1-one; (R)-(2-fluorophenyl)(3-((4-(5-(trifluoromethyl)-1,2,4-oxadiazol-3-yl)phenyl)thio)pyrrolidin-1-yl)methanone; (S)-1-(3-((5-(5-(trifluoromethyl)-1,2,4-oxadiazol-3-yl)pyridin-2-yl)oxy)pyrrolidin-1-yl)ethan-1-one; (S)-1-(3-((5-(5-(trifluoromethyl)-1,2,4-oxadiazol-3-yl)pyridin-2-yl)oxy)pyrrolidin-1-yl)propan-1-one; (R)-pyridin-4-yl(3-((4-(5-(trifluoromethyl)-1,2,4-oxadiazol-3-yl)phenyl)thio)pyrrolidin-1-yl)methanone; (R)-2-(4-chlorophenyl)-1-(3-((4-(5-(trifluoromethyl)-1,2,4-oxadiazol-3-yl)phenyl)thio)pyrrolidin-1-yl)ethan-1-one; 2-(4-methoxyphenyl)-1-(4-(4-(5-(trifluoromethyl)-1,2,4-oxadiazol-3-yl)phenoxy)piperidin-1-yl)ethan-1-one; (R)-2-(4-chlorophenyl)-1-(3-((4-(5-(trifluoromethyl)-1,2,4-oxadiazol-3-yl)phenyl)sulfonyl)pyrrolidin-1-yl)ethan-1-one; (R)-2-(4-methoxyphenyl)-1-(3-((4-(5-(trifluoromethyl)-1,2,4-oxadiazol-3-yl)phenyl)sulfonyl)pyrrolidin-1-yl)ethan-1-one; (S)-(1-methyl-1H-pyrazol-3-yl)(3-((5-(5-(trifluoromethyl)-1,2,4-oxadiazol-3-yl)pyridin-2-yl)oxy)pyrrolidin-1-yl)methanone; (S)-isoxazol-3-yl(3-((5-(5-(trifluoromethyl)-1,2,4-oxadiazol-3-yl)pyridin-2-yl)oxy)pyrrolidin-1-yl)methanone; (4-(dimethylamino)phenyl)(4-(4-(5-(trifluoromethyl)-1,2,4-oxadiazol-3-yl)phenoxy)piperidin-1-yl)methanone; N-methyl-1-(propylsulfonyl)-N-(4-(5-(trifluoromethyl)-1,2,4-oxadiazol-3-yl)phenyl)azetidin-3-amine; N-methyl-N-(4-(5-(trifluoromethyl)-1,2,4-oxadiazol-3-yl)phenyl)-1-((trifluoromethyl)sulfonyl)azetidin-3-amine; (R)-(4-(dimethylamino)phenyl)(3-((4-(5-(trifluoromethyl)-1,2,4-oxadiazol-3-yl)phenyl)thio)pyrrolidin-1-yl)methanone; 1-((3-methoxyphenyl)sulfonyl)-N-methyl-N-(4-(5-(trifluoromethyl)-1,2,4-oxadiazol-3-yl)phenyl)azetidin-3-amine; (S)-oxazol-4-yl(3-((5-(5-(trifluoromethyl)-1,2,4-oxadiazol-3-yl)pyridin-2-yl)oxy)pyrrolidin-1-yl)methanone; (S)-thiazol-4-yl(3-((5-(5-(trifluoromethyl)-1,2,4-oxadiazol-3-yl)pyridin-2-yl)oxy)pyrrolidin-1-yl)methanone; 3-(4-((1-(phenylsulfonyl)azetidin-3-yl)oxy)phenyl)-5-(trifluoromethyl)-1,2,4-oxadiazole; 3-(4-((1-(methylsulfonyl)azetidin-3-yl)oxy)phenyl)-5-(trifluoromethyl)-1,2,4-oxadiazole; 2-(4-methoxyphenyl)-1-(3-((4-(5-(trifluoromethyl)-1,2,4-oxadiazol-3-yl)phenyl)thio)pyrrolidin-1-yl)ethan-1-one; (R)-(3-((4-(5-(trifluoromethyl)-1,2,4-oxadiazol-3-yl)phenyl)thio)pyrrolidin-1-yl)(4-(trifluoromethyl)phenyl)methanone; (4-chlorophenyl)(4-(4-(5-(trifluoromethyl)-1,2,4-oxadiazol-3-yl)phenoxy)piperidin-1-yl)methanone; (R)-(3-((4-(5-(trifluoromethyl)-1,2,4-oxadiazol-3-yl)phenyl)sulfonyl)pyrrolidin-1-yl)(4-(trifluoromethyl)phenyl)methanone; (3-(methyl(4-(5-(trifluoromethyl)-1,2,4-oxadiazol-3-yl)phenyl)amino)pyrrolidin-1-yl)(phenyl)methanone; pyridin-2-yl(3-(3-(5-(trifluoromethyl)-1,2,4-oxadiazol-3-yl)phenoxy)pyrrolidin-1-yl)methanone; pyridin-4-yl(3-(3-(5-(trifluoromethyl)-1,2,4-oxadiazol-3-yl)phenoxy)pyrrolidin-1-yl)methanone; pyridin-3-yl(3-(3-(5-(trifluoromethyl)-1,2,4-oxadiazol-3-yl)phenoxy)pyrrolidin-1-yl)methanone; (S)-2-methyl-1-(3-((5-(5-(trifluoromethyl)-1,2,4-oxadiazol-3-yl)pyridin-2-yl)oxy)pyrrolidin-1-yl)propan-1-one; (S)-cyclopropyl(3-((5-(5-(trifluoromethyl)-1,2,4-oxadiazol-3-yl)pyridin-2-yl)oxy)pyrrolidin-1-yl)methanone; (4-chloro-3-(trifluoromethyl)phenyl)(4-(4-(5-(trifluoromethyl)-1,2,4-oxadiazol-3-yl)phenoxy)piperidin-1-yl)methanone; (R)-(3-methoxyphenyl)(3-(4-(5-(trifluoromethyl)-1,2,4-oxadiazol-3-yl)phenoxy)pyrrolidin-1-yl)methanone; (R)-1-(3-(4-(5-(trifluoromethyl)-1,2,4-oxadiazol-3-yl)phenoxy)pyrrolidin-1-yl)ethan-1-one; (R)-(3-bromophenyl)(3-(4-(5-(trifluoromethyl)-1,2,4-oxadiazol-3-yl)phenoxy)pyrrolidin-1-yl)methanone; (R)-4-(3-(4-(5-(trifluoromethyl)-1,2,4-oxadiazol-3-yl)phenoxy)pyrrolidine-1-carbonyl)benzonitrile; (R)-2-(3,4-dimethoxyphenyl)-1-(3-(4-(5-(trifluoromethyl)-1,2,4-oxadiazol-3-yl)phenoxy)pyrrolidin-1-yl)ethan-1-one; (R)-(2-fluorophenyl)(3-(4-(5-(trifluoromethyl)-1,2,4-oxadiazol-3-yl)phenoxy)pyrrolidin-1-yl)methanone; (R)-pyridin-2-yl(3-(4-(5-(trifluoromethyl)-1,2,4-oxadiazol-3-yl)phenoxy)pyrrolidin-1-yl)methanone; (R)-(4-(dimethylamino)phenyl)(3-(4-(5-(trifluoromethyl)-1,2,4-oxadiazol-3-yl)phenoxy)pyrrolidin-1-yl)methanone; (R)-cyclobutyl(3-(4-(5-(trifluoromethyl)-1,2,4-oxadiazol-3-yl)phenoxy)pyrrolidin-1-yl)methanone; (R)-(4-methoxyphenyl)(3-(4-(5-(trifluoromethyl)-1,2,4-oxadiazol-3-yl)phenoxy)pyrrolidin-1-yl)methanone; (R)-2-phenyl-1-(3-(4-(5-(trifluoromethyl)-1,2,4-oxadiazol-3-yl)phenoxy)pyrrolidin-1-yl)ethan-1-one; (R)-pyridin-3-yl(3-(4-(5-(trifluoromethyl)-1,2,4-oxadiazol-3-yl)phenoxy)pyrrolidin-1-yl)methanone; (R)-pyridin-4-yl(3-(4-(5-(trifluoromethyl)-1,2,4-oxadiazol-3-yl)phenoxy)pyrrolidin-1-yl)methanone; (R)-(4-fluorophenyl)(3-(4-(5-(trifluoromethyl)-1,2,4-oxadiazol-3-yl)phenoxy)pyrrolidin-1-yl)methanone; (R)-phenyl(3-(4-(5-(trifluoromethyl)-1,2,4-oxadiazol-3-yl)phenoxy)pyrrolidin-1-yl)methanone; tert-butyl (R)-3-(4-(5-(trifluoromethyl)-1,2,4-oxadiazol-3-yl)phenoxy)pyrrolidine-1-carboxylate; (R)-2-(3-methoxyphenyl)-1-(3-(4-(5-(trifluoromethyl)-1,2,4-oxadiazol-3-yl)phenoxy)pyrrolidin-1-yl)ethan-1-one; (R)-3-(4-((1-(phenylsulfonyl)pyrrolidin-3-yl)oxy)phenyl)-5-(trifluoromethyl)-1,2,4-oxadiazole; (R)-3-(4-((1-((3-fluorophenyl)sulfonyl)pyrrolidin-3-yl)oxy)phenyl)-5-(trifluoromethyl)-1,2,4-oxadiazole; tert-butyl (R)-3-((6-(5-(trifluoromethyl)-1,2,4-oxadiazol-3-yl)pyridin-3-yl)oxy)pyrrolidine-1-carboxylate; (R)-(2-fluorophenyl)(3-((6-(5-(trifluoromethyl)-1,2,4-oxadiazol-3-yl)pyridin-3-yl)oxy)pyrrolidin-1-yl)methanone; (R)-(4-methoxyphenyl)(3-((6-(5-(trifluoromethyl)-1,2,4-oxadiazol-3-yl)pyridin-3-yl)oxy)pyrrolidin-1-yl)methanone; (R)-(3-fluorophenyl)(3-((6-(5-(trifluoromethyl)-1,2,4-oxadiazol-3-yl)pyridin-3-yl)oxy)pyrrolidin-1-yl)methanone; (R)-pyridin-3-yl(3-((6-(5-(trifluoromethyl)-1,2,4-oxadiazol-3-yl)pyridin-3-yl)oxy)pyrrolidin-1-yl)methanone; (R)-pyridin-4-yl(3-((6-(5-(trifluoromethyl)-1,2,4-oxadiazol-3-yl)pyridin-3-yl)oxy)pyrrolidin-1-yl)methanone; (R)-3-(4-((1-((2-fluorophenyl)sulfonyl)pyrrolidin-3-yl)oxy)phenyl)-5-(trifluoromethyl)-1,2,4-oxadiazole; (R)-3-(4-((1-((4-methoxyphenyl)sulfonyl)pyrrolidin-3-yl)oxy)phenyl)-5-(trifluoromethyl)-1,2,4-oxadiazole; (R)-3-(4-((1-((4-fluorophenyl)sulfonyl)pyrrolidin-3-yl)oxy)phenyl)-5-(trifluoromethyl)-1,2,4-oxadiazole; (R)-(4-(trifluoromethoxy)phenyl)(3-(4-(5-(trifluoromethyl)-1,2,4-oxadiazol-3-yl)phenoxy)pyrrolidin-1-yl)methanone; (R)—N-(2-fluorophenyl)-3-(4-(5-(trifluoromethyl)-1,2,4-oxadiazol-3-yl)phenoxy)pyrrolidine-1-carboxamide; (R)—N-(4-fluorophenyl)-3-(4-(5-(trifluoromethyl)-1,2,4-oxadiazol-3-yl)phenoxy)pyrrolidine-1-carboxamide; (R)—N-(4-methoxyphenyl)-3-(4-(5-(trifluoromethyl)-1,2,4-oxadiazol-3-yl)phenoxy)pyrrolidine-1-carboxamide; (R)-3-(4-((1-(ethylsulfonyl)pyrrolidin-3-yl)oxy)phenyl)-5-(trifluoromethyl)-1,2,4-oxadiazole; (R)-3-(4-((1-(cyclopropylsulfonyl)pyrrolidin-3-yl)oxy)phenyl)-5-(trifluoromethyl)-1,2,4-oxadiazole; (R)-3-(4-((1-((2,4-difluorophenyl)sulfonyl)pyrrolidin-3-yl)oxy)phenyl)-5-(trifluoromethyl)-1,2,4-oxadiazole; (R)-cyclopropyl(3-(4-(5-(trifluoromethyl)-1,2,4-oxadiazol-3-yl)phenoxy)pyrrolidin-1-yl)methanone; (R)-(4-chlorophenyl)(3-(4-(5-(trifluoromethyl)-1,2,4-oxadiazol-3-yl)phenoxy)pyrrolidin-1-yl)methanone; (R)-(2-fluorophenyl)(3-((5-(5-(trifluoromethyl)-1,2,4-oxadiazol-3-yl)pyridin-2-yl)oxy)pyrrolidin-1-yl)methanone; (R)-(4-methoxyphenyl)(3-((5-(5-(trifluoromethyl)-1,2,4-oxadiazol-3-yl)pyridin-2-yl)oxy)pyrrolidin-1-yl)methanone; (R)-(3-fluorophenyl)(3-((5-(5-(trifluoromethyl)-1,2,4-oxadiazol-3-yl)pyridin-2-yl)oxy)pyrrolidin-1-yl)methanone; (R)-pyridin-3-yl(3-((5-(5-(trifluoromethyl)-1,2,4-oxadiazol-3-yl)pyridin-2-yl)oxy)pyrrolidin-1-yl)methanone; (R)-pyridin-4-yl(3-((5-(5-(trifluoromethyl)-1,2,4-oxadiazol-3-yl)pyridin-2-yl)oxy)pyrrolidin-1-yl)methanone; (R)-3-(6-((1-(ethylsulfonyl)pyrrolidin-3-yl)oxy)pyridin-3-yl)-5-(trifluoromethyl)-1,2,4-oxadiazole; (R)-1,1-dimethyl-3-((5-(5-(trifluoromethyl)-1,2,4-oxadiazol-3-yl)pyridin-2-yl)oxy)pyrrolidin-1-ium 2,2,2-trifluoroacetate; (R)-(3-(2-fluoro-4-(5-(trifluoromethyl)-1,2,4-oxadiazol-3-yl)phenoxy)pyrrolidin-1-yl)(2-fluorophenyl)methanone; (R)-(3-(2-fluoro-4-(5-(trifluoromethyl)-1,2,4-oxadiazol-3-yl)phenoxy)pyrrolidin-1-yl)(4-methoxyphenyl)methanone; (R)-(3-(2-fluoro-4-(5-(trifluoromethyl)-1,2,4-oxadiazol-3-yl)phenoxy)pyrrolidin-1-yl)(3-fluorophenyl)methanone; (R)-2-(pyridin-2-yl)-1-(3-(4-(5-(trifluoromethyl)-1,2,4-oxadiazol-3-yl)phenoxy)pyrrolidin-1-yl)ethan-1-one; (R)-(6-methoxypyridin-3-yl)(3-(4-(5-(trifluoromethyl)-1,2,4-oxadiazol-3-yl)phenoxy)pyrrolidin-1-yl)methanone; (R)-pyrimidin-5-yl(3-(4-(5-(trifluoromethyl)-1,2,4-oxadiazol-3-yl)phenoxy)pyrrolidin-1-yl)methanone; (R)-(3-(2-fluoro-4-(5-(trifluoromethyl)-1,2,4-oxadiazol-3-yl)phenoxy)pyrrolidin-1-yl)(pyridin-3-yl)methanone; (R)-(3-(2-fluoro-4-(5-(trifluoromethyl)-1,2,4-oxadiazol-3-yl)phenoxy)pyrrolidin-1-yl)(pyridin-4-yl)methanone; tert-butyl (R)-3-(2-fluoro-4-(5-(trifluoromethyl)-1,2,4-oxadiazol-3-yl)phenoxy)pyrrolidine-1-carboxylate; (R)-3-(4-((1-(ethylsulfonyl)pyrrolidin-3-yl)oxy)-3-fluorophenyl)-5-(trifluoromethyl)-1,2,4-oxadiazole; tert-butyl (R)-3-(3-fluoro-4-(5-(trifluoromethyl)-1,2,4-oxadiazol-3-yl)phenoxy)pyrrolidine-1-carboxylate; (R)-(3-(3-fluoro-4-(5-(trifluoromethyl)-1,2,4-oxadiazol-3-yl)phenoxy)pyrrolidin-1-yl)(2-fluorophenyl)methanone; (R)-(3-(3-fluoro-4-(5-(trifluoromethyl)-1,2,4-oxadiazol-3-yl)phenoxy)pyrrolidin-1-yl)(3-fluorophenyl)methanone; (R)-(3-(3-fluoro-4-(5-(trifluoromethyl)-1,2,4-oxadiazol-3-yl)phenoxy)pyrrolidin-1-yl)(pyridin-3-yl)methanone; (R)-(3-(3-fluoro-4-(5-(trifluoromethyl)-1,2,4-oxadiazol-3-yl)phenoxy)pyrrolidin-1-yl)(pyridin-4-yl)methanone; (R)-3-(4-((1-(ethylsulfonyl)pyrrolidin-3-yl)oxy)-2-fluorophenyl)-5-(trifluoromethyl)-1,2,4-oxadiazole; (R)-1-(3-(3-fluoro-4-(5-(trifluoromethyl)-1,2,4-oxadiazol-3-yl)phenoxy)pyrrolidin-1-yl)ethan-1-one; (R)-1-(3-(2-fluoro-4-(5-(trifluoromethyl)-1,2,4-oxadiazol-3-yl)phenoxy)pyrrolidin-1-yl)ethan-1-one; tert-butyl (S)-3-((5-(5-(trifluoromethyl)-1,2,4-oxadiazol-3-yl)pyridin-2-yl)oxy)pyrrolidine-1-carboxylate; (S)-3-(6-((1-(phenylsulfonyl)pyrrolidin-3-yl)oxy)pyridin-3-yl)-5-(trifluoromethyl)-1,2,4-oxadiazole; (S)-3-(6-((1-(ethylsulfonyl)pyrrolidin-3-yl)oxy)pyridin-3-yl)-5-(trifluoromethyl)-1,2,4-oxadiazole; (S)-3-(6-((1-((4-fluorophenyl)sulfonyl)pyrrolidin-3-yl)oxy)pyridin-3-yl)-5-(trifluoromethyl)-1,2,4-oxadiazole; (S)-(2-fluorophenyl)(3-((5-(5-(trifluoromethyl)-1,2,4-oxadiazol-3-yl)pyridin-2-yl)oxy)pyrrolidin-1-yl)methanone; (S)-(4-methoxyphenyl)(3-((5-(5-(trifluoromethyl)-1,2,4-oxadiazol-3-yl)pyridin-2-yl)oxy)pyrrolidin-1-yl)methanone; (S)-3-(6-((1-(cyclopropylsulfonyl)pyrrolidin-3-yl)oxy)pyridin-3-yl)-5-(trifluoromethyl)-1,2,4-oxadiazole; (S)-(3-fluorophenyl)(3-((5-(5-(trifluoromethyl)-1,2,4-oxadiazol-3-yl)pyridin-2-yl)oxy)pyrrolidin-1-yl)methanone; (S)-pyridin-4-yl(3-((5-(5-(trifluoromethyl)-1,2,4-oxadiazol-3-yl)pyridin-2-yl)oxy)pyrrolidin-1-yl)methanone; (S)-pyridin-3-yl(3-((5-(5-(trifluoromethyl)-1,2,4-oxadiazol-3-yl)pyridin-2-yl)oxy)pyrrolidin-1-yl)methanone; (R)-3-(6-((1-(phenylsulfonyl)pyrrolidin-3-yl)oxy)pyridin-3-yl)-5-(trifluoromethyl)-1,2,4-oxadiazole; (R)-3-(6-((1-((4-methoxyphenyl)sulfonyl)pyrrolidin-3-yl)oxy)pyridin-3-yl)-5-(trifluoromethyl)-1,2,4-oxadiazole; (R)-3-(6-((1-(cyclopropylsulfonyl)pyrrolidin-3-yl)oxy)pyridin-3-yl)-5-(trifluoromethyl)-1,2,4-oxadiazole; (R)-3-(6-((1-tosylpyrrolidin-3-yl)oxy)pyridin-3-yl)-5-(trifluoromethyl)-1,2,4-oxadiazole; (R)-3-(6-((1-((3-chlorophenyl)sulfonyl)pyrrolidin-3-yl)oxy)pyridin-3-yl)-5-(trifluoromethyl)-1,2,4-oxadiazole; (R)-5-(trifluoromethyl)-3-(6-((1-((4-(trifluoromethyl)phenyl)sulfonyl)pyrrolidin-3-yl)oxy)pyridin-3-yl)-1,2,4-oxadiazole; (R)-3-(6-((1-(propylsulfonyl)pyrrolidin-3-yl)oxy)pyridin-3-yl)-5-(trifluoromethyl)-1,2,4-oxadiazole; (R)-3-(6-((1-(methylsulfonyl)pyrrolidin-3-yl)oxy)pyridin-3-yl)-5-(trifluoromethyl)-1,2,4-oxadiazole; (R)-3-(6-((1-(m-tolylsulfonyl)pyrrolidin-3-yl)oxy)pyridin-3-yl)-5-(trifluoromethyl)-1,2,4-oxadiazole; (R)-5-(trifluoromethyl)-3-(4-((1-((trifluoromethyl)sulfonyl)pyrrolidin-3-yl)oxy)phenyl)-1,2,4-oxadiazole; (R)-3-(4-((1-(propylsulfonyl)pyrrolidin-3-yl)oxy)phenyl)-5-(trifluoromethyl)-1,2,4-oxadiazole; (R)-3-(4-((1-((4-bromophenyl)sulfonyl)pyrrolidin-3-yl)oxy)phenyl)-5-(trifluoromethyl)-1,2,4-oxadiazole; (R)-3-(4-((1-(pyridin-3-ylsulfonyl)pyrrolidin-3-yl)oxy)phenyl)-5-(trifluoromethyl)-1,2,4-oxadiazole; (R)-5-(trifluoromethyl)-3-(4-((1-((4-(trifluoromethyl)phenyl)sulfonyl)pyrrolidin-3-yl)oxy)phenyl)-1,2,4-oxadiazole; (R)-3-(4-((1-tosylpyrrolidin-3-yl)oxy)phenyl)-5-(trifluoromethyl)-1,2,4-oxadiazole; (R)-3-(4-((1-((2,4-dichlorophenyl)sulfonyl)pyrrolidin-3-yl)oxy)phenyl)-5-(trifluoromethyl)-1,2,4-oxadiazole; (R)-4-((3-(4-(5-(trifluoromethyl)-1,2,4-oxadiazol-3-yl)phenoxy)pyrrolidin-1-yl)sulfonyl)benzonitrile; (R)-3-(4-((1-((3-chlorophenyl)sulfonyl)pyrrolidin-3-yl)oxy)phenyl)-5-(trifluoromethyl)-1,2,4-oxadiazole; (R)-3-(4-((1-(isopropylsulfonyl)pyrrolidin-3-yl)oxy)phenyl)-5-(trifluoromethyl)-1,2,4-oxadiazole; (R)-3-(4-((1-benzylpyrrolidin-3-yl)oxy)phenyl)-5-(trifluoromethyl)-1,2,4-oxadiazole; (R)-3-(4-((1-((3-methylthiophen-2-yl)sulfonyl)pyrrolidin-3-yl)oxy)phenyl)-5-(trifluoromethyl)-1,2,4-oxadiazole; (R)-3-(4-((1-((1-methyl-1H-imidazol-4-yl)sulfonyl)pyrrolidin-3-yl)oxy)phenyl)-5-(trifluoromethyl)-1,2,4-oxadiazole; (R)-3-(6-((1-((3-fluorophenyl)sulfonyl)pyrrolidin-3-yl)oxy)pyridin-3-yl)-5-(trifluoromethyl)-1,2,4-oxadiazole; (R)-4-((3-((5-(5-(trifluoromethyl)-1,2,4-oxadiazol-3-yl)pyridin-2-yl)oxy)pyrrolidin-1-yl)sulfonyl)benzonitrile; (R)-5-(trifluoromethyl)-3-(6-((1-((3-(trifluoromethyl)phenyl)sulfonyl)pyrrolidin-3-yl)oxy)pyridin-3-yl)-1,2,4-oxadiazole; (R)-3-(6-((1-((2-fluorophenyl)sulfonyl)pyrrolidin-3-yl)oxy)pyridin-3-yl)-5-(trifluoromethyl)-1,2,4-oxadiazole; (R)-3-(6-((1-(pyridin-3-ylsulfonyl)pyrrolidin-3-yl)oxy)pyridin-3-yl)-5-(trifluoromethyl)-1,2,4-oxadiazole; (R)-3-(6-((1-(benzylsulfonyl)pyrrolidin-3-yl)oxy)pyridin-3-yl)-5-(trifluoromethyl)-1,2,4-oxadiazole; (R)-3-(6-((1-(isopropylsulfonyl)pyrrolidin-3-yl)oxy)pyridin-3-yl)-5-(trifluoromethyl)-1,2,4-oxadiazole; tert-butyl (S)-3-((4-(5-(trifluoromethyl)-1,2,4-oxadiazol-3-yl)phenyl)thio)pyrrolidine-1-carboxylate; (R)-3-(6-((1-((2-chlorophenyl)sulfonyl)pyrrolidin-3-yl)oxy)pyridin-3-yl)-5-(trifluoromethyl)-1,2,4-oxadiazole; (R)-3-(6-((1-((4-chlorobenzyl)sulfonyl)pyrrolidin-3-yl)oxy)pyridin-3-yl)-5-(trifluoromethyl)-1,2,4-oxadiazole; (R)-3-(6-((1-((2-methoxyethyl)sulfonyl)pyrrolidin-3-yl)oxy)pyridin-3-yl)-5-(trifluoromethyl)-1,2,4-oxadiazole; (R)-3-(4-((1-(4-methylbenzyl)pyrrolidin-3-yl)oxy)phenyl)-5-(trifluoromethyl)-1,2,4-oxadiazole; (S)-1-(3-((4-(5-(trifluoromethyl)-1,2,4-oxadiazol-3-yl)phenyl)thio)pyrrolidin-1-yl)ethan-1-one; (S)-m-tolyl(3-((4-(5-(trifluoromethyl)-1,2,4-oxadiazol-3-yl)phenyl)thio)pyrrolidin-1-yl)methanone; (R)-3-(6-((1-(phenethylsulfonyl)pyrrolidin-3-yl)oxy)pyridin-3-yl)-5-(trifluoromethyl)-1,2,4-oxadiazole; (S)-(4-methoxyphenyl)(3-((4-(5-(trifluoromethyl)-1,2,4-oxadiazol-3-yl)phenyl)thio)pyrrolidin-1-yl)methanone; (S)-phenyl(3-((4-(5-(trifluoromethyl)-1,2,4-oxadiazol-3-yl)phenyl)thio)pyrrolidin-1-yl)methanone; (S)-2-phenyl-1-(3-((4-(5-(trifluoromethyl)-1,2,4-oxadiazol-3-yl)phenyl)thio)pyrrolidin-1-yl)ethan-1-one; (R)-3-(4-((1-(4-chlorobenzyl)pyrrolidin-3-yl)oxy)phenyl)-5-(trifluoromethyl)-1,2,4-oxadiazole; (R)-3-(4-((1-isopropylpyrrolidin-3-yl)oxy)phenyl)-5-(trifluoromethyl)-1,2,4-oxadiazole; (S)-2-phenyl-1-(3-((4-(5-(trifluoromethyl)-1,2,4-oxadiazol-3-yl)phenyl)sulfonyl)pyrrolidin-1-yl)ethan-1-one; (S)-(2-fluorophenyl)(3-((4-(5-(trifluoromethyl)-1,2,4-oxadiazol-3-yl)phenyl)thio)pyrrolidin-1-yl)methanone; (R)-1-(3-((5-(5-(trifluoromethyl)-1,2,4-oxadiazol-3-yl)pyridin-2-yl)oxy)pyrrolidin-1-yl)ethan-1-one; (R)-1-(3-((5-(5-(trifluoromethyl)-1,2,4-oxadiazol-3-yl)pyridin-2-yl)oxy)pyrrolidin-1-yl)propan-1-one; (S)-pyridin-4-yl(3-((4-(5-(trifluoromethyl)-1,2,4-oxadiazol-3-yl)phenyl)thio)pyrrolidin-1-yl)methanone; (S)-2-(4-chlorophenyl)-1-(3-((4-(5-(trifluoromethyl)-1,2,4-oxadiazol-3-yl)phenyl)thio)pyrrolidin-1-yl)ethan-1-one; (S)-2-(4-chlorophenyl)-1-(3-((4-(5-(trifluoromethyl)-1,2,4-oxadiazol-3-yl)phenyl)sulfonyl)pyrrolidin-1-yl)ethan-1-one; (S)-2-(4-methoxyphenyl)-1-(3-((4-(5-(trifluoromethyl)-1,2,4-oxadiazol-3-yl)phenyl)sulfonyl)pyrrolidin-1-yl)ethan-1-one; (R)-(1-methyl-1H-pyrazol-3-yl)(3-((5-(5-(trifluoromethyl)-1,2,4-oxadiazol-3-yl)pyridin-2-yl)oxy)pyrrolidin-1-yl)methanone; (R)-isoxazol-3-yl(3-((5-(5-(trifluoromethyl)-1,2,4-oxadiazol-3-yl)pyridin-2-yl)oxy)pyrrolidin-1-yl)methanone; (S)-(4-(dimethylamino)phenyl)(3-((4-(5-(trifluoromethyl)-1,2,4-oxadiazol-3-yl)phenyl)thio)pyrrolidin-1-yl)methanone; (R)-oxazol-4-yl(3-((5-(5-(trifluoromethyl)-1,2,4-oxadiazol-3-yl)pyridin-2-yl)oxy)pyrrolidin-1-yl)methanone; (R)-thiazol-4-yl(3-((5-(5-(trifluoromethyl)-1,2,4-oxadiazol-3-yl)pyridin-2-yl)oxy)pyrrolidin-1-yl)methanone; (S)-(3-((4-(5-(trifluoromethyl)-1,2,4-oxadiazol-3-yl)phenyl)thio)pyrrolidin-1-yl)(4-(trifluoromethyl)phenyl)methanone; (S)-(3-((4-(5-(trifluoromethyl)-1,2,4-oxadiazol-3-yl)phenyl)sulfonyl)pyrrolidin-1-yl)(4-(trifluoromethyl)phenyl)methanone; (R)-2-methyl-1-(3-((5-(5-(trifluoromethyl)-1,2,4-oxadiazol-3-yl)pyridin-2-yl)oxy)pyrrolidin-1-yl)propan-1-one; (R)-cyclopropyl(3-((5-(5-(trifluoromethyl)-1,2,4-oxadiazol-3-yl)pyridin-2-yl)oxy)pyrrolidin-1-yl)methanone; (3-methoxyphenyl)(3-(4-(5-(trifluoromethyl)-1,2,4-oxadiazol-3-yl)phenoxy)pyrrolidin-1-yl)methanone; 1-(3-(4-(5-(trifluoromethyl)-1,2,4-oxadiazol-3-yl)phenoxy)pyrrolidin-1-yl)ethan-1-one; (3-bromophenyl)(3-(4-(5-(trifluoromethyl)-1,2,4-oxadiazol-3-yl)phenoxy)pyrrolidin-1-yl)methanone; 4-(3-(4-(5-(trifluoromethyl)-1,2,4-oxadiazol-3-yl)phenoxy)pyrrolidine-1-carbonyl)benzonitrile; 2-(3,4-dimethoxyphenyl)-1-(3-(4-(5-(trifluoromethyl)-1,2,4-oxadiazol-3-yl)phenoxy)pyrrolidin-1-yl)ethan-1-one; (2-fluorophenyl)(3-(4-(5-(trifluoromethyl)-1,2,4-oxadiazol-3-yl)phenoxy)pyrrolidin-1-yl)methanone; pyridin-2-yl(3-(4-(5-(trifluoromethyl)-1,2,4-oxadiazol-3-yl)phenoxy)pyrrolidin-1-yl)methanone; (4-(dimethylamino)phenyl)(3-(4-(5-(trifluoromethyl)-1,2,4-oxadiazol-3-yl)phenoxy)pyrrolidin-1-yl)methanone; cyclobutyl(3-(4-(5-(trifluoromethyl)-1,2,4-oxadiazol-3-yl)phenoxy)pyrrolidin-1-yl)methanone; (4-methoxyphenyl)(3-(4-(5-(trifluoromethyl)-1,2,4-oxadiazol-3-yl)phenoxy)pyrrolidin-1-yl)methanone; 2-phenyl-1-(3-(4-(5-(trifluoromethyl)-1,2,4-oxadiazol-3-yl)phenoxy)pyrrolidin-1-yl)ethan-1-one; pyridin-3-yl(3-(4-(5-(trifluoromethyl)-1,2,4-oxadiazol-3-yl)phenoxy)pyrrolidin-1-yl)methanone; pyridin-4-yl(3-(4-(5-(trifluoromethyl)-1,2,4-oxadiazol-3-yl)phenoxy)pyrrolidin-1-yl)methanone; (4-fluorophenyl)(3-(4-(5-(trifluoromethyl)-1,2,4-oxadiazol-3-yl)phenoxy)pyrrolidin-1-yl)methanone; phenyl(3-(4-(5-(trifluoromethyl)-1,2,4-oxadiazol-3-yl)phenoxy)pyrrolidin-1-yl)methanone; tert-butyl-3-(4-(5-(trifluoromethyl)-1,2,4-oxadiazol-3-yl)phenoxy)pyrrolidine-1-carboxylate; 2-(3-methoxyphenyl)-1-(3-(4-(5-(trifluoromethyl)-1,2,4-oxadiazol-3-yl)phenoxy)pyrrolidin-1-yl)ethan-1-one; 3-(4-((1-(phenylsulfonyl)pyrrolidin-3-yl)oxy)phenyl)-5-(trifluoromethyl)-1,2,4-oxadiazole; 3-(4-((1-((3-fluorophenyl)sulfonyl)pyrrolidin-3-yl)oxy)phenyl)-5-(trifluoromethyl)-1,2,4-oxadiazole; tert-butyl-3-((6-(5-(trifluoromethyl)-1,2,4-oxadiazol-3-yl)pyridin-3-yl)oxy)pyrrolidine-1-carboxylate; (2-fluorophenyl)(3-((6-(5-(trifluoromethyl)-1,2,4-oxadiazol-3-yl)pyridin-3-yl)oxy)pyrrolidin-1-yl)methanone; (4-methoxyphenyl)(3-((6-(5-(trifluoromethyl)-1,2,4-oxadiazol-3-yl)pyridin-3-yl)oxy)pyrrolidin-1-yl)methanone; (3-fluorophenyl)(3-((6-(5-(trifluoromethyl)-1,2,4-oxadiazol-3-yl)pyridin-3-yl)oxy)pyrrolidin-1-yl)methanone; pyridin-3-yl(3-((6-(5-(trifluoromethyl)-1,2,4-oxadiazol-3-yl)pyridin-3-yl)oxy)pyrrolidin-1-yl)methanone; pyridin-4-yl(3-((6-(5-(trifluoromethyl)-1,2,4-oxadiazol-3-yl)pyridin-3-yl)oxy)pyrrolidin-1-yl)methanone; 3-(4-((1-((2-fluorophenyl)sulfonyl)pyrrolidin-3-yl)oxy)phenyl)-5-(trifluoromethyl)-1,2,4-oxadiazole; 3-(4-((1-((4-methoxyphenyl)sulfonyl)pyrrolidin-3-yl)oxy)phenyl)-5-(trifluoromethyl)-1,2,4-oxadiazole; 3-(4-((1-((4-fluorophenyl)sulfonyl)pyrrolidin-3-yl)oxy)phenyl)-5-(trifluoromethyl)-1,2,4-oxadiazole; (4-(trifluoromethoxy)phenyl)(3-(4-(5-(trifluoromethyl)-1,2,4-oxadiazol-3-yl)phenoxy)pyrrolidin-1-yl)methanone; N-(2-fluorophenyl)-3-(4-(5-(trifluoromethyl)-1,2,4-oxadiazol-3-yl)phenoxy)pyrrolidine-1-carboxamide; N-(4-fluorophenyl)-3-(4-(5-(trifluoromethyl)-1,2,4-oxadiazol-3-yl)phenoxy)pyrrolidine-1-carboxamide; N-(4-methoxyphenyl)-3-(4-(5-(trifluoromethyl)-1,2,4-oxadiazol-3-yl)phenoxy)pyrrolidine-1-carboxamide; 3-(4-((1-(ethylsulfonyl)pyrrolidin-3-yl)oxy)phenyl)-5-(trifluoromethyl)-1,2,4-oxadiazole; 3-(4-((1-(cyclopropylsulfonyl)pyrrolidin-3-yl)oxy)phenyl)-5-(trifluoromethyl)-1,2,4-oxadiazole; 3-(4-((1-((2,4-difluorophenyl)sulfonyl)pyrrolidin-3-yl)oxy)phenyl)-5-(trifluoromethyl)-1,2,4-oxadiazole; cyclopropyl(3-(4-(5-(trifluoromethyl)-1,2,4-oxadiazol-3-yl)phenoxy)pyrrolidin-1-yl)methanone; (4-chlorophenyl)(3-(4-(5-(trifluoromethyl)-1,2,4-oxadiazol-3-yl)phenoxy)pyrrolidin-1-yl)methanone; (2-fluorophenyl)(3-((5-(5-(trifluoromethyl)-1,2,4-oxadiazol-3-yl)pyridin-2-yl)oxy)pyrrolidin-1-yl)methanone; (4-methoxyphenyl)(3-((5-(5-(trifluoromethyl)-1,2,4-oxadiazol-3-yl)pyridin-2-yl)oxy)pyrrolidin-1-yl)methanone; (3-fluorophenyl)(3-((5-(5-(trifluoromethyl)-1,2,4-oxadiazol-3-yl)pyridin-2-yl)oxy)pyrrolidin-1-yl)methanone; pyridin-3-yl(3-((5-(5-(trifluoromethyl)-1,2,4-oxadiazol-3-yl)pyridin-2-yl)oxy)pyrrolidin-1-yl)methanone; pyridin-4-yl(3-((5-(5-(trifluoromethyl)-1,2,4-oxadiazol-3-yl)pyridin-2-yl)oxy)pyrrolidin-1-yl)methanone; 3-(6-((1-(ethylsulfonyl)pyrrolidin-3-yl)oxy)pyridin-3-yl)-5-(trifluoromethyl)-1,2,4-oxadiazole; 1,1-dimethyl-3-((5-(5-(trifluoromethyl)-1,2,4-oxadiazol-3-yl)pyridin-2-yl)oxy)pyrrolidin-1-ium 2,2,2-trifluoroacetate; (3-(2-fluoro-4-(5-(trifluoromethyl)-1,2,4-oxadiazol-3-yl)phenoxy)pyrrolidin-1-yl)(2-fluorophenyl)methanone; (3-(2-fluoro-4-(5-(trifluoromethyl)-1,2,4-oxadiazol-3-yl)phenoxy)pyrrolidin-1-yl)(4-methoxyphenyl)methanone; (3-(2-fluoro-4-(5-(trifluoromethyl)-1,2,4-oxadiazol-3-yl)phenoxy)pyrrolidin-1-yl)(3-fluorophenyl)methanone; 2-(pyridin-2-yl)-1-(3-(4-(5-(trifluoromethyl)-1,2,4-oxadiazol-3-yl)phenoxy)pyrrolidin-1-yl)ethan-1-one; (6-methoxypyridin-3-yl)(3-(4-(5-(trifluoromethyl)-1,2,4-oxadiazol-3-yl)phenoxy)pyrrolidin-1-yl)methanone; pyrimidin-5-yl(3-(4-(5-(trifluoromethyl)-1,2,4-oxadiazol-3-yl)phenoxy)pyrrolidin-1-yl)methanone; (3-(2-fluoro-4-(5-(trifluoromethyl)-1,2,4-oxadiazol-3-yl)phenoxy)pyrrolidin-1-yl)(pyridin-3-yl)methanone; (3-(2-fluoro-4-(5-(trifluoromethyl)-1,2,4-oxadiazol-3-yl)phenoxy)pyrrolidin-1-yl)(pyridin-4-yl)methanone; tert-butyl-3-(2-fluoro-4-(5-(trifluoromethyl)-1,2,4-oxadiazol-3-yl)phenoxy)pyrrolidine-1-carboxylate; 3-(4-((1-(ethylsulfonyl)pyrrolidin-3-yl)oxy)-3-fluorophenyl)-5-(trifluoromethyl)-1,2,4-oxadiazole; tert-butyl-3-(3-fluoro-4-(5-(trifluoromethyl)-1,2,4-oxadiazol-3-yl)phenoxy)pyrrolidine-1-carboxylate; (3-(3-fluoro-4-(5-(trifluoromethyl)-1,2,4-oxadiazol-3-yl)phenoxy)pyrrolidin-1-yl)(2-fluorophenyl)methanone; (3-(3-fluoro-4-(5-(trifluoromethyl)-1,2,4-oxadiazol-3-yl)phenoxy)pyrrolidin-1-yl)(3-fluorophenyl)methanone; (3-(3-fluoro-4-(5-(trifluoromethyl)-1,2,4-oxadiazol-3-yl)phenoxy)pyrrolidin-1-yl)(pyridin-3-yl)methanone; (3-(3-fluoro-4-(5-(trifluoromethyl)-1,2,4-oxadiazol-3-yl)phenoxy)pyrrolidin-1-yl)(pyridin-4-yl)methanone; 3-(4-((1-(ethylsulfonyl)pyrrolidin-3-yl)oxy)-2-fluorophenyl)-5-(trifluoromethyl)-1,2,4-oxadiazole; 1-(3-(3-fluoro-4-(5-(trifluoromethyl)-1,2,4-oxadiazol-3-yl)phenoxy)pyrrolidin-1-yl)ethan-1-one; 1-(3-(2-fluoro-4-(5-(trifluoromethyl)-1,2,4-oxadiazol-3-yl)phenoxy)pyrrolidin-1-yl)ethan-1-one; tert-butyl-3-((5-(5-(trifluoromethyl)-1,2,4-oxadiazol-3-yl)pyridin-2-yl)oxy)pyrrolidine-1-carboxylate; 3-(6-((1-(phenylsulfonyl)pyrrolidin-3-yl)oxy)pyridin-3-yl)-5-(trifluoromethyl)-1,2,4-oxadiazole; 3-(6-((1-(ethylsulfonyl)pyrrolidin-3-yl)oxy)pyridin-3-yl)-5-(trifluoromethyl)-1,2,4-oxadiazole; 3-(6-((1-((4-fluorophenyl)sulfonyl)pyrrolidin-3-yl)oxy)pyridin-3-yl)-5-(trifluoromethyl)-1,2,4-oxadiazole; (2-fluorophenyl)(3-((5-(5-(trifluoromethyl)-1,2,4-oxadiazol-3-yl)pyridin-2-yl)oxy)pyrrolidin-1-yl)methanone; (4-methoxyphenyl)(3-((5-(5-(trifluoromethyl)-1,2,4-oxadiazol-3-yl)pyridin-2-yl)oxy)pyrrolidin-1-yl)methanone; 3-(6-((1-(cyclopropylsulfonyl)pyrrolidin-3-yl)oxy)pyridin-3-yl)-5-(trifluoromethyl)-1,2,4-oxadiazole; (3-fluorophenyl)(3-((5-(5-(trifluoromethyl)-1,2,4-oxadiazol-3-yl)pyridin-2-yl)oxy)pyrrolidin-1-yl)methanone; pyridin-4-yl(3-((5-(5-(trifluoromethyl)-1,2,4-oxadiazol-3-yl)pyridin-2-yl)oxy)pyrrolidin-1-yl)methanone; pyridin-3-yl(3-((5-(5-(trifluoromethyl)-1,2,4-oxadiazol-3-yl)pyridin-2-yl)oxy)pyrrolidin-1-yl)methanone; 3-(6-((1-(phenylsulfonyl)pyrrolidin-3-yl)oxy)pyridin-3-yl)-5-(trifluoromethyl)-1,2,4-oxadiazole; 3-(6-((1-((4-methoxyphenyl)sulfonyl)pyrrolidin-3-yl)oxy)pyridin-3-yl)-5-(trifluoromethyl)-1,2,4-oxadiazole; 3-(6-((1-(cyclopropylsulfonyl)pyrrolidin-3-yl)oxy)pyridin-3-yl)-5-(trifluoromethyl)-1,2,4-oxadiazole; 3-(6-((1-tosylpyrrolidin-3-yl)oxy)pyridin-3-yl)-5-(trifluoromethyl)-1,2,4-oxadiazole; 3-(6-((1-((3-chlorophenyl)sulfonyl)pyrrolidin-3-yl)oxy)pyridin-3-yl)-5-(trifluoromethyl)-1,2,4-oxadiazole; 5-(trifluoromethyl)-3-(6-((1-((4-(trifluoromethyl)phenyl)sulfonyl)pyrrolidin-3-yl)oxy)pyridin-3-yl)-1,2,4-oxadiazole; 3-(6-((1-(propylsulfonyl)pyrrolidin-3-yl)oxy)pyridin-3-yl)-5-(trifluoromethyl)-1,2,4-oxadiazole; 3-(6-((1-(methylsulfonyl)pyrrolidin-3-yl)oxy)pyridin-3-yl)-5-(trifluoromethyl)-1,2,4-oxadiazole; 3-(6-((1-(m-tolylsulfonyl)pyrrolidin-3-yl)oxy)pyridin-3-yl)-5-(trifluoromethyl)-1,2,4-oxadiazole; 5-(trifluoromethyl)-3-(4-((1-((trifluoromethyl)sulfonyl)pyrrolidin-3-yl)oxy)phenyl)-1,2,4-oxadiazole; 3-(4-((1-(propylsulfonyl)pyrrolidin-3-yl)oxy)phenyl)-5-(trifluoromethyl)-1,2,4-oxadiazole; 3-(4-((1-((4-bromophenyl)sulfonyl)pyrrolidin-3-yl)oxy)phenyl)-5-(trifluoromethyl)-1,2,4-oxadiazole; 3-(4-((1-(pyridin-3-ylsulfonyl)pyrrolidin-3-yl)oxy)phenyl)-5-(trifluoromethyl)-1,2,4-oxadiazole; 5-(trifluoromethyl)-3-(4-((1-((4-(trifluoromethyl)phenyl)sulfonyl)pyrrolidin-3-yl)oxy)phenyl)-1,2,4-oxadiazole; 3-(4-((1-tosylpyrrolidin-3-yl)oxy)phenyl)-5-(trifluoromethyl)-1,2,4-oxadiazole; 3-(4-((1-((2,4-dichlorophenyl)sulfonyl)pyrrolidin-3-yl)oxy)phenyl)-5-(trifluoromethyl)-1,2,4-oxadiazole; 4-((3-(4-(5-(trifluoromethyl)-1,2,4-oxadiazol-3-yl)phenoxy)pyrrolidin-1-yl)sulfonyl)benzonitrile; 3-(4-((1-((3-chlorophenyl)sulfonyl)pyrrolidin-3-yl)oxy)phenyl)-5-(trifluoromethyl)-1,2,4-oxadiazole; 3-(4-((1-(isopropylsulfonyl)pyrrolidin-3-yl)oxy)phenyl)-5-(trifluoromethyl)-1,2,4-oxadiazole; 3-(4-((1-benzylpyrrolidin-3-yl)oxy)phenyl)-5-(trifluoromethyl)-1,2,4-oxadiazole; 3-(4-((1-((3-methylthiophen-2-yl)sulfonyl)pyrrolidin-3-yl)oxy)phenyl)-5-(trifluoromethyl)-1,2,4-oxadiazole; 3-(4-((1-((1-methyl-1H-imidazol-4-yl)sulfonyl)pyrrolidin-3-yl)oxy)phenyl)-5-(trifluoromethyl)-1,2,4-oxadiazole; 3-(6-((1-((3-fluorophenyl)sulfonyl)pyrrolidin-3-yl)oxy)pyridin-3-yl)-5-(trifluoromethyl)-1,2,4-oxadiazole; 4-((3-((5-(5-(trifluoromethyl)-1,2,4-oxadiazol-3-yl)pyridin-2-yl)oxy)pyrrolidin-1-yl)sulfonyl)benzonitrile; 5-(trifluoromethyl)-3-(6-((1-((3-(trifluoromethyl)phenyl)sulfonyl)pyrrolidin-3-yl)oxy)pyridin-3-yl)-1,2,4-oxadiazole; 3-(6-((1-((2-fluorophenyl)sulfonyl)pyrrolidin-3-yl)oxy)pyridin-3-yl)-5-(trifluoromethyl)-1,2,4-oxadiazole; 3-(6-((1-(pyridin-3-ylsulfonyl)pyrrolidin-3-yl)oxy)pyridin-3-yl)-5-(trifluoromethyl)-1,2,4-oxadiazole; 3-(6-((1-(benzylsulfonyl)pyrrolidin-3-yl)oxy)pyridin-3-yl)-5-(trifluoromethyl)-1,2,4-oxadiazole; 3-(6-((1-(isopropylsulfonyl)pyrrolidin-3-yl)oxy)pyridin-3-yl)-5-(trifluoromethyl)-1,2,4-oxadiazole; tert-butyl 3-((4-(5-(trifluoromethyl)-1,2,4-oxadiazol-3-yl)phenyl)thio)pyrrolidine-1-carboxylate; 3-(6-((1-((2-chlorophenyl)sulfonyl)pyrrolidin-3-yl)oxy)pyridin-3-yl)-5-(trifluoromethyl)-1,2,4-oxadiazole; 3-(6-((1-((4-chlorobenzyl)sulfonyl)pyrrolidin-3-yl)oxy)pyridin-3-yl)-5-(trifluoromethyl)-1,2,4-oxadiazole; 3-(6-((1-((2-methoxyethyl)sulfonyl)pyrrolidin-3-yl)oxy)pyridin-3-yl)-5-(trifluoromethyl)-1,2,4-oxadiazole; 3-(4-((1-(4-methylbenzyl)pyrrolidin-3-yl)oxy)phenyl)-5-(trifluoromethyl)-1,2,4-oxadiazole; 1-(3-((4-(5-(trifluoromethyl)-1,2,4-oxadiazol-3-yl)phenyl)thio)pyrrolidin-1-yl)ethan-1-one; m-tolyl(3-((4-(5-(trifluoromethyl)-1,2,4-oxadiazol-3-yl)phenyl)thio)pyrrolidin-1-yl)methanone; 3-(6-((1-(phenethylsulfonyl)pyrrolidin-3-yl)oxy)pyridin-3-yl)-5-(trifluoromethyl)-1,2,4-oxadiazole; (4-methoxyphenyl)(3-((4-(5-(trifluoromethyl)-1,2,4-oxadiazol-3-yl)phenyl)thio)pyrrolidin-1-yl)methanone; phenyl(3-((4-(5-(trifluoromethyl)-1,2,4-oxadiazol-3-yl)phenyl)thio)pyrrolidin-1-yl)methanone; 2-phenyl-1-(3-((4-(5-(trifluoromethyl)-1,2,4-oxadiazol-3-yl)phenyl)thio)pyrrolidin-1-yl)ethan-1-one; 3-(4-((1-(4-chlorobenzyl)pyrrolidin-3-yl)oxy)phenyl)-5-(trifluoromethyl)-1,2,4-oxadiazole; 3-(4-((1-isopropylpyrrolidin-3-yl)oxy)phenyl)-5-(trifluoromethyl)-1,2,4-oxadiazole; 2-phenyl-1-(3-((4-(5-(trifluoromethyl)-1,2,4-oxadiazol-3-yl)phenyl)sulfonyl)pyrrolidin-1-yl)ethan-1-one; (2-fluorophenyl)(3-((4-(5-(trifluoromethyl)-1,2,4-oxadiazol-3-yl)phenyl)thio)pyrrolidin-1-yl)methanone; 1-(3-((5-(5-(trifluoromethyl)-1,2,4-oxadiazol-3-yl)pyridin-2-yl)oxy)pyrrolidin-1-yl)ethan-1-one; 1-(3-((5-(5-(trifluoromethyl)-1,2,4-oxadiazol-3-yl)pyridin-2-yl)oxy)pyrrolidin-1-yl)propan-1-one; pyridin-4-yl(3-((4-(5-(trifluoromethyl)-1,2,4-oxadiazol-3-yl)phenyl)thio)pyrrolidin-1-yl)methanone; 2-(4-chlorophenyl)-1-(3-((4-(5-(trifluoromethyl)-1,2,4-oxadiazol-3-yl)phenyl)thio)pyrrolidin-1-yl)ethan-1-one; 2-(4-chlorophenyl)-1-(3-((4-(5-(trifluoromethyl)-1,2,4-oxadiazol-3-yl)phenyl)sulfonyl)pyrrolidin-1-yl)ethan-1-one; 2-(4-methoxyphenyl)-1-(3-((4-(5-(trifluoromethyl)-1,2,4-oxadiazol-3-yl)phenyl)sulfonyl)pyrrolidin-1-yl)ethan-1-one; (1-methyl-1H-pyrazol-3-yl)(3-((5-(5-(trifluoromethyl)-1,2,4-oxadiazol-3-yl)pyridin-2-yl)oxy)pyrrolidin-1-yl)methanone; isoxazol-3-yl(3-((5-(5-(trifluoromethyl)-1,2,4-oxadiazol-3-yl)pyridin-2-yl)oxy)pyrrolidin-1-yl)methanone; (4-(dimethylamino)phenyl)(3-((4-(5-(trifluoromethyl)-1,2,4-oxadiazol-3-yl)phenyl)thio)pyrrolidin-1-yl)methanone; oxazol-4-yl(3-((5-(5-(trifluoromethyl)-1,2,4-oxadiazol-3-yl)pyridin-2-yl)oxy)pyrrolidin-1-yl)methanone; thiazol-4-yl(3-((5-(5-(trifluoromethyl)-1,2,4-oxadiazol-3-yl)pyridin-2-yl)oxy)pyrrolidin-1-yl)methanone; (3-((4-(5-(trifluoromethyl)-1,2,4-oxadiazol-3-yl)phenyl)thio)pyrrolidin-1-yl)(4-(trifluoromethyl)phenyl)methanone; (3-((4-(5-(trifluoromethyl)-1,2,4-oxadiazol-3-yl)phenyl)sulfonyl)pyrrolidin-1-yl)(4-(trifluoromethyl)phenyl)methanone; 2-methyl-1-(3-((5-(5-(trifluoromethyl)-1,2,4-oxadiazol-3-yl)pyridin-2-yl)oxy)pyrrolidin-1-yl)propan-1-one; cyclopropyl(3-((5-(5-(trifluoromethyl)-1,2,4-oxadiazol-3-yl)pyridin-2-yl)oxy)pyrrolidin-1-yl)methanone; tert-butyl (R)-3-((4-(5-(trifluoromethyl)-1,2,4-oxadiazol-3-yl)phenyl)thio)pyrrolidine-1-carboxylate; tert-butyl (R)-3-(3-(5-(trifluoromethyl)-1,2,4-oxadiazol-3-yl)phenoxy)pyrrolidine-1-carboxylate; (R)-2,2-dimethyl-1-(3-(3-(5-(trifluoromethyl)-1,2,4-oxadiazol-3-yl)phenoxy)pyrrolidin-1-yl)propan-1-one; (R)-(2-fluorophenyl)(3-(3-(5-(trifluoromethyl)-1,2,4-oxadiazol-3-yl)phenoxy)pyrrolidin-1-yl)methanone; (R)-(3-fluorophenyl)(3-(3-(5-(trifluoromethyl)-1,2,4-oxadiazol-3-yl)phenoxy)pyrrolidin-1-yl)methanone; (R)-(4-fluorophenyl)(3-(3-(5-(trifluoromethyl)-1,2,4-oxadiazol-3-yl)phenoxy)pyrrolidin-1-yl)methanone; (R)-p-tolyl(3-(3-(5-(trifluoromethyl)-1,2,4-oxadiazol-3-yl)phenoxy)pyrrolidin-1-yl)methanone; (R)-m-tolyl(3-(3-(5-(trifluoromethyl)-1,2,4-oxadiazol-3-yl)phenoxy)pyrrolidin-1-yl)methanone; (R)-(3-(3-(5-(trifluoromethyl)-1,2,4-oxadiazol-3-yl)phenoxy)pyrrolidin-1-yl)(4-(trifluoromethyl)phenyl)methanone; (R)-(3-chlorophenyl)(3-(3-(5-(trifluoromethyl)-1,2,4-oxadiazol-3-yl)phenoxy)pyrrolidin-1-yl)methanone; (R)-(4-methoxyphenyl)(3-(3-(5-(trifluoromethyl)-1,2,4-oxadiazol-3-yl)phenoxy)pyrrolidin-1-yl)methanone; tert-butyl (R)-3-(methyl(4-(5-(trifluoromethyl)-1,2,4-oxadiazol-3-yl)phenyl)amino)pyrrolidine-1-carboxylate; (R)-2-(4-methoxyphenyl)-1-(3-((4-(5-(trifluoromethyl)-1,2,4-oxadiazol-3-yl)phenyl)thio)pyrrolidin-1-yl)ethan-1-one; (R)-(3-(methyl(4-(5-(trifluoromethyl)-1,2,4-oxadiazol-3-yl)phenyl)amino)pyrrolidin-1-yl)(phenyl)methanone; (R)-pyridin-2-yl(3-(3-(5-(trifluoromethyl)-1,2,4-oxadiazol-3-yl)phenoxy)pyrrolidin-1-yl)methanone; (R)-pyridin-4-yl(3-(3-(5-(trifluoromethyl)-1,2,4-oxadiazol-3-yl)phenoxy)pyrrolidin-1-yl)methanone; (R)-pyridin-3-yl(3-(3-(5-(trifluoromethyl)-1,2,4-oxadiazol-3-yl)phenoxy)pyrrolidin-1-yl)methanone; tert-butyl (S)-3-((4-(5-(trifluoromethyl)-1,2,4-oxadiazol-3-yl)phenyl)thio)pyrrolidine-1-carboxylate; tert-butyl (S)-3-(3-(5-(trifluoromethyl)-1,2,4-oxadiazol-3-yl)phenoxy)pyrrolidine-1-carboxylate; (S)-2,2-dimethyl-1-(3-(3-(5-(trifluoromethyl)-1,2,4-oxadiazol-3-yl)phenoxy)pyrrolidin-1-yl)propan-1-one; (S)-(2-fluorophenyl)(3-(3-(5-(trifluoromethyl)-1,2,4-oxadiazol-3-yl)phenoxy)pyrrolidin-1-yl)methanone; (S)-(3-fluorophenyl)(3-(3-(5-(trifluoromethyl)-1,2,4-oxadiazol-3-yl)phenoxy)pyrrolidin-1-yl)methanone; (S)-(4-fluorophenyl)(3-(3-(5-(trifluoromethyl)-1,2,4-oxadiazol-3-yl)phenoxy)pyrrolidin-1-yl)methanone; (S)-p-tolyl(3-(3-(5-(trifluoromethyl)-1,2,4-oxadiazol-3-yl)phenoxy)pyrrolidin-1-yl)methanone; (S)-m-tolyl(3-(3-(5-(trifluoromethyl)-1,2,4-oxadiazol-3-yl)phenoxy)pyrrolidin-1-yl)methanone; (S)-(3-(3-(5-(trifluoromethyl)-1,2,4-oxadiazol-3-yl)phenoxy)pyrrolidin-1-yl)(4-(trifluoromethyl)phenyl)methanone; (S)-(3-chlorophenyl) (3-(3-(5-(trifluoromethyl)-1,2,4-oxadiazol-3-yl)phenoxy)pyrrolidin-1-yl)methanone; (S)-(4-methoxyphenyl)(3-(3-(5-(trifluoromethyl)-1,2,4-oxadiazol-3-yl)phenoxy)pyrrolidin-1-yl)methanone; tert-butyl (S)-3-(methyl(4-(5-(trifluoromethyl)-1,2,4-oxadiazol-3-yl)phenyl)amino)pyrrolidine-1-carboxylate; (S)-2-(4-methoxyphenyl)-1-(3-((4-(5-(trifluoromethyl)-1,2,4-oxadiazol-3-yl)phenyl)thio)pyrrolidin-1-yl)ethan-1-one; (S)-(3-(methyl(4-(5-(trifluoromethyl)-1,2,4-oxadiazol-3-yl)phenyl)amino)pyrrolidin-1-yl)(phenyl)methanone; (S)-pyridin-2-yl(3-(3-(5-(trifluoromethyl)-1,2,4-oxadiazol-3-yl)phenoxy)pyrrolidin-1-yl)methanone; (S)-pyridin-4-yl(3-(3-(5-(trifluoromethyl)-1,2,4-oxadiazol-3-yl)phenoxy)pyrrolidin-1-yl)methanone; and (S)-pyridin-3-yl(3-(3-(5-(trifluoromethyl)-1,2,4-oxadiazol-3-yl)phenoxy)pyrrolidin-1-yl)methanone.

The following compounds are excluded from the definition of Formula I:
1-[4-[[5-[5-(trifluoromethyl)-1,2,4-oxadiazol-3-yl]-2-pyrimidinyl]amino]-1-piperidinyl]-ethanone, (2360451-15-4);
3-[2-chloro-4-[5-(trifluoromethyl)-1,2,4-oxadiazol-3-yl]phenoxy]-4-hydroxy-4-methyl-1,1-dimethylethyl ester-1-piperidinecarboxylic acid, (2127083-53-6);
3-hydroxy-3-methyl-4-[[5-[5-(trifluoromethyl)-1,2,4-oxadiazol-3-yl]-2-pyridinyl]oxy]-1,1-dimethylethyl ester-1-piperidinecarboxylic acid, (2125466-28-4);
N-[1-[(1-methyl-1H-indol-3-yl)methyl]-4-piperidinyl]-5-[5-(trifluoromethyl)-1,2,4-oxadiazol-3-yl]-2-pyrimidinamine, (1434044-32-2);
N-[1-[(1-methyl-1H-indol-3-yl)methyl]-4-piperidinyl]-5-[5-(trifluoromethyl)-1,2,4-oxadiazol-3-yl]-2-pyridinamine, (1434044-31-1);
4-[[5-[5-(trifluoromethyl)-1,2,4-oxadiazol-3-yl]-2-pyrimidinyl]amino]-1,1-dimethylethyl ester-1-piperidinecarboxylic acid, (1433958-20-3);
4-[[5-[5-(trifluoromethyl)-1,2,4-oxadiazol-3-yl]-2-pyridinyl]amino]-1,1-dimethylethyl ester-1-piperidinecarboxylic acid, (1433958-19-0);
N-[1-[(1-methyl-1H-indol-3-yl)methyl]-4-piperidinyl]-5-[5-(trifluoromethyl)-1,2,4-oxadiazol-3-yl]-2-pyrimidinamine, hydrochloride, (1433958-05-4); and
N-[1-[(1-methyl-1H-indol-3-yl)methyl]-4-piperidinyl]-5-[5-(trifluoromethyl)-1,2,4-oxadiazol-3-yl]-2-pyridinamine, hydrochloride, (1433958-02-1).

The compound of the present invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compound of the present invention may be present as a mixture of stereoisomers, individual stereoisomers or as an optically active form.

An anion part of the salt in case the compound of Formula I is a cationic or capable of forming a cation can be inorganic or organic. Alternatively, a cation part of the salt in case the compound of Formula I is an anionic or capable of forming anion can be inorganic or organic. Examples of inorganic anion part of the salt include but are not limited to chloride, bromide, iodide, fluoride, sulphate, phosphate, nitrate, nitrite, hydrogen carbonates, hydrogen sulphate. Examples of organic anion part of the salt include but are not limited to formate, alkanoates, carbonates, acetates, trifluoroacetate, trichloroacetate, propionate, glycolate, thiocyanate, lactate, succinate, malate, citrates, benzoates, cinnamates, oxalates, alkylsulphates, alkylsulphonates, arylsulphonates aryldisulphonates, alkylphosphonates, arylphosphonates, aryldiphosphonates, p-toluenesulphonate, and salicylate. Examples of inorganic cation part of the salt include but are not limited to alkali and alkaline earth metals. Examples of organic cation part of the salt include but are not limited to pyridine, methyl amine, imidazole, benzimidazole, hitidine, phosphazene, tetramethyl ammonium, tetrabutylammonium, choline and trimethylamine.

Metal ions in metal complexes of the compound of Formula I are especially the ions of the elements of the second main group, especially calcium and magnesium, of the third and fourth main group, especially aluminium, tin and lead, and also of the first to eighth transition groups, especially chromium, manganese, iron, cobalt, nickel, copper, zinc and others. Particular preference is given to the metal ions of the elements of the fourth period and the first to eighth transition groups. Here, the metals can be present in the various valencies that they can assume.

The compound selected from Formula I, (including all stereoisomers, N-oxides, and salts thereof), typically may exist in more than one form. Formula I thus includes all crystalline and non-crystalline forms of the compound that Formula I represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula I can exhibit beneficial effects (e.g., suitability for preparation of useful formulations, improved biological performance) relative to another polymorph or a mixture of polymorphs of the same compound represented by Formula I. Preparation and isolation of a particular polymorph of a compound represented by Formula I can be achieved by methods known to those skilled in the art including, for example, crystallization using selected solvents and temperatures.

In another embodiment the present invention relates to a composition comprising the compound of Formula I, agriculturally acceptable salts, metal complexes, constitutional isomers, stereo-isomers, diastereoisomers, enantiomers, chiral isomers, atropisomers, conformers, rotamers, tautomers, optical isomers, polymorphs, geometric isomers, or N-oxides thereof optionally with one or more additional active ingredient with the auxiliary such as inert carrier or any other essential ingredient such as surfactants, additives, solid diluents and liquid diluents.

The compound of Formula I and the composition according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compound of Formula I and the composition according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants. Particularly, the compound of Formula I and the composition according to the invention are important in the control of phytopathogenic fungi on soybeans and on the plant propagation material, such as seeds, and the crop material of soybeans. Accordingly, the present invention also includes a composition comprising at least one compound of Formula I and seed. The amount of the compound of Formula I in the composition ranges from 0.1 gai (gram per active ingredient) to 10 kgai (kilogram per active ingredient) per 100 kg of seeds.

Preferably, the compound of Formula I and composition thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative or reproductive parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts, twigs, flowers, and other parts of plants, including seedlings and young plants, which are to be transplanted after germination or after emergence from soil.

These young plants may also be protected before transplantation by a total or partial treatment by immersion or pouring.

Preferably, treatment of plant propagation materials with the compound of Formula I, the combination and or the composition thereof, respectively, is used for controlling a multitude of fungi on cereals, such as wheat, rye, barley and oats; rice, corn, cotton, fruits, coffee, sugarcane and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties. Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1 185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants capable to synthesize one or more insecticidal proteins, especially those known from the bacterial genus (Bacillus), by the use of recombinant DNA techniques are within the scope of the present invention. The Bacillus are particularly from Bacillus thuringiensis, such as δ-endotoxins, e. g. Cry1A(b), Cry1A(c), Cry1F, Cry1F(a2), Cry1lA(b), Cry1llA, Cry1llB (b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO02/015701). Further examples of such toxins or genetically modified plants capable of synthesizing such toxins are disclosed, e. g., in EP374753, WO93/007278, WO95/34656, EP427 529, EP451 878, WO03/18810 and WO03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme phosphinothricin-N-acetyltransferase [PAT]); NuCOTN® 33B (cotton cultivars producing the Cry1Ac toxin), Bollgard® I (cotton cultivars producing the Cry1 Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt1 1 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb 1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1 F toxin and PAT enzyme).

Furthermore, plants capable to synthesize one or more proteins to increase the resistance or tolerance of those plants to bacterial, viral or fungal pathogens by the use of recombinant DNA techniques are also within the scope of the present invention. Examples of such proteins are the so-called “pathogenesis-related proteins” (PR proteins, see, e. g. EP392225), plant disease resistance genes (e. g. potato cultivars, which express resistance genes acting against Phytophthora infestans derived from the Mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants capable to synthesize one or more proteins, by the use of recombinant DNA techniques, to increase the productivity (e. g. bio mass production, grain yield, starch content, oil content or protein content), tolerance to drought, salinity or other growth-limiting environmental factors or tolerance to pests and fungal, bacterial or viral pathogens of those plants are within the scope of the present invention.

Furthermore, plants that contain a modified amount of substances of content or new substances of content, by the use of recombinant DNA techniques, to improve human or animal nutrition, e. g. oil crops that produce health-promoting long-chain omega-3 fatty acids or unsaturated omega-9 fatty acids (e. g. Nexera® rape, DOW Agro Sciences, Canada) are also within the scope of the present invention.

Furthermore, plants that contain a modified amount of substances of content or new substances of content, by the use of recombinant DNA techniques, to improve raw material production, e. g. potatoes that produce increased amounts of amylopectin (e. g. Amflora® potato, BASF SE, Germany) are also within the scope of the present invention.

The present invention also relates to a method for controlling or preventing infestation of plants by phytopathogenic micro-organisms in agricultural crops and or horticultural crops wherein an effective amount of at least one compound of Formula I or the combination of the present invention or the composition of the present invention, is applied to the seeds of plants. The compound, the combination and the composition of the present invention can be used for controlling or preventing plant diseases. The compound of Formula I, the combination and or the composition thereof, respectively, are particularly suitable for controlling the following plant diseases:

Albugo spp. (white rust) on ornamentals, vegetables (e. g. A. Candida) and sunflowers (e. g. A. tragopogonis); Altemaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (C. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (£. pyri), soft fruits (£. veneta: anthracnose) and vines (£. ampelina: anthracnose); Entyloma oryzae (leaf smut) on rice; Epicoccum spp. (black mold) on wheat; Erysiphe spp. (powdery mildew) on sugar beets (£. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), soybeans, potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or .rotbrenner′, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuligiuea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compound of Formula I, the combination or the composition thereof may be used to treat several fungal pathogens. Non-limiting examples of pathogens of fungal diseases which can be treated in accordance with the invention include:

Ustilaginales such as Ustilaginoidea vireos, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries. Blastocladiomycetes, such as Physoderma maydis. Mucoromycetes, such as Choanephora cucurbitarum; Mucor spp.; and Rhizopus arrhizus,

In another embodiment diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

In particular, Cronartium ribicola (White pine blister rust); Gymnosporangium juniperi-virginianae (Cedar-apple rust); Hemileia vastatrix (Coffee rust); Phakopsora meibomiae and P. pachyrhizi (Soybean rust); Puccinia coronata (Crown Rust of Oats and Ryegrass); Puccinia graminis (Stem rust of wheat and Kentucky bluegrass, or black rust of cereals); Puccinia hemerocallidis (Daylily rust); Puccinia persisters subsp. triticina (wheat rust or ‘brown or red rust’); Puccinia sorghi (rust in corn); Puccinia striiformis (‘Yellow rust’ in cereals); Uromyces appendiculatus (rust of beans); Uromyces phaseoli (Bean rust); Puccinia melanocephala (‘Brown rust’ in sugarcane); Puccinia kuehnii (‘Orange rust’ in sugarcane).

Plants which can be treated in accordance with the invention include the following: cotton, flax, grapevine, fruits, vegetables, such as Rosaceae sp (for example pome fruits such as apples, pears, apricots, cherries, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Vitaceae sp. (for example grapes); Solanaceae sp. (for example tomatoes, peppers), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as PoaceaelGramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); Malvaceae (for example cotton); useful plants and ornamental plants for gardens and wooded areas; and genetically modified varieties of each of these plants.

More preference is given to controlling the following diseases of soya beans: Fungal diseases on leaves, stems, pods and seeds caused, for example, by Altemaria leaf spot (Altemaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infimdibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectiia crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidennatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The present invention also relates to the use of the compound of Formula I, the combination or the composition thereof for controlling or preventing the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye and Phakopsoraceae spp. on various plants, in particular Phakopsora pachyrhizi and P. meibomiae (soybean rust) on soybeans, Hemileia vastatrix (Coffee rust), Uromyces appendiculatus, Uromyces fabae and Uromyces phaseoli (rust of beans).

The present invention further relates to the use of the compound of Formula I, the combination or the composition thereof for controlling or preventing against phytopathogenic fungi such as Phakopsora pachyrhizi, Phakopsora meibomiae, of agricultural crops and or horticultural crops.

The compound of Formula I, the combination and the composition thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria.

As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Pora spp., Serpula spp. and Tyromyces spp., Deuteromycetes such as Aspergillus spp., Cladosporium spp., Penicillium spp., Trichoderma spp., Altemaria spp., Paecilomyces spp. and Zygomycetes such as Mucor spp., and in addition in the protection of stored products and harvest the following yeast fungi are worthy of note: Candida spp. and Saccharomyces cerevisae.

In one embodiment the compound of Formula I, the combination and the composition thereof, respectively, are particularly suitable for controlling the following plant diseases: Phakopsora pachyrhizi and P. meibomiae (soybean rust) on soybeans.

The present invention further relates to a method for controlling or preventing phytopathogenic fungi. The method comprises treating the fungi or the materials, plants, plant parts, locus thereof, soil or seeds to be protected against fungal attack, with an effective amount of at least one compound of Formula I or the combination or the composition comprising at least one compound of Formula I.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combination according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compound of Formula I, the combination and the composition thereof, respectively, may be used for improving the health of a plant. The invention also relates to a method for improving plant health by treating a plant, its propagation material and/or the locus where the plant is growing or is to grow with an effective amount of compound I and the composition thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compound of Formula I can be present in different crystal modifications or polymorphs whose biological activity may differ. They are likewise subject matter of the present invention.

The compound of Formula I are employed as such or in the form of composition for treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with a compound of Formula I, the combination and the composition thereof protectively either at or before planting or transplanting.

The invention also relates to agrochemical composition comprising an auxiliary and at least one compound of Formula I.

An agrochemical composition comprises a fungicidally effective amount of a compound of Formula I. The term “effective amount” denotes an amount of the composition or of the compound of Formula I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound of Formula I used.

The compound of Formula I, their oxides, metal complexes, isomers, polymorphs or the agriculturally acceptable salts thereof can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel Formulations for the treatment of plant propagation materials such as seeds (e. g. GF). These and further compositions types are defined in the “Catalogue of pesticide Formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product Formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof. Suitable solid carriers or fillers are mineral earths, e. g. silicates, silica gels, talc, kaolins, limestone, lime, chalk, clays, dolomite, diatomaceous earth, bentonite, calcium sulphate, magnesium sulphate, magnesium oxide; polysaccharides, e. g. cellulose, starch; fertilizers, e. g ammonium sulphate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulphates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulphates are sulphates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide.

Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines

Suitable adjuvants are compounds, which have a negligible or even no pesticidal activity themselves, and which improve the biological performance of the compound of Formula I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound of Formula I and 5-15 wt % wetting agent (e. g. alcohol alkoxylates) are dissolved in water and/or in a water-soluble solvent (e. g. alcohols) ad 100 wt %. The active substance dissolves upon dilution with water.

ii) Dispersible Concentrates (DC)

5-25 wt % of a compound of Formula I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound of Formula I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

iv) Emulsions (EW, EO, ES)

5-40 wt % of a compound of Formula I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound of Formula I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

vi) Water-Dispersible Granules and Water-Soluble Granules (WG, SG) 50-80 wt % of a compound of Formula I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance. vii) Water-dispersible powders and water-soluble powders (WP, SP, WS) 50-80 wt % of a compound of Formula I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound of Formula I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

ix) Microemulsion (ME)

5-20 wt % of a compound of Formula I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound of Formula I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound of Formula I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

xi) Dustable Powders (DP, DS)

1-10 wt % of a compound of Formula I are ground finely and mixed intimately with solid carrier (e. g. finely divided kaolin) ad 100 wt %.

xii) Granules (GR, FG)

0.5-30 wt % of a compound of Formula I are ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound of Formula I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active ingredient (ai). The active ingredients (ai) are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations.

Application can be carried out before or during sowing. Methods for applying the compound of Formula I, the combination and the composition thereof, respectively, onto plant propagation material, especially seeds, include dressing, coating, pelleting, dusting, and soaking as well as in-furrow application methods. Preferably, the compound of Formula I, the combination and the composition thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.05 to 1 kg per ha, more preferably from 0.1 to 1.0 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kg of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be mixed with the composition according to the invention in a weight ratio of 1:100 to 100:1, preferably 1:20 to 20:1.

A pesticide is generally a chemical or biological agent (such as pesticidally active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

The compound of Formula I, the combination and the composition thereof comprising them in the use as fungicides with other fungicides may result in an expansion of the fungicidal spectrum of activity being obtained or in a prevention of fungicide resistance development. Furthermore, in many cases, extraordinary effects are obtained.

The present invention also relates to the combination comprising at least one compound of Formula I and at least one further pesticidally active substance selected from the group of fungicides, insecticides, nematicides, acaricides, biopesticides, herbicides, safeners, plant growth regulators, antibiotics, fertiliers and nutrients. The pesticidally active substances reported in WO2015185485 pages 36-43 and WO2017093019 pages 42-56 can be used in conjunction with which the compound of Formula I.

The active substances referred to as component 2, their preparation and their activity e. g. against harmful fungi is known (cf.: http://www.alanwood.net/pesticides/); these substances are commercially available. The compounds described by IU PAC nomenclature, their preparation and their pesticidal activity are also known (cf. Can. J. Plant Sci. 48(6), 587-94, 1968; EP141317; EP152031; EP226917; EP243970; EP256503; EP428941; EP532022; EP1028125; EP1035122; EP1201648; EP1122244, JP2002316902; DE19650197; DE10021412; DE102005009458; U.S. Pat. Nos. 3,296,272; 3,325,503; WO9846608; WO9914187; WO9924413; WO9927783; WO0029404; WO0046148; WO0065913; WO0154501; WO 0156358; WO0222583; WO0240431; WO0310149; WO0311853; WO0314103; WO0316286; WO0353145; WO0361388; WO0366609; WO0374491; WO0449804; WO0483193; WO05120234; WO05123689; WO05123690; WO0563721; WO0587772; WO0587773; WO0615866; WO0687325; WO0687343; WO0782098; WO0790624; WO11028657; WO2012168188; WO2007006670; WO201177514; WO13047749; WO10069882; WO13047441; WO0316303; WO0990181; WO13007767; WO1310862; WO13127704; WO13024009; WO13024010; WO13047441; WO13162072; WO13092224 and WO11135833.

The present invention furthermore relates to agrochemical mixtures comprising at least one compound of Formula I (component 1) and at least one further active substance useful for plant protection.

By applying the compound of Formula I together with at least one pesticidally active compound an additional effect can be obtained.

This can be obtained by applying the compound of Formula I and at least one further pesticidally active substance simultaneously, either jointly (e. g. as tank-mix) or separately, or in succession, wherein the time interval between the individual applications is selected to ensure that the active substance applied first still occurs at the site of action in a sufficient amount at the time of application of the further pesticidally active substance(s). The order of application is not essential for working of the present invention.

When applying the compound of Formula I and a pesticidally active substance sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In the binary mixtures and the composition according to the invention the weight ratio of the component 1) and the component 2) generally depends from the properties of the active components used, usually it is in the range of 1:1000 to 1000:1, often in the range of 1:100 to 100:1, regularly in the range of 1:50 to 50:1, preferably in the range of 1:20 to 20:1, more preferably in the range of 1:10 to 10:1, even more preferably in the range of 1:4 to 4:1 and in particular in the range of 1:2 to 2:1.

According to a further embodiment of the binary mixtures and the composition thereof, the weight ratio of the component 1) and the component 2) usually is in the range of 1000:1 to 1:1000, often in the range of 100:1 to 1:100, regularly in the range of 50:1 to 1:50, preferably in the range of 20:1 to 1:20, more preferably in the range of 10:1 to 1:10, even more preferably in the range of 4:1 to 1:4 and in particular in the range of 2:1 to 1:2.

In the ternary mixtures, i.e. the composition according to the invention comprising the component 1) and component 2) and a compound III (component 3), the weight ratio of component 1) and component 2) depends from the properties of the active substances used, usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1, and the weight ratio of component 1) and component 3) usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1.

Any further active components are, if desired, added in a ratio of 20:1 to 1:20 to the component 1).

These ratios are also suitable for inventive mixtures applied by seed treatment.

The present invention also relates to a process for preparing the compound of the present invention. The process for preparing the compound of the present invention is described in the experimental section in more detail.

The invention disclosed in the present invention shall now be elaborated with the help of non-limiting schemes and examples.

General Schemes:

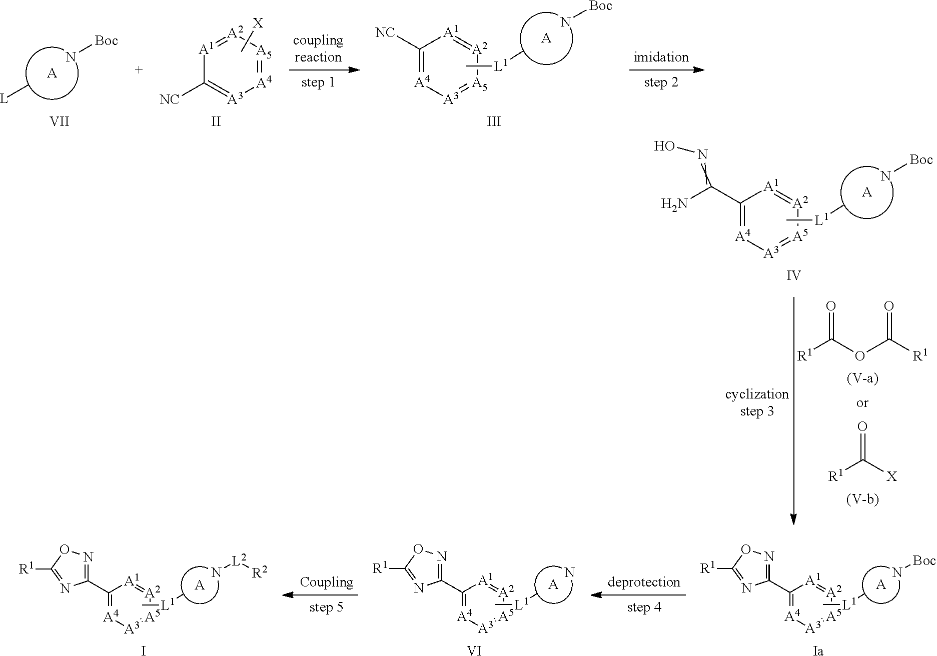

Step 1:

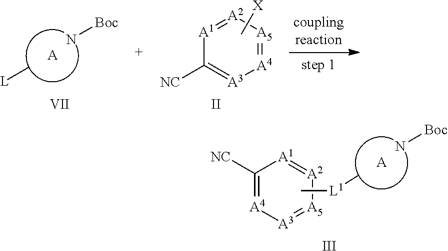

A compound of Formula III, wherein L¹ is O, S, or NR⁶, can be prepared by reacting a compound of Formula VIII, wherein L is OH, SH or NHR⁶, with a compound of Formula II, wherein X is I, Br or Cl, by using Buchwald reaction conditions in the presence of palladium catalyst such as palladium diacetate or tris(dibenzylideneacetone)dipalladium (O) and ligands such as BINAP or xanthophos.

This reaction can be carried out in the presence of inorganic base such as cesium carbonate or potassium carbonate, and typically in solvents such as toluene, 1,4-dioxane, DMF or DMSO at 25 to 100° C.

Alternatively, the compound of Formula III, wherein L¹ is O, S, or NR⁶, can also be prepared by reacting the compound of Formula VIII, wherein L is OH, SH or NHR⁶, with the compound of Formula II, wherein X is F, Br, Cl or I and is attached to A⁵, in the presence of a base such as cesium carbonate, sodium hydride, potassium tert-butoxide or sodium tert-butoxide in solvents such as tetrahydrofuran, dimethyl formamide or dimethyl sulphoxide at 0 to 90° C.

Alternatively, the compound of Formula III, wherein L¹ is O, S, or NR⁶, can also be prepared by reacting the compound of Formula VIII, wherein L is OH, with the compound of Formula II, wherein X is OH, SH or NHR⁶, in Mitsunobou reaction condition by using reagents such as diethyl azodicarboxylate (DEAD) or diisopropyl azodicarboxylate (DIAD). This reaction can be carried out typically in solvents such as tetrahydrofuran in presence of triphenylphosphine at 0 to 40° C.

Step 2:

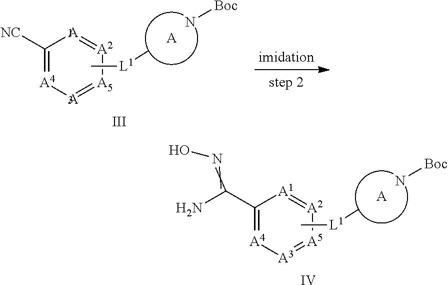

The nitrile derivative III is treated with hydroxylamine hydrochloride in the presence of a base such as sodium bicarbonate to provide the hydroxy imidamide derivative of Formula IV. This reaction can also be carried out in the presence of aqueous solution of hydroxyl amine. This reaction is typically carried out in solvents such as methanol, ethanol or tetrahydrofuran at 25-65° C.

Step 3:

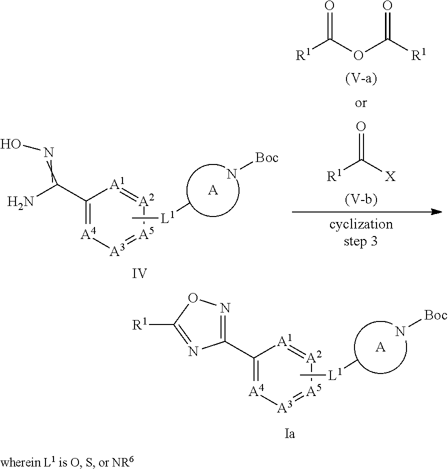

A compound of Formula Ia can be prepared by reacting the hydroxy imidamide derivative of IV with an anhydride of Formula V-a. This reaction can be carried out in solvents such as tetrahydrofuran at 0-25° C.

This reaction can also be carried out by using the compound of Formula V-b, wherein (X═Cl or Br), instead of the anhydride of Formula V-a in the presence of an organic base such as triethyl amine, diisopropyl ethyl amine or pyridine in a solvent such as tetrahydrofuran at 0-70° C.

Step 4:

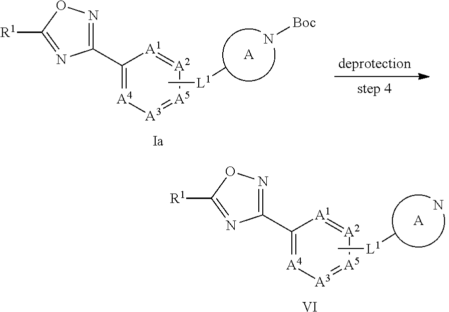

A salt of compound of Formula VI can be obtained by deprotecting the compound of Formula Ia in the presence of acid such as hydrochloric acid or trifluoroacetic acid. This reaction is typically carried out in solvents such as dichloromethane, tetrahydrofuran, 1,4-dioxane or diethyl ether at 0-40° C. The acid salt of compound of Formula VI can be reacted with aqueous solution of base such as sodium bicarbonate in solvents such as dichloromethane to obtain free amine compound of Formula VI at 5-25° C.

Step 5:

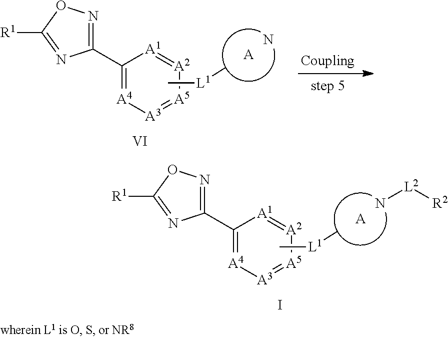

A compound of Formula I, wherein L² is (C═O), can be obtained by reacting an amine compound of Formula VI or its corresponding salt with acid chlorides in the presence of a base such as triethyl amine, diisopropylethylamine or pyridine. This reaction can be carried out in solvents such as dichloromethane, tetrahydrofuran or toluene at 0-35° C.

Alternatively, the compound of Formula I, wherein, L² is (C═O), can be obtained by reacting the amine compound of Formula VI or its corresponding salt with an acid in the presence of a coupling reagent such as n-(3-Dimethylaminopropyl)-N′-ethylcarbodiimide hydrochloride, 1-hydroxybenzotriazole or 1-[Bis(dimethylamino)methylene]-1H-1,2,3-triazolo[4,5-b]pyridinium 3-oxid hexafluorophosphate. This reaction can be typically carried out in the presence of organic bases such as triethyl amine or diisopropylethylamine in solvents such as dichloromethane, tetrahydrofuran, dimethylformamide or toluene at 0-35° C.

The compound of Formula I, wherein L² is S(═O)₂, can be obtained by reacting the amine compound of Formula VI or its corresponding salt with sulphonyl chlorides in the presence of base such as triethyl amine, diisopropylethylamine or pyridine. This reaction can be carried out in solvents such as dichloromethane, tetrahydrofuran or toluene at 0-35° C.

The compound of Formula I, wherein L² is (CR⁸R⁹)₁₃, can be obtained by reacting the amine compound of Formula VI or its corresponding salt with alkyl or benzyl halides in the presence of base such as triethyl amine, diisopropylethylamine or pyridine. This reaction can be carried out in solvents such as dichloromethane, dimethyl formamide or tetrahydrofuran at 0-35° C.

The compound of Formula I, wherein L² is (C═O), and R² is C₁-C₆-alkylamino, C₄-C₈-heterocyclylamino, heteroarylamino, arylamino, C₁-C₆-dialkylamino, C₃-C₈-cycloalkylamino or C₁-C₆-alkyl-C₃-C₈-cycloalkylamino can be obtained by reacting the amine compound of Formula VI or its corresponding salt with the mentioned respective amine in the presence of 1,1′-carbonyldiimidazole, triphosgene or diphosgene. This reaction can be typically carried out in solvent such as dichloromethane, toluene, acetonitrile, tetrahydrofuran or dimethylformamide at 0-50° C. optionally in the presence of base such as triethyl amine, diisopropylethylamine or pyridine. Alternatively, this compound can also be obtained by reacting the compound of Formula VI with corresponding isocyanides in the presence of the base such as triethylamine or diisopropylamine

Step 6:

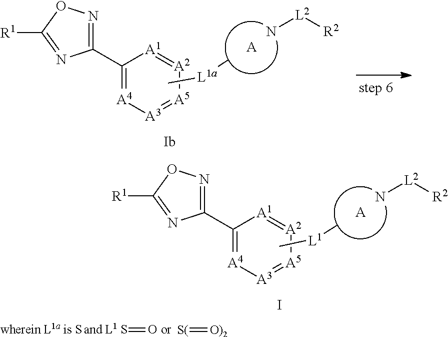

The compound of Formula I, wherein L¹ is S(═O)₁₋₂, can be obtained by reacting the compound of Formula Ib with an oxidizing reagent such as m-CPBA or oxone. This reaction can be carried out in the presence of solvents such as dichloromethane or methanol at 0-25° C.

Step 7:

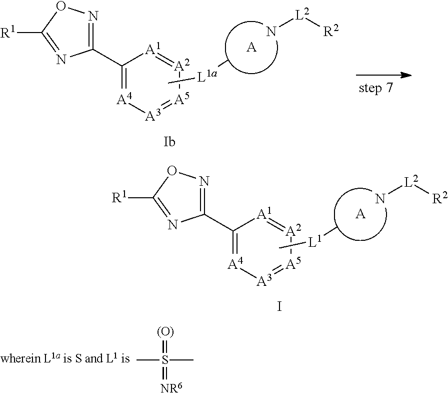

The compound of Formula I, wherein L¹ is

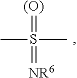

can be obtained by reacting the compound of Formula Ib with an oxidizing reagent such as iodobenzene diacetate in the presence of ammonia source such as ammonium carbamate. This reaction can be carried out in the presence of solvents such as methanol at 0-50° C.

### CHEMISTRY EXAMPLES

### Example 1: —Preparation of (S)-4-(3-(4-(5-(trifluoromethyl)-1,2,4-oxadiazol-3-yl)phenoxy)pyrrolidine-1-carbonyl)benzonitrile (Compound No. 4)

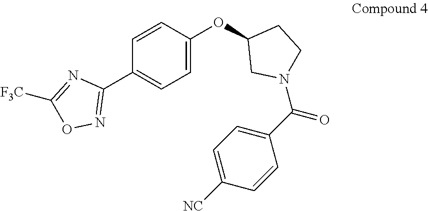 [SMILES: N#Cc1ccc(C(=O)N2CC[C@H](Oc3ccc(-c4noc(C(F)(F)F)n4)cc3)C2)cc1]

### Step 1: —Preparation of tert-butyl (S)-3-hydroxypyrrolidine-1-carboxylate

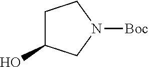 [SMILES: CC(C)(C)OC(=O)N1CC[C@H](O)C1]

To a stirred solution of (S)-3-pyrrolidinol hydrochloride (9.5 g, 77 mmol) in a mixture of dichloromethane (90 mL):methanol (22 mL), triethylamine (21.4 mL, 154 mmol) was added followed by the addition of di-tert-butyl dicarbonate (21.4 mL, 92 mmol) drop wise at 0° C. The resulting reaction mixture was stirred at 0° C. for 1 h. The reaction mixture was diluted with ethyl acetate (100 mL), and washed twice with water (150 mL). The ethyl acetate layer was dried over anhydrous sodium sulphate and concentrated under reduced pressure to obtain tert-butyl (S)-3-hydroxypyrrolidine-1-carboxylate (13.8 g, 73.7 mmol, 96% yield).

### Step 2: —Preparation of tert-butyl (S)-3-(4-cyanophenoxy)pyrrolidine-1-carboxylate

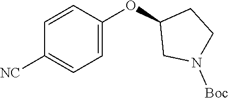 [SMILES: CC(C)(C)OC(=O)N1CC[C@H](Oc2ccc(C#N)cc2)C1]

The reaction mixture containing tert-butyl (S)-3-hydroxypyrrolidine-1-carboxylate (13.5 g, 72 mmol), 4-bromobenzonitrile (15.7 g, 87 mmol), cesium carbonate (47 g, 144 mmol) in toluene (150 mL) was degassed with nitrogen for 10 min at 25° C. (±)-2,2′-Bis(diphenylphosphino)-1,1′-binaphthalene (6.7 g, 10.8 mmol) and palladium (II) acetate (1.2 g, 5.4 mmol) were added to the reaction mixture. The resulting reaction mixture was again degassed with nitrogen for 10 min and heated to 110° C. for 18 h. The reaction mixture was cooled to 25° C. and diluted with ethyl acetate (120 mL). The ethyl acetate layer was washed twice with water (150 mL), dried over anhydrous sodium sulphate and concentrated under reduced pressure. The crude residue was purified by column chromatography using 50% ethyl acetate in hexane as an eluent on silica gel to obtain pure tert-butyl (S)-3-(4-cyanophenoxy)pyrrolidine-1-carboxylate (10.6 g, 36 mmol, 51% yield).

### Step 3: —Preparation of tert-butyl (S)-3-(4-(N′-hydroxycarbamimidoyl)phenoxy)pyrrolidine-1-carboxylate

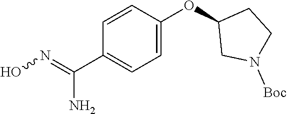 [SMILES: CC(C)(C)OC(=O)N1CC[C@H](Oc2ccc(C(N)=NO)cc2)C1]

To a stirred solution of tert-butyl (S)-3-(4-cyanophenoxy)pyrrolidine-1-carboxylate (10.5 g, 36 mmol) in ethanol (120 mL), sodium bicarbonate (6.1 g, 73 mmol) and hydroxylamine hydrochloride (5 g, 73 mmol) were added at 0° C. and stirred at 65° C. for 4 h. After completion of the reaction, the reaction mixture was filtered and concentrated under reduced pressure to obtain tert-butyl tert-butyl (S)-3-(4-(N′-hydroxycarbamimidoyl)phenoxy)pyrrolidine-1-carboxylate (11.5 g, 35.8 mmol, 98% yield).

### Step 4: —Preparation of tert-butyl (S)-3-(4-(5-(trifluoromethyl)-1,2,4-oxadiazol-3-yl)phenoxy)pyrrolidine-1-carboxylate

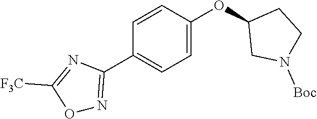 [SMILES: CC(C)(C)OC(=O)N1CC[C@H](Oc2ccc(-c3noc(C(F)(F)F)n3)cc2)C1]

To a stirred solution of tert-butyl (S)-3-(4-(N′-hydroxycarbamimidoyl)phenoxy)pyrrolidine-1-carboxylate (11.5 g, 35.8 mmol) in tetrahydrofuran (100 mL), trifluoroacetic anhydride (6.6 mL, 46.5 mmol) was added at 0° C. under nitrogen atmosphere and then stirred at 25° C. for 16 h. The reaction mixture was extracted with ethyl acetate (200 mL), the ethyl acetate layer washed twice with sodium bicarbonate solution (150 mL). The organic layer was dried over anhydrous sodium sulphate, concentrated under reduced pressure and purified by column chromatography using 40% ethyl acetate in hexane as an eluent on silica gel to obtain pure tert-butyl (S)-3-(4-(5-(trifluoromethyl)-1,2,4-oxadiazol-3-yl)phenoxy)pyrrolidine-1-carboxylate (8 g, 20 mmol, 56% yield).

### Step 5: —Preparation of (S)-3-(4-(pyrrolidin-3-yloxy)phenyl)-5-(trifluoromethyl)-1,2,4-oxadiazole

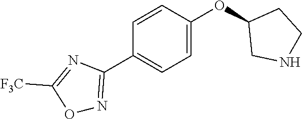 [SMILES: FC(F)(F)c1nc(-c2ccc(O[C@H]3CCNC3)cc2)no1]

To a stirred solution of tert-butyl (S)-3-(4-(5-(trifluoromethyl)-1,2,4-oxadiazol-3-yl)phenoxy)pyrrolidine-1-carboxylate (4.5 g, 11.3 mmol) in dichloromethane (53 mL), trifluoroacetic acid (13.3 mL, 172 mmol) was added at 0° C. under nitrogen atmosphere. The resulting reaction mixture was stirred at 25° C. for 2 h. The reaction mixture was concentrated under reduced pressure. The residue was dissolved in ethyl acetate (200 mL). The ethyl acetate layer was washed twice with saturated sodium bicarbonate solution (150 mL), dried over anhydrous sodium sulphate and concentrated under reduced pressure to get the crude product. The crude compound was purified by column chromatography using 60% ethyl acetate in hexane as an eluent on silica gel to obtain pure (S)-3-(4-(pyrrolidin-3-yloxy)phenyl)-5-(trifluoromethyl)-1,2,4-oxadiazole (3.2 g, 10.6 mmol, 95% yield).

### Step 6: —Preparation of (S)-phenyl(3-(4-(5-(trifluoromethyl)-1,2,4-oxadiazol-3-yl)phenoxy)pyrrolidin-1-yl)methanone

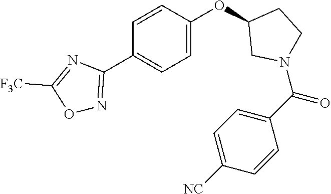 [SMILES: N#Cc1ccc(C(=O)N2CC[C@H](Oc3ccc(-c4noc(C(F)(F)F)n4)cc3)C2)cc1]

To a stirred solution of (S)-3-(4-(pyrrolidin-3-yloxy)phenyl)-5-(trifluoromethyl)-1,2,4-oxadiazole (0.3 g, 1.0 mmol) in dichloromethane (5 mL), 1-[Bis(dimethylamino)methylene]-1H-1,2,3-triazolo[4,5-b]pyridinium 3-oxide hexafluorophosphate (0.6 g, 1.5 mmol) was added, followed by the addition of 4-cyanobenzoic acid (0.2 g, 1.2 mmol) and triethylamine (0.35 mL, 2.5 mmol) at 0-5° C. under nitrogen atmosphere. The resulting reaction mixture was stirred at 25° C. for 16 h. After completion of the reaction, the reaction mixture was diluted with dichloromethane (20 mL). The dichloromethane layer was washed by sodium bicarbonate solution and dried over anhydrous sodium sulphate and then concentrated under reduced pressure to obtain a crude product. The crude product was purified by preparative HPLC to obtain (S)-4-(3-(4-(5-(trifluoromethyl)-1,2,4-oxadiazol-3-yl)phenoxy)pyrrolidine-1-carbonyl)benzonitrile (0.16 g, 0.37 mmol, 37% yield).

1H-NMR @80° C. (400 MHz, DMSO-D6) δ 7.99 (d, 2H), 7.87 (d, 2H), 7.69 (d, 2H), 7.19 (s, 2H), 5.20 (s, 1H), 3.87 (s, 1H), 3.55-3.70 (m, 3H), 2.25-2.34 (m, 1H), 2.15-2.17 (m, 1H); (M+1): 428.75

TABLE 1
The following compounds were prepared by the procedure
analogous to that for the Compound No. 4

| Compound No. | Compound Name | ¹H-NMR | Yield |
|---|---|---|---|
| 5 | (S)-2-(3,4-dimethoxyphenyl)-1- | ¹H-NMR @80° C. (400 MHz, | 0.23 g, |
| | (3-(4-(5-(trifhroromethyl)- | DMSO-D6) δ 7.99 (d, 2H), 7.15 | 0.47 mmol, |
| | 1,2,4-oxadiazol-3- | (d, 2H), 6.82-6.89 (m, 2H), | 47% yield |
| | yl)phenoxy)pyrrolidin-1- | 6.72-6.78 (m, 1H), 5.14-5.19 | |
| | yl)ethan-1-one | (m, 1H), 3.46-3.87 (m, 12H), | |
| | | 2.08-2.32 (m, 2H); (M + 1): | |
| | | 478.15 | |
| 6 | (S)-(2-fluorophenyl)(3-(4-(5- | ¹H-NMR @80° C. (400 MHz, | 0.24 g, |
| | (trifluoromethyl)-1,2,4- | DMSO-D6) δ 7.98-8.00 (m, 2H), | 0.5 mmol, |
| | oxadiazol-3- | 7.40-7.50 (s, 2H), 7.15-7.26 | 56% yield |
| | yl)phenoxy)pyrrolidin-1- | (m, 4H), 5.26-5.24 (m, 1H), | |
| | yl)methanone | 3.38-3.91 (m, 4H), 2.16-2.32 | |
| | | (m, 2H); (M + 1): 422.05 | |
| 7 | (S)-pyridin-2-yl(3-(4-(5- | ¹H-NMR @80° C. (400 MHz, | 0.2 g, |
| | (trifluoromethyl)-1,2,4- | DMSO-D6) δ 8.58-8.60 (m, 1H), | 0.50 mmol, |
| | oxadiazol-3- | 7.90-8.02 (m, 3H), 7.75-7.77 | 50% yield |
| | yl)phenoxy)pyrrolidin-1- | (m, 1H), 7.47 (s, 1H), 7.15- | |
| | yl)methanone | 7.22 (m, 2H), 5.22 (s, 1H), | |
| | | 3.71-4.10 (m, 4H), 2.26-2.33 | |
| | | (m, 1H), 2.18 (s, 1H); (M + | |
| | | 1): 405.15 | |
| 8 | (S)-(4- | ¹H-NMR @80° C. (400 MHz, | 0.26 g, |
| | (dimethylamino)phenyl)(3-(4- | DMSO-D6) δ 7.96-8.01 (m, | 0.58 mmol, |
| | (5-(trifluoromethyl)-1,2,4- | 2H), 7.41-7.44 (m, 2H), 7.15- | 58% yield |
| | oxadiazol-3- | 7.21 (m, 2H), 6.68-6.71 (m, | |
| | yl)phenoxy)pyrrolidin-1- | 2H), 5.15-5.18 (m, 1H), 3.91- | |
| | yl)methanone | 3.95 (m, 1H), 3.62-3.74 (m, | |
| | | 3H), 2.94 (s, 6H), 2.07-2.32 | |
| | | (m, 2H); (M + 1): 447.2 | |
| 9 | (S)-cyclobutyl(3-(4-(5- | ¹H-NMR @80° C. (400 MHz, | 0.22 g, |
| | (trifluoromethyl)-1,2,4- | DMSO-D6) δ 7.97-8.01 (m, | 0.58 mmol, |
| | oxadiazol-3- | 2H), 7.16 (d, J = 8.8 Hz, 2H), | 58% yield |
| | yl)phenoxy)pyrrolidin-1- | 5.13-5.18 (m, 1H), 3.63-3.73 | |
| | yl)methanone | (m, 1H), 3.28-3.58 (m, 3H), | |
| | | 2.08-2.32 (m, 7H), 1.90-1.95 | |
| | | (m, 1H), 1.74-1.82 (s, 1H); | |
| | | (M + 1): 382.35 | |
| 10 | (S)-(4-methoxyphenyl)(3-(4- | ¹H-NMR @80° C. (400 MHz, | 0.22 g, |
| | (5-(trifluoromethyl)-1,2,4- | DMSO-D6) δ 7.97-7.99 (m, 2H), | 0.51 mmol, |
| | oxadiazol-3- | 7.49-7.52 (m, 2H), 7.17 (d, | 51% yield |
| | yl)phenoxy)pyrrolidin-1- | 2H), 6.94-6.98 (m, 2H), 5.19- | |
| | yl)methanone | 5.20(m, 1H), 3.89-3.98 (m, | |
| | | 1H), 3.8 (s, 3H), 3.61-3.72 | |
| | | (m, 3H), 2.22-2.29 (m, 1H), | |
| | | 2.11-2.15 (m, 1H); (M + 1): | |
| | | 434.4 | |
| 11 | (S)-2-phenyl-1-(3-(4-(5- | ¹H-NMR @80° C. (400 MHz, | 0.23 g, |
| | (trifluoromethyl)-1,2,4- | DMSO-D6) δ 7.99 (d, 2H), | 0.54 mmol, |
| | oxadiazol-3- | 7.18-7.32 (m, 5H), 7.15 (d, | 54% yield |
| | yl)phenoxy)pyrrolidin-1- | 2H), 5.14-5.20 (m, 1H), 3.43- | |
| | yl)ethan-1-one | 3.90 (m, 6H), 2.08-2.32 (m, | |
| | | 2H); (M + 1): 418.15 | |
| 12 | (S)-pyridin-3-yl(3-(4-(5- | ¹H-NMR @80° C. (400 MHz, | 0.11 g, |
| | (trifluoromethyl)-1,2,4- | DMSO-D6) δ 8.72 (s, 1H), 8.64 | 0.27 mmol, |
| | oxadiazol-3- | (d, 1H), 7.99 (d, 2H), 7.93 | 20% yield |
| | yl)phenoxy)pyrrolidin-1- | (d, 1H), 7.45 (dd, 1H), 7.18- | |
| | yl)methanone | 7.20 (m, 2H), 5.19-5.22 (m, | |
| | | 1H), 03.90-3.94 (m, 1H), 3.67- | |
| | | 3.78 (m, 3H), 2.25-2.35 (m, | |
| | | 1H), 2.18 (s, 1H); (M + 1): | |
| 13 | (S)-pyridin-4-yl(3-(4-(5- | ¹H-NMR @80° C. (400 MHz, | 0.11 g, |
| | (trifluoromethyl)-1,2,4- | DMS0-D6) δ 8.64-8.68 (m, 2H), | 0.27 mmol, |
| | oxadiazol-3- | 7.94.8.04 (m, 2H), 7.41-7.51 | 20% yield |
| | yl)phenoxy)pyrrolidin-1- | (m, 2H), 7.13-7.24 (m, 2H), | |
| | yl)methanone | 5.16-5.24 (m, 1H), 3.44-3.92 | |
| | | (m, 4H), 2.10-2.33 (m, 2H); | |
| | | (M + 1): 405.1 | |
| 14 | (S)-(4-fluorophenyl)(3-(4- | ¹H-NMR @80° C. (400 MHz, | 0.33 g, |
| | (5-(trifluoromethyl)-1,2,4- | DMSO-D6) δ 7.99 (d, 2H), | 0.78 mmol, |
| | oxadiazol-3- | 7.58-7.62 (m, 2H), 7.16-7.26 | 58% yield |
| | yl)phenoxy)pyrrolidin-1- | (m, 4H), 5.19 (s, 1H), 3.90 | |
| | yl)methanone | (dd, 1H), 3.63-3.70 (m, 3H), | |
| | | 2.23-2.33 (m, 1H), 2.16 (s, | |
| | | 1H); (M + 1): 421.7 | |
| 15 | (S)-phenyl(3-(4-(5- | ¹H-NMR @80° C. (400 MHz, | 0.22 g, |
| | (trifluoromethyl)-1,2,4- | DMSO-D6) δ 7.97-8.00 (m, 2H), | 0.55 mmol, |
| | oxadiazol-3- | 7.48-7.51(m, 2H), 7.41-7.46 | 41% yield |
| | yl)phenoxy)pyrrolidin-1- | (m, 3H), 7.18 (d, 2H), 5.19 | |
| | yl)methanone | (s, 1H), 3.89 (dd, 1H), 3.63- | |
| | | 3.70 (m, 3H), 2.24-2.33 (m, | |
| | | 1H), 2.17 (s, 1H); (M + 1): | |
| | | 404.1 | |
| 24 | (S)-2-(3-methoxyphenyl)-1- | 1H-NMR (400 MHz, DMSO-D6) δ | 0.093 g; |
| | (3-(4-(5-(trifluoromethyl)- | 8.01-7.96 (m, 2H), 7.23-7.14 | 25% yield |
| | 1,2,4-oxadiazol-3- | (m, 3H), 6.81-6.74 (m, 3H), | |
| | yl)phenoxy)pyrrolidin-1- | 5.20-5.10 (m, 1H), 3.72 (s, | |
| | yl)ethan-l-one | 3H), 3.69 (s, 2H), 3.64-3.55 | |
| | | (m, 4H), 2.32-2.07 (m, 2H); | |
| | | LCMS (M + H): 448.05 | |
| 36 | (S)-(4- | 1H-NMR (400 MHz, DMSO-D6) δ | 0.15 g; |
| | (trifluoromethoxy)phenyl)(3- | 8.01 (dd, 2H), 7.70 (q, 2H), | 52% yield |
| | (4-(5-(trifluoromethyl)- | 7.44 (t, 2H), 7.20 (dd, 2H), | |
| | 1,2,4-oxadiazol-3- | 5.24-5.18 (m, 1H), 3.93 (dd, | |
| | yl)phenoxy)pyrrolidin-1- | 1H), 3.72-3.47 (m, 3H), 2.34- | |
| | yl)methanone | 2.16 (m, 2H); LCMS (M + H): | |
| | | 488.05 | |
| 43 | (S)-cyclopropyl(3-(4-(5- | 1H-NMR (400 MHz, DMSO-D6, at | 0.08 g; |
| | (trifluoromethyl)-1,2,4- | 80° C.) δ 8.01-7.98 (m, 2H), | 39% yield |
| | oxadiazol-3- | 7.19 (d, 2H), 5.20 (d, 1H), | |
| | yl)phenoxy)pyrrolidin-1- | 4.00-3.42 (m, 4H), 2.32-2.12 | |
| | yl)methanone | (m, 2H), 1.76 (s, 1H), 0.80- | |
| | | 0.71 (m, 4H); LCMS (M + H): | |
| | | 368.35 | |
| 44 | (S)-(4-chlorophenyl)(3-(4- | 1H-NMR (400 MHz, DMSO-D6, at | 0.1 g; |
| | (5-(trifluoromethyl)-1,2,4- | 80° C.) δ 7.99 (d, 2H), 7.55 | 31% yield |
| | oxadiazol-3- | (d, 2H), 7.47 (d, 2H), 7.18 | |
| | yl)phenoxy)pyrrolidin-1- | (d, 2H), 5.19 (s, 1H), 3.89 | |
| | yl)methanone | (dd, 1H), 3.67-3.53 (m, 3H), | |
| | | 2.32-2.23 (m, 1H), 2.18-2.12 | |
| | | (m, 1H); LCMS (M+): 438.20 | |
| 55 | (S)-2-(pyridin-2-yl)-1- | 1H-NMR (400 MHz, DMSO-D6, at | 0.17 g; |
| | (3-(4-(5-(trifluoromethyl)- | 80° C.) δ 8.47-8.37 (m, 1H), | 61% yield |
| | 1,2,4-oxadiazol-3- | 7.99 (d, 2H), 7.72-7.66 (m, | |
| | yl)phenoxy)pyrrolidin-1- | 1H), 7.30-7.27 (m, 1H), 7.23- | |
| | yl)ethan-1-one | 7.15 (m, 3H), 5.19 (d, 1H), | |
| | | 3.95-3.44 (m, 6H), 2.32-2.11 | |
| | | (m, 2H); LCMS (M + H): 418.90 | |
| 56 | (S)-(6-methoxypyridin-3- | 1H-NMR (400 MHz, DMSO-D6, at | 0.1 g; |
| | yl)(3-(4-(5- | 80° C.) δ 8.39 (d, 1H), 7.99 | 35% yield |
| | (trifluoromethyl)-1,2,4- | (d, 2H), 7.87 (dd, 1H), 7.18 | |
| | oxadiazol-3- | (d, 2H), 6.83 (d, 1H), 5.22- | |
| | yl)phenoxy)pyrrolidin-1- | 5.16 (m, 1H), 3.96-3.91 (m, | |
| | yl)methanone | 4H), 3.74-3.63 (m, 3H), 2.33- | |
| | | 2.24 (m, 1H), 2.18-2.13 (m, | |
| | | 1H); LCMS (M + H): 435.10 | |
| 57 | (S)-pyrimidin-5-yl(3-(4-(5- | 1H-NMR (400 MHz, DMSO-D6, at | 0.09 g; |
| | (trifluoromethyl)-1,2,4- | 80° C.) δ 9.24 (s, 1H), 8.97 | 33% yield |
| | oxadiazol-3- | (s, 2H), 8.00 (d, 2H), 7.19 | |
| | yl)phenoxy)pyrrolidin-1- | (d, 2H), 5.22 (s, 1H), 3.99- | |
| | yl)methanone | 3.93 (m, 1H), 3.77-3.66 (m, | |
| | | 3H), 2.34-2.26 (m, 1H), 2.19 | |
| | | 2.16 (m, 1H); LCMS (M + H): | |
| | | 406.10 | |

### Example 2: —Preparation of (S)-(3-methoxyphenyl)(3-(4-(5-(trifluoromethyl)-1,2,4-oxadiazol-3-yl)phenoxy)pyrrolidin-1-yl)methanone (Compound 1)

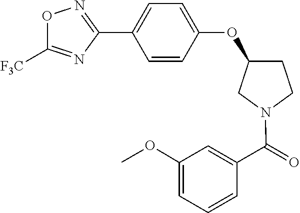 [SMILES: COc1cccc(C(=O)N2CC[C@H](Oc3ccc(-c4noc(C(F)(F)F)n4)cc3)C2)c1]

To a stirred solution of (S)-3-(4-(pyrrolidin-3-yloxy)phenyl)-5-(trifluoromethyl)-1,2,4-oxadiazole (0.4 g, 1.34 mmol) in dichloromethane (10 mL) was added triethylamine (0.93 mL, 6.7 mmol) at 0-5° C. followed by the addition of m-anisoyl chloride (0.27 g, 1.6 mmol). The resulting reaction mixture was stirred at 25° C. for 3 h. After completion of the reaction, the reaction mixture was extracted twice with dichloromethane (30 mL). The dichloromethane layer was washed with saturated sodium bicarbonate solution, dried over anhydrous sodium sulphate and concentrated under reduced pressure. The crude compound was purified by column chromatography on silica gel using hexane to 50% ethyl acetate in hexane as an eluent to obtain pure (S)-(3-methoxyphenyl)(3-(4-(5-(trifluoromethyl)-1,2,4-oxadiazol-3-yl)phenoxy)pyrrolidin-1-yl)methanone (0.25 g, 0.59 mmol, 44%).

1H-NMR @80° C. (400 MHz, DMSO-D6) δ 7.98 (d, 2H), 7.33 (t, 1H), 7.17 (d, 2H), 7.06 (d, 1H), 6.99-7.02 (m, 2H), 5.18 (s, 1H), 3.85-3.89 (m, 1H), 3.78 (s, 3H), 3.62-3.66 (m, 3H), 2.23-2.33 (m, 1H), 2.12-2.16 (m, 1H); (M+1): 434.15

TABLE 2
The following compounds were prepared by the procedure
analogous to that for the Compound No. 1

| | Compound | | |
|---|---|---|---|
| Compound No. | Name | ¹H-NMR | Yield |
| 2 | (S)-1-(3-(4-(5- | ¹H-NMR @80° C. (400 MHz, | 258 mg, |
| | (trifluoromethyl)- | DMSO-D6) δ 7.96-8.01 (m, | 0.76 mmol, |
| | 1,2,4-oxadiazol-3- | 2H), 7.16-7.18 (m, 2H), | 57% yield |
| | yl)phenoxy)pyrrolidin- | 5.13-5.20 (m, 1H), 3.55- | |
| | 1-yl)ethan-1-one | 3.64 (m, 3H), 2.06-2.30 | |
| | | (m, 3H), 1.94-1.97 (m, | |
| | | 3H); (M + 1): 341.65 | |
| 3 | (S)-(3-bromophenyl)(3- | ¹H-NMR @80° C. (400 MHz, | 380 mg, |
| | (4-(5-(trifluoromethyl)- | DMSO-D6) δ 7.99 (d, 2H), | 0.79 mmol, |
| | 1,2,4-oxadiazol-3- | 7.63-7.66 (m, 2H), 7.51 | 59% yield |
| | yl)phenoxy)pyrrolidin- | (d, 1H), 7.39 (t, 1H), 7.18 | |
| | 1-yl)methanone | (d, 2H), 5.19 (s, 1H), 3.86- | |
| | | 3.90 (m, 1H), 3.62-3.66 | |
| | | (m, 3H), 2.24-2.33 (m, | |
| | | 1H), 2.17 (s, 1H); (M + | |
| | | 1): 483.6 | |

### Example 3: —Preparation of (3-(4-(5-(trifluoromethyl)-1,2,4-oxadiazol-3-yl)phenoxy)azetidin-1-yl)(4-(trifluoromethyl)phenyl)methanone (Compound 19)

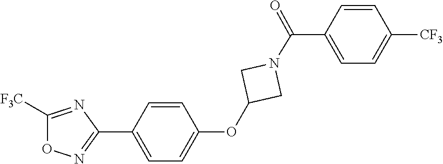 [SMILES: O=C(c1ccc(C(F)(F)F)cc1)N1CC(Oc2ccc(-c3noc(C(F)(F)F)n3)cc2)C1]

### Step 1: Preparation of tert-butyl 3-(4-cyanophenoxy)azetidine-1-carboxylate

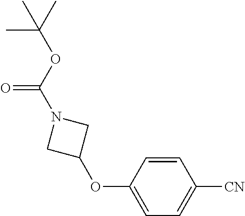 [SMILES: CC(C)(C)OC(=O)N1CC(Oc2ccc(C#N)cc2)C1]

The reaction mixture containing tert-butyl 3-hydroxyazetidine-1-carboxylate (1.1 g, 6.6 mmol), 4-bromobenzonitrile (1 g, 5.5 mmol) and cesium carbonate (3.6 g, 11 mmol) in toluene (15 mL) was degassed with nitrogen for 10 min. (±)-2,2′-Bis(diphenylphosphino)-1,1′-binaphthalene (0.5 g, 0.8 mmol) and palladium diacetate (0.1 g, 0.4 mmol) were added and the contents were again degassed with nitrogen for 10 minutes. The resulting reaction mixture was heated to 110° C. for 18 h. After completion of the reaction, the reaction mixture was cooled and diluted with ethyl acetate (50 mL). The ethyl acetate layer was washed with water (150 mL). The ethyl acetate layer was separated, dried over anhydrous sodium sulphate and concentrated under reduced pressure. The crude compound was purified by column chromatography on silica gel using 40% ethyl acetate in hexane as an eluent to obtain pure tert-butyl 3-(4-cyanophenoxy)azetidine-1-carboxylate (0.9 g, 3.3 mmol, 58% yield).

### Step 2: —Preparation of tert-butyl-3-(4-(N′-hydroxycarbamimidoyl)phenoxy)azetidine-1-carboxylate

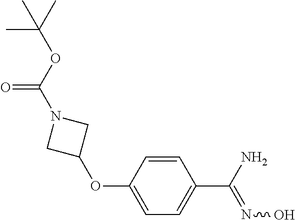 [SMILES: CC(C)(C)OC(=O)N1CC(Oc2ccc(C(N)=NO)cc2)C1]

To a solution of tert-butyl 3-(4-cyanophenoxy)azetidine-1-carboxylate (3 g, 10.6 mmol) in ethanol (30 mL), hydroxylamine hydrochloride (1.5 g, 21 mmol) and sodium bicarbonate (1.8 g, 21 mmol) were added at 25° C. The resulting reaction mixture was stirred at 65° C. for 16 h. Ethanol was distilled to obtain tert-butyl-3-(4-(N′-hydroxycarbamimidoyl)phenoxy)azetidine-1-carboxylate (3.2 g, 10.6 mmol, 100% yield).

### Step 3: —Preparation of tert-butyl 3-(4-(5-(trifluoromethyl)-1,2,4-oxadiazol-3-yl)phenoxy)azetidine-1-carboxylate

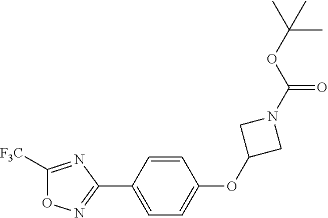 [SMILES: CC(C)(C)OC(=O)N1CC(Oc2ccc(-c3noc(C(F)(F)F)n3)cc2)C1]

To the heterogeneous reaction mixture containing tert-butyl-3-(4-(N′-hydroxycarbamimidoyl)phenoxy)azetidine-1-carboxylate (3.2 g, 10.4 mmol) in tetrahydrofuran (30 mL), trifluoroacetic anhydride (2.2 mL, 15.6 mmol) was added at 0° C. and stirred at 25° C. for 24 h. The resulting reaction mixture was poured into a mixture containing ethyl acetate (100 mL) and aqueous saturated sodium bicarbonate solution (100 mL) with stirring. The ethyl acetate layer was separated, dried over anhydrous sodium sulphate and concentrated under reduced pressure. The residue was purified by column chromatography on silica gel using hexane to 20% ethyl acetate in hexane to obtain pure tert-butyl 3-(4-(5-(trifluoromethyl)-1,2,4-oxadiazol-3-yl)phenoxy)azetidine-1-carboxylate (2.6 g, 6.7 mmol, 64% yield).

### Step 4: —Preparation of 3-(4-(azetidin-3-yloxy)phenyl)-5-(trifluoromethyl)-1,2,4-oxadiazole 2,2,2-trifluoroacetate

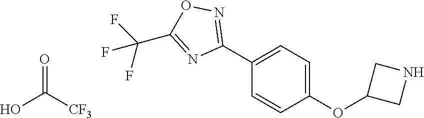 [SMILES: O=C(O)C(F)(F)F ; FC(F)(F)c1nc(-c2ccc(OC3CNC3)cc2)no1]

To a solution of tert-butyl 3-(4-(5-(trifluoromethyl)-1,2,4-oxadiazol-3-yl)phenoxy)azetidine-1-carboxylate (2.5 g, 6.49 mmol) in dichloromethane (25 mL), trifluoroacetic acid (8 mL, 105 mmol) was added under nitrogen atmosphere at 0-5° C. The resulting reaction mixture was stirred at 25° C. for 1 h. Dichloromethane was distilled under reduced pressure to obtain 3-(4-(azetidin-3-yloxy)phenyl)-5-(trifluoromethyl)-1,2,4-oxadiazole 2,2,2-trifluoroacetate (1.85 g, 6.46 mmol, 100% yield).

### Step 5: —Preparation of (3-(4-(5-(trifluoromethyl)-1,2,4-oxadiazol-3-yl)phenoxy)azetidin-1-yl)(4-(trifluoromethyl)phenyl)methanone (Compound No. 19)

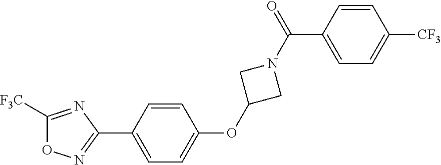 [SMILES: O=C(c1ccc(C(F)(F)F)cc1)N1CC(Oc2ccc(-c3noc(C(F)(F)F)n3)cc2)C1]

To a stirred solution of 3-(4-(azetidin-3-yloxy)phenyl)-5-(trifluoromethyl)-1,2,4-oxadiazole 2,2,2-trifluoroacetate (0.25 g, 0.63 mmol) in dichloromethane (10 mL), triethylamine (0.5 mL, 3.5 mmol) was added followed by the addition of 4-(trifluoromethyl)benzoyl chloride (0.33 g, 1.6 mmol) at 0-5° C. under nitrogen atmosphere. The resulting reaction mixture was stirred at 25° C. for 3 h. After completion of the reaction, the reaction mixture was diluted with dichloromethane (30 mL) and saturated aqueous sodium bicarbonate (10 mL) solution. The dichloromethane layer was separated, dried over anhydrous sodium sulphate and concentrated under reduced pressure. The residue was purified preparative HPLC to obtain pure (3-(4-(5-(trifluoromethyl)-1,2,4-oxadiazol-3-yl)phenoxy)azetidin-1-yl)(4-(trifluoromethyl)phenyl)methanone (0.15 g, 0.34 mmol, 39% yield).

1H-NMR (400 MHz, DMSO-D6) δ 8.00 (d, 2H), 7.87 (d, 2H), 7.82 (d, 2H), 7.08 (d, 2H), 5.18-5.22 (m, 1H), 4.73-4.77 (m, 1H), 4.58-4.62 (m, 1H), 4.38-4.40 (m, 1H), 4.06-4.09 (m, 1H); (M+1): 458.10

TABLE 3
The following compounds were prepared by the procedure
analogous to that for the Compound No. 19

| Compound No. | Compound Name | ¹H-NMR | Yield |
|---|---|---|---|
| 16 | (3-bromophenyl)(3- | ¹H-NMR (400 MHz, DMSO-D6) | 0.290 g, |
| | (4-(5-(trifluoromethyl)- | δ 7.90-8.02 (m, 2H), 7.80 (t, | 0.62 mmol, |
| | 1,2,4-oxadiazol-3- | 1H), 7.73 (dq, 1H), 7.65 | 71% yield |
| | yl)phenoxy)azetidin- | (dt, 1H), 7.40-7.45 (m, | |
| | 1-yl)methanone | 1H), 7.06-7.10 (m, 2H), 5.17- | |
| | | 5.22 (m, 1H), 4.73-4.77 (m, | |
| | | 1H), 4.55-4.60 (m, 1H), 4.37- | |
| | | 4.39 (m, 1H), 4.04-4.06 | |
| | | (m, 1H); (M + 1): 468 | |
| 17 | (3-methoxyphenyl)(3- | ¹H-NMR (400 MHz, DMSO-D6) | 0.216 g, |
| | (4-(5-(trifluoromethyl)- | δ 7.92-8.02 (m, 2H), 7.34-7.39 | 0.52 mmol, |
| | 1,2,4-oxadiazol-3- | (m, 1H), 7.20 (dt, 1H), 7.15 | 59% yield |
| | yl)phenoxy)azetidin- | (q, 1H), 7.06-7.09 (m, 3H), | |
| | 1-yl)methanone | 5.16-5.21 (m, 1H), 4.71- | |
| | | 4.75 (m, 1H), 4.54-4.58 (m, | |
| | | 1H), 4.33-4.35 (m, 1H), 3.96- | |
| | | 4.04 (m, 1H), 3.78 (s, 3H); | |
| | | (M + 1): 420.15 | |
| 18 | (3-fluorophenyl)(3- | ¹H-NMR (400 MHz, DMSO-D6) | 0.242 g, |
| | (4-(5-(trifluoromethyl)- | δ 7.99-8.02 (m, 2H), 7.49-7.52 | 0.59 mmol, |
| | 1,2,4-oxadiazol-3- | (m, 2H), 7.44-7.47 (m, 1H), | 68% yield |
| | yl)phenoxy)azetidin-1- | 7.34-7.40 (m, 1H), 7.06- | |
| | yl)methanone | 7.10 (m, 2H), 5.17-5.22 | |
| | | (m, 1H), 4.74-4.78 (m, 1H), | |
| | | 4.56-4.60 (m, 1H), 4.39- | |
| | | 7.41 (m, 1H), 4.03-4.05 | |
| | | (m, 1H); (M + 1): 408.10 | |
| 20 | (2-fluorophenyl)(3- | ¹H-NMR (400 MHz, DMSO-D6) | 0.220 g, |
| | (4-(5-(trifluoromethyl)- | δ 7.96-8.01 (m, 2H), 7.51-7.57 | 0.54 mmol, |
| | 1,2,4-oxadiazol-3- | (m, 2H), 7.27-7.33 (m, 2H), | 62% yield |
| | yl)phenoxy)azetidin- | 7.06-7.10 (m, 2H), 5.18- | |
| | 1-yl)methanone | 5.23 (m, 1H), 4.54-4.59 | |
| | | (m, 1H), 4.47-4.51 (dd, | |
| | | 1H), 4.02-4.05 (m, 2H); (M + | |
| | | 1):408.15 | |
| 21 | phenyl(3-(4-(5- | ¹H-NMR (400 MHz, DMSO-D6) | 0.192 g, |
| | (trifluoromethyl)- | δ 8.00-7.96 (m, 2H), 7.63-7.65 | 0.49 mmol, |
| | 1,2,4-oxadiazol-3- | (m, 2H), 7.48-7.52 (m, 1H), | 56% yield |
| | yl)phenoxy)azetidin- | 7.41-7.45 (m, 2H), 7.04- | |
| | 1-yl)methanone | 7.08 (m, 2H), 5.15-5.20 | |
| | | (m, 1H), 4.70-4.74 (m, 1H), | |
| | | 4.53-4.64 (m, 1H), 4.34- | |
| | | 4.36 (m, 1H), 4.03-4.05 | |
| | | (m, 1H); (M + 1): 390.1 | |

### Example-4: —Preparation of 3-(4-((1-(benzylsulfonyl)azetidin-3-yl)oxy)phenyl)-5-(trifluoromethyl)-1,2,4-oxadiazole (Compound 22)

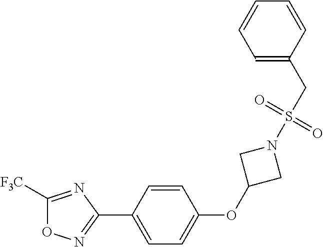 [SMILES: O=S(=O)(Cc1ccccc1)N1CC(Oc2ccc(-c3noc(C(F)(F)F)n3)cc2)C1]

To a stirred solution of 3-(4-(azetidin-3-yloxy)phenyl)-5-(trifluoromethyl)-1,2,4-oxadiazole 2,2,2-trifluoroacetate (0.25 g, 0.63 mmol) in dichloromethane (10 mL), triethylamine (0.49 ml, 3.51 mmol) was added followed by the addition of benzylsulfonyl chloride (0.25 g, 1.31 mmol) at 0-5° C. under nitrogen atmosphere. The resulting reaction mixture was stirred at 25° C. for 3 h. After completion of the reaction, the contents were mixed with dichloromethane (30 mL) and saturated aqueous sodium bicarbonate solution (10 mL). The dichloromethane layer was separated, dried over anhydrous sodium sulphate and concentrated under reduced pressure. The residue was purified by preparative HPLC to obtain pure 3-(4-((1-(benzylsulfonyl)azetidin-3-yl)oxy)phenyl)-5-(trifluoromethyl)-1,2,4-oxadiazole (0.18 g, 0.42 mmol, 47% yield).

1H-NMR (400 MHz, DMSO-D6) δ 7.97-8.02 (m, 2H), 7.42-7.57 (m, 2H), 7.31-7.39 (m, 3H), 7.07-7.09 (m, 2H), 5.11-5.17 (m, 1H), 4.59 (s, 2H), 4.27-4.41 (m, 2H), 3.91-3.95 (m, 2H); (M−1): 438

TABLE 4
The following compounds were prepared by the procedure
analogous to that for the compound No. 22

| Compound No. | Compound Name | 1H-NMR & LCMS | Yield |
|---|---|---|---|
| 201 | 3-(4-((1- | 1H-NMR (400 MHz, DMSO-D6) δ | 210 mg, |
| | (phenylsulfonyl)azetidin- | 7.93 (dt, 2H), 7.86-7.77 (m, 3H), | 70.4% yield |
| | 3-yl)oxy)phenyl)-5- | 7.72-7.68 (m, 2H), 6.96-6.92 | |
| | (trifluoromethyl)-1,2,4- | (m, 2H), 5.00-4.95 (m, 1H), | |
| | oxadiazole | 4.26 (dd, 2H), 3.65 (dd, 2H); | |
| | | LCMS (M + H): 425.85 | |
| 202 | 3-(4-((1- | 1H-NMR (400 MHz, DMSO-D6) δ | 161 mg, |
| | (methylsulfonyl)azetidin- | 8.00 (dd,2H), 7.10 (dd, 2H), 5.14- | 52% yield |
| | 3-yl)oxy)phenyl)-5- | 5.17 (m, 1H), 4.34 (dd, 2H), | |
| | (trifluoromethyl)-1,2,4- | 3.96 (q, 2H), 3.07 (s, 3H) | |
| | oxadiazole | | |

### Example 5: —Preparation of (S)-(4-methoxyphenyl)(3-((6-(5-(trifluoromethyl)-1,2,4-oxadiazol-3-yl)pyridin-3-yl)oxy)pyrrolidin-1-yl)methanone (Compound No. 29)

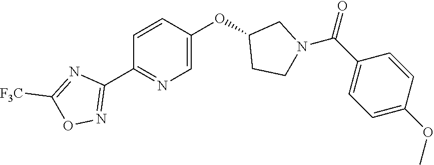 [SMILES: COc1ccc(C(=O)N2CC[C@H](Oc3ccc(-c4noc(C(F)(F)F)n4)nc3)C2)cc1]

### Step 1: —Preparation of tert-butyl (S)-3-((6-cyanopyridin-3-yl)oxy)pyrrolidine-1-carboxylate

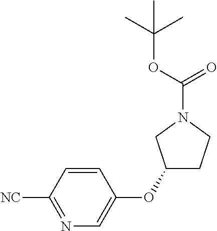 [SMILES: CC(C)(C)OC(=O)N1CC[C@H](Oc2ccc(C#N)nc2)C1]

To a stirred solution of tert-butyl (S)-3-hydroxypyrrolidine-1-carboxylate (3 g, 16 mmol) in N,N-dimethylformamide (30 mL), sodium hydride (1.1 g, 27 mmol) was added under nitrogen atmosphere at 0° C. The reaction was stirred for 30 min at 25° C., and then 5-bromopicolinonitrile (2.5 g, 13.7 mmol) was added at 0° C. The reaction mixture was stirred for 2 h at 25° C. The reaction mixture was quenched by adding ice water cautiously. The reaction mixture was diluted with ethyl acetate (50 mL) and washed thrice with water (30 mL); ethyl acetate layer was dried over anhydrous sodium sulphate and concentrated under reduced pressure to obtain the crude product. The crude product was purified by flash column chromatography on silica gel using eluent 50% ethyl acetate in hexane to obtain tert-butyl (S)-3-((6-cyanopyridin-3-yl)oxy)pyrrolidine-1-carboxylate (3.6 g, 12.4 mmol, 91% yield).

### Step 2: —Preparation of tert-butyl (S)-3-((6-(N′-hydroxycarbamimidoyl)pyridin-3-yl)oxy)pyrrolidine-1-carboxylate

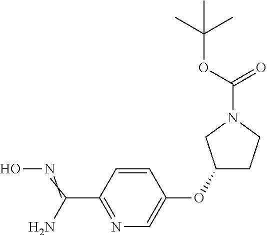 [SMILES: CC(C)(C)OC(=O)N1CC[C@H](Oc2ccc(C(N)=NO)nc2)C1]

To a stirred solution of tert-butyl (S)-3-((6-cyanopyridin-3-yl)oxy)pyrrolidine-1-carboxylate (3.8 g, 13.1 mmol) in ethanol (45 mL), sodium bicarbonate (2.2 g, 26.3 mmol) and hydroxylamine hydrochloride (1.8 g, 26.3 mmol) were added at 0° C. The reaction mixture was stirred for 4 h at 65° C. The reaction mixture was filtered, the filtrate was concentrated under reduced pressure to obtain tert-butyl (S)-3-((6-(N′-hydroxycarbamimidoyl)pyridin-3-yl)oxy)pyrrolidine-1-carboxylate (4.1 g, 12.7 mmol, 97% yield).

### Step 3: —Preparation of tert-butyl (S)-3-((6-(5-(trifluoromethyl)-1,2,4-oxadiazol-3-yl)pyridin-3-yl)oxy)pyrrolidine-1-carboxylate (Compound No. 27)

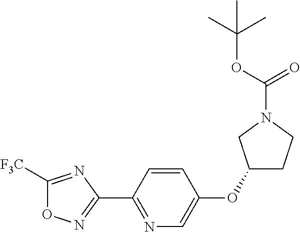 [SMILES: CC(C)(C)OC(=O)N1CC[C@H](Oc2ccc(-c3noc(C(F)(F)F)n3)nc2)C1]

To a stirred solution of tert-butyl (S)-3-((6-(N′-hydroxycarbamimidoyl)pyridin-3-yl)oxy)pyrrolidine-1-carboxylate (4.1 g, 12.7 mmol) in tetrahydrofuran (40 mL), trifluoroacetic anhydride (2.3 mL, 16.5 mmol) was added at 0° C. under nitrogen atmosphere. The reaction mixture was stirred for 16 h at 25° C. The reaction mixture was diluted with ethyl acetate (40 mL) and washed with ice cold saturated sodium bicarbonate (40 mL) solution. The ethyl acetate layer was dried over anhydrous sodium sulphate and concentrated under reduced pressure to obtain the crude product. The crude product was purified by flash column chromatography on silica gel using eluent 35% ethyl acetate in hexane to obtain tert-butyl (S)-3-((6-(5-(trifluoromethyl)-1,2,4-oxadiazol-3-yl)pyridin-3-yl)oxy)pyrrolidine-1-carboxylate (3.3 g, 8.2 mmol, 65% yield).

1H-NMR (400 MHz, DMSO-D6) δ 8.48 (d, 1H), 8.07 (d, 1H), 7.63 (dd, 1H), 5.19 (br, 1H), 3.60-3.56 (m, 1H), 3.45-3.40 (m, 2H), 3.54-3.31 (m, 1H), 2.20-2.08 (m, 2H), 1.36 (d, 9H); LCMS (M−57): 345.10

### Step 4: —Preparation of (S)-3-(5-(pyrrolidin-3-yloxy)pyridin-2-yl)-5-(trifluoromethyl)-1,2,4-oxadiazole

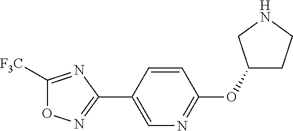 [SMILES: FC(F)(F)c1nc(-c2ccc(O[C@H]3CCNC3)nc2)no1]

To a stirred solution of tert-butyl (S)-3-((6-(5-(trifluoromethyl)-1,2,4-oxadiazol-3-yl)pyridin-3-yl)oxy)pyrrolidine-1-carboxylate (3.3 g, 8.2 mmol) in dichloromethane (35 mL), trifluoroacetic acid (7.5 mL, 97 mmol) was added at 0° C. under nitrogen atmosphere. The reaction mixture was stirred for 16 h at 25° C. The reaction mixture was concentrated under reduced pressure to obtain the crude product, the crude product was diluted with dichloromethane (40 mL) and washed with saturated sodium bicarbonate solution (40 mL), dichloromethane layer was separated, dried over anhydrous sodium sulphate and concentrated under reduced pressure to obtain (S)-3-(5-(pyrrolidin-3-yloxy)pyridin-2-yl)-5-(trifluoromethyl)-1,2,4-oxadiazole (2.1 g, 7 mmol, 36% yield).

### Step 6: —Preparation of (S)-(4-methoxyphenyl)(3-((6-(5-(trifluoromethyl)-1,2,4-oxadiazol-3-yl)pyridin-3-yl)oxy)pyrrolidin-1-yl)methanone (Compound No. 29)

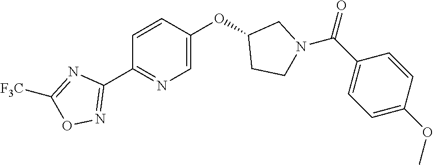 [SMILES: COc1ccc(C(=O)N2CC[C@H](Oc3ccc(-c4noc(C(F)(F)F)n4)nc3)C2)cc1]

To a stirred solution of (S)-3-(5-(pyrrolidin-3-yloxy)pyridin-2-yl)-5-(trifluoromethyl)-1,2,4-oxadiazole (0.5 g, 1.7 mmol) in dichloromethane (5 mL), was added N,N-diisopropylethylamine (1.8 mL, 10 mmol). The reaction mixture was stirred for 10 min at 25° C., then 4-methoxybenzoyl chloride (0.34 g, 2 mmol) was added at 0° C. The reaction mixture was stirred for 16 h at 25° C. The reaction mixture was diluted with dichloromethane (20 mL) and washed with water (20 mL), dichloromethane layer was dried over anhydrous sodium sulphate and concentrated under reduced pressure to obtain the crude product. The crude product was purified by preparative HPLC to obtain (S)-(4-methoxyphenyl)(3-((6-(5-(trifluoromethyl)-1,2,4-oxadiazol-3-yl)pyridin-3-yl)oxy)pyrrolidin-1-yl)methanone (0.31 g, 0.72 mmol, 43% yield).

1H-NMR (400 MHz, DMSO-D6, at 80° C.) δ 8.50 (d, 1H), 8.08 (d, 1H), 7.65-7.63 (m, 1H), 7.51 (d, 2H), 6.96 (d, 2H), 5.28 (s, 1H), 3.93 (dd, 1H), 3.80 (s, 3H), 3.73-3.62 (m, 3H), 2.34-2.25 (m, 1H), 2.18 (s, 1H); LCMS (M+H): 435.00

TABLE 5
The following compounds were prepared by the procedure
analogous to that for the compound No. 29

| Compound No. | Compound Name | | Yield |
|---|---|---|---|
| 28 | (S)-(2-fluorophenyl)(3- | H-NMR (400 MHz, DMSO-D6, at | 292 mg, |
| | ((6-(5-(trifluoromethyl)- | 80° C.) δ 8.51 (s, 1H), 8.08 (d, | 69% yield |
| | 1,2,4-oxadiazol-3- | 1H), 7.65 (d, 1H), 7.51-7.46 | |
| | yl)pyridin-3- | (m, 1H), 7.37-7.25 (m, 3H), | |
| | yl)oxy)pyrrolidin-1- | 5.29 (s, 1H), 3.92 (dd, 1H), | |
| | yl)methanone | 3.66 (t, 3H), 2.35-2.26 (m, | |
| | | 1H), 2.19-2.16 (t, 1H); LCMS | |
| | | (M + H): 423.00 | |
| 30 | (S)-(3-fluorophenyl)(3- | H-NMR (400 MHz, DMSO-D6, at | 333 mg, |
| | ((6-(5-(trifluoromethyl)- | 80° C.) δ 8.50 (d, 1H), 8.11-8.04 | 47% yield |
| | 1,2,4-oxadiazol-3- | (m, 1H), 7.68-7.59 (m, 1H), | |
| | yl)pyridin-3- | 7.48-7.42 (m, 2H), 7.31-7.21 | |
| | yl)oxy)pyrrolidin-1- | (m, 2H), 5.29 (d, 1H), 3.92- | |
| | yl)methanone | 3.36 (m, 4H), 2.34-2.18 (m, | |
| | | 2H);LCMS (M + H): 423.00 | |
| 31 | (S)-pyridin-3-yl(3-((6- | H-NMR (400 MHz, DMSO-D6, at | 221 mg, |
| | (5-(trifluoromethyl)- | 80° C.) δ 8.65 (s, 2H), 8.48 (s, | 55% yield |
| | 1,2,4-oxadiazol-3- | 1H), 8.05 (s, 1H), 7.63 (s, | |
| | yl)pyridin-3- | 1H), 7.44 (s, 2H), 5.28 (s, | |
| | yl)oxy)pyrrolidin-1- | 1H), 3.86-3.50 (m, 4H), 2.33- | |
| | yl)methanone | 2.27 (m, 1H), 2.21-2.13 (s, | |
| | | 1H); LCMS (M + H): 406.20 | |
| 32 | (S)-pyridin-4-yl(3-((6-(5- | H-NMR (400 MHz, DMSO-D6, at | 177 mg, |
| | (trifluoromethyl)-1,2,4- | 80° C.) δ 8.73 (s, 1H), 8.64 (dd, | 22% yield |
| | oxadiazol-3-yl)pyridin-3- | 1H), 8.51 (s, 1H), 8.08 (d, | |
| | yl)oxy)pyrrolidin-1- | 1H), 7.94 (d, 1H), 7.65 (d, | |
| | yl)methanone | 1H), 7.46 (dd, 1H), 5.30 (s, | |
| | | 1H), 3.95 (dd, 1H), 3.75-3.62 | |
| | | (m, 3H), 2.37-2.28 (m, 1H), | |
| | | 2.22-2.16 (m, 1H); LCMS (M + | |
| | | H): 406.20 | |

### Example 6: —Preparation of (S)—N-(2-fluorophenyl)-3-(4-(5-(trifluoromethyl)-1,2,4-oxadiazol-3-yl)phenoxy)pyrrolidine-1-carboxamide (Compound No. 37)

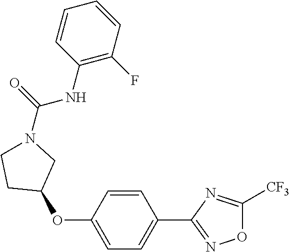 [SMILES: O=C(Nc1ccccc1F)N1CC[C@H](Oc2ccc(-c3noc(C(F)(F)F)n3)cc2)C1]

To a stirred solution of 1,1′-carbonyldiimidazole (0.3 g, 1.8 mmol) in dichloromethane (5 mL) 2-fluoroaniline (0.2 g, 1.8 mmol) was added dropwise at 0° C., the reaction mixture was stirred for 30 min. and then added triethylamine (0.42 mL, 3 mmol) drop wise, followed by the addition of (S)-3-(4-(pyrrolidin-3-yloxy)phenyl)-5-(trifluoromethyl)-1,2,4-oxadiazole 2,2,2-trifluoroacetate (0.5 g, 1.2 mmol) at 0° C. for 2 h. The reaction mixture was diluted with dichloromethane (10 mL) and washed twice with water (10 mL), dried over anhydrous sodium sulphate and concentrated under reduced pressure to obtain a crude product. The crude product was purified by flash column chromatography on silica gel using eluent 60% ethyl acetate in hexane to obtain (S)—N-(2-fluorophenyl)-3-(4-(5-(trifluoromethyl)-1,2,4-oxadiazol-3-yl)phenoxy)pyrrolidine-1-carboxamide (87 mg, 0.2 mmol, 16% yield).

1H-NMR (400 MHz, DMSO-D6) δ 8.01-7.97 (m, 3H), 7.50 (dq, 1H), 7.20-7.14 (m, 3H), 7.11-7.07 (m, 2H), 5.20 (br, 1H), 3.73 (dd, 1H), 3.64-3.60 (m, 2H), 3.49 (dd, 1H), 2.27-2.12 (m, 2H); LCMS (M+H): 437.10

TABLE 6
The following compounds were prepared by the procedure
analogous to that for the Compound No. 37

| Compound No. | Compound Name | 1H-NMR and LCMS | Yield |
|---|---|---|---|
| 38 | (S)-N-(4-fluorophenyl)- | 1H-NMR (400 MHz, DMSO-D6) δ | 141 mg, |
| | 3-(4-(5-(trifluoromethyl)- | 8.27 (s, 1H), 8.01 (dd, 2H), 7.50- | 27% yield |
| | 1,2,4-oxadiazol- | 7.45 (m, 2H), 7.20 (d, 2H), 7.08- | |
| | 3-yl)phenoxy)pyrrolidine- | 7.03 (m, 2H), 5.21 (br, 1H), 3.72 | |
| | 1-carboxamide | (dd, 1H), 3.65-3.60 (m, 2H), 3.51- | |
| | | 3.42 (m, 1H), 2.27-2.18 (m, 2H); | |
| | | LCMS (M + H): 437.15 | |
| 39 | (S)-N-(4-methoxyphenyl)- | 1H-NMR (400 MHz, DMSO-D6) δ | 65 mg, |
| | 3-(4-(5-(trifluoromethyl)- | 8.07-8.00 (m, 3H), 7.37-7.33 (m, | 12% yield |
| | 1,2,4-oxadiazol-3- | 2H), 7.19 (d, 2H), 6.80 (dd, 2H), | |
| | yl)phenoxy)pyrrolidine-1- | 5.21 (br, 1H), 3.73-3.45 (m, 7H), | |
| | carboxamide | 2.26-2.17 (m, 2H); LCMS (M + H): | |
| | | 449.15 | |

### Example 7: —Preparation of (S)-3-(4-((1-(phenylsulfonyl)pyrrolidin-3-yl)oxy)phenyl)-5-(trifluoromethyl)-1,2,4-oxadiazole (Compound No. 25)

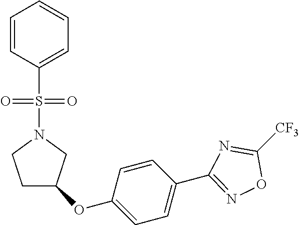 [SMILES: O=S(=O)(c1ccccc1)N1CC[C@H](Oc2ccc(-c3noc(C(F)(F)F)n3)cc2)C1]

To a solution of (S)-3-(4-(pyrrolidin-3-yloxy)phenyl)-5-(trifluoromethyl)-1,2,4-oxadiazole (0.25 g, 0.8 mmol) in dichloromethane (5 mL), triethylamine (0.47 mL, 3.3 mmol) was added at 0° C. under nitrogen atmosphere. The reaction mixture was stirred at 25° C. for 16 h. The reaction mixture was diluted with ethyl acetate (50 mL) and washed twice with saturated sodium bicarbonate solution (40 mL), dried over anhydrous sodium sulphate and concentrated under reduced pressure to obtain the crude product. The crude product was purified by flash column chromatography on silica gel using eluent 30% ethyl acetate in hexane to obtain (S)-3-(4-((1-(phenylsulfonyl)pyrrolidin-3-yl)oxy)phenyl)-5-(trifluoromethyl)-1,2,4-oxadiazole (0.1 g, 0.24 mmol, 29% yield).

1H-NMR (400 MHz, DMSO-D6) δ 7.93 (dd, 2H), 7.80-7.71 (m, 3H), 7.61 (t, 2H), 6.86 (d, 2H), 5.05 (br, 1H), 3.54 (dd, 1H), 3.43-3.37 (m, 2H), 3.26 (td, 1H), 2.11-2.02 (m, 2H); LCMS (M+H): 440.15

TABLE 7
The following compounds were prepared by the procedure
analogous to that for the Compound No. 25

| Compound No. | Compound Name | 1H-NMR and LCMS | Yield |
|---|---|---|---|
| 26 | (S)-3-(4-((1-((3- | 1H-NMR (400 MHz, DMSO-D6) δ | 82 mg, |
| | fluorophenyl)sulfonyl)pyrrolidin- | 7.94-7.91 (m, 2H), 7.68-7.57 | 21% yield |
| | 3-yl)oxy)phenyl)-5- | (m, 4H), 6.85 (dd, 2H), 5.05 | |
| | (trifluoromethyl)-1,2,4- | (br, 1H), 3.57 (dd, 1H), 3.47- | |
| | oxadiazole | 3.41 (m, 2H), 3.28-3.24 (m, | |
| | | 1H), 2.13-2.03 (m, 1H), 1.22 | |
| | | (d, 1H); LCMS (M + H): 458.15 | |
| 33 | (S)-3-(4-((1-((2- | 1H-NMR (400 MHz, DMSO-D6) δ | 93 mg, |
| | fluorophenyl)sulfonyl)pyrrolidin- | 7.94 (dt, 2H), 7.81 (td, 1H), | 24% yield |
| | 3-yl)oxy)phenyl)-5- | 7.78-7.72 (m, 1H), 7.45 (m, | |
| | (trifluoromethyl)-1,2,4- | 1H), 7.38 (td, 1H), 6.93-6.89 | |
| | oxadiazole | (m, 2H), 5.11 (br, 1H), 3.62 | |
| | | (dd, 1H), 3.54-3.46 (m, 2H), | |
| | | 3.38-3.33 (m, 1H), 2.24-2.15 | |
| | | (m, 1H), 2.10-2.05 (m, 1H); | |
| | | LCMS (M + H): 458.15 | |
| 34 | (S)-3-(4-((l-((4- | 1H-NMR (400 MHz, DMSO-D6) δ | 88 mg, |
| | methoxyphenyl)sulfonyl)pyrrolidin- | 7.94-7.91 (m, 2H), 7.70 (dt, | 22% yield |
| | 3-yl)oxy)phenyl)-5- | 2H), 7.10-7.06 (m, 2H), 6.88 | |
| | (trifluoromethyl)-1,2,4- | (dt, 2H), 5.03 (br, 1H), 3.85 | |
| | oxadiazole | (s, 3H), 3.49 (dd, 1H), 3.39- | |
| | | 3.33 (m, 2H), 3.25 (td, 1H), | |
| | | 2.14-2.05 (m, 1H), 1.99 (dd, | |
| | | 1H); LCMS (M + H): 470.00 | |
| 35 | (S)-3-(4-((1-((4- | 1H-NMR (400 MHz, DMSO-D6) δ | 91 mg, |
| | fluorophenyl)sulfonyl)pyrrolidin- | 7.94-7.91 (m, 2H), 7.87-7.82 | 23% yield |
| | 3-yl)oxy)phenyl)-5- | (m, 2H), 7.44-7.38 (m, 2H), | |
| | (trifluoromethyl)-1,2,4- | 6.87 (dt, 2H), 5.04 (br, 1H), | |
| | oxadiazole | 3.53 (dd, 1H), 3.43-3.35 (m, | |
| | | 2H), 3.28-3.23 (m, 1H), 2.13- | |
| | | 2.06 (m, 1H), 2.00 (dd, 1H); | |
| | | LCMS (M + H): 457.95 | |
| 40 | (S)-3-(4-((1- | 1H-NMR (400 MHz, DMSO-D6) δ | 129 mg, |
| | (ethylsulfonyl)pyrrolidin-3- | 8.00 (dt, 2H), 7.18 (dt, 2H), | 54% yield |
| | yl)oxy)phenyl)-5- | 5.19 (t, 1H), 3.65 (dd, 1H), | |
| | (trifluoromethyl)-1,2,4- | 3.47-3.38 (m, 3H), 3.18-3.05 | |
| | oxadiazole | (m, 2H), 2.31-2.22 (m, 1H), | |
| | | 2.15-2.11 (m, 1H), 1.24-1.16 | |
| | | (m, 3H); LCMS (M − 2): 389.10 | |
| 41 | (S)-3-(4-((1- | 1H-NMR (400 MHz, DMSO-D6) δ | 134 mg, |
| | (cyclopropylsulfonyl)pyrrolidin- | 7.98-8.02 (m, 2H), 7.18 (dt, | 55% yield |
| | 3-yl)oxy)phenyl)-5- | 2H), 5.20 (t, 1H), 3.67 (dd, | |
| | (trifluoromethyl)-1,2,4- | 1H), 3.48-3.44 (m, 3H), 2.70- | |
| | oxadiazole | 2.64 (m, 1H), 2.28 (m, 1H), | |
| | | 2.16-2.07 (m, 1H), 1.02-0.86 | |
| | | (m, 4H); LCMS (M + H): | |
| | | 404.00 | |
| 42 | (S)-3-(4-((1-((2,4- | 1H-NMR (400 MHz, DMSO-D6) δ | 107 mg, |
| | difluorophenyl)sulfonyl)pyrrolidin- | 7.96-7.92 (m, 2H), 7.86 (td, | 37% yield |
| | 3-yl)oxy)phenyl)-5- | 1H), 7.54 (ddd, 1H), 7.24 | |
| | (trifluoromethyl)-1,2,4- | (td, 1H), 6.96-6.92 (m, 2H), | |
| | oxadiazole | 5.10 (br, 1H), 3.61 (dd, 1H), | |
| | | 3.51-3.45 (m, 2H), 3.39-3.34 | |
| | | (m, 1H), 2.25-2.16 (m, 1H), | |
| | | 2.11-2.06 (m, 1H); LCMS (M + | |
| | | H): 475.90 | |
| 114 | (S)-5-(trifluoromethyl)- | 1H-NMR (400 MHz, DMSO-D6) δ | 156 mg, |
| | 3-(4-((1- | 8.03-8.00 (m, 2H), 7.21-7.18 | 43% yield |
| | ((trifluoromethyl)sulfonyl)pyrrolidin- | (m, 2H), 5.30 (br, 1H), 3.89- | |
| | 3-yl)oxy)phenyl)-1,2,4- | 3.61 (m, 4H), 2.41-2.24 (m, | |
| | oxadiazole | 2H); LCMS (M + H): 430.9 | |
| 115 | (S)-3-(4-((1- | 1H-NMR (400 MHz, DMSO-D6) δ | 224 mg, |
| | (propylsulfonyl)pyrrolidin- | 8.01-7.98 (m, 2H), 7.18-7.14 | 66% yield |
| | 3-yl)oxy)phenyl)-5- | (m, 2H), 5.17 (t, 1H), 3.66- | |
| | (trifluoromethyl)-1,2,4- | 3.36 (m, 4H), 3.14-2.97 (m, | |
| | oxadiazole | 2H), 2.30-2.10 (m, 2H), 1.88- | |
| | | 1.63 (m, 2H), 1.00 (m, 3H); | |
| | | LCMS (M+): 405.0 | |
| 116 | (S)-3-(4-((1-((4- | 1H-NMR (400 MHz, DMSO-D6) δ | 286 mg, |
| | bromophenyl)sulfonyl)pyrrolidin- | 7.94-7.89 (m, 2H), 7.83-7.79 | 66% yield |
| | 3-yl)oxy)phenyl)-5- | (m, 2H), 7.71-7.68 (m, 2H), | |
| | (trifluoromethyl)-1,2,4- | 6.83 (dd, 2H), 5.03 (br, 1H), | |
| | oxadiazole | 3.53 (dd, 1H), 3.44-3.35 (m, | |
| | | 2H), 3.27 (dd, 1H), 2.13- 1.98 | |
| | | (m, 2H); LCMS (M + H): 519.70 | |
| 117 | (S)-3-(4-((1-(pyridin-3- | 1H-NMR (400 MHz, DMSO-D6) δ | 251 mg, |
| | ylsulfonyl)pyrrolidin-3- | 8.95-8.87 (m, 2H), 8.20 (dt, | 68% yield |
| | yl)oxy)phenyl)-5- | 1H), 7.91 (d, 2H), 7.63 (dd, | |
| | (trifluoromethyl)-1,2,4- | 1H), 6.81 (d, 2H), 5.04 (br, | |
| | oxadiazole | 1H), 3.60-3.44 (m, 4H), 2.13- | |
| | | 2.02 (m, 2H); LCMS (M − H): | |
| | | 438.95 | |
| 118 | (S)-5-(trifluoromethyl)-3- | 1H-NMR (400 MHz, DMSO-D6) δ | 263 mg, |
| | (4-((1-((4- | 7.97 (dd, 4H), 7.90-7.86 (m, | 62% yield |
| | (trifluoromethyl)phenyl) | 2H), 6.78-6.74 (m, 2H), 5.04 | |
| | sulfonyl)pyrrolidin-3- | (br, 1H), 3.60-3.41 (m, 4H), | |
| | yl)oxy)phenyl)-1,2,4- | 2.18-2.09 (m, 1H), 2.05-1.98 | |
| | oxadiazole | (m, 1H); LCMS (M − H): 506.40 | |
| 119 | (S)-3-(4-((1- | 1H-NMR (400 MHz, DMSO-D6) δ | 259 mg, |
| | tosylpyrrolidin-3- | 7.92 (dt, 2H), 7.64 (dd, 2H), | 68% yield |
| | yl)oxy)phenyl)-5- | 7.37 (d, 2H), 6.86-6.82 (m, | |
| | (trifluoromethyl)-1,2,4- | 2H), 5.02 (br, 1H), 3.50 (dd, | |
| | oxadiazole | 1H), 3.39-3.33 (m, 2H), 3.28- | |
| | | 3.20 (m, 1H), 2.41 (s, 3H), | |
| | | 2.12-2.03 (m, 1H), 2.00-1.95 | |
| | | (m, 1H); LCMS (M + H): 454.05 | |
| 120 | (S)-3-(4-((1-((2,4- | 1H-NMR (400 MHz, DMSO-D6) δ | 334 mg, |
| | dichlorophenyl)sulfonyl)pyrrolidin- | 7.97-7.88 (m, 4H), 7.60 (dd, | 79% yield |
| | 3-yl)oxy)phenyl)- | 1H), 7.03 (d, 2H), 5.16 (br, | |
| | 5-(trifluoromethyl)-1,2,4- | 1H), 3.68-3.49 (m, 4H), 2.27 | |
| | oxadiazole | (s, 2H); LCMS (M+): 507.85 | |
| 121 | (S)-4-((3-(4-(5- | 1H-NMR (400 MHz, DMSO-D6) δ | 155 mg, |
| | (trifluoromethyl)-1,2,4- | 8.05-8.03 (m, 2H), 7.96-7.91 | 40% yield |
| | oxadiazol-3- | (m, 4H), 6.83 (dd, 2H), 5.04 | |
| | yl)phenoxy)pyrrolidin-1- | (br, 1H), 3.59-3.28 (m, 4H), | |
| | yl)sulfonyl)benzonitrile | 2.15-1.98 (m, 2H); LCMS | |
| | | (M − H): 462.95 | |
| 122 | (S)-3-(4-((1-((3- | 1H-NMR (400 MHz, DMSO-D6) δ | 116 mg, |
| | chlorophenyl)sulfonyl)pyrrolidin- | 7.93-7.90 (m, 2H), 7.81-7.74 | 29% yield |
| | 3-yl)oxy)phenyl)-5- | (m, 3H), 7.65-7.61 (m, 1H), | |
| | (trifluoromethyl)-1,2,4- | 6.83 (dd, 2H), 5.04 (br, 1H), | |
| | oxadiazole | 3.58-3.27 (m, 4H), 2.17-1.97 | |
| | | (m, 2H); LCMS (M − 2): 471.95 | |
| 136 | (S)-3-(4-((1- | 1H-NMR (400 MHz, DMSO-D6) δ | 195 mg, |
| | (isopropylsulfonyl)pyrrolidin- | 8.00 (dt, 2H), 7.19-7.16 (m, | 58% yield |
| | 3-yl)oxy)phenyl)-5- | 2H), 5.19 (t, 1H), 3.67 (dd, | |
| | (trifluoromethyl)-1,2,4- | 1H), 3.52-3.37 (m, 4H), 2.27 | |
| | oxadiazole | (m, 1H), 2.16-2.11 (m, 1H), | |
| | | 1.23 (t, 6H); LCMS (M − H): | |
| | | 403.95 | |
| 147 | (S)-3-(4-((1-((3-methylthiophen- | 1H-NMR (400 MHz, DMSO-D6) δ | 255 mg, |
| | 2-yl)sulfonyl)pyrrolidin- | 7.96-7.84 (m, 3H), 7.09-6.93 | 66% yield |
| | 3-yl)oxy)phenyl)-5- | (m, 3H), 5.10 (br, 1H), 3.60- | |
| | (trifluoromethyl)-1,2,4- | 3.34 (m, 4H), 2.36 (d, 3H), | |
| | oxadiazole | 2.21-2.03 (m, 2H); LCMS | |
| | | (M + H): 460.05 | |
| 148 | (S)-3-(4-((1-((1-methyl- | 1H-NMR (400 MHz, DMSO-D6) δ | 212 mg, |
| | 1H-imidazol-4- | 7.97 (dd, 2H), 7.77-7.76 (m, | 57% yield |
| | yl)sulfonyl)pyrrolidin-3- | 2H), 6.99 (d, 2H), 5.05 (br, | |
| | yl)oxy)phenyl)-5- | 1H), 3.65 (s, 3H), 3.56-3.61 | |
| | (trifluoromethyl)-1,2,4- | (m, 1H), 3.36-3.46 (m, 3H), | |
| | oxadiazole | 1.98-2.11 (m, 2H) LCMS | |
| | | (M + H): 444.15 | |

### Example 8: —Preparation of (S)-3-(4-((1-(4-methylbenzyl)pyrrolidin-3-yl)oxy)phenyl)-5-(trifluoromethyl)-1,2,4-oxadiazole (Compound No. 163)

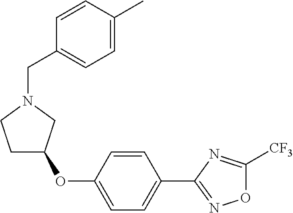 [SMILES: Cc1ccc(CN2CC[C@H](Oc3ccc(-c4noc(C(F)(F)F)n4)cc3)C2)cc1]

To a stirred solution of (S)-3-(4-(pyrrolidin-3-yloxy)phenyl)-5-(trifluoromethyl)-1,2,4-oxadiazole (0.3 g, 1 mmol) in acetonitrile (6 mL), N,N-diisopropylethylamine (0.4 mL, 2.6 mmol) was added at 0° C. followed by the addition of alpha-bromo-p-xylene (0.23 g, 1.3 mmol) at 0° C. The reaction mixture was stirred at 25° C. for 16 h. The reaction mixture was diluted with dichloromethane (10 mL) and washed with water (10 mL), dried over anhydrous sodium sulphate and concentrated under reduced pressure to obtain the crude product. The crude product was purified by flash column chromatography on silica gel using eluent 40% ethyl acetate in hexane to obtain (S)-3-(4-((1-(4-methylbenzyl)pyrrolidin-3-yl)oxy)phenyl)-5-(trifluoromethyl)-1,2,4-oxadiazole (0.13 g, 0.32 mmol, 32% yield).

1H-NMR (400 MHz, DMSO-D6) δ 7.97 (dt, 2H), 7.20 (d, 2H), 7.17-7.08 (m, 4H), 5.02-4.97 (m, 1H), 3.57 (s, 2H), 2.88-2.63 (m, 3H), 2.46-2.32 (m, 1H), 2.29 (d, 3H), 1.85-1.78 (m, 1H), 1.35-1.23 (m, 1H); LCMS (M+H): 404.55

TABLE 8
The following compounds were prepared by the procedure
analogous to that for the Compound No. 163

| Compound no. | Compound Name | 1H-NMR and LCMS | Yield |
|---|---|---|---|
| 137 | (S)-3-(4-((1- | 1H-NMR (400 MHz, DMSO-D6) δ | 108 mg, |
| | benzylpyrrolidin-3- | 7.96 (dt, 2H), 7.34-7.28 (m, | 28% yield |
| | yl)oxy)phenyl)-5- | 4H), 7.26-7.21 (m, 1H), 7.11- | |
| | (trifluoromethyl)-1,2,4- | 7.07 (m, 2H), 5.01-4.96 (m, | |
| | oxadiazole | 1H), 3.60 (s, 2H), 2.86 (dd, | |
| | | 1H), 2.74-2.64 (m, 2H), 2.43 | |
| | | (dd, 1H), 2.37-2.29 (m, 1H), | |
| | | 1.84-1.76 (m, 1H); LCMS | |
| | | (M + H): 390.35 | |
| 180 | (S)-3-(4-((1-(4- | 1H-NMR (400 MHz, DMSO-D6) δ | 150 mg, |
| | chlorobenzyl)pyrrolidin- | 7.95 (dt, 2H), 7.34 (m, 4H), | 35% yield |
| | 3-yl)oxy)phenyl)-5- | 7.10-7.06 (m, 2H), 5.00-4.96 | |
| | (trifluoromethyl)-1,2,4- | (m, 1H), 3.59 (s, 2H), 2.86 | |
| | oxadiazole | (dd, 1H), 2.73-2.63 (m, 1H), | |
| | | 2.45-2.30 (m, 2H), 1.80 (dd, | |
| | | 1H), 1.22 (dd, 1H); LCMS | |
| | | (M+): 423.95 | |
| 181 | (S)-3-(4-((1- | 1H-NMR (400 MHz, DMSO-D6) δ | 130 mg, |
| | isopropylpyrrolidin-3- | 8.01-7.98 (m, 2H), 7.17 (dd, | 46% yield |
| | yl)oxy)phenyl)-5- | 2H), 5.24 (br, 1H), 3.52 (d, | |
| | (trifluoromethyl)-1,2,4- | 5H), 2.16 (d, 1H), 1.32-1.26 | |
| | oxadiazole | (m, 6H), 1.23 (d, 1H); LCMS | |
| | | (M + H): 342.00 | |

### Example 9: —Preparation of (S)-(3-(2-fluoro-4-(5-(trifluoromethyl)-1,2,4-oxadiazol-3-yl)phenoxy)pyrrolidin-1-yl)(2-fluorophenyl)methanone (Compound No. 52)

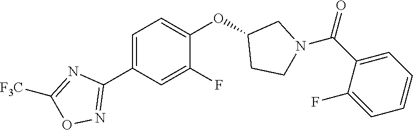 [SMILES: O=C(c1ccccc1F)N1CC[C@H](Oc2ccc(-c3noc(C(F)(F)F)n3)cc2F)C1]

### Step 1: —Preparation of tert-Butyl (S)-3-(4-cyano-2-fluorophenoxy)pyrrolidine-1-carboxylate

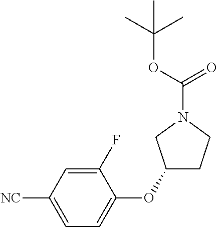 [SMILES: CC(C)(C)OC(=O)N1CC[C@H](Oc2ccc(C#N)cc2F)C1]

To a solution of tert-butyl-(S)-3-hydroxypyrrolidine-1-carboxylate (4.44 g, 23.7 mmol) in N,N-dimethylformamide (40 mL), sodium hydride (1.72 g, 43.1 mmol) was added under nitrogen atmosphere at 0° C. and stirred for 30 min. To the resulting reaction mixture, 3,4-difluorobenzonitrile (3 g, 21.6 mmol) in N,N-dimethylformamide (10 mL) was added slowly at 0° C. and stirred at 25° C. for 2 h. The reaction mixture was quenched with ammonium chloride solution and diluted with ethyl acetate (100 mL). The dichloromethane layer was collected and washed twice with water (80 mL), dried over anhydrous sodium sulphate and concentrated under reduced pressure to obtain the crude product. The crude product was purified by flash column chromatography on silica gel by eluent 50% ethyl acetate in hexane to obtain tert-butyl (S)-3-(4-cyano-2-fluorophenoxy)pyrrolidine-1-carboxylate (5.3 g, 17.3 mmol, 80% yield).

### Step 2: —Preparation of tert-Butyl (S)-3-(2-fluoro-4-(N′-hydroxycarbamimidoyl)phenoxy)pyrrolidine-1-carboxylate

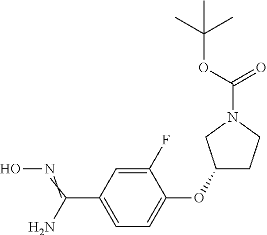 [SMILES: CC(C)(C)OC(=O)N1CC[C@H](Oc2ccc(C(N)=NO)cc2F)C1]

To a solution of tert-butyl-(S)-3-(4-cyano-2-fluorophenoxy)pyrrolidine-1-carboxylate (5 g, 16.3 mmol) in ethanol (55 mL), sodium bicarbonate (2.5 g, 29.4 mmol) and hydroxylamine hydrochloride (2.1 g, 29.4 mmol) were added and stirred at 65° C. for 16 h. The reaction mixture was filtered by using sintered funnel and washed twice with ethyl acetate (20 mL). The combined organic layer was concentrated under reduced pressure to obtain the compound of tert-butyl-(S)-3-(2-fluoro-4-(N′-hydroxycarbamimidoyl)phenoxy)pyrrolidine-1-carboxylate (5.3 g, 15.6 mmol, 96% yield).

### Step 3: —Preparation of tert-butyl-(S)-3-(2-fluoro-4-(5-(trifluoromethyl)-1,2,4-oxadiazol-3-yl)phenoxy)pyrrolidine-1-carboxylate (compound no 60)

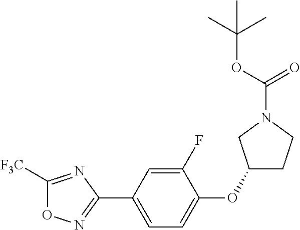 [SMILES: CC(C)(C)OC(=O)N1CC[C@H](Oc2ccc(-c3noc(C(F)(F)F)n3)cc2F)C1]

To a solution of tert-butyl-(S)-3-(2-fluoro-4-(N′-hydroxycarbamimidoyl)phenoxy)pyrrolidine-1-carboxylate (0.5 g, 1.5 mmol) in tetrahydrofuran (10 mL), trifluoroacetic anhydride (0.27 mL, 1.9 mmol) was added at 0° C. under nitrogen atmosphere and stirred at 25° C. for 16 h. The reaction mixture was diluted with ethyl acetate (25 mL) and washed with cold sodium bicarbonate solution (20 mL). The ethyl acetate layer was collected and dried over anhydrous sodium sulphate, concentrated under reduced pressure to obtain the crude product. The crude product was purified by flash column chromatography on silica gel using eluent 50% ethyl acetate in hexane to obtain tert-butyl (S)-3-(2-fluoro-4-(5-(trifluoromethyl)-1,2,4-oxadiazol-3-yl)phenoxy)pyrrolidine-1-carboxylate (216 mg, 0.52 mmol, 35% yield).

1H-NMR (400 MHz, DMSO-D6, at 80° C.) δ 7.89-7.80 (m, 2H), 7.47-7.40 (m, 1H), 5.19-5.17 (m, 1H), 3.64-3.60 (m, 1H), 3.50-3.36 (m, 3H), 2.26-2.17 (m, 1H), 2.13-2.08 (m, 1H), 1.41 (s, 9H); GCMS (M+H): 417.1

### Step 4: —Preparation of (S)-3-(3-fluoro-4-(pyrrolidin-3-yloxy)phenyl)-5-(trifluoromethyl)-1,2,4-oxadiazole

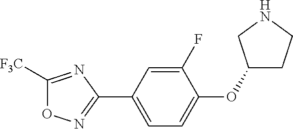 [SMILES: Fc1cc(-c2noc(C(F)(F)F)n2)ccc1O[C@H]1CCNC1]

To a solution of tert-butyl-(S)-3-(2-fluoro-4-(5-(trifluoromethyl)-1,2,4-oxadiazol-3-yl)phenoxy)pyrrolidine-1-carboxylate (0.12 g, 0.29 mmol) in dichloromethane (4 mL), trifluoroacetic acid (0.5 mL, 6.49 mmol) was added under nitrogen atmosphere at 0° C. and stirred for 4 h at 25° C. The reaction mixture was diluted with dichloromethane (10 mL) and washed with saturated sodium bicarbonate solution (10 mL), dichloromethane layer was separated, dried over anhydrous sodium sulphate and concentrated under reduced pressure to obtain (S)-3-(3-fluoro-4-(pyrrolidin-3-yloxy)phenyl)-5-(trifluoromethyl)-1,2,4-oxadiazole (0.08 g, 0.25 mmol, 88% yield).

### Step 5: —Preparation of (S)-(3-(2-fluoro-4-(5-(trifluoromethyl)-1,2,4-oxadiazol-3-yl)phenoxy)pyrrolidin-1-yl)(4-methoxyphenyl)methanone (Compound No. 52)

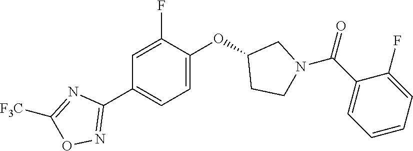 [SMILES: O=C(c1ccccc1F)N1CC[C@H](Oc2ccc(-c3noc(C(F)(F)F)n3)cc2F)C1]

To a solution of (S)-3-(3-fluoro-4-(pyrrolidin-3-yloxy)phenyl)-5-(trifluoromethyl)-1,2,4-oxadiazole (0.45 g, 1.4 mmol) in dichloromethane (6 mL), N,N-diisopropylethylamine (1.5 mL, 8.5 mmol) was added and stirred for 10 min at 25° C. To the resulting solution, 2-fluorobenzoyl chloride (0.2 mL, 1.7 mmol) was added and stirred at 25° C. for 16 h. The reaction mixture was diluted with dichloromethane (20 mL) and washed with water (20 mL), dichloromethane layer was dried over anhydrous sodium sulphate and concentrated under reduced pressure to obtain the crude product. The crude product was purified by preparative HPLC to obtain (S)-(3-(2-fluoro-4-(5-(trifluoromethyl)-1,2,4-oxadiazol-3-yl)phenoxy)pyrrolidin-1-yl)(2-fluorophenyl)methanone (0.45 mg, 1 mmol, 73% yield).

1H-NMR (400 MHz, DMSO-D6, at 80° C.) δ 7.85-7.76 (m, 2H), 7.46-7.37 (m, 3H), 7.23 (d, 2H), 5.22 (d, 1H), 3.87-3.38 (m, 4H), 2.28-2.16 (m, 2H); LCMS (M+H): 440.10

TABLE 9
The following compounds were prepared by the procedure
analogous to that for the Compound No. 52

| Compound no. | Compound Name | 1H-NMR and LCMS | Yield |
|---|---|---|---|
| 53 | (S)-(3-(2-fluoro-4-(5- | 1H-NMR (400 MHz, DMSO-D6, | 510 mg, |
| | (trifluoromethyl)-1,2,4- | at 80° C.) δ 7.85-7.80 (m, 2H), | 80% yield |
| | oxadiazol-3- | 7.51 (d, 2H), 7.43 (t, 1H), | |
| | yl)phenoxy)pyrrolidin-1- | 6.96 (d, 2H), 5.24 (s, 1H), | |
| | yl)(4- | 3.93-3.89 (m, 1H), 3.82 (d, | |
| | methoxyphenyl)methanone | 3H), 3.73-3.61 (m, 3H), 2.28- | |
| | | 2.23 (m, 1H), 2.20-2.15 (m, | |
| | | 1H); LCMS (M + H): 452.15 | |
| 54 | (S)-(3-(2-fluoro-4-(5- | 1H-NMR (400 MHz, DMSO-D6, | 398 mg, |
| | (trifluoromethyl)-1,2,4- | at 80° C.) δ 7.85-7.80 (m, 2H), | 63.9% yield |
| | oxadiazol-3- | 7.51-7.44 (m, 2H), 7.36-7.25 | |
| | yl)phenoxy)pyrrolidin-1- | (m, 3H), 5.25 (s, 1H), 3.91- | |
| | yl)(3- | 3.87 (m, 1H), 3.65 (s, 3H), | |
| | fluorophenyl)methanone | 2.34-2.16 (m, 2H); LCMS | |
| | | (M + H): 440.10 | |
| 58 | (S)-(3-(2-fluoro-4-(5- | 1H-NMR (400 MHz, DMSO-D6, | 458 mg, |
| | (trifluoromethyl)-1,2,4- | at 80° C.) δ 8.72 (d, 1H), | 76% yield |
| | oxadiazol-3- | 8.64 (dd, 1H), 7.93 (d, 1H), | |
| | yl)phenoxy)pyrrolidin-1- | 7.83-7.80 (m, 2H), 7.46-7.43 | |
| | yl)(pyridin-3-yl)methanone | (m, 2H), 5.26 (s, 1H), 3.95- | |
| | | 3.92 (m, 1H), 3.68 (br, 3H), | |
| | | 2.36-2.26 (m, 1H), 2.21-2.17 | |
| | | (m, 1H); LCMS (M + H): | |
| | | 423.10 | |
| 59 | (S)-(3-(2-fluoro-4-(5- | 1H-NMR (400 MHz, DMSO-D6, | 415 mg, |
| | (trifluoromethyl)-1,2,4- | at 80° C.) δ 8.67 (d, 2H), 7.82 | 69.3% yield |
| | oxadiazol-3- | (d, 2H), 7.46 (s, 3H), 5.28 | |
| | yl)phenoxy)pyrrolidin-1- | (br, 1H), 3.88-3.54 (m, 4H), | |
| | yl)(pyridin-4-yl)methanone | 2.35-2.26 (m, 1H), 2.21-2.07 | |
| | | (m, 1H); LCMS (M + H): 423.10 | |
| 61 | (S)-3-(4-((l- | 1H-NMR (400 MHz, DMSO-D6) δ | 84 mg, |
| | (ethylsulfonyl)pyrrolidin-3- | 7.87 (d, 2H), 7.45 (t, 1H), | 14.47% yield |
| | yl)oxy)-3-fluorophenyl)-5- | 5.26 (s, 1H), 3.69-3.64 (m, | |
| | (trifluoromethyl)-1,2,4- | 1H), 3.52-3.38 (m, 3H), 3.18- | |
| | oxadiazole | 3.07 (m, 2H), 2.32-2.23 (m, | |
| | | 1H), 2.19-2.14 (m, 1H), 1.22 | |
| | | (t, 3H); LCMS (M + H): 410.10 | |
| 69 | (S)-l-(3-(2-fluoro-4-(5- | 1H-NMR (400 MHz, DMSO-D6, | 236 mg, |
| | (trifluoromethyl)-1,2,4- | at 80° C.) δ 7.87-7.81 (m, 2H), | 41.7% yield |
| | oxadiazol-3- | 7.47-7.41 (m, 1H), 5.23 (d, | |
| | yl)phenoxy)pyrrolidin-1- | 1H), 3.89-3.36 (m, 4H), 2.35- | |
| | yl)ethan-l-one | 2.11 (m, 2H), 1.98-1.94 (m, | |
| | | 3H); LCMS (M + H): 360.00 | |

### Example 10: —Preparation of (S)-(3-(3-fluoro-4-(5-(trifluoromethyl)-1,2,4-oxadiazol-3-yl)phenoxy)pyrrolidin-1-yl)(2-fluorophenyl)methanone (Compound No. 63)

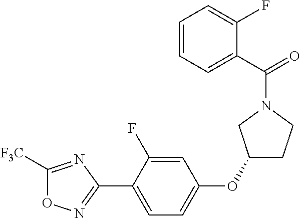 [SMILES: O=C(c1ccccc1F)N1CC[C@H](Oc2ccc(-c3noc(C(F)(F)F)n3)c(F)c2)C1]

### Step 1: —Preparation of tert-Butyl-(S)-3-(4-cyano-3-fluorophenoxy)pyrrolidine-1-carboxylate

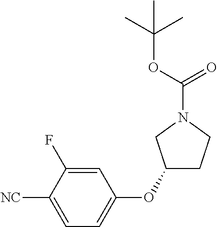 [SMILES: CC(C)(C)OC(=O)N1CC[C@H](Oc2ccc(C#N)c(F)c2)C1]

To a solution of tert-butyl (S)-3-hydroxypyrrolidine-1-carboxylate (1.85 g, 9.9 mmol) in toluene (20 mL) cesium carbonate (4.4 g, 13.5 mmol) and 4-bromo-2-fluorobenzonitrile (1.8 g, 9 mmol) were added under nitrogen atmosphere. The reaction mixture was degassed with nitrogen for 10 min at 25° C. To the above mixture (±)-2,2′-Bis(diphenylphosphino)-1,1′-binaphthalene (0.84 g, 1.3 mmol) and palladium(II) acetate (0.16 g, 0.7 mmol) were added and again degassed the reaction mixture for 15 min. The reaction mixture was stirred at 120° C. for 12 h. The reaction mixture was diluted with ethyl acetate (30 mL) and washed twice with water (25 mL). The ethyl acetate layer was collected and dried over anhydrous sodium sulphate and concentrated under reduced pressure to obtain the crude product. The crude residue was purified by column chromatography by eluting in 50% ethyl acetate in hexane on silica gel to obtain tert-butyl (S)-3-(4-cyano-3-fluorophenoxy)pyrrolidine-1-carboxylate (2.5 g, 8.2 mmol, 91% yield).

### Step 2: Preparation of tert-butyl (S)-3-(3-fluoro-4-(N′-hydroxycarbamimidoyl)phenoxy)pyrrolidine-1-carboxylate

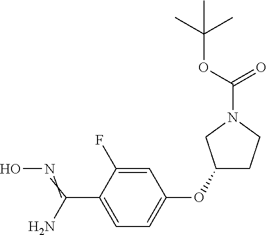 [SMILES: CC(C)(C)OC(=O)N1CC[C@H](Oc2ccc(C(N)=NO)c(F)c2)C1]

This compound was prepared according to the procedure mentioned for step 2 of Compound No. 52

### Step 3: Preparation of tert-butyl (S)-3-(3-fluoro-4-(5-(trifluoromethyl)-1,2,4-oxadiazol-3-yl)phenoxy)pyrrolidine-1-carboxylate (Compound No. 62)

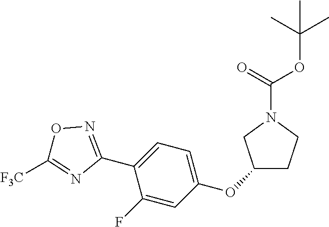 [SMILES: CC(C)(C)OC(=O)N1CC[C@H](Oc2ccc(-c3noc(C(F)(F)F)n3)c(F)c2)C1]

The compound was prepared according to the procedure mentioned for step 3 of Compound No. 52

### Step 4: Preparation of (S)-3-(2-fluoro-4-(pyrrolidin-3-yloxy)phenyl)-5-(trifluoromethyl)-1,2,4-oxadiazole

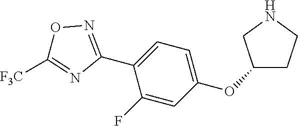 [SMILES: Fc1cc(O[C@H]2CCNC2)ccc1-c1noc(C(F)(F)F)n1]

The compound was prepared according to the procedure mentioned for step 4 of Compound No. 52

### Step 5: Preparation of (S)-(3-(3-fluoro-4-(5-(trifluoromethyl)-1,2,4-oxadiazol-3-yl)phenoxy)pyrrolidin-1-yl)(2-fluorophenyl)methanone (Compound No. 63)

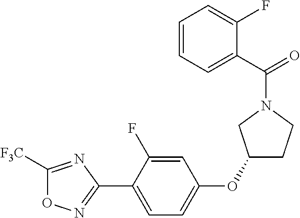 [SMILES: O=C(c1ccccc1F)N1CC[C@H](Oc2ccc(-c3noc(C(F)(F)F)n3)c(F)c2)C1]

The compound was prepared according to the procedure mentioned for step 5 of Compound No. 52

1H-NMR (400 MHz, DMSO-D6, at 80° C.) δ 8.00-7.92 (m, 1H), 7.48-7.41 (m, 2H), 7.26 (d, 2H), 7.17-6.99 (m, 2H), 5.23 (d, 1H), 3.91-3.34 (m, 4H), 2.32-2.16 (m, 2H); LCMS (M+H): 440.30

TABLE 10
The following compounds were prepared by the procedure
analogous to that for the Compound No. 63

| Compound no. | Compound Name | 1H-NMR and LCMS | Yield |
|---|---|---|---|
| 64 | (S)-(3-(3-fluoro-4-(5- | 1H-NMR (400 MHz, DMSO-D6, | 531 mg, |
| | (trifluoromethyl)-1,2,4- | at 80° C.) δ 8.02-7.91 (m, 1H), | 59% yield |
| | oxadiazol-3- | 7.56-7.44 (m, 1H), 7.40-7.27 | |
| | yl)phenoxy)pyrrolidin-1- | (m, 3H), 7.25-7.15 (m, 1H), | |
| | yl)(3- | 7.09-6.99 (m, 1H), 5.22 (d, | |
| | fluorophenyl)methanone | 1H), 3.94-3.87 (m, 1H), 3.70- | |
| | | 3.45 (m, 3H), 2.32-2.09 (m, | |
| | | 2H); LCMS (M + H): 439.85 | |
| 65 | (S)-(3-(3-fluoro-4-(5- | 1H-NMR (400 MHz, DMSO-D6, | 301 mg, |
| | (trifluoromethyl)-1,2,4- | at 80° C.) δ 8.72 (s, 1H), 8.64 | 34.8% yield |
| | oxadiazol-3- | (dd, 1H), 7.98-7.92 (m, 2H), | |
| | yl)phenoxy)pyrrolidin-1- | 7.45 (dd, 1H), 7.15-7.05 (m, | |
| | yl)(pyridin-3-yl)methanone | 2H), 5.24 (br, 1H), 3.95- | |
| | | 3.91 (m, 1H), 3.68 (br, 3H), | |
| | | 2.35-2.26 (m, 1H), 2.18-2.13 | |
| | | (m, 1H); LCMS (M + H): | |
| | | 423.05 | |
| 66 | (S)-(3-(3-fluoro-4-(5- | 1H-NMR (400 MHz, DMSO-D6, | 249 mg, |
| | (trifluoromethyl)-1,2,4- | at 80° C.) δ 8.66 (s, 2H), 7.96 | 28.8% yield |
| | oxadiazol-3- | (s, 1H), 7.46 (s, 2H), 7.13- | |
| | yl)phenoxy)pyrrolidin-1- | 7.05 (m, 2H), 5.25 (br, 1H), | |
| | yl)(pyridin-4-yl)methanone | 3.88-3.53 (m, 4H), 2.31-2.17 | |
| | | (m, 2H); LCMS (M + H): 423.05 | |
| 67 | (S)-3-(4-((1- | 1H-NMR (400 MHz, DMSO-D6, | 313 mg |
| | (ethylsulfonyl)pyrrolidin-3- | at 80° C.) δ 7.98 (t, 1H), 7.20 | 48.5% yield |
| | yl)oxy)-2-fluorophenyl)-5- | (dd, 1H), 7.04 (dd, 1H), 5.21 | |
| | (trifluoromethyl)-1,2,4- | (t, 1H), 3.65 (dd, 1H), 3.48- | |
| | oxadiazole | 3.37 (m, 3H), 3.20-3.03 (m, | |
| | | 2H), 2.32-2.11 (m, 2H), 1.24- | |
| | | 1.05 (m, 3H); LCMS (M + H): | |
| | | 410.11 | |
| 68 | (S)-1-(3-(3-fluoro-4-(5- | 1H-NMR (400 MHz, DMSO-D6, | 265 mg, |
| | (trifluoromethyl)-1,2,4- | at 80° C.) δ 7.99-7.95 (m, 1H), | 58.5% yield |
| | oxadiazol-3- | 7.14-7.02 (m, 2H), 5.21 (d, | |
| | yl)phenoxy)pyrrolidin-1- | 1H), 3.88-3.37 (m, 4H), 2.32- | |
| | yl)ethan-l-one | 2.10 (m, 2H), 1.97-1.90 (m, | |
| | | 3H); LCMS (M + H): 360.05 | |

### Example 11: —Preparation of (S)-(2-fluorophenyl)(3-((5-(5-(trifluoromethyl)-1,2,4-oxadiazol-3-yl)pyridin-2-yl)oxy)pyrrolidin-1-yl)methanone (Compound No. 45)

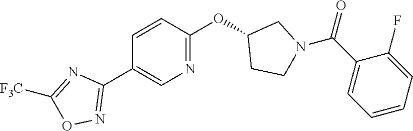 [SMILES: O=C(c1ccccc1F)N1CC[C@H](Oc2ccc(-c3noc(C(F)(F)F)n3)cn2)C1]

### Step 1: —Preparation of tert-butyl (S)-3-((5-cyanopyridin-2-yl)oxy)pyrrolidine-1-carboxylate

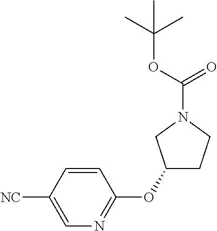 [SMILES: CC(C)(C)OC(=O)N1CC[C@H](Oc2ccc(C#N)cn2)C1]

To a stirred solution of tert-butyl (S)-3-hydroxypyrrolidine-1-carboxylate (3.6 g, 19 mmol) in dimethylformamide (30 mL), sodium hydride (1.3 g, 31.7 mmol) was added under nitrogen atmosphere at 0° C. The reaction was stirred for 30 min at 25° C., and then 2-bromo-5-cyanopyridine (2.9 g, 15.8 mmol) was added at 0° C. The reaction mixture was stirred for 2 h at 25° C. The reaction mixture was quenched by cautiously adding ice water. The reaction mixture was diluted with ethyl acetate (50 mL) and washed thrice with water (30 mL), dichloromethane layer was dried over anhydrous sodium sulphate and concentrated under reduced pressure to obtain the crude product. The crude product was purified by flash column chromatography on silica gel using eluent 50% ethyl acetate in hexane to obtain tert-butyl (S)-3-((5-cyanopyridin-2-yl)oxy)pyrrolidine-1-carboxylate (4.3 g, 14.9 mmol, 94% yield).

### Step 2: —Preparation of tert-butyl (S)-3-((5-(N′-hydroxycarbamimidoyl)pyridin-2-yl)oxy)pyrrolidine-1-carboxylate

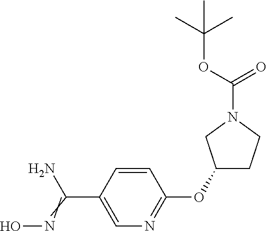 [SMILES: CC(C)(C)OC(=O)N1CC[C@H](Oc2ccc(C(N)=NO)cn2)C1]

To a stirred solution of tert-butyl (S)-3-((5-cyanopyridin-2-yl)oxy)pyrrolidine-1-carboxylate (4.2 g, 14.5 mmol) in ethanol (50 mL), sodium bicarbonate (2.4 g, 29 mmol) and hydroxylamine hydrochloride (2 g, 29 mmol) were added at 0° C. The reaction mixture was stirred for 12 h at 65° C. The reaction mixture was filtered, the filtrate was concentrated under reduced pressure to obtain tert-butyl (S)-3-((5-(N′-hydroxycarbamimidoyl)pyridin-2-yl)oxy)pyrrolidine-1-carboxylate (4.5 g, 14 mmol, 96% yield).

### Step 3: —Preparation of tert-butyl (S)-3-((5-(5-(trifluoromethyl)-1,2,4-oxadiazol-3-yl)pyridin-2-yl)oxy)pyrrolidine-1-carboxylate

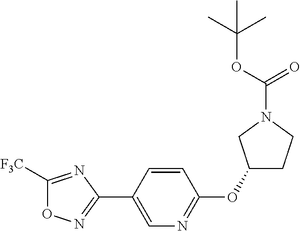 [SMILES: CC(C)(C)OC(=O)N1CC[C@H](Oc2ccc(-c3noc(C(F)(F)F)n3)cn2)C1]

To a stirred solution of tert-butyl (S)-3-((5-(N′-hydroxycarbamimidoyl)pyridin-2-yl)oxy)pyrrolidine-1-carboxylate (4.5 g, 14 mmol) in tetrahydrofuran (60 mL), trifluoroacetic anhydride (2.6 mL, 18.1 mmol) was added at 0° C. under nitrogen atmosphere. The reaction mixture was stirred for 16 h at 25° C. The reaction mixture was diluted with ethyl acetate (40 mL) and washed with ice cold saturated sodium bicarbonate solution (40 mL), ethyl acetate layer was dried over anhydrous sodium sulphate and concentrated under reduced pressure to obtain the crude product. The crude product was purified by flash column chromatography on silica gel using eluent 35% ethyl acetate in hexane to obtain tert-butyl (S)-3-((5-(5-(trifluoromethyl)-1,2,4-oxadiazol-3-yl)pyridin-2-yl)oxy)pyrrolidine-1-carboxylate (5.5 g, 13.7 mmol, 98% yield).

### Step 4: —Preparation of (S)-3-(6-(pyrrolidin-3-yloxy)pyridin-3-yl)-5-(trifluoromethyl)-1,2,4-oxadiazole

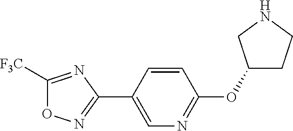 [SMILES: FC(F)(F)c1nc(-c2ccc(O[C@H]3CCNC3)nc2)no1]

To a stirred solution of tert-butyl (S)-3-((5-(5-(trifluoromethyl)-1,2,4-oxadiazol-3-yl)pyridin-2-yl)oxy)pyrrolidine-1-carboxylate (5.2 g, 13 mmol) in dichloromethane (40 mL), trifluoroacetic acid (2 mL, 26.mmol) was added at 0° C. under nitrogen atmosphere. The reaction mixture was stirred for 16 h at 25° C. The reaction mixture was concentrated under reduced pressure to obtain the crude product, the crude product was diluted with dichloromethane (40 mL) and washed with saturated sodium bi carbonate solution (40 mL), dichloromethane layer was separated, dried over anhydrous sodium sulphate and concentrated under reduced pressure to obtain (S)-3-(6-(pyrrolidin-3-yloxy)pyridin-3-yl)-5-(trifluoromethyl)-1,2,4-oxadiazole (3.8 g, 12.7 mmol, 97% yield).

### Step 5: —Preparation of (S)-(2-fluorophenyl)(3-((5-(5-(trifluoromethyl)-1,2,4-oxadiazol-3-yl)pyridin-2-yl)oxy)pyrrolidin-1-yl)methanone (Method A)

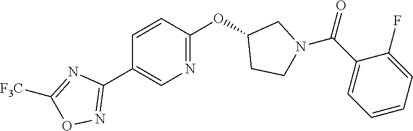 [SMILES: O=C(c1ccccc1F)N1CC[C@H](Oc2ccc(-c3noc(C(F)(F)F)n3)cn2)C1]

To a stirred solution of (S)-3-(6-(pyrrolidin-3-yloxy)pyridin-3-yl)-5-(trifluoromethyl)-1,2,4-oxadiazole (0.6 g, 2 mmol) in dichloromethane (6 mL), was added N,N-diisopropylethylamine (2.1 mL, 12 mmol) and stirred for 10 min at 25° C. followed by the addition of 2-fluorobenzoyl chloride (0.4 g, 2.4 mmol) at 0° C. The reaction mixture was stirred for 16 h at 25° C. The reaction mixture was diluted with dichloromethane (20 mL) and washed with water (20 mL), dichloromethane layer was dried over anhydrous sodium sulphate and concentrated under reduced pressure to obtain the crude product. The crude product was purified by preparative HPLC to obtain (S)-(2-fluorophenyl)(3-((5-(5-(trifluoromethyl)-1,2,4-oxadiazol-3-yl)pyridin-2-yl)oxy)pyrrolidin-1-yl)methanone (0.34 g, 0.8 mmol, 40% yield).

1H-NMR (400 MHz, DMSO-D6, at 80° C.) δ 8.82 (s, 1H), 8.29 (dd, 1H), 7.48 (dd, 1H), 7.36-7.24 (m, 3H), 7.05 (d, 1H), 5.67 (s, 1H), 3.93 (dd, 1H), 3.75-3.52 (m, 3H), 2.37-2.27 (m, 1H), 2.21-2.14 (m, 1H); LCMS (M+H): 423.35

### Step 5: —Preparation of (S)-(2-fluorophenyl)(3-45-(5-(trifluoromethyl)-1,2,4-oxadiazol-3-yl)pyridin-2-yl)oxy)pyrrolidin-1-yl)methanone (Compound No. 184) (Method B)

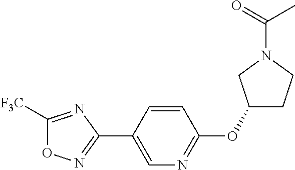 [SMILES: CC(=O)N1CC[C@H](Oc2ccc(-c3noc(C(F)(F)F)n3)cn2)C1]

To a stirred solution of (S)-3-(6-(pyrrolidin-3-yloxy)pyridin-3-yl)-5-(trifluoromethyl)-1,2,4-oxadiazole (0.2 g, 0.8 mmol) in dichloromethane (6 mL), triethylamine (0.3 mL, 2.5 mmol) was added. The reaction mixture was stirred for 10 min at 25° C., then acetyl chloride (0.1 g, 1.2 mmol)) was added at 0° C. The reaction mixture was stirred for 16 h at 25° C. The reaction mixture was diluted with dichloromethane (20 mL) and washed with water (20 mL), dichloromethane layer was dried over anhydrous sodium sulphate and concentrated under reduced pressure to obtain the crude product. The crude product was purified by preparative HPLC to obtain (S)-1-(3-((5-(5-(trifluoromethyl)-1,2,4-oxadiazol-3-yl)pyridin-2-yl)oxy)pyrrolidin-1-yl)ethan-1-one (0.2 g, 0.5 mmol, 67% yield).

1H-NMR (400 MHz, DMSO-D6) δ 8.84 (d, 1H), 8.29 (d, 1H), 7.04 (dd, 1H), 5.70-5.62 (m, 1H), 3.90-3.37 (m, 4H), 2.35-2.12 (m, 2H), 1.95 (d, 3H); LCMS (M+H): 343.25

### Step 5: —Preparation of (S)-(1-methyl-1H-pyrazol-3-yl)(3-45-(5-(trifluoromethyl)-1,2,4-oxadiazol-3-yl)pyridin-2-yl)oxy)pyrrolidin-1-yl)methanone (Compound No. 192) (Method C)

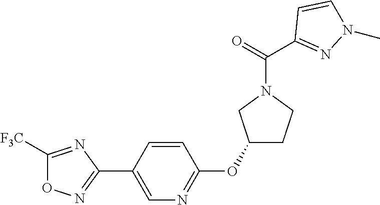 [SMILES: Cn1ccc(C(=O)N2CC[C@H](Oc3ccc(-c4noc(C(F)(F)F)n4)cn3)C2)n1]

To a stirred solution of 1-methyl-1H-pyrazole-3-carboxylic acid (0.1 g, 0.9 mmol) in dichloromethane (5 mL) under nitrogen atmosphere, 4-dimethylaminopyridine (0.3 g, 2.2 mmol) and 1-ethyl-3-(3-dimethylaminopropyl)carbodiimide (0.3 g, 1.5 mmol) were added at 0° C., then (S)-3-(6-(pyrrolidin-3-yloxy)pyridin-3-yl)-5-(trifluoromethyl)-1,2,4-oxadiazole (0.2 g, 0.7 mmol) was added at 0° C. The reaction mixture was stirred for 16 h at 25° C. The reaction mixture was diluted with dichloromethane (20 mL) and washed with water (30 mL), dichloromethane layer was dried over anhydrous sodium sulphate and concentrated under reduced pressure to obtain the crude product. The crude product was purified by flash column chromatography on silica gel using eluent 30% ethyl acetate in hexane to obtain (S)-(1-methyl-1H-pyrazol-3-yl)(3-((5-(5-(trifluoromethyl)-1,2,4-oxadiazol-3-yl)pyridin-2-yl)oxy)pyrrolidin-1-yl)methanone (0.24 g, 0.6 mmol, 82% yield.

1H-NMR @80° C. (400 MHz, DMSO-D6) δ 8.85 (s, 1H), 8.29 (d, 1H), 7.69 (s, 1H), 7.04 (d, 1H), 6.61 (d, 1H), 5.72 (br, 1H), 4.17 (s, 2H), 3.88-3.66 (m, 5H), 2.32-2.08 (m, 2H); LCMS (M+H): 409.50

### Step 5: —Preparation of (S)-2-methyl-1-(3-((5-(5-(trifluoromethyl)-1,2,4-oxadiazol-3-yl)pyridin-2-371)oxy)pyrrolidin-1-yl)propan-1-one: —(Compound No. 211) (Method D)

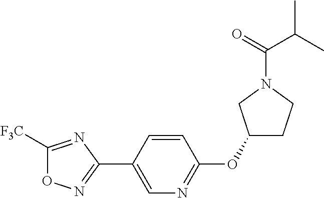 [SMILES: CC(C)C(=O)N1CC[C@H](Oc2ccc(-c3noc(C(F)(F)F)n3)cn2)C1]

To a stirred solution of (S)-3-(6-(pyrrolidin-3-yloxy)pyridin-3-yl)-5-(trifluoromethyl)-1,2,4-oxadiazole (0.2 g, 0.7 mmol) in dichloromethane (5 mL) under nitrogen atmosphere, triethylamine (0.3 mL, 2.2 mmol), 0-(7-Azabenzotriazol-1-yl)-N,N,N′,N′-tetramethyluronium hexafluorophosphate (0.3 g mg, 0.8 mmol) and isobutyric acid (84 mg, 0.9 mmol) were added at 0° C. The reaction mixture was stirred for 16 h at 25° C. The reaction mixture was diluted with dichloromethane (20 mL) and washed with water (30 mL), dichloromethane layer was dried over anhydrous sodium sulphate and concentrated under reduced pressure to obtain the crude product. The crude product was purified by flash column chromatography on silica gel using eluent 30% ethyl acetate in hexane to obtain (S)-2-methyl-1-(3-((5-(5-(trifluoromethyl)-1,2,4-oxadiazol-3-yl)pyridin-2-yl)oxy)pyrrolidin-1-yl)propan-1-one (0.2 g, 0.6 mmol, 84% yield).

1H-NMR (400 MHz, DMSO-D6, at 80° C.) δ 8.84 (d, 1H), 8.29 (dd, 1H), 7.04 (d, 1H), 5.66 (d, 1H), 3.70-3.47 (m, 4H), 2.72 (br, 1H), 2.32-2.12 (m, 2H), 1.03-0.99 (m, 6H); LCMS (M+H): 371.35

TABLE 11
The following compounds were prepared by the procedure analogous to
that for the Compound No. 45, Compound No 184, and Compound No 192

| Compound No. | Compound Name | 1H-NMR and LCMS | Yield | Method |
|---|---|---|---|---|
| 46 | (S)-(4- | 1H-NMR (400 MHz, DMSO-D6, at | 267 mg, | A |
| | methoxyphenyl)(3-((5- | 80° C.) δ 8.82 (d, 1H), 8.29 | 31% yield | |
| | (5-(trifluoromethyl)- | (dd, 1H), 7.52-7.49 (m, 2H), | | |
| | 1,2,4-oxadiazol-3- | 7.05 (d, 1H), 6.97-6.94 (m, | | |
| | yl)pyridin-2- | 2H), 5.68-5.64 (m, 1H), 3.95 | | |
| | yl)oxy)pyrrolidin-1- | (dd, 1H), 3.80 (s, 3H), 3.73- | | |
| | yl)methanone | 3.61 (m, 3H), 2.35-2.25 (m, | | |
| | | 1H), 2.20-2.13 (m, 1H); LCMS | | |
| | | (M + H): 435.40 | | |
| 47 | (S)-(3-fluorophenyl)(3- | 1H-NMR (400 MHz, DMSO-D6, at | 415 mg, | A |
| | ((5-(5-(trifluoromethyl)- | 80° C.) δ 8.82 (s, 1H), 8.29 | 49% yield | |
| | 1,2,4-oxadiazol-3- | (dd, 1H), 7.51-7.45 (m, 1H), | | |
| | yl)pyridin-2- | 7.36-7.24 (m, 3H), 7.05 (d, | | |
| | yl)oxy)pyrrolidin-1- | 1H), 5.67 (s, 1H), 3.93 (dd, | | |
| | yl)methanone | 1H), 3.72-3.54 (m, 3H), 2.36- | | |
| | | 2.27 (m, 1H), 2.21-2.14 (m, | | |
| | | 1H); LCMS (M + H): 423.35 | | |
| 48 | (S)-pyridin-3-yl(3-((5- | 1H-NMR (400 MHz, DMSO-D6, at | 226 mg, | A |
| | (5-(trifluoromethyl)- | 80° C.) δ 8.82 (s, 1H), 8.72 | 56% yield | |
| | 1,2,4-oxadiazol-3- | (s, 1H), 8.64 (d, 1H), 8.30 | | |
| | yl)pyridin-2- | (d, 1H), 7.93 (d, 1H), 7.45 | | |
| | yl)oxy)pyrrolidin-1- | (dd, 1H), 7.06 (d, 1H), 5.68 | | |
| | yl)methanone | (s, 1H), 3.96 (dd, 1H), 3.75- | | |
| | | 3.61 (m, 3H), 2.38-2.28 (m, | | |
| | | 1H), 2.23-2.15 (m, 1H); LCMS | | |
| | | (M + H): 406.15 | | |
| 49 | (S)-pyridin-4-yl(3-((5- | 1H-NMR (400 MHz, DMSO-D6, at | 134 mg, | A |
| | (5-(trifluoromethyl)- | 80° C.) δ 8.86-8.73 (m, 1H), | 33% yield | |
| | 1,2,4-oxadiazol-3- | 8.66-8.60 (m, 2H), 8.31-8.23 | | |
| | yl)pyridin-2- | (m, 1H), 7.43 (s, 2H), 7.02 | | |
| | yl)oxy)pyrrolidin-1- | (s, 1H), 5.65 (s, 1H), 3.88 | | |
| | yl)methanone | (s, 1H), 3.50-3.71 (m, 3H), | | |
| | | 2.32-2.26 (m, 1H), 2.21-2.11 | | |
| | | (m, 1H); LCMS (M + H): 406.20 | | |
| 185 | (S)-1-(3-((5-(5- | 1H-NMR (400 MHz, DMSO-D6) δ | 160 mg, | B |
| | (trifluoromethyl)-1,2,4- | 8.84 (d, 1H), 8.30 (dd, 1H), | 54% yield | |
| | oxadiazol-3-yl)pyridin- | 7.04 (d, 1H), 5.66 (d, 1H), | | |
| | 2-yl)oxy)pyrrolidin-1- | 3.89-3.42 (m, 4H), 2.35-2.12 | | |
| | yl)propan-1-one | (m, 4H), 1.01 (q, 3H); LCMS | | |
| | | (M + H): 357.20 | | |
| 193 | (S)-isoxazol-3-yl(3-((5- | 1H-NMR (400 MHz, DMSO-D6) δ | 227 mg | C |
| | (5-(trifluoromethyl)- | 9.07 (dd, 1H), 8.86 (m, 1H), | 78% | |
| | 1,2,4-oxadiazol-3- | 8.33-8.29 (m, 1H), 7.08 (td, | | |
| | yl)pyridin-2- | 1H), 6.87 (dd, 1H), 5.70 (q, | | |
| | yl)oxy)pyrrolidin-1- | 1H), 4.12-3.60 (m, 4H), 2.36- | | |
| | yl)methanone | 2.17 (m, 2H); LCMS (M + H): | | |
| | | 396.10 | | |
| 199 | (S)-oxazol-4-yl(3-((5-(5- | 1H-NMR (400 MHz, DMSO-D6) δ | 94 mg | C |
| | (trifluoromethyl)-1,2,4- | 8.86 (dd, 1H), 8.64 (dd, 1H), | 32% | |
| | oxadiazol-3-yl)pyridin- | 8.48 (dd, 1H), 8.33-8.28 (m, | | |
| | 2-yl)oxy)pyrrolidin-1- | 1H), 7.07 (t, 1H), 5.71-5.66 | | |
| | yl)methanone | (m, 1H), 4.19-3.59 (m, 4H), | | |
| | | 2.37-2.13 (m, 2H); LCMS (M + | | |
| | | H): 396.20 | | |
| 200 | (S)-thiazol-4-yl(3-((5- | 1H-NMR (400 MHz, DMSO-D6) δ | 201 mg | C |
| | (5-(trifluoromethyl)- | 9.16 (dd, 1H), 8.88-8.83 (m, | 67% | |
| | 1,2,4-oxadiazol-3- | 1H), 8.33-8.27 (m, 2H), 7.07 | | |
| | yl)pyridin-2- | (m, 1H), 5.68 (br, 1H), 4.20- | | |
| | yl)oxy)pyrrolidin-1- | 3.61 (m, 4H), 2.32-2.18 (m, | | |
| | yl)methanone | 2H); LCMS (M + H): 412.10 | | |
| 212 | (S)-cyclopropyl(3-((5- | 1H-NMR (400 MHz, DMSO-D6, at | 178 mg | B |
| | (5 -(trifluoromethyl)- | 80° C.) δ 8.84 (d, 1H), 8.30 | 66% | |
| | 1,2,4-oxadiazol-3- | (dd, 1H), 7.05 (d, 1H), 5.70 | | |
| | yl)pyridin-2- | (d, 1H), 4.03-3.46 (m, 4H), | | |
| | yl)oxy)pyrrolidin-1- | 2.39-2.14 (m, 2H), 1.79 (s, | | |
| | yl)methanone | 1H), 0.72-0.80 (m, 4H); LCMS | | |
| | | (M + H): 369.25 | | |

### Example 12: —Preparation of R)-(3-fluorophenyl)(3-((5-(5-(trifluoromethyl)-1,2,4-oxadiazol-3-yl)pyridin-2-yl)oxy)pyrrolidin-1-yl)methanone (compound no. 77)

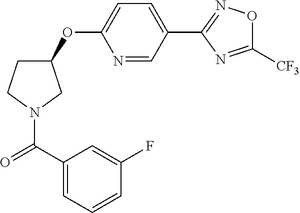 [SMILES: O=C(c1cccc(F)c1)N1CC[C@@H](Oc2ccc(-c3noc(C(F)(F)F)n3)cn2)C1]

### Step 1: —Preparation of tert-butyl (R)-3-hydroxypyrrolidine-1-carboxylate

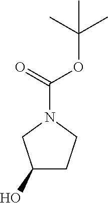

To the stirred solution of (R)-pyrrolidin-3-ol hydrochloride (10 g, 81 mmol) in dichloromethane (80 ml):methanol (20 ml) mixture at 0° C., triethylamine (22.6 ml, 160 mmol) was added and stirred for 10 min. Boc-anhydride (22.5 ml, 97 mmol) was added drop wise at 0° C. The reaction mixture heated to 25° C. and stirred for 16 h. Upon completion of the reaction, the reaction mixture was evaporated to dryness under reduced pressure. The residue was diluted with water (50 ml) and the product was extracted thrice with ethyl acetate (150 mL). The combined ethyl acetate layer was washed once with water (20 mL). The ethyl acetate layer was dried over anhydrous sodium sulphate and evaporated under reduced pressure to obtain tert-butyl (R)-3-hydroxypyrrolidine-1-carboxylate (15 g, 80 mmol, 99% yield).

### Step 2: —Preparation of tert-butyl (R)-3-((5-cyanopyridin-2-yl)oxy)pyrrolidine-1-carboxylate

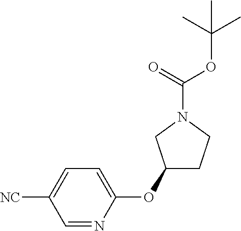 [SMILES: CC(C)(C)OC(=O)N1CC[C@@H](Oc2ccc(C#N)cn2)C1]

To the stirred solution of tert-butyl (R)-3-hydroxypyrrolidine-1-carboxylate (15 g, 80 mmol) in toluene (160 ml), 6-bromonicotinonitrile (17.6 g, 96 mmol), 2,2′-bis(diphenylphosphino)-1,1′-binaphthyl (7.5 g, 12 mmol) and cesium carbonate (52 g, 160 mmol) was added at 25° C. under nitrogen atmosphere. The reaction mixture was degassed with nitrogen for 10 min and added palladium (II) acetate (1.35 g, 6 mmol). The reaction mixture was further degassed with nitrogen gas for 10 min and heated to 100° C. in a sealed tube for 18 h. Upon completion of the reaction, the reaction mixture was diluted with water (100 mL) and product was extracted thrice by ethyl acetate (150 ml). The ethyl acetate layer was dried over anhydrous sodium sulphate and evaporated under reduced pressure. The crude compound was purified by flash column chromatography on silica gel using 60% ethyl acetate in hexane as an eluent to obtain tert-butyl (R)-3-((5-cyanopyridin-2-yl)oxy)pyrrolidine-1-carboxylate (17.25 g, 60 mmol, 74% yield).

### Step 3: —Preparation of tert-butyl (R)-3-((5-(N′-hydroxycarbamimidoyl)pyridin-2-yl)oxy)pyrrolidine-1-carboxylate

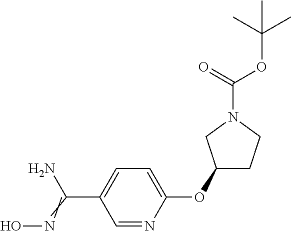 [SMILES: CC(C)(C)OC(=O)N1CC[C@@H](Oc2ccc(C(N)=NO)cn2)C1]

To the stirred solution of tert-butyl (R)-3-((5-cyanopyridin-2-yl)oxy)pyrrolidine-1-carboxylate (17.25 g, 60 mmol) in ethanol (170 mL), sodium bicarbonate (10 g, 120 mmol) and hydroxylamine hydrochloride (8.3 g, 120 mmol) were added under nitrogen atmosphere. The reaction mixture was heated to 80° C. for 16 h. Upon completion of the reaction, the reaction mixture was evaporated to dryness under reduced pressure to obtain tert-butyl(R)-3-((5-(N′-hydroxycarbamimidoyl)pyridin-2-yl)oxy)pyrrolidine-1-carboxylate (19.20 g, 59.6 mmol, 100% yield) as white solid.

### Step 4: —Preparation of tert-butyl (R)-3-((5-(5-(trifluoromethyl)-1,2,4-oxadiazol-3-yl)pyridin-2-yl)oxy)pyrrolidine-1-carboxylate (Compound 70)

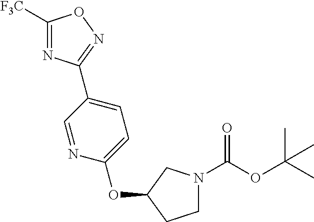 [SMILES: CC(C)(C)OC(=O)N1CC[C@@H](Oc2ccc(-c3noc(C(F)(F)F)n3)cn2)C1]

To the stirred solution of tert-butyl (R)-3-((5-(N′-hydroxycarbamimidoyl)pyridin-2-yl)oxy)pyrrolidine-1-carboxylate (21 g, 65 mmol) in tetrahydrofuran (210 mL) under nitrogen atmosphere, trifluoroacetic anhydride (9.2 mL, 65 mmol) was added at 0° C. The reaction mixture was stirred at 25° C. for 16 h. Upon completion of the reaction, the reaction mixture was quenched with saturated sodium bicarbonate solution (80 mL) till the pH 8.5. The product was extracted thrice with ethyl acetate (150 mL). The combined ethyl acetate layer was dried over anhydrous sodium sulphate and concentrated under reduced pressure. The crude compound was purified by flash column chromatography on silica gel using 65% ethyl acetate in hexane as an eluent to obtain tert-butyl (R)-3-((5-(5-(trifluoromethyl)-1,2,4-oxadiazol-3-yl)pyridin-2-yl)oxy)pyrrolidine-1-carboxylate (24 g, 60 mmol, 93% yield).

NMR data 1H-NMR (400 MHz, DMSO-D6) δ 8.85-8.84 (m, 1H), 8.30 (dd, 1H), 7.06 (d, 1H), 5.59 (s, 1H), 3.62 (td, 1H), 3.33-3.48 (m, 3H), 2.09-2.24 (m, 2H), 1.32-1.40 (m, 9H); LCMS (M): 400.36

### Step 5: —Preparation of (R)-3-(6-(pyrrolidin-3-yloxy)pyridin-3-yl)-5-(trifluoromethyl)-1,2,4-oxadiazole Hydrochloride

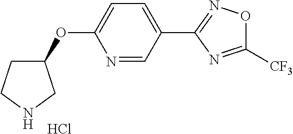 [SMILES: FC(F)(F)c1nc(-c2ccc(O[C@@H]3CCNC3)nc2)no1 ; Cl]

To the stirred solution of tert-butyl (R)-3-((5-(5-(trifluoromethyl)-1,2,4-oxadiazol-3-yl)pyridin-2-yl)oxy)pyrrolidine-1-carboxylate (24 g, 60 mmol) in dichloromethane (150 mL) at 0° C., 4M hydrochloric acid in 1,4-dioxane (181 mL, 724 mmol) was added drop wise under nitrogen atmosphere. The reaction mixture was stirred at 25° C. for 16 h. Upon completion of the reaction, the reaction mixture was evaporated to dryness under reduced pressure to obtain (R)-3-(6-(pyrrolidin-3-yloxy)pyridin-3-yl)-5-(trifluoromethyl)-1,2,4-oxadiazole hydrochloride (18 g, 53 mmol, 88% yield) as white solid.

### Step 6: —Preparation of (R)-(3-fluorophenyl)(3-((5-(5-(trifluoromethyl)-1,2,4-oxadiazol-3-yl)pyridin-2-yl)oxy)pyrrolidin-1-yl)methanone (Compound No. 77)

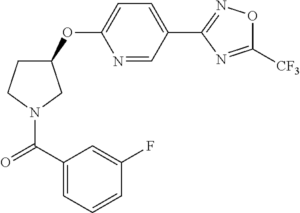 [SMILES: O=C(c1cccc(F)c1)N1CC[C@@H](Oc2ccc(-c3noc(C(F)(F)F)n3)cn2)C1]

To the stirred solution of (R)-3-(6-(pyrrolidin-3-yloxy)pyridin-3-yl)-5-(trifluoromethyl)-1,2,4-oxadiazole (0.4 g, 1.3 mmol) in dichloromethane (10 mL), triethylamine (0.93 ml, 6.7 mmol) was added slowly under nitrogen atmosphere. The reaction mixture was cooled to 0° C. and 3-fluorobenzoyl chloride (0.32 mL, 1.998 mmol) was added drop wise under nitrogen atmosphere. The reaction mixture was stirred at 25° C. for 16 h. Upon completion of the reaction, the reaction mixture was diluted with water (10 mL) and product was extracted thrice with dichloromethane (40 mL). The combined dichloromethane layer was dried over anhydrous sodium sulphate and concentrated under reduced pressure. The crude product was purified by flash column chromatography on silica gel using 65% ethyl acetate in hexane as an eluent to obtain (R)-(3-fluorophenyl)(3-((5-(5-(trifluoromethyl)-1,2,4-oxadiazol-3-yl)pyridin-2-yl)oxy)pyrrolidin-1-yl)methanone (0.47 g, 1.115 mmol, 84% yield).

1H-NMR (400 MHz, DMSO-D6) δ 8.89-8.75 (m, 1H), 8.37-8.26 (m, 1H), 7.58-7.25 (m, 4H), 7.14-7.00 (m, 1H), 5.71-5.56 (m, 1H), 4.00-3.87 (m, 1H), 3.74-3.60 (m, 2H), 3.58-3.45 (m, 1H), 2.36-2.08 (m, 2H); LCMS (M+H): 423

TABLE 12
The following compounds were prepared by the procedure
analogous to that for the Compound No. 77

| Comound No. | Compound Name | 1H-NMR and LCMS | Yield |
|---|---|---|---|
| 74 | (R)-(2-fluorophenyl)(3-((5- | NMR data 1H-NMR (400 MHz, | 0.3 g, 74% |
| | (5-(trifluoromethyl)-1,2,4- | DMSO-D6) δ 8.87-8.77 (m, 1H), 8.32- | |
| | oxadiazol-3-yl)pyridin-2- | 8.31 (m, 1H), 7.52-7.39 (m, 2H), 7.33- | |
| | yl)oxy)pyrrolidin-1- | 7.32 (m, 2H), 7.10-7.04 (m, 1H), 5.70- | |
| | yl)methanone | 5.58 (m, 1H), 3.91-3.33 (m, 4H), 2.36- | |
| | | 2.10 (m, 2H); LCMS (M + H): 423 | |
| 75 | (R)-(4-methoxyphenyl)(3- | 1H-NMR (400 MHz, DMSO-D6) δ | 0.3 g, 68% |
| | ((5-(5-(trifluoromethyl)- | 8.89-8.79 (m, 1H), 8.36-8.28 (m, 1H), | |
| | 1,2,4-oxadiazol-3-yl)pyridin- | 7.57-7.49 (m, 2H), 7.12-6.95 (m, 3H), | |
| | 2-yl)oxy)pyrrolidin-1- | 5.72-5.61 (m, 1H), 4.04-3.55 (m, 7H), | |
| | yl)methanone | 2.35-2.31 (m, 2H); LCMS(M + H): 435 | |

### Example 13: —Preparation of (R)-3-(6-((1-(phenylsulfonyl)pyrrolidin-3-yl)oxy)pyridin-3-yl)-5-(trifluoromethyl)-1,2,4-oxadiazole. (Compound No. 71)

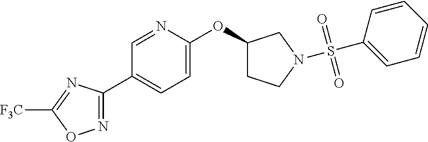 [SMILES: O=S(=O)(c1ccccc1)N1CC[C@@H](Oc2ccc(-c3noc(C(F)(F)F)n3)cn2)C1]

To the stirred solution of (R)-3-(6-(pyrrolidin-3-yloxy)pyridin-3-yl)-5-(trifluoromethyl)-1,2,4-oxadiazole (0.3 g, 1 mmol) in dichloromethane (10 mL), triethylamine (0.5 mL, 3.5 mmol) was added under nitrogen atmosphere. The reaction mixture was cooled to 0° C. and benzenesulfonyl chloride (0.2 mL, 1.5 mmol) was added. The reaction mixture was stirred at 25° C. for 16 h. Upon completion of the reaction, the reaction mixture was diluted with water (10 mL) and the product was extracted thrice with dichloromethane (45 mL). The combined dichloromethane layer was dried over anhydrous sodium sulphate and concentrated under reduced pressure. The crude compound was purified by flash column chromatography on silica gel using 65% ethyl acetate in hexane as an eluent to obtain (R)-3-(6-((1-(phenylsulfonyl)pyrrolidin-3-yl)oxy)pyridin-3-yl)-5-(trifluoromethyl)-1,2,4-oxadiazole (0.41 g, 0.9 mmol, 94% yield).

NMR data 1H-NMR (400 MHz, DMSO-D6) δ 8.78 (d, 1H), 8.23 (dd, 1H), 7.77 (dd, 2H), 7.68-7.72 (m, 1H), 7.56-7.60 (m, 2H), 6.56-6.58 (m, 1H), 5.44 (t, 1H), 3.55 (dd, 1H), 3.37-3.43 (m, 2H), 3.28 (dd, 1H), 2.11 (tt, 1H), 1.98-2.04 (m, 1H); LCMS (M+H): 441

TABLE 13
The following compounds were prepared by the procedure analogous to that for the Compound No. 71

| Compound No. | Compound Name | 1H-NMR and LCMS | Yield |
|---|---|---|---|
| 72 | (R)-3-(6-((1- | NMR data 1H-NMR (400 MHz, | 0.3 g, 55% yield |
| | (ethylsulfonyl)pyrrolidin- | DMSO-D6) δ 8.82 (dd, 1H), 8.30 (dd, | |
| | 3-yl)oxy)pyridin-3-yl)-5- | 1H), 7.04 (dd, 1H), 5.59-5.61 (m, 1H), | |
| | (trifluoromethyl)-1,2,4- | 3.65 (dd, 1H), 3.57-3.50 (m, 3H), 3.28- | |
| | oxadiazole | 3.04 (m, 2H), 2.11-2.31 (m, 2H), 1.19 | |
| | | (t, 3H); LCMS (M + H): 393 | |
| 73 | (R)-3-(6-((1-((4- | 1H-NMR (400 MHz, DMSO-D6) δ | 0.44 g, 72% yield |
| | fluorophenyl)sulfonyl)pyrrolidin- | 8.78-8.79 (m, 1H), 8.24 (dd, 1H), 7.82- | |
| | 3-yl)oxy)pyridin- | 7.88 (m, 2H), 7.36-7.43 (m, 2H), 6.62- | |
| | 3-yl)-5-(trifluoromethyl)- | 6.64 (m, 1H), 5.55-5.53 (m, 1H), 3.56 | |
| | 1,2,4-oxadiazole | (dd, 1H), 3.38-3.44 (m, 2H), 3.32-3.31 | |
| | | (m, 1H) 2.08-2.18 (m, 1H), 1.99-2.05 | |
| | | (m, 1H); LCMS(M + H): 459 | |
| 76 | (R)-3-(6-((1- | 1H-NMR (400 MHz, DMSO-D6) δ | 0.35 g, 65% yield |
| | (cyclopropylsulfonyl)pyrrolidin- | 8.85-8.85 (m, 1H), 8.32 (dd, 1H), 7.07- | |
| | 3-yl)oxy)pyridin-3- | 7.09 (m, 1H), 5.62-5.64 (m, 1H), 3.70 | |
| | yl)-5-(trifluoromethyl)- | (dd, 1H), 3.44-3.52 (m, 3H), 2.66-2.73 | |
| | 1,2,4-oxadiazole | (m, 1H), 2.26-2.35 (m, 1H), 2.14-2.19 | |
| | | (m, 1H), 0.86-1.02 (m, | |
| | | 4H); LCMS(M + H): 405 | |

### Example 14: —Preparation of (S)-3-(6-((1-(ethylsulfonyl)pyrrolidin-3-yl)oxy)pyridin-3-yl)-5-(trifluoromethyl)-1,2,4-oxadiazole (Compound No. 50)

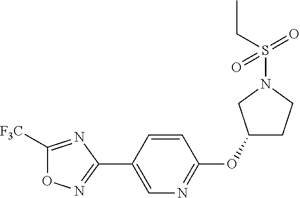 [SMILES: CCS(=O)(=O)N1CC[C@H](Oc2ccc(-c3noc(C(F)(F)F)n3)cn2)C1]

### Step 1: —Preparation of (S)-3-(6-((1-(ethylsulfonyl)pyrrolidin-3-yl)oxy)pyridin-3-yl)-5-(trifluoromethyl)-1,2,4-oxadiazole

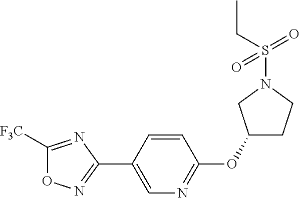 [SMILES: CCS(=O)(=O)N1CC[C@H](Oc2ccc(-c3noc(C(F)(F)F)n3)cn2)C1]

To a stirred solution of (S)-3-(6-(pyrrolidin-3-yloxy)pyridin-3-yl)-5-(trifluoromethyl)-1,2,4-oxadiazole (0.3 g, 1.1 mmol) in dichloromethane (5 mL), N,N-diisopropylethylamine (1.1 mL, 6.4 mmol) was added. The reaction mixture was stirred for 10 min at 25° C., then ethanesulfonyl chloride (0.2 g, 1.3 mmol) was added at 0° C. The reaction mixture was stirred for 16 h at 25° C. The reaction mixture was diluted with dichloromethane (20 mL) and washed with water (20 mL), dichloromethane layer was dried over anhydrous sodium sulphate and concentrated under reduced pressure to obtain the crude product. The crude product was purified by preparative HPLC to obtain (S)-3-(6-((1-(ethylsulfonyl)pyrrolidin-3-yl)oxy)pyridin-3-yl)-5-(trifluoromethyl)-1,2,4-oxadiazole (0.15 g, 0.4 mmol, 37% yield).

1H-NMR (400 MHz, DMSO-D6, at 80° C.) δ 8.84 (d, 1H), 8.31 (dd, 1H), 7.05 (d, 1H), 5.67-5.63 (m, 1H), 3.71 (dd, 1H), 3.49-3.45 (m, 3H), 3.16-3.07 (m, 2H), 2.36-2.28 (m, 1H), 2.20-2.14 (m, 1H), 1.25 (t, 3H); LCMS (M+H): 392.85

TABLE 14
The following compounds were prepared by the procedure analogous to that for the Compound No. 50

| Compound no. | Compound Name | 1H-NMR and LCMS | Yield |
|---|---|---|---|
| 104 | (S)-3-(6-((1-(phenylsulfonyl)pyrrolidin-3- | 1H-NMR (400 MHz, | 0.24 g, 83% yield |
| | yl)oxy)pyridin-3-yl)-5-(trifluoromethyl)- | DMSO-D6) δ 8.78 (dd, | |
| | 1,2,4-oxadiazole | 1H), 8.23 (dd, 1H), 7.78- | |
| | | 7.76 (m, 2H), 7.72-7.68 | |
| | | (m, 1H), 7.60-7.56 (m, | |
| | | 2H), 6.57 (dd, 1H), 5.45- | |
| | | 5.42 (m, 1H), 3.56 (dd, | |
| | | 1H), 3.43-3.26 (m, 3H), | |
| | | 2.16-1.99 (m, 2H); LCMS | |
| | | (M + H): 441.30 | |
| 105 | (S)-3-(6-((1-((4- | 1H-NMR (400 MHz, | 261 mg, 83% yield |
| | methoxyphenyl)sulfonyl)pyrrolidin-3- | DMSO-D6) δ 8.80 (dd, | |
| | yl)oxy)pyridin-3-yl)-5-(trifluoromethyl)- | 1H), 8.24 (dd, 1H), 7.70 | |
| | 1,2,4-oxadiazole | (dt, 2H), 7.07 (dt, 2H), | |
| | | 6.65 (dd, 1H), 5.43-5.41 | |
| | | (m, 1H), 3.87 (d, 3H), | |
| | | 3.53 (dd, 1H), 3.41-3.28 | |
| | | (m, 3H), 2.18-2.00 (m, | |
| | | 2H); LCMS (M + H): | |
| | | 471.35 | |
| 106 | (S)-3-(6-((1- | 1H-NMR (400 MHz, | 229 mg, 85% yield |
| | (cyclopropylsulfonyl)pyrrolidin- | DMSO-D6) δ 8.87 (dd, | |
| | 3-yl)oxy)pyridin-3-yl)-5- | 1H), 8.34 (dd, 1H), 7.10 | |
| | (trifluoromethyl)-1,2,4-oxadiazole | (dd, 1H), 5.66-5.54 (m, | |
| | | 1H), 3.72 (dd, 1H), 3.54- | |
| | | 3.46 (m, 3H), 2.78-2.68 | |
| | | (m, 1H), 2.37-2.28 (m, | |
| | | 1H), 2.23-2.15 (m, 1H), | |
| | | 1.04-0.86 (m, 4H); LCMS | |
| | | (M + H): 405.15 | |
| 107 | (S)-3-(6-((1-tosylpyrrolidin-3- | 1H-NMR (400 MHz, | 211 mg, 63% yield |
| | yl)oxy)pyridin-3-yl)-5-(trifluoromethyl)- | DMSO-D6) δ 8.80 (dd, | |
| | 1,2,4-oxadiazole | 1H), 8.27 (dd, 1H), 7.66- | |
| | | 7.64 (m, 2H), 7.36 (d, | |
| | | 2H), 6.58 (dd, 1H), 5.44- | |
| | | 5.42 (m, 1H), 3.54 (dd, | |
| | | 1H), 3.44-3.37 (m, 3H), | |
| | | 2.41 (s, 3H), 2.17-2.00 | |
| | | (m, 2H); LCMS (M + H): | |
| | | 455.55 | |
| 108 | (S)-3-(6-((1-((3- | 1H-NMR (400 MHz, | 221 mg, 63% yield |
| | chlorophenyl)sulfonyl)pyrrolidin-3- | DMSO-D6) δ 8.79 (dd, | |
| | yl)oxy)pyridin-3-yl)-5-(trifluoromethyl)- | 1H), 8.25 (dd, 1H), 7.79- | |
| | 1,2,4-oxadiazole | 7.73 (m, 3H), 7.62 (t, 1H), | |
| | | 6.54 (dd, 1H), 5.44 (br, | |
| | | 1H), 3.59 (dd, 1H), 3.49- | |
| | | 3.43 (m, 2H), 3.35 (dd, | |
| | | 1H), 2.20-2.11 (m, 1H), | |
| | | 2.08-2.03 (m, 1H); LCMS | |
| | | (M+): 475.00 | |
| 109 | (S)-5-(trifluoromethyl)-3-(6-((1-((4- | 1H-NMR (400 MHz, | 234 mg, 62% yield |
| | (trifluoromethyl)phenyl)sulfonyl)pyrrolidin- | DMSO-D6) δ 8.78-8.77 | |
| | 3-yl)oxy)pyridin-3-yl)-1,2,4-oxadiazole | (m, 1H), 8.17 (dd, 1H), | |
| | | 7.99 (d, 2H), 7.92 (d, 2H), | |
| | | 6.49-6.47 (m, 1H), 5.43 | |
| | | (br, 1H), 3.60 (dd, 1H), | |
| | | 3.50-3.36 (m, 3H), 2.18- | |
| | | 2.11 (m, 1H), 2.07-2.03 | |
| | | (m, 1H); LCMS (M + H): | |
| | | 509.05 | |
| 110 | (S)-3-(6-((1-(propylsulfonyl)pyrrolidin-3- | 1H-NMR (400 MHz, | 198 mg, 66% yield |
| | yl)oxy)pyridin-3-yl)-5-(trifluoromethyl)- | DMSO-D6) δ 8.85 (dd, | |
| | 1,2,4-oxadiazole | 1H), 8.33 (dd, 1H), 7.05 | |
| | | (dd, 1H), 5.63-5.61 (m, | |
| | | 1H), 3.67 (dd, 1H), 3.49- | |
| | | 3.41 (m, 3H), 3.15-3.00 | |
| | | (m, 2H), 2.34-2.13 (m, | |
| | | 2H), 1.73-1.64 (m, 2H), | |
| | | 0.98 (t, 3H); LCMS | |
| | | (M + H): 407.15 | |
| 111 | (S)-3-(6-((1-(methylsulfonyl)pyrrolidin-3- | 1H-NMR (400 MHz, | 192 mg, 69% yield |
| | yl)oxy)pyridin-3-yl)-5-(trifluoromethyl)- | DMSO-D6) δ 8.85 (t, | |
| | 1,2,4-oxadiazole | 1H), 8.33 (dd, 1H), 7.07 | |
| | | (dd, 1H), 5.63-5.61 (m, | |
| | | 1H), 3.65 (dd, 1H), 3.46- | |
| | | 3.35 (m, 3H), 2.92 (s, | |
| | | 3H), 2.33-2.24 (m, 1H), | |
| | | 2.18-2.12 (m, 1H); LCMS | |
| | | (M + H): 378.95 | |
| 112 | (S)-3-(6-((1-(m-tolylsulfonyl)pyrrolidin-3- | 1H-NMR (400 MHz, | 272 mg, 82% yield |
| | yl)oxy)pyridin-3-yl)-5-(trifluoromethyl)- | DMSO-D6) δ 8.78 (dd, | |
| | 1,2,4-oxadiazole | 1H), 8.24 (dd, 1H), 7.56- | |
| | | 7.54 (m, 2H), 7.51-7.44 | |
| | | (m, 2H), 6.55 (dd, 1H), | |
| | | 5.42 (br, 1H), 3.55 (dd, | |
| | | 1H), 3.44-3.26 (m, 3H), | |
| | | 2.31 (s, 3H), 2.13-1.98 | |
| | | (m, 2H); LCMS (M + H): | |
| | | 455.05 | |
| 149 | (S)-3-(6-((1-((3- | 1H-NMR (400 MHz, | 210 mg, 62% yield |
| | fluorophenyl)sulfonyl)pyrrolidin-3- | DMSO-D6) δ 8.79 (dd, | |
| | yl)oxy)pyridin-3-yl)-5-(trifluoromethyl)- | 1H), 8.24 (dd, 1H), 7.67- | |
| | 1,2,4-oxadiazole | 7.54 (m, 4H), 6.57 (dd, | |
| | | 1H), 5.4 (br, 1H), 3.59 | |
| | | (dd, 1H), 3.49-3.42 (m, | |
| | | 2H), 3.36-3.32 (m, 1H), | |
| | | 2.19-1.98 (m, 2H); | |
| | | LCMS (M + H): 459.50 | |
| 150 | (S)-4-((3-((5-(5-(trifluoromethyl)-1,2,4- | 1H-NMR (400 MHz, | 234 mg, 69% yield |
| | oxadiazol-3-yl)pyridin-2-yl)oxy)pyrrolidin- | DMSO-D6) δ 8.79 (t, | |
| | 1-yl)sulfonyl)benzonitrile | 1H), 8.24 (dd, 1H), 8.02 | |
| | | (dd, 2H), 7.95-7.93 (m, | |
| | | 2H), 6.61-6.59 (m, 1H), | |
| | | 5.43 (br, 1H), 3.60 (dd, | |
| | | 1H), 3.50-3.44 (m, 2H), | |
| | | 3.39-3.34 (m, 1H), 2.19- | |
| | | 2.10 (m, 1H), 2.07-2.02 | |
| | | (m, 1H); LCMS (M + H): | |
| | | 466.05 | |
| 151 | (S)-5-(trifluoromethyl)-3-(6-((1-((3- | 1H-NMR (400 MHz, | 278 mg, 75% yield |
| | (trifluoromethyl)phenyl)sulfonyl)pyrrolidin- | DMSO-D6) δ 8.79 (dd, | |
| | 3-yl)oxy)pyridin-3-yl)-1,2,4-oxadiazole | 1H), 8.23 (dd, 1H), 8.13 | |
| | | (q, 2H), 7.98 (s, 1H), 7.87 | |
| | | (t, 1H), 6.48 (dd, 1H), | |
| | | 5.45 (br, 1H), 3.63 (dd, | |
| | | 1H), 3.53-3.48 (m, 2H), | |
| | | 3.37 (dd, 1H), 2.34-2.05 | |
| | | (m, 2H); LCMS (M + H): | |
| | | 508.90 | |
| 152 | (S)-3-(6-((1-((2- | 1H-NMR (400 MHz, | 251 mg, 75% yield |
| | fluorophenyl)sulfonyl)pyrrolidin-3- | DMSO-D6) δ 8.80 (dd, | |
| | yl)oxy)pyridin-3-yl)-5-(trifluoromethyl)- | 1H), 8.26 (dd, 1H), 7.82- | |
| | 1,2,4-oxadiazole | 7.70 (m, 2H), 7.45-7.35 | |
| | | (m, 2H), 6.66 (dd, 1H), | |
| | | 5.51 (br, 1H), 3.65 (dd, | |
| | | 1H), 3.57 (d, 1H), 3.48 | |
| | | (td, 1H), 3.38 (td, 1H), | |
| | | 2.27-1.98 (m, 2H); LCMS | |
| | | (M + H): 459.50 | |
| 153 | (S)-3-(6-((1-(pyridin-3- | 1H-NMR (400 MHz, | 197 mg, 61% yield |
| | ylsulfonyl)pyrrolidin-3-yl)oxy)pyridin-3- | DMSO-D6) δ 8.95 (dd, | |
| | yl)-5-(trifluoromethyl)-1,2,4-oxadiazole | 1H), 8.87 (dd, 1H), 8.78 | |
| | | (dd, 1H), 8.25-8.19 (m, | |
| | | 2H), 7.62 (ddd, 1H), 6.51 | |
| | | (dd, 1H), 5.46 (br, 1H), | |
| | | 3.63-3.32 (m, 4H), 2.20- | |
| | | 1.95 (m, 2H); LCMS | |
| | | (M + H): 442.10 | |
| 154 | (S)-3-(6-((1-(benzylsulfonyl)pyrrolidin-3- | 1H-NMR (400 MHz, | 176 mg, 53% yield |
| | yl)oxy)pyridin-3-yl)-5-(trifluoromethyl)- | DMSO-D6) δ 8.85 (dd, | |
| | 1,2,4-oxadiazole | 1H), 8.35 (dd, 1H), 7.44 | |
| | | (dd, 2H), 7.39-7.31 (m, | |
| | | 3H), 7.11 (dd, 1H), 5.62 | |
| | | (br, 1H), 4.50 (dd, 2H), | |
| | | 3.63 (dd, 1H), 3.43-3.36 | |
| | | (m, 3H), 2.34-2.09 (m, | |
| | | 2H); LCMS (M + H): | |
| | | 454.95 | |
| 155 | (S)-3-(6-((1-(isopropylsulfonyl)pyrrolidin- | 1H-NMR (400 MHz, | 198 mg, 66% yield |
| | 3-yl)oxy)pyridin-3-yl)-5-(trifluoromethyl)- | DMSO-D6) δ 8.87 (dd, | |
| | 1,2,4-oxadiazole | 1H), 8.34 (dd, 1H), 7.08 | |
| | | (dd, 1H), 5.65 (br, 1H), | |
| | | 3.73 (dd, 1H), 3.54-3.40 | |
| | | (m, 4H), 2.36-2.16 (m, | |
| | | 2H), 1.25 (t, 6H); LCMS | |
| | | (M + H): 407.20 | |
| 160 | (S)-3-(6-((1-((2- | 1H-NMR (400 MHz, | 302 mg, 87% yield |
| | chlorophenyl)sulfonyl)pyrrolidin-3- | DMSO-D6) δ 8.83 (dd, | |
| | yl)oxy)pyridin-3-yl)-5-(trifluoromethyl)- | 1H), 8.31 (dd, 1H), 8.00- | |
| | 1,2,4-oxadiazole | 7.97 (m, 1H), 7.70-7.64 | |
| | | (m, 2H), 7.57-7.53 (m, | |
| | | 1H), 6.87 (dd, 1H), 5.60 | |
| | | (br, 1H), 3.72 (dd, 1H), | |
| | | 3.65-3.62 (m, 1H), 3.58- | |
| | | 3.48 (m, 2H), 2.35-2.15 | |
| | | (m, 2H); LCMS (M + H): | |
| | | 476.00 | |
| 161 | (S)-3-(6-((1-((4- | 1H-NMR (400 MHz, | 165 mg, 46% yield |
| | chlorobenzyl)sulfonyl)pyrrolidin-3- | DMSO-D6) δ 8.85 (dd, | |
| | yl)oxy)pyridin-3-yl)-5-(trifluoromethyl)- | 1H), 8.35 (dd, 1H), 7.47- | |
| | 1,2,4-oxadiazole | 7.42 (m, 4H), 7.11 (dd, | |
| | | 1H), 5.61 (br, 1H), 4.53 | |
| | | (dd, 2H), 3.64 (dd, 1H), | |
| | | 3.46-3.36 (m, 3H), 2.34- | |
| | | 2.00 (m, 2H); LCMS | |
| | | (M + H): 490.00 | |
| 162 | (S)-3-(6-((1-((2- | 1H-NMR (400 MHz, | 254 mg, 72% yield |
| | methoxyethyl)sulfonyl)pyrrolidin-3- | DMSO-D6) δ 8.87 (dd, | |
| | yl)oxy)pyridin-3-yl)-5-(trifluoromethyl)- | 1H), 8.35 (dd, 1H), 7.08 | |
| | 1,2,4-oxadiazole | (dd, 1H), 5.65-5.63 (m, | |
| | | 1H), 3.73-3.66 (m, 3H), | |
| | | 3.49-3.38 (m, 5H), 3.26 | |
| | | (s, 3H), 2.34 -1.92 (m, | |
| | | 2H); LCMS (M + H): | |
| | | 423.50 | |
| 175 | (S)-3-(6-((1-(phenethylsulfonyl)pyrrolidin- | 1H-NMR (400 MHz, | 284 mg, 84% yield |
| | 3-yl)oxy)pyridin-3-yl)-5-(trifluoromethyl)- | DMSO-D6) δ 8.84 (dd, | |
| | 1,2,4-oxadiazole | 1H), 8.29 (dd, 1H), 7.33- | |
| | | 7.28 (m, 4H), 7.25-7.20 | |
| | | (m, 1H), 6.99 (dd, 1H), | |
| | | 5.62-5.60 (m, 1H), 3.70 | |
| | | (dd, 1H), 3.52-3.36 (m, | |
| | | 5H), 3.02-2.95 (m, 2H), | |
| | | 2.32-2.12 (m, 2H); LCMS | |
| | | (M + H): 468.95 | |

### Example 15: —Preparation of p-tolyl(4-(4-(5-(trifluoromethyl)-1,2,4-oxadiazol-3-yl)phenoxy)piperidin-1-yl)methanone (Compound No-103)

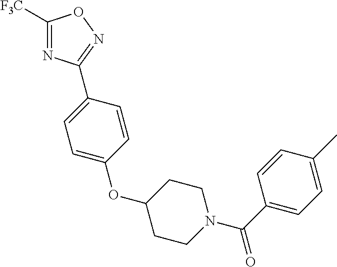 [SMILES: Cc1ccc(C(=O)N2CCC(Oc3ccc(-c4noc(C(F)(F)F)n4)cc3)CC2)cc1]

### Step 1: —Preparation of tert-butyl 4-(4-cyanophenoxy)piperidine-1-carboxylate

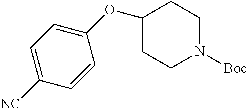 [SMILES: CC(C)(C)OC(=O)N1CCC(Oc2ccc(C#N)cc2)CC1]

To the stirred solution of tert-butyl 4-hydroxypiperidine-1-carboxylate (26 g, 130 mmol) in N,N-dimethyl formamide (100 mL) under nitrogen atmosphere at 0° C., sodium hydride (6 g, 149 mmol) was added in portions and reaction mixture was stirred at 0° C. for 20 min. 4-fluorobenzonitrile (15 g, 124 mmol) was dissolved in 20 mL of N,N-dimethyl formamide and added drop wise at 0° C. The reaction mixture was heated to 25° C. and stirred for 16 h, then cooled to 0° C. and quenched by adding water (100 mL) drop wise. The product was extracted twice by ethyl acetate (200 mL). The combined ethyl acetate layer was washed with ice cold water (300 mL), dried over anhydrous sodium sulphate and concentrated under reduced pressure to obtain a crude product. The crude product was purified by flash column chromatography using 40% ethyl acetate in hexane as an eluent to obtain tert-butyl 4-(4-cyanophenoxy)piperidine-1-carboxylate (37.2 g, 123 mmol, 99% yield).

### Step 2: —Preparation of tert-butyl 4-(4-(N′-hydroxycarbamimidoyl)phenoxy)piperidine-1-carboxylate

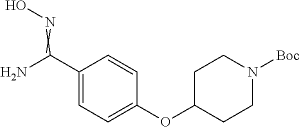 [SMILES: CC(C)(C)OC(=O)N1CCC(Oc2ccc(C(N)=NO)cc2)CC1]

To the stirred solution of tert-butyl 4-(4-cyanophenoxy)piperidine-1-carboxylate (37 g, 123 mmol) in ethanol (400 mL) under inert atmosphere, hydroxylamine hydrochloride (17.10 g, 246 mmol) and sodium bicarbonate (20.67 g, 246 mmol) were added. The resulting reaction mixture was heated to 80° C. for 16 h. Upon completion of the reaction, the reaction mixture was filtered through celite bed. Celite bed was washed twice with ethyl acetate (50 mL), the filtrate was distilled to dryness to obtain tert-butyl 4-(4-(N′-hydroxycarbamimidoyl)phenoxy)piperidine-1-carboxylate (41 g, 122 mmol, 99% yield).

### Step 3: —Preparation of tert-butyl 4-(4-(5-(trifluoromethyl)-1,2,4-oxadiazol-3-yl)phenoxy)piperidine-1-carboxylate (Compound No. 84)

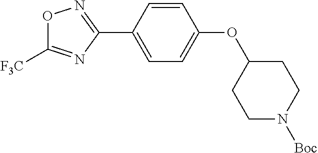 [SMILES: CC(C)(C)OC(=O)N1CCC(Oc2ccc(-c3noc(C(F)(F)F)n3)cc2)CC1]

To the stirred solution of tert-butyl 4-(4-(N′-hydroxycarbamimidoyl)phenoxy)piperidine-1-carboxylate (41 g, 122 mmol) in tetrahydrofuran (400 mL) at 0° C., trifluoroacetic anhydride (25.9 mL, 183 mmol) was added drop wise. The resulting reaction mixture was heated to 25° C. and stirred for 16 h. Upon completion of the reaction, the reaction mixture was cooled to 0° C. and saturated sodium bicarbonate solution (80 mL) was added drop wise to the reaction mixture till pH 7.5 to 8 was attained. The product was extracted thrice with ethyl acetate (200 mL). The combined ethyl acetate layer was dried over anhydrous sodium sulphate and evaporated under reduced pressure to obtain a crude compound. The crude compound was purified by flash column chromatography using 20% ethyl acetate in hexane as an eluent to obtain tert-butyl 4-(4-(5-(trifluoromethyl)-1,2,4-oxadiazol-3-yl)phenoxy)piperidine-1-carboxylate (39 g, 94 mmol, 77% yield) as white solid.

1H-NMR (400 MHz, CHLOROFORM-D) δ 8.09-8.05 (m, 2H), 7.04 (dt, 2H), 4.64-4.58 (m, 1H), 3.76-3.70 (m, 2H), 3.41 (dq, 2H), 2.03-1.95 (m, 2H), 1.85-1.66 (m, 2H), 1.50 (s, 9H); LCMS (M+H): 413.9

### Step 4: —Preparation of 3-(4-(piperidin-4-yloxy)phenyl)-5-(trifluoromethyl)-1,2,4-oxadiazole Hydrochloride (Compound No. 85)

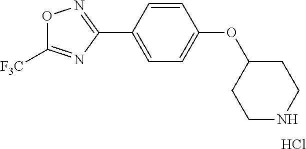 [SMILES: FC(F)(F)c1nc(-c2ccc(OC3CCNCC3)cc2)no1 ; Cl]

To the stirred solution of tert-butyl 4-(4-(5-(trifluoromethyl)-1,2,4-oxadiazol-3-yl)phenoxy)piperidine-1-carboxylate (20 g, 48 mmol) in dichloromethane (200 mL) under nitrogen atmosphere at 0° C., hydrogen chloride in 1, 4-dioxane (60 mL, 240 mmol) was added. The resulting reaction mixture was stirred at 25° C. for 16 h. Upon completion of the reaction, the reaction mixture was evaporated to dryness. The residual solid was washed with n-hexane. The suspension was filtered and washed once with n-hexane (20 mL). The solid obtained was dried under reduced pressure to obtain 3-(4-(piperidin-4-yloxy)phenyl)-5-(trifluoromethyl)-1,2,4-oxadiazole hydrochloride (17 g, 48 mmol, 100% yield).

1H-NMR (400 MHz, CHLOROFORM-D) δ 8.09-8.05 (m, 2H), 7.04 (dt, 2H), 4.64-4.58 (m, 1H), 3.76-3.70 (m, 2H), 3.41 (dq, 2H), 2.03-1.95 (m, 2H), 1.85-1.66 (m, 2H), 1.50 (s, 9H); LCMS (M+H): 413.9

### Step 5: —Preparation of p-tolyl(4-(4-(5-(trifluoromethyl)-1,2,4-oxadiazol-3-yl)phenoxy)piperidin-1-yl)methanone (Compound No. 103) (Method-1

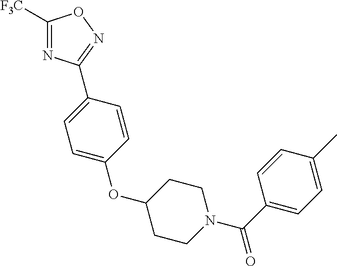 [SMILES: Cc1ccc(C(=O)N2CCC(Oc3ccc(-c4noc(C(F)(F)F)n4)cc3)CC2)cc1]

To the stirred solution of 3-(4-(piperidin-4-yloxy)phenyl)-5-(trifluoromethyl)-1,2,4-oxadiazole hydrochloride (0.3 g, 0.86 mmol) in dichloromethane (10 mL) under nitrogen atmosphere at 25° C., triethylamine (0.5 mL, 3.43 mmol) was added drop wise. The resulting reaction mixture was cooled to 0° C. and p-toluoyl chloride (0.14 mL, 1 mmol) was added drop wise. The reaction mixture was heated to 25° C. and stirred for 3 h. Upon completion of the reaction, the reaction mixture was diluted with water (10 mL) and the product was extracted twice by dichloromethane (40 mL). The combined dichloromethane layer was washed with water (10 mL), dried over anhydrous sodium sulphate and evaporated under reduced pressure to obtain a crude compound. The crude compound was purified by flash column chromatography using 60% ethyl acetate in hexane as an eluent to obtain p-tolyl(4-(4-(5-(trifluoromethyl)-1,2,4-oxadiazol-3-yl)phenoxy)piperidin-1-yl)methanone (0.35 g, 0.8 mmol, 95% yield).

1H-NMR (400 MHz, CHLOROFORM-D) δ 8.09-7.98 (m, 2H), 7.54-7.34 (m, 2H), 7.24 (d, 2H), 7.07-7.02 (m, 2H), 4.74-4.69 (m, 1H), 3.89 (d, 2H), 2.41 (s, 3H), 2.07-1.94- (m, 4H); LCMS (M+H): 432.05

TABLE 15
The following compounds were prepared by the procedure analogous to that for the Compound No. 103

| Compound No. | Compound Name | 1H-NMR and LCMS | Yield |
|---|---|---|---|
| 81 | phenyl(4-(4-(5-(trifluoromethyl)- | 1H-NMR (400 MHz, DMSO-D6) δ | 0.24 g, 72% |
| | 1,2,4-oxadiazol-3- | 8.02-7.99 (m, 2H), 7.47-7.41 (m, | |
| | yl)phenoxy)piperidin-1- | 5H), 7.25-7.21 (m, 2H), 4.86-4.81 | |
| | yl)methanone | (m, 1H), 3.59-3.38 (m, 2H), 2.05 | |
| | | (d, 2H), 1.69 (s, 2H); LCMS | |
| | | (M + H): 418.1 | |
| 92 | (2-fluorophenyl)(4-(4-(5- | 1H-NMR (400 MHz, | 0.34 g, 91% yield |
| | (trifluoromethyl)-1,2,4-oxadiazol- | CHLOROFORM-D) δ 8.05 (dt, | |
| | 3-yl)phenoxy)piperidin-1- | 2H), 7.43-7.37 (m, 2H), 7.24-7.19 | |
| | yl)methanone | (m, 1H), 7.14-7.08 (m, 1H), 7.05- | |
| | | 7.00 (m, 2H), 4.71 (s, 1H), 3.94 (s, | |
| | | 2H), 3.59 (s, 1H), 3.32 (d, 1H), | |
| | | 2.12-1.87 (m, 4H); LCMS (M + H): | |
| | | 436.10 | |
| 95 | l-(4-(4-(5-(trifluoromethyl)-1,2,4- | 1H-NMR (400 MHz, | 0.28 g, 92% yield |
| | oxadiazol-3- | CHLOROFORM-D) δ 8.10-8.01 | |
| | yl)phenoxy)piperidin-1-yl)ethan- | (m, 2H), 7.07-7.02 (m, 2H), 4.71- | |
| | 1-one | 4.66 (m, 1H), 3.74 (s, 3H), 3.58- | |
| | | 3.49 (m, 1H), 2.31-2.07 (m, 3H), | |
| | | 1.94 (d, 3H), 1.78 (d, −2H); LCMS | |
| | | (M + H): 356.10 | |
| 102 | (4-(4-(5-(trifluoromethyl)-1,2,4- | 1H-NMR (400 MHz, | 0.11 g, 26% yield |
| | oxadiazol-3- | CHLOROFORM-D) δ 8.06 (dt, | |
| | yl)phenoxy)piperidin-1-yl)(4- | 2H), 7.71 (t, 2H), 7.54 (d, 2H), 7.03 | |
| | (trifluoromethyl)phenyl)methanone | (dt, 2H), 4.75-4.70 (m, 1H), 3.92 (s, | |
| | | 2H), 3.65 (s, 1H), 3.40 (s, 1H), | |
| | | 2.09-1.86 (m, 4H); LCMS (M + H): | |
| | | 486.05 | |
| 131 | (3-chlorophenyl)(4-(4-(5- | 1H-NMR (400 MHz, | 320 mg, 83% |
| | (trifluoromethyl)-1,2,4-oxadiazol- | CHLOROFORM-D) δ 8.10-7.98 | |
| | 3-yl)phenoxy)piperidin-1- | (m, 2H), 7.45-7.31 (m, 4H), 7.07- | |
| | yl)methanone | 7.02 (m, 2H), 4.76-4.72 (m, 1H), | |
| | | 4.03-3.48 (m, 4H), 2.04 (d, 4H); | |
| | | LCMS (M + H): 452 | |
| 138 | 2-phenyl-1-(4-(4-(5- | 1H-NMR (400 MHz, | 0.26 g, 70% yield |
| | (trifluoromethyl)-1,2,4-oxadiazol- | CHLOROFORM-D) δ 8.05 (dt, | |
| | 3-yl)phenoxy)piperidin-1- | 2H), 7.55-7.34 (m, 3H), 7.30 (s, | |
| | yl)ethan-1-one | 2H), 7.00 (dt, 2H), 4.64-4.59(m, | |
| | | 1H), 3.80-3.65 (m, 5H), 3.50-3.44 | |
| | | (m, 1H), 1.99-1.91 (m, 1H), 1.85 | |
| | | (dt, 1H), 1.71-1.64 (m, 2H); LCMS | |
| | | (M + H): 432.30 | |
| 139 | 2,2-dimethyl-1-(4-(4-(5- | 1H-NMR (400 MHz, | 0.2 g, 60% yield |
| | (trifluoromethyl)-1,2,4-oxadiazol- | CHLOROFORM-D) δ 8.09-8.06 | |
| | 3-yl)phenoxy)piperidin-1- | (m, 2H), 7.05 (dt, 2H), 4.71-4.66 | |
| | yl)propan-1-one | (m, 1H), 3.92-3.86 (m, 2H), 3.66 | |
| | | (dq, 2H), 2.05-1.83 (m, 4H), 1.43- | |
| | | 1.17 (m, 9H); LCMS | |
| | | (M + H): 398.30 | |
| 140 | (4-methoxyphenyl)(4-(4-(5- | 1H-NMR (400 MHz, | 0.3 g, 92% yield |
| | (trifluoromethyl)-1,2,4-oxadiazol- | CHLOROFORM-D) δ 8.14-8.06 | |
| | 3-yl)phenoxy)piperidin-1- | (m, 2H), 7.65-7.41 (m, 2H), 7.16- | |
| | yl)methanone | 6.92 (m, 4H), 4.80-4.69 (m, 1H), | |
| | | 4.04-3.82 (m, 4H), 4.04-3.86 (m, | |
| | | 3H), 2.17-1.93 (m, 4H); LCMS | |
| | | (M + H): 448.20 | |
| 142 | (4-fluorophenyl)(4-(4-(5- | 1H-NMR (400 MHz, | 0.26 g, 70% yield |
| | (trifluoromethyl)-1,2,4-oxadiazol- | CHLOROFORM-D) δ 8.10-8.06 | |
| | 3-yl)phenoxy)piperidin-1- | (m, 2H), 7.54-7.44 (m, 2H), 7.15- | |
| | yl)methanone | 7.10 (m, 2H), 7.06-7.02 (m, 2H), | |
| | | 4.76-4.71- (m, 1H), 3.94-3.52 (m, | |
| | | 4H), 2.00 (d, 4H); LCMS | |
| | | (M + H): 436.25 | |
| 145 | 2-(4-chlorophenyl)-1-(4-(4-(5- | 1H-NMR (400 MHz, | 340 mg, 85% |
| | (trifluoromethyl)-1,2,4-oxadiazol- | CHLOROFORM-D) δ 8.06 (dt, | |
| | 3-yl)phenoxy)piperidin-1- | 2H), 7.33 (dt, 2H), 7.23 (d, 2H), | |
| | yl)ethan-1-one | 7.03-7.00 (m, 2H), 4.66-4.62 (m, | |
| | | 1H), 3.91-3.65 (m, 5H), 3.50-3.44 | |
| | | (m, 1H), 2.03-1.76(m, 4H); LCMS | |
| | | (M + H): 466.2 | |
| 159 | m-tolyl(4-(4-(5-(trifluoromethyl)- | 1H-NMR (400 MHz, | 0.35 g, 95% yield |
| | 1,2,4-oxadiazol-3- | CHLOROFORM-D) δ 8.05 (dt, | |
| | yl)phenoxy)piperidin-1- | 2H), 7.29 (dd, 1H), 7.21 (q, 3H), | |
| | yl)methanone | 7.04-7.01 (m, 2H), 4.72-4.67 (m, | |
| | | 1H), 3.93-3.70 (m, 3H), 3.43 (s, | |
| | | 1H), 2.38 (s, 3H), 2.04-1.94- (m, | |
| | | 4H); LCMS (M + H): 432.60 | |
| 179 | pyridin-4-yl(4-(4-(5- | 1H-NMR (400 MHz, DMSO-D6) δ | 130 mg, 36% |
| | (trifluoromethyl)-1,2,4-oxadiazol- | 8.68 (dd, 2H), 8.02-7.99 (m, 2H), | |
| | 3-yl)phenoxy)piperidin-1- | 7.43 (dd, 2H), 7.23 (dt, 2H), 4.87- | |
| | yl)methanone | 4.82 (m, 1H), 4.04 (s, 1H), 3.53- | |
| | | 3.48 (m, 2H), 3.28 (s, 1H), 2.09- | |
| | | 1.96 (m, 2H), 1.77-1.66 (m, 2H); | |
| | | LCMS (M + H): 419.2 | |
| 188 | 2-(4-methoxyphenyl)-1-(4-(4-(5- | 1H-NMR (400 MHz, DMSO-D6) δ | 390 mg, 99% |
| | (trifluoromethyl)-1,2,4-oxadiazol- | 7.97 (dd, 2H), 7.19-7.12 (m, 4H), | |
| | 3-yl)phenoxy)piperidin-1- | 6.89-6.84 (m, 2H), 4.75-4.69 (m, | |
| | yl)ethan-1-one | 1H), 3.92-3.74 (m, 5H), 3.65 (s, | |
| | | 2H), 3.33 (d, 2H), 1.98-1.89 (m, | |
| | | 2H), 1.53 (d, 2H); LCMS (M + H): | |
| | | 462.4 | |
| 194 | (4-(dimethylamino)phenyl)(4-(4- | 1H-NMR (400 MHz, DMSO-D6) δ | 250 mg, 63% |
| | (5-(trifluoromethyl)-1,2,4- | 7.99-7.97 (m, 2H), 7.30-7.27 (m, | |
| | oxadiazol-3- | 2H), 7.20 (dd, 2H), 6.72-6.69 (m, | |
| | yl)phenoxy)piperidin-1- | 2H), 4.79 (td, 1H), 3.85-3.79 (m, | |
| | yl)methanone | 2H), 3.44-3.38 (m, 2H), 2.94 (d, | |
| | | 6H), 2.04-1.98 (m, 2H), 1.67 (tt, | |
| | | 2H); LCMS (M + H): 461.45 | |
| 205 | (4-chlorophenyl)(4-(4-(5- | 1H-NMR (400 MHz, DMSO-D6) δ | 0.35 g, 90% yield |
| | (trifluoromethyl)-1,2,4-oxadiazol- | 7.98 (dt, 2H), 7.50-7.43 (m, 4H), | |
| | 3-yl)phenoxy)piperidin-1- | 7.20 (dt, 2H), 4.82-4.79 (m, 1H), | |
| | yl)methanone | 3.76 (s, 2H), 3.41 (t, 2H), 2.02 (d, | |
| | | 2H), 1.74-1.66 (m, 2H); LCMS | |
| | | (M + H): 452.10 | |

### Step 1: —Preparation of (4-chloro-3-(trifluoromethyl)phenyl)(4-(4-(5-(trifluoromethyl)-1,2,4-oxadiazol-3-yl)phenoxy)piperidin-1-yl)methanone (Compound No.-213) (Method-2)

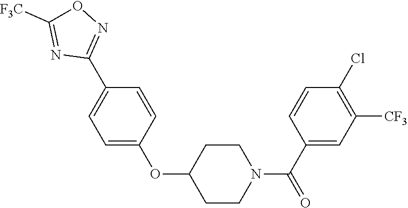 [SMILES: O=C(c1ccc(Cl)c(C(F)(F)F)c1)N1CCC(Oc2ccc(-c3noc(C(F)(F)F)n3)cc2)CC1]

To the stirred solution of 3-(4-(piperidin-4-yloxy)phenyl)-5-(trifluoromethyl)-1,2,4-oxadiazole hydrochloride (0.25 g, 0.7 mmol) in N,N-dimethyl formamide (7 mL) under nitrogen atmosphere 4-chloro-3-(trifluoromethyl)benzoic acid (0.2 g, 0.86 mmol) and triethylamine (0.4 ml, 2.9 mmol) were added. The reaction mixture was stirred at 25° C. for 5 min and then HATU (0.408 g, 1.072 mmol) was added. The reaction mixture was stirred at 25° C. for 16 h. Upon completion of the reaction, the reaction mixture was diluted with water (10 mL) and product was extracted twice by ethyl acetate (50 mL). The combined ethyl acetate layer was washed with ice cold water (50 mL), dried over anhydrous sodium sulphate and evaporated under reduced pressure to obtain a crude compound. The crude compound was purified by flash column chromatography using 60% ethyl acetate in hexane as an eluent to obtain (4-chloro-3-(trifluoromethyl)phenyl)(4-(4-(5-(trifluoromethyl)-1,2,4-oxadiazol-3-yl)phenoxy)piperidin-1-yl)methanone (0.2 g, 0.4 mmol, 54% yield).

1H-NMR (400 MHz, DMSO-D6) δ 7.97-8.00 (m, 2H), 7.86 (d, 1H), 7.78 (d, 1H), 7.74 (dd, 1H), 7.23-7.19 (m, 2H), 4.84-4.79 (m, 1H), 3.78 (d, 2H), 3.45 (s, 2H), 2.03 (s, 2H), 1.78-1.69 (m, 2H); LCMS (M+H): 519.95

### Example 16: —Preparation of (R)-1-(3-((4-(5-(trifluoromethyl)-1,2,4-oxadiazol-3-yl)phenyl)thio)pyrrolidin-1-yl)ethan-1-one (Compound No.-165)

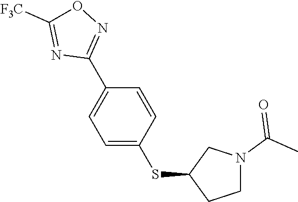 [SMILES: CC(=O)N1CC[C@@H](Sc2ccc(-c3noc(C(F)(F)F)n3)cc2)C1]

### Step 1: —Preparation of 4-mercaptobenzonitrile

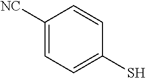

To the stirred solution of 4-bromobenzonitrile (5 g, 27.5 mmol) in dimethyl sulfoxide (100 mL), copper (II) sulfate (0.2 g, 1.4 mmol) and cesium carbonate (45 g, 137 mmol) were added. The reaction mixture was degassed with nitrogen for 10 min, 1,2-ethanedithiol (4.6 mL, 55 mmol) was added and the reaction mixture was heated to 100° C. for 20 h. Upon completion of the reaction, the reaction mixture was heated to 25° C. and quenched with 10% hydrochloric acid solution till the pH 1-2 was attained. Ethyl acetate (40 mL) was added to aqueous layer and biphasic mixture was filtered through celite bed to remove insoluble inorganic junk. Aqueous layer was extracted thrice with ethyl acetate (180 mL). The combined ethyl acetate layer was washed with ice cold water (90 mL), dried over anhydrous sodium sulphate and concentrated under reduced pressure to obtain 4-mercaptobenzonitrile (3.7 g, 27 mmol, 100% yield).

### Step 2: —Preparation of tert-butyl (R)-3-((4-cyanophenyl)thio)pyrrolidine-1-carboxylate

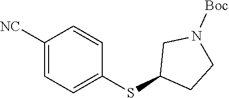 [SMILES: CC(C)(C)OC(=O)N1CC[C@@H](Sc2ccc(C#N)cc2)C1]

To the stirred solution of 4-mercaptobenzonitrile (7.4 g, 49 mmol) and tert-butyl (S)-3-hydroxypyrrolidine-1-carboxylate (9.2 g, 49 mmol) in tetrahydrofuran (70 mL), triphenylphosphine (19.4 g, 74 mmol) was added. The reaction mixture was degassed with nitrogen for 10 min and cooled to 0° C. Diisopropyl azodicarboxylate (14 mL, 74 mmol) was added to reaction mixture at 0° C. drop wise. The reaction mixture was stirred at 25° C. for 16 h. Upon completion of the reaction, the reaction mixture was diluted with water (100 mL) and product was extracted twice by dichloromethane (100 mL). The combined dichloromethane layer was dried over anhydrous sodium sulphate and evaporated under reduced pressure to obtain a crude compound. The crude compound was purified by flash column chromatography using 20% ethyl acetate in hexane as an eluent to obtain tert-butyl (R)-3-((4-cyanophenyl)thio)pyrrolidine-1-carboxylate (9.2 g, 30.2 mmol, 61.3% yield) as colorless oil.

### Step 3: —Preparation of tert-butyl (R)-3-((4-(N′-hydroxycarbamimidoyl)phenyl)thio)pyrrolidine-1-carboxylate

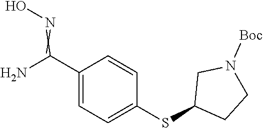 [SMILES: CC(C)(C)OC(=O)N1CC[C@@H](Sc2ccc(C(N)=NO)cc2)C1]

To the stirred solution of tert-butyl (R)-3-((4-cyanophenyl)thio)pyrrolidine-1-carboxylate (9 g, 30 mmol) in ethanol (100 mL) under nitrogen atmosphere, hydroxylamine hydrochloride (4 g, 60 mmol) and sodium bicarbonate (5.1 g, 60 mmol) were added. The resulting reaction mixture was heated at 70° C. for 16 h. Upon completion of the reaction, the reaction mixture was filtered through celite bed. The celite bed was washed with ethyl acetate (20 mL) and the filtrate was evaporated under reduced pressure. The residue was mixed in dichloromethane (40 mL) and stirred at 25° C. for 30 min. The solid formed was filtered and the filtrate obtained was evaporated to dryness to obtain tert-butyl (R)-3-((4-(N′-hydroxycarbamimidoyl)phenyl)thio)pyrrolidine-1-carboxylate (9.9 g, 29 mmol, 97% yield).

### Step 4: —Preparation of tert-butyl (R)-3-((4-(5-(trifluoromethyl)-1,2,4-oxadiazol-3-yl)phenyl)thio)pyrrolidine-1-carboxylate (Compound No. 157)

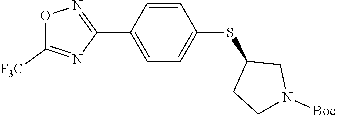 [SMILES: CC(C)(C)OC(=O)N1CC[C@@H](Sc2ccc(-c3noc(C(F)(F)F)n3)cc2)C1]

To the stirred solution of tert-butyl (R)-3-((4-(N′-hydroxycarbamimidoyl)phenyl)thio)pyrrolidine-1-carboxylate (9.9 g, 29 mmol) in tetrahydrofuran (100 mL) at 0° C. under nitrogen atmosphere, trifluoroacetic anhydride (6.2 mL, 44 mmol) was added drop wise. The resulting reaction mixture was heated to 25° C. and stirred for 16 h. Upon completion of the reaction, the reaction mixture was cooled to 0° C. and saturated sodium bicarbonate solution (50 mL) was added drop wise till the pH 7.5 to 8 was attained. The product was extracted twice with ethyl acetate (80 mL). The ethyl acetate layer was dried over anhydrous sodium sulphate and evaporated under reduced pressure to obtain a crude compound. The crude compound was purified by flash column chromatography using 40% ethyl acetate in hexane as an eluent to obtain tert-butyl (R)-3-((4-(5-(trifluoromethyl)-1,2,4-oxadiazol-3-yl)phenyl)thio)pyrrolidine-1-carboxylate (7.6 g, 18 mmol, 63% yield).

1H-NMR (400 MHz, CHLOROFORM-D) δ 8.04 (d, 2H), 7.46-7.44 (m, 2H), 3.94-3.84 (m, 2H), 3.59-3.35 (m, 3H), 2.33 (td, 1H), 1.97 (q, 1H), 1.46 (s, 9H); LCMS (M+H): 416.20

### Step 5: —Preparation of (R)-3-(4-(pyrrolidin-3-ylthio)phenyl)-5-(trifluoromethyl)-1,2,4-oxadiazole Hydrochloride (Compound No. 158)

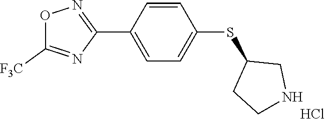 [SMILES: FC(F)(F)c1nc(-c2ccc(S[C@@H]3CCNC3)cc2)no1 ; Cl]

To the stirred solution of tert-butyl (R)-3-((4-(5-(trifluoromethyl)-1,2,4-oxadiazol-3-yl)phenyl)thio)pyrrolidine-1-carboxylate (7.6 g, 18 mmol) in dichloromethane (200 mL) at 0° C., 4M hydrogen chloride in 1,4-dioxane (46.0 mL, 184 mmol) was added under nitrogen atmosphere and the reaction mixture was stirred at 25° C. for 16 h. Upon completion of the reaction, the reaction mixture volatiles were evaporated, residual solid was washed twice with n-hexane (50 mL), filtered and dried to obtain (R)-3-(4-(pyrrolidin-3-ylthio)phenyl)-5-(trifluoromethyl)-1,2,4-oxadiazole hydrochloride (6.4 g, 18 mmol, 99% yield).

### Step 5: —(R)-1-(3-((4-(5-(trifluoromethyl)-1,2,4-oxadiazol-3-yl)phenyl)thio)pyrrolidin-1-yl)ethan-1-one (Compound No-165)

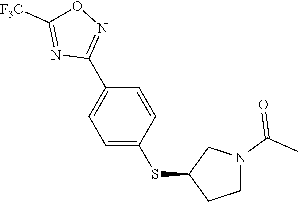 [SMILES: CC(=O)N1CC[C@@H](Sc2ccc(-c3noc(C(F)(F)F)n3)cc2)C1]

To the stirred solution of (R)-3-(4-(pyrrolidin-3-ylthio)phenyl)-5-(trifluoromethyl)-1,2,4-oxadiazole hydrochloride (0.25 g, 0.7 mmol) in dichloromethane (10 mL) at 0° C. under nitrogen atmosphere, triethylamine (0.4 mL, 3 mmol) and acetyl chloride (0.1 mL, 1 mmol) were added and the reaction mixture was heated to 25° C. and stirred for 1 h. Upon completion of the reaction, the reaction mixture was diluted with water (10 mL) and the product was extracted twice with dichloromethane (30 mL). The dichloromethane layer was dried over anhydrous sodium sulphate and evaporated under reduced pressure. The crude compound was purified by flash column chromatography using 60% ethyl acetate in hexane as an eluent to obtain (R)-1-(3-((4-(5-(trifluoromethyl)-1,2,4-oxadiazol-3-yl)phenyl)thio)pyrrolidin-1-yl)ethan-1-one (0.2 g, 0.6 mmol, 79% yield).

1H-NMR (400 MHz, CHLOROFORM-D) δ 8.18-7.97 (m, 2H), 7.54-7.39 (m, 2H), 4.15-3.89 (m, 2H), 3.78-3.45 (m, 3H), 2.50-1.89 (m, 5H); LCMS (M+H): 357.90

TABLE 16
The following compounds were prepared by the procedure analogous to that for the Compound No. 165

| Compound No. | Compound Name | 1H-NMR and LCMS | Yield |
|---|---|---|---|
| 172 | (R)-m-tolyl(3-((4-(5- | 1H-NMR (400 MHz, CHLOROFORM- | 0.26 g, 84% yield |
| | (trifluoromethyl)-1,2,4- | D) δ 8.08 (d, 2H), 7.54-7.44 (m, 2H), | |
| | oxadiazol-3- | 7.35 (d, 3H), 7.27-7.17 (1H), 4.18-3.77 | |
| | yl)phenyl)thio)pyrrolidin-1- | (m, 5H), 2.40 (s, 4H), 2.05 (d, 1H); | |
| | yl)methanone | LCMS (M + H): 434.5 | |
| 176 | (R)-(4-methoxyphenyl)(3-((4- | 1H-NMR (400 MHz, CHLOROFORM- | 0.23 g, 72% yield |
| | (5-(trifluoromethyl)-1,2,4- | D) δ 8.03 (d, 2H), 7.51 (t, 2H), 7.45 (d, | |
| | oxadiazol-3- | 2H), 6.90 (d, 2H), 4.00 (d, 2H), 3.84 (d, | |
| | yl)phenyl)thio)pyrrolidin-1- | 4H), 3.65 (s, 2H), 2.39 (dd, 1H), 2.05 | |
| | yl)methanone | (td, 1H); LCMS (M + H): 450.55 | |
| 177 | (R)-phenyl(3-((4-(5- | 1H-NMR @80° C. (400 MHz, DMSO- | 0.25 g, 85% yield |
| | (trifluoromethyl)-1,2,4- | D6) δ 7.97 (d, 2H), 7.58 (d, 2H), 7.48 (d, | |
| | oxadiazol-3- | 2H), 7.42 (d, 3H), 4.18 (t, 1H), 3.95 (s, | |
| | yl)phenyl)thio)pyrrolidin-1- | 1H), 3.57 (t, 3H), 2.42 (q, 1H), 1.95 (td, | |
| | yl)methanone | 1H); LCMS (M + H): 420.55 | |
| 178 | (R)-2-phenyl-1-(3-((4-(5- | 1H-NMR (400 MHz, CHLOROFORM- | 0.45 g, 73% yield |
| | (trifluoromethyl)-1,2,4- | D) δ 8.18-8.04 (m, 2H), 7.54-7.29 (m, | |
| | oxadiazol-3- | 6H), 7.26 (d, 1H), 4.00-3.44 (m, 7H), | |
| | yl)phenyl)thio)pyrrolidin-1- | 2.45-2.30 (m, 1H), 2.12-1.96 (m, 1H); | |
| | yl)ethan-1-one | LCMS (M + H): 434.05 | |
| 183 | (R)-(2-fluorophenyl)(3-((4-(5- | 1H-NMR (400 MHz, DMSO-D6) δ 7.98 | 0.23 g, 74% yield |
| | (trifluoromethyl)-1,2,4- | (dd, 2H), 7.58 (dd, 2H), 7.43 (dd, 2H), | |
| | oxadiazol-3- | 7.26 (d, 2H), 4.23-4.15 (m, 1H), 4.01 (s, | |
| | yl)phenyl)thio)pyrrolidin-1- | 1H), 3.76-3.71 (m, 1H), 3.62-3.53 (m, | |
| | yl)methanone | 1H), 3.44-3.37 (m, 1H), 3.19 (d, 1H), | |
| | | 2.43 (d, 1H), 1.96 (s, 1H); LCMS | |
| | | (M + H): 438.05 | |
| 197 | (R)-(4- | 1H-NMR (400 MHz, DMSO-D6) δ | 420 mg, 80% |
| | (dimethylamino)phenyl)(3-((4- | 8.02-7.92 (2H), 7.62-7.53 (2H), 7.45- | |
| | (5-(trifluoromethyl)-1,2,4- | 7.36 (2H), 6.72-6.64 (2H), 4.21-4.11 | |
| | oxadiazol-3- | (1H), 4.04-3.95 (1H), 3.76-3.66 (1H), | |
| | yl)phenyl)thio)pyrrolidin-1- | 3.64-3.55 (1H), 3.52-3.45 (1H), 2.97- | |
| | yl)methanone | 2.89 (6H), 2.45-2.35 (1H), 1.99-1.87 | |
| | | (1H); LCMS (M + H): 463.35 | |
| 204 | (R)-(3-((4-(5- | 1H-NMR (400 MHz, DMSO-D6) δ | 0.5 g, 90% yield |
| | (trifluoromethyl)-1,2,4- | 7.98 (s, 2H), 7.73 (dd, 4H), 7.61 (d, 2H), | |
| | oxadiazol-3- | 4.20 (s, 1H), 4.10-3.79 (1H), 3.59 (d, | |
| | yl)phenyl)thio)pyrrolidin-1- | 3H), 2.46-2.41 (m, 1H), 1.97 (t, 1H); | |
| | yl)(4- | LCMS (M + H): 488.05 | |
| | (trifluoromethyl)phenyl)methanone | | |

### Example 17: —Preparation of (R)-pyridin-4-yl(3-((4-(5-(trifluoromethyl)-1,2,4-oxadiazol-3-yl)phenyl)thio)pyrrolidin-1-yl)methanone (Compound 186) (Method-2)

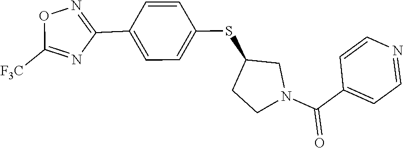 [SMILES: O=C(c1ccncc1)N1CC[C@@H](Sc2ccc(-c3noc(C(F)(F)F)n3)cc2)C1]

To the stirred solution of (R)-3-(4-(pyrrolidin-3-ylthio)phenyl)-5-(trifluoromethyl)-1,2,4-oxadiazole hydrochloride (0.25 g, 0.7 mmol) in N,N-dimethyl formamide (5 mL), triethylamine (0.4 mL, 2.9 mmol) and isonicotinic acid (0.1 g, 0.85 mmol) were added under inert atmosphere. The resulting reaction mixture was stirred at 25° C. for 5 min and HATU (0.4 g, 1 mmol) was added to the reaction mixture. The reaction mixture was stirred at 25° C. for 16 h. Upon completion of the reaction, the reaction mixture was diluted with water (10 mL) and the product was extracted twice by dichloromethane (40 mL). The dichloromethane layer was dried over anhydrous sodium sulphate and evaporated under reduced pressure. The crude compound was purified by preparative HPLC to obtain (R)-pyridin-4-yl(3-((4-(5-(trifluoromethyl)-1,2,4-oxadiazol-3-yl)phenyl)thio)pyrrolidin-1-yl)methanone (0.24 g, 0.58 mmol, 81% yield).

1H-NMR (400 MHz, DMSO-D6) δ 8.76-8.56 (2H), 8.10-7.86 (2H), 7.72-7.49 (2H), 7.49-7.35 (2H), 4.29-4.10 (1H), 4.09-3.94 (1H), 3.94-3.82 (1H), 3.81-3.67 (1H), 3.67-3.42 (2H), 3.41-3.22 (1H), 2.46-2.36 (1H), 2.05-1.87 (1H); LCMS (M+H): 421.05

TABLE 17
The following compounds were prepared by the procedure
analogous to that for the Compound No. 186

| Compound No. | Compound Name | 1H-NMR and LCMS | Yield |
|---|---|---|---|
| 187 | (R)-2-(4-chlorophenyl)-1-(3-((4- | 1H-NMR (400 MHz, DMSO-D6) δ | 0.56 g, 84% yield |
| | (5-(trifluoromethyl)-1,2,4- | 7.99 (d, 2H), 7.58 (t, 2H), 7.28 | |
| | oxadiazol-3- | (ddd, 4H), 4.20-4.12 (m, 1H), 3.83 | |
| | yl)phenyl)thio)pyrrolidin-1- | (q, 1H), 3.69-3.60 (m, 3H), 3.54- | |
| | yl)ethan-1-one | 3.35 (m, 2H), 2.46-2.32 (m, 1H), | |
| | | 2.01-1.88 (m, 1H); LCMS | |
| | | (M + H): 467.95 | |
| 203 | 2-(4-methoxyphenyl)-1-(3-((4-(5- | 1H-NMR (400 MHz, DMSO-D6) δ | 0.43 g, 93% yield |
| | (trifluoromethyl)-1,2,4-oxadiazol- | 7.96 (d, 2H), 7.54 (t, 2H), 7.10 (q, | |
| | 3-yl)phenyl)thio)pyrrolidin-1- | 2H), 6.81 (dd, 2H), 4.11 (dd, 1H), | |
| | yl)ethan-1-one | 3.97-3.79 (m, 1H), 3.70 (d, 3H), | |
| | | 3.65-3.56 (m, 1H), 3.53 (s, 1H), | |
| | | 3.49 (s, 1H), 3.44-3.33 (m, 2H), | |
| | | 2.41 (q, 1H), 1.95 (t, 1H), LCMS | |
| | | (M + H): 464.3 | |

### Example 18: —Preparation of (R)-(3-((4-(5-(trifluoromethyl)-1,2,4-oxadiazol-3-yl)phenyl)sulfonyl)pyrrolidin-1-yl)(4-(trifluoromethyl)phenyl)methanone (Compound-206)

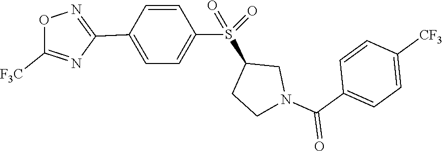 [SMILES: O=C(c1ccc(C(F)(F)F)cc1)N1CC[C@@H](S(=O)(=O)c2ccc(-c3noc(C(F)(F)F)n3)cc2)C1]

To the stirred solution of (R)-(3-((4-(5-(trifluoromethyl)-1,2,4-oxadiazol-3-yl)phenyl)thio)pyrrolidin-1-yl)(4-(trifluoromethyl)phenyl)methanone (0.2 g, 0.41 mmol) in dichloromethane (10 mL), meta chloro perbenzoic acid (0.2 g, 1.1 mmol) was added at 0° C. under nitrogen atmosphere. The resulting reaction mixture was heated to 25° C. and stirred for 2 h. Upon completion of the reaction, the reaction mixture was quenched with saturated sodium bicarbonate solution (10 mL). The product was extracted thrice with dichloromethane (60 mL). The dichloromethane layer was dried over anhydrous sodium sulphate and evaporated under reduced pressure to obtain a crude compound. The crude compound was purified by flash column chromatography using 80% ethyl acetate in hexane as an eluent to obtain (R)-(3-((4-(5-(trifluoromethyl)-1,2,4-oxadiazol-3-yl)phenyl)sulfonyl)pyrrolidin-1-yl)(4-(trifluoromethyl)phenyl)methanone (0.12 g, 0.23 mmol, 56% yield).

1H-NMR (400 MHz, DMSO-D6) δ 8.33 (d, 2H), 8.15 (s, 2H), 7.78 (d, 2H), 7.66 (d, 2H), 4.29 (d, 1H), 3.51-3.94 (m, 4H), 2.33-2.26 (m, 2H); LCMS (M+H): 520.10

TABLE 18
The following compounds were prepared by the procedure analogous to that for the Compound No. 206

| Compound No. | Compound Name | 1H-NMR and LCMS | Yield |
|---|---|---|---|
| 182 | (R)-2-phenyl-1-(3-((4-(5- | 1H-NMR (400 MHz, DMSO-D6) δ | 0.125 g, 86% yield |
| | (trifluoromethyl)-1,2,4-oxadiazol- | 8.33 (d, 2H), 8.14 (d, 2H), 7.27-7.20 | |
| | 3-yl)phenyl)sulfonyl)pyrrolidin-1- | (m, 5H), 4.29-4.22 (m, 1H), 2.26 (d, | |
| | yl)ethan-1-one | 2H), 2.08 (s, 1H); LCMS | |
| | | (M + H): 466.55 | |
| 189 | (R)-2-(4-chlorophenyl)-1-(3-((4- | 1H-NMR (400 MHz, DMSO-D6) δ | 0.33 g, 77% yield |
| | (5-(trifluoromethyl)-1,2,4- | 8.33 (d, 2H), 8.14 (d, 2H), 7.31 (d, | |
| | oxadiazol-3- | 2H), 7.22 (s, 2H), 4.30-4.23 (m, 1H), | |
| | yl)phenyl)sulfonyl)pyrrolidin-1- | 3.84-3.74 (m, 1H), 3.63-3.57 (m, | |
| | yl)ethan-1-one | 4H), 3.41 (s, 1H), 2.32-2.21 (m, 2H); | |
| | | LCMS (M + H): 500.05 | |
| 191 | (R)-2-(4-methoxyphenyl)-1-(3-((4- | 1H-NMR (400 MHz, DMSO-D6) δ | 0.18 g, 67% yield |
| | (5-(trifluoromethyl)-1,2,4- | 8.32 (d, 2H), 8.13 (d, 2H), 7.09 (d, | |
| | oxadiazol-3- | 2H), 6.83 (d, 2H), 4.25 (d, 1H), 3.80- | |
| | yl)phenyl)sulfonyl)pyrrolidin-1- | 3.64 (m, 5H), 3.60-3.38 (m, 4H), | |
| | yl)ethan-1-one | 2.32-2.20 (m, 2H); LCMS | |
| | | (M + H): 496.15 | |

### Example 19: Preparation of tert-butyl 3-((4-(5-(trifluoromethyl)-1,2,4-oxadiazol-3-yl)phenyl)thio)pyrrolidine-1-carboxylate (Compound No. 93)

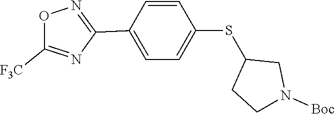 [SMILES: CC(C)(C)OC(=O)N1CCC(Sc2ccc(-c3noc(C(F)(F)F)n3)cc2)C1]

### Step 1: —Preparation of tert-butyl 3-((4-cyanophenyl)thio)pyrrolidine-1-carboxylate

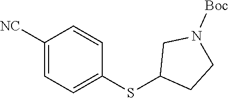 [SMILES: CC(C)(C)OC(=O)N1CCC(Sc2ccc(C#N)cc2)C1]

To the stirred solution of 4-mercaptobenzonitrile (0.2 g, 1.5 mmol) and tert-butyl 3-hydroxypyrrolidine-1-carboxylate (0.3 g, 1.5 mmol) in tetrahydrofuran (10 mL), triphenylphosphine (0.6 g, 2.2 mmol) followed by diethyl azodicarboxylate (0.35 mL, 2.2 mmol) were added under inert atmosphere at 0° C. The resulting reaction mixture was stirred at 25° C. under inert atmosphere for 16 h. Upon completion of the reaction, the reaction mixture was diluted with water (10 mL) and product was extracted twice by dichloromethane (40 mL). The dichloromethane layer was dried over anhydrous sodium sulphate and evaporated under reduced pressure to obtain a crude compound. The crude compound was purified by flash column chromatography using 20% ethyl acetate in hexane as an eluent to obtain tert-butyl 3-((4-cyanophenyl)thio)pyrrolidine-1-carboxylate (0.17 g, 0.56 mmol, 38% yield).

### Step 2: —Preparation of tert-butyl 3-((4-(N′-hydroxycarbamimidoyl)phenyl)thio)pyrrolidine-1-carboxylate

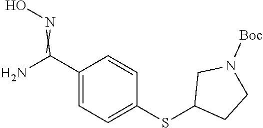 [SMILES: CC(C)(C)OC(=O)N1CCC(Sc2ccc(C(N)=NO)cc2)C1]

To the stirred solution of tert-butyl 3-((4-cyanophenyl)thio)pyrrolidine-1-carboxylate (0.17 g, 0.558 mmol) in ethanol (10 mL), hydroxylamine hydrochloride (0.08 g, 1.117 mmol) and sodium bicarbonate (0.1 g, 1.117 mmol) were added under nitrogen atmosphere. The resulting reaction mixture was heated at 70° C. for 16 h under inert atmosphere. Upon completion of the reaction, the reaction mixture was filtered through celite bed. Celite bed was washed with ethyl acetate (15 mL) and ethyl acetate was evaporated under reduced pressure to obtain tert-butyl 3-((4-(N′-hydroxycarbamimidoyl)phenyl)thio)pyrrolidine-1-carboxylate (0.17 g, 90% yield, 0.56 mmol).

### Step 3: —Preparation of tert-tert-butyl 3-44-(5-(trifluoromethyl)-1,2,4-oxadiazol-3-yl)phenyl)thio)pyrrolidine-1-carboxylate (Compound No. 93)

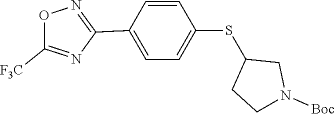 [SMILES: CC(C)(C)OC(=O)N1CCC(Sc2ccc(-c3noc(C(F)(F)F)n3)cc2)C1]

To the solution of tert-butyl (R)-3-((4-(N′-hydroxycarbamimidoyl)phenyl)thio)pyrrolidine-1-carboxylate (0.23 g, 0.7 mmol) in tetrahydrofuran (5 mL), trifluoroacetic anhydride (0.15 mL, 1.00 mmol) was added drop wise at 0° C. under inert atmosphere. The resulting reaction mixture was heated to 25° C. and stirred for 16 h. Upon completion of the reaction, the reaction mixture was cooled to 0° C. and saturated sodium bicarbonate solution (10 mL) was added drop wise to the reaction mixture. The product was extracted twice with ethyl acetate (40 mL). The ethyl acetate layer was dried over anhydrous sodium sulphate and evaporated under reduced pressure. The crude product was purified by flash column chromatography using 30% ethyl acetate in hexane as an eluent to obtain tert-butyl 3-((4-(5-(trifluoromethyl)-1,2,4-oxadiazol-3-yl)phenyl)thio)pyrrolidine-1-carboxylate (0.15 g, 0.36 mmol, 53% yield).

1H-NMR (400 MHz, CHLOROFORM-D) δ 8.06, 2H), 7.54-7.46 (m, 2H), 3.96-3.84 (m, 2H), 3.59-3.44 (m, 3H), 2.35 (td, 1H), 2.00 (td, 1H), 1.49 (s, 9H); LCMS (M+H): 416.1

### Example 20: Preparation of (3-(methyl(4-(5-(trifluoromethyl)-1,2,4-oxadiazol-3-yl)phenyl)amino)pyrrolidin-1-yl)(phenyl)methanone (Compound No. 207)

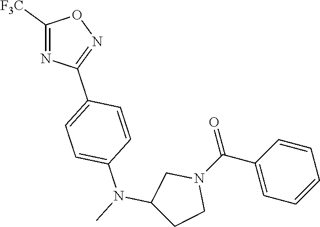 [SMILES: CN(c1ccc(-c2noc(C(F)(F)F)n2)cc1)C1CCN(C(=O)c2ccccc2)C1]

### Step 1: —Preparation of tert-butyl 3-((4-cyanophenyl)(methyl)amino)pyrrolidine-1-carboxylate

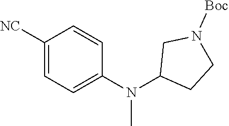 [SMILES: CN(c1ccc(C#N)cc1)C1CCN(C(=O)OC(C)(C)C)C1]

To the stirred solution of 4-bromobenzonitrile (5 g, 27.5 mmol) in toluene (50 mL), tert-butyl 3-(methylamino)pyrrolidine-1-carboxylate (6 g, 30.2 mmol), (2,2′-bis(diphenylphosphino)-1,1′-binaphthyl) (2.6 g, 4 mmol) and cesium carbonate (22.4 g, 69 mmol) were added at 25° C. under nitrogen atmosphere. The reaction mixture was degassed with nitrogen for 10 minutes and palladium (II) acetate (0.46 g, 2 mmol) was added to the reaction mixture and again degassed for 10 min. The reaction mixture was heated to 122° C. for 16 h. Upon completion of the reaction, the reaction mixture was cooled to 25° C. and diluted with water (50 mL). Ethyl acetate (50 mL) was added to the reaction mixture and biphasic solution was filtered through sintered funnel and the filtrate was extracted thrice with ethyl acetate (120 mL). The combined ethyl acetate layer was dried over anhydrous sodium sulphate and evaporated under reduced pressure to obtain a crude compound. The crude compound was purified by flash column chromatography using 40% ethyl acetate in hexane as an eluent to obtain tert-butyl 3-((4-cyanophenyl)(methyl)amino)pyrrolidine-1-carboxylate (6.2 g, 20.6 mmol, 75% yield).

### Step 2: —Preparation of tert-butyl 3-((4-(N′-hydroxycarbamimidoyl)phenyl)(methyl)amino)pyrrolidine-1-carboxylate

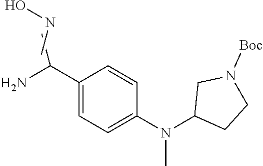

To the stirred solution of tert-butyl 3-((4-cyanophenyl)(methyl)amino)pyrrolidine-1-carboxylate (10 g, 34 mmol) in ethanol (100 mL), hydroxylamine hydrochloride (4.7 g, 67 mmol) and sodium bicarbonate (5.7 g, 67 mmol) were added at 25° C. under inert atmosphere. The reaction mixture was heated to 80° C. for 16 h. Upon completion of the reaction, the reaction mixture was filtered through celite bed. Celite bed was washed with ethyl acetate (30 mL) and ethyl acetate was evaporated under reduced pressure to obtain a residue. The residue was dissolved in dichloromethane (40 mL) and stirred at 25° C. for 30 min, filtered and the dichloromethane was evaporated under reduced pressure to obtain tert-butyl 3-((4-(N′-hydroxycarbamimidoyl)phenyl)(methyl)amino)pyrrolidine-1-carboxylate (9.6 g, 29 mmol, 85% yield).

### Step 3: —Preparation of tert-butyl 3-(methyl(4-(5-(trifluoromethyl)-1,2,4-oxadiazol-3-yl)phenyl)amino)pyrrolidine-1-carboxylate (Compound No 167)

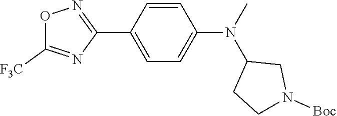 [SMILES: CN(c1ccc(-c2noc(C(F)(F)F)n2)cc1)C1CCN(C(=O)OC(C)(C)C)C1]

To the stirred solution of tert-butyl 3-((4-(N′-hydroxycarbamimidoyl)phenyl)(methyl)amino)pyrrolidine-1-carboxylate (9.6 g, 29 mmol) in tetrahydrofuran (100 mL) at 0° C., trifluoroacetic anhydride (6.1 mL, 43 mmol) was added drop wise. The resulting reaction mixture was heated to 25° C. and stirred for 16 h. Upon completion of the reaction, the reaction mixture was cooled to 0° C. and saturated sodium bicarbonate solution (50 mL) was added drop wise. The product was extracted thrice with ethyl acetate (90 mL). The ethyl acetate layer was dried over anhydrous sodium sulphate and evaporated under reduced pressure. The crude product was purified by flash column chromatography using 30% ethyl acetate in hexane as an eluent to obtain tert-butyl 3-(methyl(4-(5-(trifluoromethyl)-1,2,4-oxadiazol-3-yl)phenyl)amino)pyrrolidine-1-carboxylate (8.2 g, 20 mmol, 69.5% yield).

1H-NMR (400 MHz, CHLOROFORM-D) δ 8.02-7.99 (m, 2H), 7.02-6.93 (m, 2H), 4.52 (s, 1H), 3.57-3.39 (d, 1H), 3.69 (m, 4H), 2.97 (s, 3H), 2.18-2.03 (m, 2H), 1.50-1.26 (m, 10H); LCMS (M+H): 413.20

### Step 4: —Preparation of N-methyl-N-(4-(5-(trifluoromethyl)-1,2,4-oxadiazol-3-yl)phenyl)pyrrolidin-3-amine hydrochloride

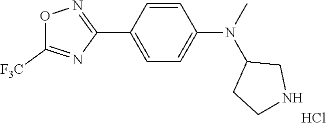 [SMILES: CN(c1ccc(-c2noc(C(F)(F)F)n2)cc1)C1CCNC1 ; Cl]

To the stirred solution of tert-butyl 3-(methyl(4-(5-(trifluoromethyl)-1,2,4-oxadiazol-3-yl)phenyl)amino)pyrrolidine-1-carboxylate (8 g, 19.4 mmol) in dichloromethane (100 mL) at 0° C., 4M hydrogen chloride in 1,4-dioxane (32 mL, 128 mmol) was added and the resulting reaction mixture was stirred at 25° C. for 16 h. Upon completion of the reaction, the volatiles were evaporated and the residue was washed with n-hexane (40 mL), filtered and washed further with n-hexane (20 mL) to obtain N-methyl-N-(4-(5-(trifluoromethyl)-1,2,4-oxadiazol-3-yl)phenyl)pyrrolidin-3-amine dihydrochloride (4.5 g, 11.7 mmol, 91% yield).

### Step 5: —Preparation of (3-(methyl(4-(5-(trifluoromethyl)-1,2,4-oxadiazol-3-yl)phenyl)amino)pyrrolidin-1-yl)(phenyl)methanone (Compound No. 207)

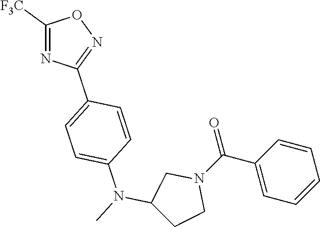 [SMILES: CN(c1ccc(-c2noc(C(F)(F)F)n2)cc1)C1CCN(C(=O)c2ccccc2)C1]

To the stirred solution of N-methyl-N-(4-(5-(trifluoromethyl)-1,2,4-oxadiazol-3-yl)phenyl)pyrrolidin-3-amine dihydrochloride (0.3 g, 0.8 mmol) in dichloromethane (10 mL) under nitrogen atmosphere, triethylamine (0.9 mL, 6.2 mmol) was added. The reaction mixture was cooled to 0° C., benzoyl chloride (0.14 mL, 1.2 mmol) was added drop wise under nitrogen atmosphere and the reaction mixture was heated to 25° C. and stirred for 2 h. Upon completion of the reaction, the reaction mixture was diluted with water (10 mL) and product was extracted thrice with dichloromethane (60 mL). The dichloromethane layer was dried over anhydrous sodium sulphate and evaporated under reduced pressure to obtain a crude compound. The crude compound was purified by flash column chromatography using 60% ethyl acetate in hexane as an eluent to obtain (3-(methyl(4-(5-(trifluoromethyl)-1,2,4-oxadiazol-3-yl)phenyl)amino)pyrrolidin-1-yl)(phenyl)methanone (0.32 g, 0.77 mmol, 99% yield). 1H-NMR (400 MHz, DMSO-D6) δ 7.84 (dd, 2H), 7.54-7.43 (m, 5H), 7.01 (dd, 2H), 4.69 (dt, 1H), 3.81-3.44 (m, 4H), 2.90 (d, 3H), 2.17-2.05 (m, 2H); LCMS (M+H): 417.30

### Example 21: —Preparation of (3-(methyl(4-(5-(trifluoromethyl)-1,2,4-oxadiazol-3-yl)phenyl)amino)azetidin-1-yl)(phenyl)methanone (Compound No. 87)

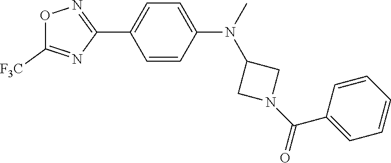 [SMILES: CN(c1ccc(-c2noc(C(F)(F)F)n2)cc1)C1CN(C(=O)c2ccccc2)C1]

### Step 1: —Preparation of tert-butyl 3-((4-cyanophenyl)amino)azetidine-1-carboxylate

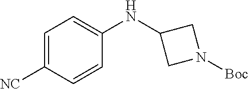 [SMILES: CC(C)(C)OC(=O)N1CC(Nc2ccc(C#N)cc2)C1]

To the stirred solution of tert-butyl 3-aminoazetidine-1-carboxylate (10 g, 58.1 mmol) in toluene (100 mL), 4-bromobenzonitrile (10.6 g, 58 mmol), cesium carbonate (42 g, 128 mmol), and 2,2′-bis(diphenylphosphino)-1,1′-binaphthyl (5.4 g, 8.7 mmol) were added and the reaction mixture was degassed with nitrogen for 15 minutes followed by addition of palladium (II) acetate (2 g, 8.7 mmol). The resulting reaction mixture was again degassed with nitrogen for another 15 minutes and was heated at 110° C. for 12 h. Upon completion of the reaction, the reaction mixture was filtered through celite bed and washed thrice with ethyl acetate (50 ml). The combined ethyl acetate layer was dried over anhydrous sodium sulphate and evaporated under reduced pressure to obtain a crude compound. The crude compound was purified by flash column chromatography using 20% Ethyl acetate in hexane as an eluent to obtain tert-butyl 3-((4-cyanophenyl)amino)azetidine-1-carboxylate (10.5 g, 38.4 mmol, 66% yield).

### Step 2: —Preparation of tert-butyl 3-((4-cyanophenyl)(methyl)amino)azetidine-1-carboxylate

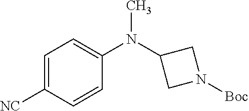 [SMILES: CN(c1ccc(C#N)cc1)C1CN(C(=O)OC(C)(C)C)C1]

To a stirred slurry of sodium hydride (2.2 g, 91 mmol) in dimethyl formamide (80 mL), tert-butyl 3-((4-cyanophenyl)amino)azetidine-1-carboxylate (10 g, 37 mmol) was added at 0° C. The reaction mixture was stirred for 10 min at the same the temperature, iodomethane (6.9 mL, 110 mmol) was added and stirred at 25° C. for 2 h. The reaction mixture was quenched by addition of ice cold saturated ammonium chloride solution until effervescence ceases. The reaction mixture was extracted thrice with ethyl acetate (75 mL). The combined ethyl acetate layer was washed thrice with brine solution (50 mL), dried over anhydrous sodium sulfate and concentrated under reduced pressure to obtain a crude product. The crude product was purified by column chromatography to obtain tert-butyl 3-((4-cyanophenyl)(methyl)amino)azetidine-1-carboxylate (6.5 g, 22.6 mmol, 62% yield) and an impurity from the previous step tert-butyl 3-(bis(4-cyanophenyl)amino)azetidine-1-carboxylate (2.1 g).

### Step 3: —Preparation of tert-butyl 3-((4-(N′-hydroxycarbamimidoyl)phenyl)(methyl)amino)azetidine-1-carboxylate (4)

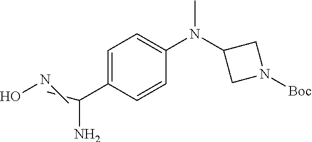

To the stirred solution of tert-butyl 3-((4-cyanophenyl)(methyl)amino)azetidine-1-carboxylate (6.5 g, 23 mmol) in ethanol (70 mL), hydroxylamine (2 mL, 34 mmol) was added at 25° C. The resulting reaction mixture was stirred at 65° C. for 16 h. Upon completion of the reaction, the volatiles were evaporated under reduced pressure to obtain a crude product. The crude product was crystallized using dichloromethane:hexane (1:8 v/v) to obtain tert-butyl 3-((4-(N′-hydroxycarbamimidoyl)phenyl)(methyl)amino)azetidine-1-carboxylate (6.7 g, 21 mmol, 92% yield).

### Step 4: —Preparation of tert-butyl 3-(methyl(4-(5-(trifluoromethyl)-1,2,4-oxadiazol-3-yl)phenyl)amino)azetidine-1-carboxylate (Compound No 80)

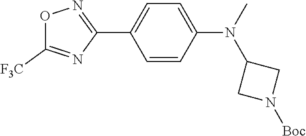 [SMILES: CN(c1ccc(-c2noc(C(F)(F)F)n2)cc1)C1CN(C(=O)OC(C)(C)C)C1]

To the stirred solution of tert-butyl 3-((4-(N′-hydroxycarbamimidoyl)phenyl)(methyl)amino)azetidine-1-carboxylate (6.5 g, 18.26 mmol) in tetrahydrofuran (50 mL), trifluoroacetic anhydride (4 mL, 27 mmol) was added at 0° C. under nitrogen atmosphere. The reaction mixture was stirred at 25° C. for 6 h. Upon completion of the reaction, ethyl acetate (50 mL) was added to the reaction mixture followed by slow addition of saturated sodium bicarbonate solution until the effervesce ceases. The ethyl acetate layer was separated, washed with water (100 mL) followed by washing with brine solution (50 mL), dried over anhydrous sodium sulphate and evaporated under reduced pressure to obtain the crude product which was purified by flash column chromatography in silica gel using 60% ethyl acetate in hexane as an eluent to obtain tert-butyl 3-(methyl(4-(5-(trifluoromethyl)-1,2,4-oxadiazol-3-yl)phenyl)amino)azetidine-1-carboxylate (3.5 g, 8.8 mmol, 48% yield).

1H-NMR (400 MHz, CHLOROFORM-D) δ 7.99-7.95 (m, 2H), 6.79-6.76 (m, 2H), 4.58-4.52 (m, 1H), 4.24 (dd, 2H), 4.02 (dd, 2H), 3.05 (s, 3H), 1.45 (s, 9H); LCMS (M+H): 399.05

### Step 5: —Preparation of N-methyl-N-(4-(5-(trifluoromethyl)-1,2,4-oxadiazol-3-yl)phenyl)azetidin-3-amine hydrochloride

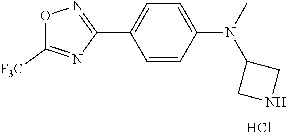 [SMILES: CN(c1ccc(-c2noc(C(F)(F)F)n2)cc1)C1CNC1 ; Cl]

To the stirred solution of tert-butyl3-(methyl(4-(5-(trifluoromethyl)-1,2,4-oxadiazol-3-yl)phenyl)amino)azetidine-1-carboxylate (0.8 g, 1.6 mmol) in dichloromethane (15 mL) at 0° C. 4M hydrochloric acid in 1,4-dioxane (0.7 mL, 3 mmol) was added. The resulting reaction mixture was stirred at 25° C. for 12 h. Upon completion of the reaction, the volatiles were evaporated under reduced pressure to obtain a crude product. The crude product obtained was washed with n-hexane (50 mL), filtered and dried to obtain N-methyl-N-(4-(5-(trifluoromethyl)-1,2,4-oxadiazol-3-yl)phenyl)azetidin-3-amine hydrochloride (0.27 g, 0.8 mmol, 50% yield).

### Step 6: —Preparation of (3-(methyl(4-(5-(trifluoromethyl)-1,2,4-oxadiazol-3-yl)phenyl)amino)azetidin-1-yl)(phenyl)methanone (87)

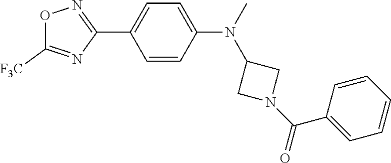 [SMILES: CN(c1ccc(-c2noc(C(F)(F)F)n2)cc1)C1CN(C(=O)c2ccccc2)C1]

To the stirred solution of N-methyl-N-(4-(5-(trifluoromethyl)-1,2,4-oxadiazol-3-yl)phenyl)azetidin-3-amine hydrochloride (0.25 g, 0.75 mmol) in dichloromethane (10 mL), triethylamine (0.4 ml, 3 mmol) was added at 25° C. The resulting reaction mixture was cooled to 0° C. and benzoyl chloride (0.13 mL, 1.1 mmol) was added drop wise. The reaction mixture was stirred at 25° C. for 2 h. Upon completion of the reaction, the reaction mixture was diluted with water (10 mL) and product was extracted thrice with dichloromethane (45 mL). The dichloromethane layer was dried over anhydrous sodium sulphate and evaporated under reduced pressure. The crude product was purified by using flash column chromatography in silica gel using 70% ethyl acetate in hexane as an eluent to obtain (3-(methyl(4-(5-(trifluoromethyl)-1,2,4-oxadiazol-3-yl)phenyl)amino)azetidin-1-yl)(phenyl)methanone (0.23 g, 0.6 mmol, 77% yield).

1H-NMR (400 MHz, CHLOROFORM-D) δ 8.02-7.97 (m, 2H), 7.70-7.67 (m, 2H), 7.54-7.42 (m, 3H), 6.82-6.78 (m, 2H), 4.72-4.66 (m, 1H), 4.59 (d, 2H), 4.35 (q, 2H), 3.11 (s, 3H); LCMS (M+H): 403.0

TABLE 19
The following compounds were prepared by the procedure analogous to that for the Compound No. 87

| Compound No. | Compound Name | 1H-NMR and LCMS | Yield |
|---|---|---|---|
| 88 | (3-chlorophenyl)(3-(methyl(4- | 1H-NMR (400 MHz, | 0.22 g, 67% yield |
| | (5-(trifluoromethyl)-1,2,4- | CHLOROFORM-D) δ 7.98 (dt, 2H), | |
| | oxadiazol-3- | 7.64 (t, 1H), 7.53 (dt, 1H), 7.46 (dq, | |
| | yl)phenyl)amino)azetidin-1- | 1H), 7.38 (q, 1H), 6.78 (dt, 2H), | |
| | yl)methanone | 4.71-4.64 (m, 1H), 4.56 (s, 2H), 4.32 | |
| | | (q, 2H), 3.08 (s, 3H); LCMS (M + H): | |
| | | 436.9 | |
| 89 | 1-(3-(methyl(4-(5- | 1H-NMR (400 MHz, | 0.2 g, 69% yield |
| | (trifluoromethyl)-1,2,4- | CHLOROFORM-D) δ 7.99-8.03 (m, | |
| | oxadiazol-3- | 2H), 6.81 (dt, 2H), 4.67-4.61 (m, | |
| | yl)phenyl)amino)azetidin-1- | 1H), 4.50-4.32 (m, 2H), 4.24-4.13 | |
| | yl)ethan-1-one | (m, 2H), 3.08 (s, 3H), 1.95 (s, 3H); | |
| | | LCMS (M + H): 341.0 | |
| 96 | (3-fluorophenyl)(3-(methyl(4- | 1H-NMR (400 MHz, | 0.2 g, 84% yield |
| | (5-(trifluoromethyl)-1,2,4- | CHLOROFORM-D) δ 8.01 (dt, 2H), | |
| | oxadiazol-3- | 7.54-7.38 (m, 3H), 7.25-7.18 (m, | |
| | yl)phenyl)amino)azetidin-1- | 1H), 6.80 (dt, 2H), 4.73-4.66 (m, | |
| | yl)methanone | 1H), 4.59 (s, 2H), 4.35 (q, 2H), 3.11 | |
| | | (s, 3H); LCMS (M + H): 421.2 | |
| 97 | (3-(methyl(4-(5- | 1H-NMR (400 MHz, | 0.12 g, 67% yield |
| | (trifluoromethyl)-1,2,4- | CHLOROFORM-D) δ 8.02-7.97 (m, | |
| | oxadiazol-3- | 2H), 7.60-7.57 (m, 2H), 7.33-7.23 | |
| | yl)phenyl)amino)azetidin-1- | (m, 2H), 6.82-6.78 (m, 2H), 4.71- | |
| | yl)(p-tolyl)methanone | 4.65 (m, 1H), 4.57 (t, 2H), 4.34 (q, | |
| | | 2H), 3.10 (s, 3H), 2.42 (s, 3H); | |
| | | LCMS (M + H): 417.3 | |
| 98 | (4-methoxyphenyl)(3- | 1H-NMR (400 MHz, | 0.16 g, 77% yield |
| | (methyl(4-(5- | CHLOROFORM-D) δ 7.97 (dt, 2H), | |
| | (trifluoromethyl)-1,2,4- | 7.67-7.63 (m, 2H), 6.95-6.90 (m, | |
| | oxadiazol-3- | 2H), 6.77 (dt, 2H), 4.69-4.62 (m, | |
| | yl)phenyl)amino)azetidin-1- | 1H), 4.56 (s, 2H), 4.32 (q, 2H), 3.87- | |
| | yl)methanone | 3.85 (m, 3H), 3.08 (s, 3H); LCMS | |
| | | (M + H): 433.3 | |
| 99 | 1-(3-(methyl(4-(5- | 1H-NMR (400 MHz, | 0.1 g, 60% yield |
| | (trifluoromethyl)-1,2,4- | CHLOROFORM-D) δ 8.01-7.97 (m, | |
| | oxadiazol-3- | 2H), 7.38-7.29 (m, 5H), 6.78 (dd, | |
| | yl)phenyl)amino)azetidin-1- | 2H), 4.65-4.58 (m, 1H), 4.42-4.33 | |
| | yl)-2-phenylethan-1-one | (m, 2H), 4.21-4.11 (m, 2H), 3.57 (d, | |
| | | 2H), 3.02 (s, 3H); LCMS (M + H): | |
| | | 417.1 | |
| 100 | 1-(3-(methyl(4-(5- | 1H-NMR (400 MHz, | 0.15 g, 83% yield |
| | (trifluoromethyl)-1,2,4- | CHLOROFORM-D) δ 8.02-7.98 (m, | |
| | oxadiazol-3- | 2H), 7.02-6.78 (m, 2H), 4.68-4.61 | |
| | yl)phenyl)amino)azetidin-1- | (m, 1H), 4.46-4.32 (m, 2H), 4.16 (t, | |
| | yl)propan-1-one | 2H), 3.24-2.89 (m, 3H), 2.34-2.01 | |
| | | (m, 2H), 1.21-0.90 (m, 3H); LCMS | |
| | | (M + H): 355.1 | |
| 101 | (3-(methyl(4-(5- | 1H-NMR (400 MHz, | 0-2 g, 85% yield |
| | (trifluoromethyl)-1,2,4- | CHLOROFORM-D) δ 8.02-7.98 (m, | |
| | oxadiazol-3- | 2H), 7.81-7.79 (m, 2H), 7.73-7.70 | |
| | yl)phenyl)amino)azetidin-1- | (m, 2H), 6.80 (dt, 2H), 4.73-4.67 (m, | |
| | yl)(4- | 1H), 4.60-4.56 (m, 2H), 4.35 (s, 2H), | |
| | (trifluoromethyl)phenyl)methanone | 3.11 (s, 3H); LCMS (M + H): 470.9 | |
| 123 | (4-(dimethylamino)phenyl)(3- | 1H-NMR (400 MHz, | 0.09 g, 44% yield |
| | (methyl(4-(5- | CHLOROFORM-D) δ 8.01-7.98 (m, | |
| | (trifluoromethyl)-1,2,4- | 2H), 7.65-7.54 (m, 2H), 6.81-6.69 | |
| | oxadiazol-3- | (m, 4H), 4.71-4.59 (m, 3H), 4.35 (q, | |
| | yl)phenyl)amino)azetidin-1- | 2H), 3.10 (s, 3H), 3.05 (s, 6H); | |
| | yl)methanone | LCMS (M + H): 446.4 | |
| 124 | (4-fluorophenyl)(3-(methyl(4- | 1H-NMR (400 MHz, | 0.16 g, 88% yield |
| | (5-(trifluoromethyl)-1,2,4- | CHLOROFORM-D) δ 7.99-8.02 (m, | |
| | oxadiazol-3- | 2H), 7.73-7.68 (m, 2H), 7.16-7.02 | |
| | yl)phenyl)amino)azetidin-1- | (m, 2H), 6.82-6.78 (m, 2H), 4.72- | |
| | yl)methanone | 4.58 (m, 3H), 4.34 (q, 2H), 3.11 (s, | |
| | | 3H); LCMS (M + H): 421.1 | |
| 125 | (2-fluorophenyl)(3-(methyl(4- | 1H-NMR (400 MHz, | 0.17 g, 82% yield |
| | (5-(trifluoromethyl)-1,2,4- | CHLOROFORM-D) δ 8.01-7.98 (m, | |
| | oxadiazol-3- | 2H), 7.61 (td, 1H), 7.50-7.44 (m, | |
| | yl)phenyl)amino)azetidin-1- | 1H), 7.25 (td, 1H), 7.16-7.02 (m, | |
| | yl)methanone | 1H), 6.82-6.78 (m, 2H), 4.74-4.67 | |
| | | (m, 1H), 4.58-4.53 (m, 1H), 4.41- | |
| | | 4.31 (m, 2H), 4.21 (dd, 1H), 3.11 (s, | |
| | | 3H); LCMS (M + H): 421.0 | |
| 126 | (3-(methyl(4-(5- | 1H-NMR (400 MHz, | 0.14 g, 66% yield |
| | (trifluoromethyl)-1,2,4- | CHLOROFORM-D) δ 8.00 (dt, 2H), | |
| | oxadiazol-3- | 7.53 (d, 1H), 7.46-7.43 (m, 1H), | |
| | yl)phenyl)amino)azetidin-1- | 7.35-7.30 (m, 2H), 6.82-6.78 (m, | |
| | yl)(m-tolyl)methanone | 2H), 4.71-4.57 (m, 3H), 4.33 (dd, | |
| | | 2H), 3.11 (s, 3H), 2.42 (s, 3H); | |
| | | LCMS (M + H): 417.1 | |
| 129 | 2,2-dimethyl-1-(3-(methyl(4- | 1H-NMR (400 MHz, | 0.13 g, 79% yield |
| | (5-(trifluoromethyl)-1,2,4- | CHLOROFORM-D) δ 8.02-7.99 (m, | |
| | oxadiazol-3- | 2H), 6.82-6.78 (m, 2H), 4.57-4.35 | |
| | yl)phenyl)amino)azetidin-1- | (m, 5H), 3.08 (s, 3H), 1.25 (s, 9H); | |
| | yl)propan-1-one | LCMS (M + H): 383.2 | |

### Example 22: N-methyl-1-(phenylsulfonyl)-N-(4-(5-(trifluoromethyl)-1,2,4-oxadiazol-3-yl)phenyl)azetidin-3-amine (164)

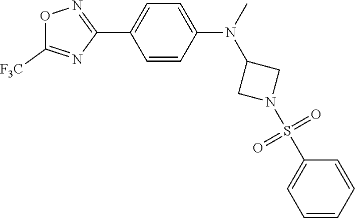 [SMILES: CN(c1ccc(-c2noc(C(F)(F)F)n2)cc1)C1CN(S(=O)(=O)c2ccccc2)C1]

### Step 1: Preparation of N-methyl-1-(phenylsulfonyl)-N-(4-(5-(trifluoromethyl)-1,2,4-oxadiazol-3-yl)phenyl)azetidin-3-amine (164)

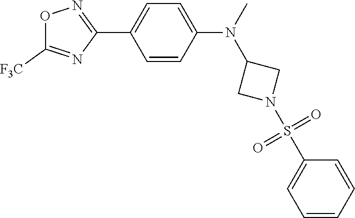 [SMILES: CN(c1ccc(-c2noc(C(F)(F)F)n2)cc1)C1CN(S(=O)(=O)c2ccccc2)C1]

To the stirred solution of N-methyl-N-(4-(5-(trifluoromethyl)-1,2,4-oxadiazol-3-yl)phenyl)azetidin-3-amine hydrochloride (0.2 g, 0.6 mmol) in dichloromethane (5 mL), triethylamine (0.3 ml, 2.4 mmol) was added at 0° C., followed by addition of benzenesulfonyl chloride (0.12 ml, 0.9 mmol). The resulting reaction mixture was stirred for 12 h at 25° C. Upon completion of the reaction, the reaction mixture was quenched by saturated sodium bicarbonate solution (20 mL) and extracted thrice with dichloromethane (30 mL). The dichloromethane layer was separated, dried over anhydrous sodium sulfate and concentrated under reduced pressure to obtain a crude product which was further purified by flash column chromatography using 60% ethyl acetate in hexane as an eluent to obtain N-methyl-1-(phenylsulfonyl)-N-(4-(5-(trifluoromethyl)-1,2,4-oxadiazol-3-yl)phenyl)azetidin-3-amine (0.2 g, 0.5 mmol, 76% yield).

1H-NMR (400 MHz, DMSO-D6) δ 7.91-7.88 (m, 2H), 7.85-7.81 (m, 3H), 7.76-7.72 (m, 2H), 6.88-6.86 (m, 2H), 4.68-4.61 (m, 1H), 4.11 (dd, 2H), 3.75 (dd, 2H), 2.61 (s, 3H); LCMS (M+H): 439.1

TABLE 20
The following compounds were prepared by the procedure analogous to that for the Compound No. 164

| Compound No. | Compound Name | 1H-NMR and LCMS | Yield |
|---|---|---|---|
| 168 | N-methyl-1-tosyl-N-(4-(5- | 1H-NMR (400 MHz, DMSO-D6) δ | 0.16 g, 81% yield |
| | (trifluoromethyl)-1,2,4- | 7.76-7.83 (m, 4H), 7.57-7.52 (m, | |
| | oxadiazol-3- | 2H), 6.89-6.85 (m, 2H), 4.65-4.58 | |
| | yl)phenyl)azetidin-3-amine | (m, 1H), 4.08 (dd, 2H), 3.73 (dd, | |
| | | 2H), 2.65 (s, 3H), 2.46 (s, 3H); | |
| | | LCMS (M + H): 453.2 | |
| 169 | 1-((2-fluorophenyl)sulfonyl)- | 1H-NMR (400 MHz, DMSO-D6) δ | 0.1 g, 51% yield |
| | N-methyl-N-(4-(5- | 7.82-7.90 (m, 4H), 7.49-7.61 (m, | |
| | (trifluoromethyl)-1,2,4- | 2H), 6.92-6.89 (m, 2H), 4.76-4.69 | |
| | oxadiazol-3- | (m, 1H), 4.22 (t, 2H), 4.05-3.88 (m, | |
| | yl)phenyl)azetidin-3-amine | 2H), 2.80 (s, 3H); LCMS (M + H): | |
| | | 457.2 | |
| 170 | 1-((4- | 1H-NMR (400 MHz, DMSO-D6) δ | 0.16 g, 80% yield |
| | methoxyphenyl)sulfonyl)-N- | 7.84-7.81 (m, 4H), 7.25-7.21 (m, | |
| | methyl-N-(4-(5- | 2H), 6.89-6.86 (m, 2H), 4.65-4.58 | |
| | (trifluoromethyl)-1,2,4- | (m, 1H), 4.09-4.04 (m, 2H), 3.89 (s, | |
| | oxadiazol-3- | 3H), 3.72 (dd, 2H), 2.67 (s, 3H); | |
| | yl)phenyl)azetidin-3-amine | LCMS (M + H): 469.0 | |
| 171 | N-methyl-N-(4-(5- | 1H-NMR (400 MHz, DMSO-D6) δ | 0.14 g, 62% yield |
| | (trifluoromethyl)-1,2,4- | 8.23-7.97 (m, 4H), 7.82 (d, 2H), | |
| | oxadiazol-3-yl)phenyl)-1-((3-(tri- | 6.87 (d, 2H), 4.71-4.64 (m, 1H), | |
| | fluoromethyl)phenyl)sulfonyl)azetidin- | 4.17 (t, 2H), 3.86 (dd, 2H), 2.70 (s, | |
| | 3-amine | 3H); LCMS (M + H): 507.1 | |
| 173 | N-methyl-N-(4-(5- | 1H-NMR (400 MHz, DMSO-D6) δ | 0.12 g, 57% yield |
| | (trifluoromethyl)-1,2,4- | 8.13-8.01 (m, 4H), 7.84-7.80 (m, | |
| | oxadiazol-3-yl)phenyl)-1-((4-(tri- | 2H), 6.89-6.84 (m, 2H), 4.69-4.63 | |
| | fluoromethyl)phenyl)sulfonyl)azetidin- | (m, 1H), 4.16 (t, 2H), 3.85 (dd, 2H), | |
| | 3-amine | 2.71 (s, 3H); LCMS (M + H): 507.1 | |
| 174 | 1-((3-chlorophenyl)sulfonyl)- | 1H-NMR (400 MHz, DMSO-D6) δ | 0.1 g, 50% yield |
| | N-methyl-N-(4-(5- | 7.92-7.81 (m, 5H), 7.78-7.74 (m, | |
| | (trifluoromethyl)-1,2,4- | 1H), 6.90-6.87 (m, 2H), 4.70-4.63 | |
| | oxadiazol-3- | (m, 1H), 4.17-4.13 (m, 2H), 3.84 | |
| | yl)phenyl)azetidin-3-amine | (dd, 2H), 2.72 (s, 3H); LCMS | |
| | | (M + H): 473.1 | |
| 195 | N-methyl-1-(propylsulfonyl)- | 1H-NMR (400 MHz, DMSO-D6) δ | 110 mg, 91% yield |
| | N-(4-(5-(trifluoromethyl)- | 7.88-7.84 (m, 2H), 6.97-6.93 (m, | |
| | 1,2,4-oxadiazol-3- | 2H), 4.85-4.73 (m, 1H), 4.15 (dd, | |
| | yl)phenyl)azetidin-3-amine | 2H), 4.01 (dd, 2H), 3.18-3.14 (m, | |
| | | 2H), 3.00 (s, 3H), 1.76-1.67 (m, | |
| | | 2H), 1.00 (t, 3H); LCMS (M + H): | |
| | | 405.05 | |
| 196 | N-methyl-N-(4-(5- | 1H-NMR (400 MHz, DMSO-D6) δ | 115 mg, 74.5% yield |
| | (trifluoromethyl)-1,2,4- | 7.88-7.85 (m, 2H), 7.01-6.98 (m, | |
| | oxadiazol-3-yl)phenyl)-1-((tri- | 2H), 5.10-5.03 (m, 1H), 4.56 (t, | |
| | fluoromethyl)sulfonyl)azetidin- | 2H), 4.42-4.39 (m, 2H), 3.04 (s, | |
| | 3-amine | 3H); LCMS (M + H): 431.10 | |
| 198 | 1-((3- | 1H-NMR (400 MHz, DMSO-D6) δ | 110 mg, 79% yield |
| | methoxyphenyl) sulfonyl)-N- | 7.82-7.78 (m, 2H), 7.63 (t, 1H), 7.43 | |
| | methyl-N-(4-(5- | (dq, 1H), 7.36 (dq, 1H), 7.30 (t, 1H), | |
| | (trifluoromethyl)-1,2,4- | 6.88-6.84 (m, 2H), 4.65-4.61 (m, | |
| | oxadiazol-3- | 1H), 4.12-4.08 (m, 2H), 3.86 (s, | |
| | yl)phenyl)azetidin-3-amine | 3H), 3.78 (dd, 2H), 2.63 (s, 3H); | |
| | | LCMS (M + H): 469.15 | |

### Example 23: —Preparation of 2,2-dimethyl-1-(3-(3-(5-(trifluoromethyl)-1,2,4-oxadiazol-3-yl)phenoxy)pyrrolidin-1-yl)propan-1-one (Compound No. 130)

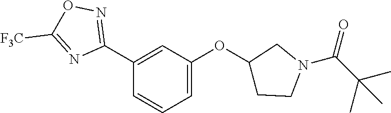 [SMILES: CC(C)(C)C(=O)N1CCC(Oc2cccc(-c3noc(C(F)(F)F)n3)c2)C1]

### Step 1: —Preparation of tert-butyl 3-(3-cyanophenoxy)pyrrolidine-1-carboxylate

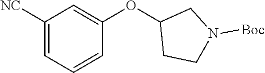 [SMILES: CC(C)(C)OC(=O)N1CCC(Oc2cccc(C#N)c2)C1]

To a solution of tert-butyl 3-hydroxypyrrolidine-1-carboxylate (17.3 g, 92 mmol), 3-hydroxybenzonitrile (10 g, 84 mmol), and triphenylphosphine (28.6 g, 109 mmol) in dry tetrahedron (150 mL), diisopropyl azodicarboxylate (21.2 mL, 109 mmol) was added at 0° C. The resulting reaction mixture was stirred for 12 h at 25° C. After completion of the reaction, the reaction mixture was diluted with dichloromethane (75 mL) and the dichloromethane layer was washed twice with water (50 mL) and brine (50 mL). The dichloromethane layer was separated, dried over anhydrous sodium sulphate, and concentrated to give the crude product. The crude product was purified by column chromatography using 10% ethyl acetate in hexane as an eluent to obtain tert-butyl 3-(3-cyanophenoxy)pyrrolidine-1-carboxylate (18 g, 62.4 mmol, 74% yield).

### Step 2: —Preparation of tert-butyl 3-(3-(N′-hydroxycarbamimidoyl)phenoxy)pyrrolidine-1-carboxylate

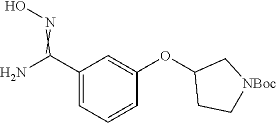 [SMILES: CC(C)(C)OC(=O)N1CCC(Oc2cccc(C(N)=NO)c2)C1]

To a stirred solution of tert-butyl 3-(3-cyanophenoxy)pyrrolidine-1-carboxylate (13 g, 45.1 mmol) in ethanol (80 mL), hydroxylamine (4.2 ml, 67.6 mmol) was added at 25° C. The resulting reaction mixture was stirred at 65° C. for 12 h. After completion of the reaction, ethanol in the reaction mixture was evaporated under reduced pressure. The crude product was co-distilled thrice with dichlormethane (25 mL) to obtain tert-butyl 3-(3-(N′-hydroxycarbamimidoyl)phenoxy)pyrrolidine-1-carboxylate (13 g, 40.5 mmol, 90% yield).

### Step 3: —Preparation of tert-butyl 3-(3-(5-(trifluoromethyl)-1,2,4-oxadiazol-3-yl)phenoxy)pyrrolidine-1-carboxylate (Compound No. 127)

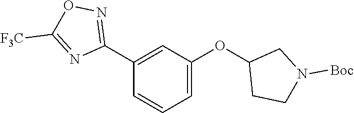 [SMILES: CC(C)(C)OC(=O)N1CCC(Oc2cccc(-c3noc(C(F)(F)F)n3)c2)C1]

To a solution of tert-butyl 3-(3-(N′-hydroxycarbamimidoyl)phenoxy)pyrrolidine-1-carboxylate (13 g, 40.5 mmol) in dry tetrahedron (100 mL), trifluoroacetic anhydride (17.1 mL, 121 mmol) was added at 0° C. under nitrogen atmosphere. The resulting reaction mixture was stirred at 25° C. for 12 h. After completion of the reaction, ethyl acetate (75 mL) was added to the reaction mixture at 0° C. followed by saturated sodium bicarbonate solution until the effervence ceases. Organic layer was washed twice with water (50 mL) and brine (50 mL), dried over sodium sulphate and concentrated. The crude product was purified by column chromatography using 12% ethyl acetate in hexane as an eluent to obtain tert-butyl 3-(3-(5-(trifluoromethyl)-1,2,4-oxadiazol-3-yl)phenoxy)pyrrolidine-1-carboxylate (11.5 g, 28.8 mmol, 71% yield).

1H-NMR (400 MHz, CHLOROFORM-D) δ 7.78 (dd, 1H), 7.66-7.54 (m, 1H), 7.51-7.38 (m, 1H), 7.10 (dd, 1H), 5.17-5.01 (m, 1H), 3.69-3.45 (m, 4H), 2.23-2.19 (m, 2H), 1.50-1.28 (m, 9H); LCMS: −299.90 (M−100)

### Step 4: —Preparation of 3-(3-(pyrrolidin-3-yloxy)phenyl)-5-(trifluoromethyl)-1,2,4-oxadiazole hydrochloride (Compound No. 128)

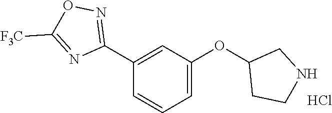 [SMILES: FC(F)(F)c1nc(-c2cccc(OC3CCNC3)c2)no1 ; Cl]

To the solution of tert-butyl 3-(3-(5-(trifluoromethyl)-1,2,4-oxadiazol-3-yl)phenoxy)pyrrolidine-1-carboxylate (11.5 g, 28.8 mmol) in dichloromethane (100 mL) at 0° C., 4M hydrochloric acid in dioxane (46.8 mL, 187 mmol) was added and the resulting reaction mixture was stirred at 25° C. for 12 h. After completion of the reaction, the volatiles were evaporated to obtain a solid mass which was stirred in hexane (100 mL) for 30 min to obtain 3-(3-(pyrrolidin-3-yloxy)phenyl)-5-(trifluoromethyl)-1,2,4-oxadiazole hydrochloride (8.5 g, 25.3 mmol, 88% yield).

1H-NMR (400 MHz, DMSO-D6) δ 9.54 (d, 2H), 7.71 (dq, 1H), 7.62-7.58 (m, 2H), 7.33 (dq, 1H), 5.31 (t, 1H), 3.57-3.47 (m, 1H), 3.39-3.35 (m, 2H), 3.31-3.26 (m, 1H), 2.18-2.14 (m, 2H); LCMS (M+H): 300.0

### Step 5: —Preparation of 2,2-dimethyl-1-(3-(3-(5-(trifluoromethyl)-1,2,4-oxadiazol-3-yl)phenoxy)pyrrolidin-1-yl)propan-1-one (Compound No. 130)

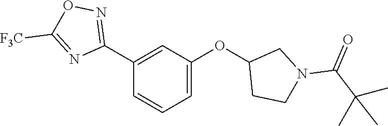 [SMILES: CC(C)(C)C(=O)N1CCC(Oc2cccc(-c3noc(C(F)(F)F)n3)c2)C1]

To the solution of 3-(3-(pyrrolidin-3-yloxy)phenyl)-5-(trifluoromethyl)-1,2,4-oxadiazole hydrochloride (150 mg, 0.4 mmol) in dichloromethane (10 mL), triethylamine (0.3 mL, 1.8 mmol) was added at 25° C. After 10 min of stirring, trimethylacetyl chloride (0.1 mL, 0.5 mmol) was added dropwise at 0° C. The resulting reaction mixture was stirred at 25° C. for 3 h. After completion of the reaction, the reaction mixture was diluted with water (10 mL) and the crude product was extracted thrice with dichloromethane (20 mL). The combined dichloromethane layer was dried over anhydrous sodium sulphate and evaporated in vacuo. The crude product was purified in column to obtain 2,2-dimethyl-1-(3-(3-(5-(trifluoromethyl)-1,2,4-oxadiazol-3-yl)phenoxy)pyrrolidin-1-yl)propan-1-one (130 mg, 0.3 mmol, 76% yield).

1H-NMR (400 MHz, CHLOROFORM-D) δ 7.76 (d, 1H), 7.63 (q, 1H), 7.46 (t, 1H), 7.10 (ddd, 1H), 5.03 (s, 1H), 3.89-3.74 (m, 4H), 2.23 (d, 2H), 1.29-1.13 (m, 9H); LCMS: −384.2 (M+H)

TABLE 21
The following compounds were prepared by the procedure analogous to that for the Compound No. 130

| Compound No. | Compound Name | 1H-NMR and LCMS | Yield |
|---|---|---|---|
| 127 | tert-butyl 3-(3-(5- | 1H-NMR (400 MHz, | 190 mg, 71% |
| | (trifluoromethyl)-1,2,4- | CHLOROFORM-D) δ 7.78 (dd, 1H), | |
| | oxadiazol-3- | 7.66-7.54 (m, 1H), 7.51-7.38 (m, 1H), | |
| | yl)phenoxy)pyrrolidine-1- | 7.10 (dd, 1H), 5.17-5.01 (m, 1H), | |
| | carboxylate | 3.69-3.45 (m, 4H), 2.23-2.19 (m, 2H), | |
| | | 1.50-1.28 (m, 9H); LCMS: −299.90 | |
| | | (M-100) | |
| 130 | 2,2-dimethyl-1-(3-(3-(5- | 1H-NMR (400 MHz, | 130 mg, 76% |
| | (trifluoromethyl)-1,2,4- | CHLOROFORM-D) δ 7.76 (d, 1H), | |
| | oxadiazol-3- | 7.63 (q, 1H), 7.46 (t, 1H), 7.10 (ddd, | |
| | yl)phenoxy)pyrrolidin-1- | 1H), 5.03 (s, 1H), 3.89-3.74 (m, 4H), | |
| | yl)propan-1-one | 2.23 (d, 2H), 1.29-1.13 (m, 9H); | |
| | | LCMS: −384.2 (M + H) | |
| 132 | (2-fluorophenyl)(3-(3-(5- | 1H-NMR @80° C. (400 MHz, DMSO- | 200 mg, 94% |
| | (trifluoromethyl)-1,2,4- | D6) δ 7.69-7.63 (m, 1H), 7.58-7.40 | |
| | oxadiazol-3- | (m, 4H), 7.28-7.23 (m, 3H), 5.24-5.15 | |
| | yl)phenoxy)pyrrolidin-1- | (m, 1H), 3.76-3.69(m, 2H), 3.50-3.33 | |
| | yl)methanone | (m, 2H), 2.32-2.08 (m, 2H); | |
| | | LCMS: −422.2 (M + H) | |
| 133 | (3-fluorophenyl)(3-(3-(5- | 1H-NMR @80° C. (400 MHz, DMSO- | 190 mg, 92% |
| | (trifluoromethyl)-1,2,4- | D6) δ 7.65 (dd, 1H), 7.59-7.43 (m, | |
| | oxadiazol-3- | 3H), 7.41-7.23 (m, 4H), 5.25 (s, 1H), | |
| | yl)phenoxy)pyrrolidin-1- | 3.90-3.85 (m, 1H), 3.69-3.60 (m, 2H), | |
| | yl)methanone | 3.52 (t, 1H), 3.44 (d, 1H), 2.08-2.27 | |
| | | (m, 2H); LCMS: −382 (M-41) | |
| 134 | (4-fluorophenyl)(3-(3-(5- | 1H-NMR @80° C. (400 MHz, DMSO- | 100 mg, 44% |
| | (trifluoromethyl)-1,2,4- | D6) δ 7.66 (d, 1H), 7.62-7.52 (m, 4H), | |
| | oxadiazol-3- | 7.28-7.20 (m, 3H), 5.19 (s, 1H), 3.88 | |
| | yl)phenoxy)pyrrolidin-1- | (dd, 1H), 3.71-3.61 (m, 3H), 2.32-2.21 | |
| | yl)methanone | (m, 1H), 2.17-2.11 (m, 1H); | |
| | | LCMS: −422.1 (M + H) | |
| 135 | p-tolyl(3-(3-(5- | 1H-NMR @80° C. (400 MHz, DMSO- | 160 mg, 80% |
| | (trifluoromethyl)-1,2,4- | D6) δ 7.65 (d, 1H), 7.55-7.52(m, 2H), | |
| | oxadiazol-3- | 7.41 (d, 2H), 7.24 (dd, 3H), 5.18 (s, | |
| | yl)phenoxy)pyrrolidin-1- | 1H), 3.86 (dd, 1H), 3.70-3.60 (m, 3H), | |
| | yl)methanone | 2.33 (s, 3H), 2.25 (tt, 1H), 2.15-2.11 | |
| | | (m, 1H); LCMS: −418.15 (M + H) | |
| 141 | m-tolyl(3-(3-(5- | 1H-NMR @80° C. (400 MHz, DMSO- | 165 mg, 83% |
| | (trifluoromethyl)-1,2,4- | D6) δ 7.66 (d, 1H), 7.53 (d, 2H), 7.30- | |
| | oxadiazol-3- | 7.26 (m, 5H), 5.20 (d, 1H), 3.85 (dd, | |
| | yl)phenoxy)pyrrolidin-1- | 1H), 3.66 (d, 3H), 2.33 (s, 3H), 2.25 | |
| | yl)methanone | (tt, 1H), 2.16 (s, 1H); LCMS: −418.2 | |
| | | (M + H) | |
| 143 | (3-(3-(5-(trifluoromethyl)- | 1H-NMR (400 MHz, DMSO-D6) δ | 100 mg, 44% |
| | 1,2,4-oxadiazol-3- | 7.79-7.65 (m, 5H), 7.56 (s, 2H), 7.28 | |
| | yl)phenoxy)pyrrolidin-1-yl)(4- | (s, 1H), 5.32-5.08 (m, 1H), 3.85 (s, | |
| | (trifluoromethyl)phenyl)methanone | 1H), 3.61 (d, 3H), 2.27 (s, 1H), 2.17 | |
| | | (s, 1H); LCMS: −472.05 (M + H) | |
| 144 | (3-chlorophenyl)(3-(3-(5- | 1H-NMR (400 MHz, DMSO-D6) δ | 160 mg, 77% |
| | (trifluoromethyl)-1,2,4- | 7.66 (d, 1H), 7.54-7.47 (m, 6H), 7.29 | |
| | oxadiazol-3- | (s, 1H), 5.20 (s, 1H), 3.89-3.84 (m, | |
| | yl)phenoxy)pyrrolidin-1- | 1H), 3.67 (s, 3H), 2.26 (q, 1H), 2.17 | |
| | yl)methanone | (s, 1H); LCMS: −438 (M + H) | |
| 146 | (4-methoxyphenyl)(3-(3-(5- | 1H-NMR (400 MHz, DMSO-D6) δ | 150 mg, 73% |
| | (trifluoromethyl)-1,2,4- | 7.65 (d, 1H), 7.53 (q, 4H), 7.28-7.26 | |
| | oxadiazol-3- | (m, 1H), 6.96 (d, 2H), 5.18 (s, 1H), | |
| | yl)phenoxy)pyrrolidin-1- | 3.91-3.87 (m, 1H), 3.80 (s, 3H), 3.73- | |
| | yl)methanone | 3.59 (m, 3H), 2.29-2.20 (m, 1H), 2.16 | |
| | | (s, 1H); LCMS: −434 (M + H) | |
| 208 | pyridin-2-yl(3-(3-(5- | 1H-NMR (400 MHz, DMSO-D6) δ | 160 mg, 74% |
| | (trifluoromethyl)-1,2,4- | 8.62-8.57 (m, 1H), 7.90 (t, 1H), 7.76 | |
| | oxadiazol-3- | (d, 1H), 7.68-7.63 (m, 1H), 7.58-7.44 | |
| | yl)phenoxy)pyrrolidin-1- | (m, 3H), 7.32-7.24 (m, 1H), 5.21 (d, | |
| | yl)methanone | 1H), 4.05-3.71 (m, 4H), 2.28-2.12 (m, | |
| | | 2H); LCMS: −405.2 (M + H) | |
| 209 | pyridin-4-yl(3-(3-(5- | 1H-NMR @80° C. (400 MHz, DMSO- | 190 mg, 88% |
| | (trifluoromethyl)-1,2,4- | D6) δ 8.66 (s, 2H), 7.66 (s, 1H), 7.53 | |
| | oxadiazol-3- | (d, 4H), 7.27 (d, 1H), 5.25 (s, 1H), | |
| | yl)phenoxy)pyrrolidin-1- | 3.88 (s, 1H), 3.62 (d, 3H), 2.27 (d, | |
| | yl)methanone | 1H), 2.17 (s, 1H); LCMS: −405 (M + H) | |
| 210 | pyridin-3-yl(3-(3-(5- | 1H-NMR @80° C. (400 MHz, DMSO- | 190 mg, 88% |
| | (trifluoromethyl)-1,2,4- | D6) δ 8.72 (s, 1H), 8.63 (d, 1H), 7.94 | |
| | oxadiazol-3- | (s, 1H), 7.66 (d, 1H), 7.56 (s, 2H), | |
| | yl)phenoxy)pyrrolidin-1- | 7.45 (dd, 1H), 7.29 (s, 1H), 5.22 (s, | |
| | yl)methanone | 1H), 3.90 (dd, 1H), 3.69 (d, 3H), 2.29- | |
| | | 2.24 (m, 1H), 2.17 (s, 1H); | |
| | | LCMS: −405 (M + H) | |

### Example 24: Preparation of 1-(3-(bis(4-(5-(trifluoromethyl)-1,2,4-oxadiazol-3-yl)phenyl)amino)azetidin-1-yl)ethan-1-one (Compound No. 90)

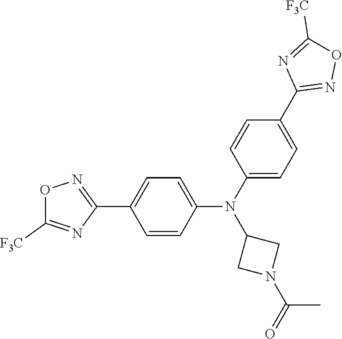 [SMILES: CC(=O)N1CC(N(c2ccc(-c3noc(C(F)(F)F)n3)cc2)c2ccc(-c3noc(C(F)(F)F)n3)cc2)C1]

### Step 1: Preparation of tert-butyl 3-(bis(4-(N′-hydroxycarbamimidoyl)phenyl)amino)azetidine-1-carboxylate

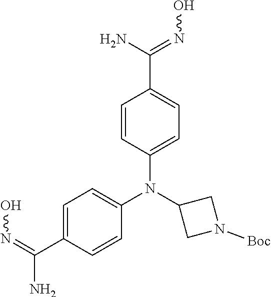 [SMILES: CC(C)(C)OC(=O)N1CC(N(c2ccc(C(N)=NO)cc2)c2ccc(C(N)=NO)cc2)C1]

To a stirred solution of tert-butyl 3-(bis(4-cyanophenyl)amino)azetidine-1-carboxylate (2.1 g, 5.61 mmol) (obtained in step 2 of example 21) in ethanol (70 mL), hydroxylamine (50% solution in water) (1.03 mL, 16.8 mmol) was added at 25° C. The resulting reaction mixture was stirred at 65° C. for 6 h. The reaction mixture was concentrated under reduced pressure to obtain tert-butyl 3-(bis(4-(N′-hydroxycarbamimidoyl)phenyl)amino)azetidine-1-carboxylate (2.1 g, 4.77 mmol, 85% yield).

### Step 2: Preparation of tert-butyl 3-(bis(4-(5-(trifluoromethyl)-1,2,4-oxadiazol-3-yl)phenyl)amino)azetidine-1-carboxylate (Compound No. 82)

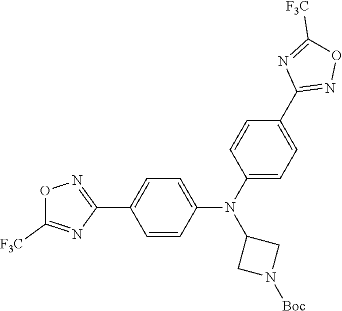 [SMILES: CC(C)(C)OC(=O)N1CC(N(c2ccc(-c3noc(C(F)(F)F)n3)cc2)c2ccc(-c3noc(C(F)(F)F)n3)cc2)C1]

To a stirred solution of tert-butyl 3-(bis(4-(N′-hydroxycarbamimidoyl)phenyl)amino)azetidine-1-carboxylate (2.1 g, 4.8 mmol) and tetrahydrofuran (50 ml), trifluoroacetic anhydride (2.020 ml, 14.30 mmol) was added at 0° C. under nitrogen atmosphere. The reaction mixture was stirred at 25° C. for 6 hours. Ethyl acetate (50 mL) and saturated sodium bicarbonate solution were added to the reaction mixture and stirred until effervescence ceases. The ethyl acetate layer was isolated, washed with water (10 mL), dried over anhydrous sodium sulphate and concentrated under reduced pressure. The obtained crude product was purified by column chromatography to get tert-butyl 3-(bis(4-(5-(trifluoromethyl)-1,2,4-oxadiazol-3-yl)phenyl)amino)azetidine-1-carboxylate (2 g, 3.35 mmol, 70% yield).

1H-NMR (400 MHz, CHLOROFORM-D) δ 8.12 (dt, J=9.2, 2.3 Hz, 4H), 7.01 (dt, J=9.1, 2.2 Hz, 4H), 4.77-4.71 (m, 1H), 4.29-4.12 (m, 2H), 3.88 (dd, J=9.4, 5.7 Hz, 2H), 1.47 (d, J=20.1 Hz, 9H); LCMS (M+H): 596.5

### Step 3: Preparation of N,N-bis(4-(5-(trifluoromethyl)-1,2,4-oxadiazol-3-yl)phenyl)azetidin-3-amine Hydrochloride (Compound No. 86)

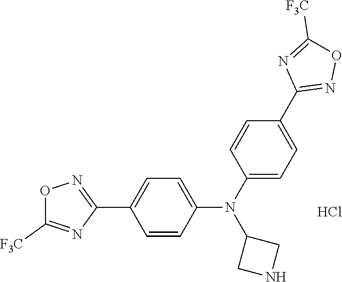 [SMILES: FC(F)(F)c1nc(-c2ccc(N(c3ccc(-c4noc(C(F)(F)F)n4)cc3)C3CNC3)cc2)no1 ; Cl]

N,N-bis(4-(5-(trifluoromethyl)-1,2,4-oxadiazol-3-yl)phenyl)azetidin-3-amine hydrochloride was prepared by procedure analogous to step 5 of example 21.

### Step 4: Preparation of 1-(3-(bis(4-(5-(trifluoromethyl)-1,2,4-oxadiazol-3-yl)phenyl)amino)azetidin-1-yl)ethan-1-one (Compound No. 90)

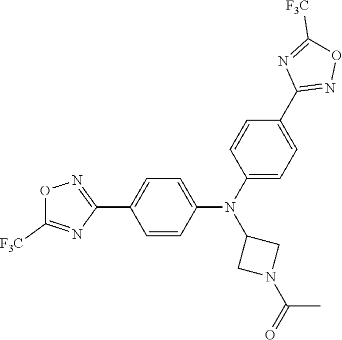 [SMILES: CC(=O)N1CC(N(c2ccc(-c3noc(C(F)(F)F)n3)cc2)c2ccc(-c3noc(C(F)(F)F)n3)cc2)C1]

1-(3-(bis(4-(5-(trifluoromethyl)-1,2,4-oxadiazol-3-yl)phenyl)amino)azetidin-1-yl)ethan-1-one (230 mg, 76%) was prepared by procedure analogues to step 6 of example 21

1H-NMR (400 MHz, CHLOROFORM-D) δ 8.10 (d, J=8.6 Hz, 4H), 6.99 (d, J=8.6 Hz, 4H), 4.82-4.75 (m, 1H), 4.47-4.29 (m, 2H), 3.99 (d, J=51.0 Hz, 2H), 1.86 (s, 3H); LCMS (M+H): 539.0

### Example 27: Preparation of 1-(methylsulfonyl)-N,N-bis(4-(5-(trifluoromethyl)-1,2,4-oxadiazol-3-yl)phenyl)azetidin-3-amine (Compound No. 91)

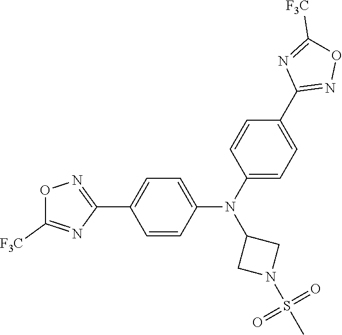 [SMILES: CS(=O)(=O)N1CC(N(c2ccc(-c3noc(C(F)(F)F)n3)cc2)c2ccc(-c3noc(C(F)(F)F)n3)cc2)C1]

1-(methylsulfonyl)-N,N-bis(4-(5-(trifluoromethyl)-1,2,4-oxadiazol-3-yl)phenyl)azetidin-3-amine (140 mg, 49% yield) was prepared by procedure analogous to step 1 of example 22.

1H-NMR (400 MHz, CHLOROFORM-D) δ 8.12-8.18 (m, 4H), 7.02 (dt, J=9.1, 2.3 Hz, 4H), 4.84-4.77 (m, 1H), 4.22-4.18 (m, 2H), 3.97-3.93 (m, 2H), 2.85 (s, 3H); LCMS (M+H): 575.0

As described herein the compounds of general Formula I show an extremely high fungicidal activity which is exerted with respect to numerous phytopathogenic fungi which attacks on important agricultural crops. Compounds of present invention were assessed for activity against one or more of the following:

### BIOLOGY EXAMPLES

### Biological Test Examples (In Vitro Test)

### Example 1: Pyricularia oryzae (Rice Blast)

Compounds were dissolved in 0.3% DMSO and then added to Potato Dextrose Agar medium just prior to dispensing it into petri dishes. 5 mL medium with a compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds

| | | | | | | | | | | | |
|---|---|---|---|---|---|---|---|---|---|---|---|
| | 2 | 5 | 11 | 12 | 13 | 17 | 40 | 45 | 48 | 49 | 55 |
| 56 | 58 | 59 | 60 | 61 | 66 | 68 | 69 | 78 | 83 | 85 | 89 |
| 90 | 92 | 95 | 96 | 97 | 98 | 99 | 100 | 133 | 134 | 135 | 137 |

| | | | | | | | | | |
|---|---|---|---|---|---|---|---|---|---|
| 163 | 165 | 167 | 172 | 177 | 179 | 180 | 198 | 199 | and 200 showed >70% |

at 300 ppm control in these tests when compared to the
untreated check which showed extensive disease development.

### Example 2: Rhizoctonia solani (Rice Sheath Blight/Potato Black Scurf)

Compounds were dissolved in 0.3% DMSO and then added to Potato Dextrose Agar medium just prior to dispensing it into petri dishes. 5 mL medium with a compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds 2

| | | | | | | | | | | |
|---|---|---|---|---|---|---|---|---|---|---|
| 12 | 13 | 48 | 49 | 55 | 59 | 66 | 68 | 69 | 83 | 85 |

| | | | | | |
|---|---|---|---|---|---|
| 89 | 95 | 100 | 137 | 163 | and 165 showed >70% at 300 ppm |

control in these tests when compared to the untreated
check which showed extensive disease development.

### Example 3: Botrytis cinerea (Gray Mold)

Compounds were dissolved in 0.3% DMSO and then added to Potato Dextrose Agar medium just prior to dispensing it into petri dishes. 5 mL medium with compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 22° C. temperature and 90% relative humidity for seven days and radial growth was measured.

Compounds 2

| | | | | | | | | | | |
|---|---|---|---|---|---|---|---|---|---|---|
| 13 | 48 | 55 | 59 | 66 | 68 | 69 | 83 | 89 | 95 | 100 |

| | | |
|---|---|---|
| 163 | 165 | and 180 showed >70% at 300 ppm control in |

these tests when compared to the untreated check
which showed extensive disease development.

### Example 4: Alternaria solani (Early Blight of Tomato/Potato)

Compounds were dissolved in 0.3% DMSO and then added to Potato Dextrose Agar medium just prior to dispensing it into petri dishes. 5 mL medium with a compound in the desired concentration was dispensed into 6 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds 1

| | | | | | | | | | | | |
|---|---|---|---|---|---|---|---|---|---|---|---|
| | 2 | 6 | 12 | 13 | 24 | 48 | 49 | 58 | 59 | 66 | 68 |
| 69 | 85 | 86 | 89 | 94 | 95 | 96 | 97 | 98 | 99 | 100 | 132 |

| | | | | | | | | |
|---|---|---|---|---|---|---|---|---|
| 133 | 134 | 137 | 158 | 163 | 165 | 180 | 199 | and 200 showed >70% |

at 300 ppm control in these tests when compared to the
untreated check which showed extensive disease development.

### Example 5: Colletotrichum capsici (Anthracnose)

Compounds were dissolved in 0.3% DMSO and then added to Potato Dextrose Agar medium just prior to dispensing it into petri dishes. 5 mL medium with compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds 2

| | | | | | | | | | | |
|---|---|---|---|---|---|---|---|---|---|---|
| 12 | 13 | 49 | 55 | 59 | 66 | 68 | 69 | 83 | 87 | 89 |

| | | | | | | | |
|---|---|---|---|---|---|---|---|
| 95 | 96 | 100 | 136 | 165 | 180 | 199 | and 200 showed >70% at |

300 ppm control in these tests when compared to the untreated
check which showed extensive disease development.

### Example 6: Fusarium culmorum (Foot Rot of Cereals)

Compounds were dissolved in 0.3% DMSO and then added to Potato Dextrose Agar medium just prior to dispensing it into petri dishes. 5 mL medium with compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 60% relative humidity for seven days and radial growth was measured.

Compounds 2

| | | | | | | | | |
|---|---|---|---|---|---|---|---|---|
| 12 | 13 | 55 | 66 | 69 | 89 | 95 | 100 | and 199 |

showed >70% at 300 ppm control in these tests when compared to
the untreated check which showed extensive disease development.

### Example 7: Corynespora cassicola (Leaf Spot of Tomato)

Compounds were dissolved in 0.3% DMSO and then added to Potato Dextrose Agar medium just prior to dispensing it into petri dishes. 5 mL medium with compound in the desired concentration was dispensed into 60 mm sterile petri-plates. After solidification, each plate was seeded with a 5 mm size mycelial disc taken form the periphery of an actively growing virulent culture plate. Plates were incubated in growth chambers at 25° C. temperature and 70% relative humidity for seven days and radial growth was measured.

Compounds 2

| | | | | | | | | | | |
|---|---|---|---|---|---|---|---|---|---|---|
| 55 | 66 | 69 | 83 | 85 | 89 | 95 | 100 | 137 | 158 | 163 |

| | |
|---|---|
| 165 | and 180 showed >70% at 300 ppm control in these |

tests when compared to the untreated check which showed
extensive disease development.

### Biological Test Examples (Greenhouse)

### Example A: Phakopsora pachyrhizi Test in Soybean

Compounds were dissolved in 2% DMSO/Acetone and then diluted with water containing emulsifier to the desired test concentration.

To test the preventive activity of compounds, healthy young soybean plants, raised in the greenhouse, were sprayed with the active compound solution at the stated application rates inside spray cabinets using hallowcone nozzles. One day after treatment, the plants were inoculated with a suspension containing 2.1×10⁶ Phakopsora pachyrhizi spores. The inoculated plants were then kept in the greenhouse chamber at 25° C. temperature and 90% relative humidity for disease expression.

A visual assessment of the compound's performance was carried out by rating the disease severity (0-100% scale) on treated plants on 3, 7, 10 and 15 days after application. Efficacy (% control) of the compounds was calculated by comparing the disease rating in the treatment with the one of the untreated control. The treated plants were also assessed for plant compatibility by recording symptoms like necrosis, chlorosis and stunting.

Compounds 1

| | | | | | | | | | | | |
|---|---|---|---|---|---|---|---|---|---|---|---|
| | 2 | 3 | 5 | 8 | 9 | 11 | 12 | 13 | 14 | 15 | 16 |
| 17 | 18 | 20 | 22 | 23 | 24 | 25 | 26 | 27 | 28 | 29 | 30 |
| 31 | 32 | 33 | 34 | 35 | 36 | 37 | 38 | 41 | 42 | 43 | 44 |
| 45 | 46 | 47 | 48 | 49 | 50 | 51 | 52 | 53 | 54 | 56 | 57 |
| 59 | 60 | 61 | 62 | 63 | 64 | 65 | 71 | 74 | 79 | 80 | 81 |
| 82 | 83 | 84 | 85 | 86 | 87 | 88 | 100 | 101 | 102 | 104 | 105 |
| 106 | 107 | 108 | 109 | 110 | 111 | 112 | 113 | 114 | 115 | 116 | 120 |
| 121 | 122 | 124 | 127 | 128 | 129 | 130 | 142 | 143 | 144 | 145 | 146 |

| | | | | | | | | |
|---|---|---|---|---|---|---|---|---|
| 147 | 148 | 149 | 150 | 151 | 152 | 153 | 155 | and 157 showed >70 |

% at 500 ppm control in these tests when compared to the
untreated check which showed extensive disease development.

1. A compound of Formula I, 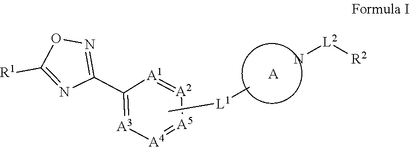
wherein,
R¹ is selected from the group consisting of C₁-C₂-monohaloalkyl, C₁-C₂-dihaloalkyl, C₁-C₂-trihaloalkyl, C₁-C₂-tetrahaloalkyl, and C₁-C₂-pentahaloalkyl;
A¹ is C or N;
A² is C or N;
A³ is C or N;
A⁴ is C or N; and
A⁵ is C or N; wherein no more than two of A¹, A², A³, A⁴, and A⁵ are nitrogen,
wherein, A¹, A², A³, A⁴, and A⁵ are independently and optionally substituted with one or more R^{G} selected from the group consisting of hydrogen, halogen, cyano, nitro, amino, hydroxy, C₃-C₆-cycloalkyl, C₁-C₆-haloalkyl, C₁-C₆-hydroxyalkyl, C₁-C₆-alkoxy, C₁-C₆-alkoxy-C₁-C₆-alkyl, and C₁-C₆-haloalkoxy;
L¹ is O, S(═O)₀₋₂, NR⁶, or 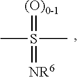
wherein, L¹ may be attached to either of A², A⁴, or A⁵;
A is a nitrogen containing 3-, 4-, 5- or 6-membered nonaromatic heterocyclic ring; wherein, ring A additionally may comprise a hetero atom selected from N, O, and S(═O)₀₋₂; wherein, ring A may be optionally substituted with one or more R^{A},
wherein, R^{A} is independently selected from hydrogen, halogen, cyano, nitro, amino, hydroxy, oxo, C₂-C₆-alkenyl, C₂-C₆-alkynyl, C₃-C₈-cycloalkyl, C₃-C₈-cycloalkylalkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, C₁-C₆-haloalkoxycarbonyl, C₁-C₆-haloalkylthio, C₁-C₆-haloalkylsulfinyl, C₁-C₆-haloalkylsulfonyl, C₁-C₆-alkylsulfonyl, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, C₁-C₆-dialkylaminocarbonyl, C₁-C₆-alkoxycarbonyloxy, C₁-C₆-alkylaminocarbonyloxy, C₁-C₆-dialkylaminocarbonyloxy, sulfilimines, sulfoximines, sulfonamide, and sulfinamide;
L² is (C(═O))₁₋₂, (CR⁸R⁹)₁₋₃, S(═O)₀₋₂, NR¹⁸, or 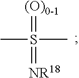
wherein, R² is selected from hydrogen, halogen, cyano, nitro, amino, hydroxy, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, aryloxy, heteroaryloxy, C₄-C₈-heterocyclyloxy, C₃-C₈-cycloalkyloxy, C₁-C₆-haloalkoxycarbonyl, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, C₄-C₈-heterocyclylamino, heteroarylamino, arylamino, C₁-C₆-dialkylamino, C₃-C₈-cycloalkylamino, C₁-C₆-alkyl-C₃-C₈-cycloalkylamino, C₁-C₆-alkylcarbonyl, C₁-C₆-alkoxycarbonyl, C₁-C₆-alkylaminocarbonyl, C₁-C₆-dialkylaminocarbonyl, C₁-C₆-alkoxycarbonyloxy, C₁-C₆-alkylaminocarbonyloxy, or C₁-C₆-dialkylaminocarbonyloxy, sulfilimines, sulfoximines, sulfonamide, and sulfinamide; R² may optionally further be substituted with one or more R⁷; or
R² is phenyl, benzyl, naphthyl, a 5- or 6-membered aromatic ring, an 8- to 11-membered aromatic multi-cyclic ring system, an 8- to 11-membered aromatic fused ring system, a 5- or 6-membered heteroaromatic ring, an 8- to 11-membered heteroaromatic multi-cyclic ring system or an 8- to 11-membered heteroaromatic fused ring system; wherein heteroatom of the heteroaromatic ring or ring system is selected from N, O or S, and each aromatic or heteroaromatic ring or ring system may be optionally substituted with one or more substituents selected from R³; or
R² is a 3- to 7-membered nonaromatic carbocyclic ring, a 4-, 5-, 6- or 7-membered nonaromatic heterocyclic ring, an 8- to 15-membered nonaromatic multi-cyclic ring system, an 5- to 15 membered spirocyclic ring system, or an 8- to 15-membered nonaromatic fused ring system, wherein, the heteroatom of the nonaromatic heterocyclic ring or ring system is selected from N, O or S(O)₀₋₂, and C ring member of the nonaromatic carbocyclic or nonaromatic heterocyclic ring or ring system may be replaced with C(═O), C(═S), C(═CR²⁰R²¹) or C(═NR¹⁹) and each nonaromatic carbocyclic or nonaromatic heterocyclic ring or ring system may be optionally substituted with one or more substituents selected from R³ wherein, R³ is independently selected from halogen, cyano, nitro, hydroxy, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₈-cycloalkyl, C₃-C₈-halocycloalkyl, C₃-C₈-cycloalkyl-C₁-C₆-alkyl, C₃-C₈-cycloalkyl-C₃-C₈-cycloalkyl, C₃-C₈-cycloalkenyl, C₁-C₆-alkoxy-C₁-C₆-alkyl, C₃-C₈-cycloalkoxy-C₁-C₆-alkyl, C₁-C₆-alkylsulfinyl-C₁-C₆-alkyl, C₁-C₆-alkylsulfonyl-C₁-C₆-alkyl, C₁-C₆-alkylamino, di-C₁-C₆-alkylamino, C₁-C₆-alkylamino-C₁-C₆-alkyl, di-C₁-C₆-alkylamino-C₁-C₆-alkyl, C₁-C₆-haloalkylamino-C₁-C₆-alkyl, C₃-C₈-cycloalkylamino, C₃-C₈-cycloalkylamino-C₁-C₆-alkyl, C₁-C₆-alkylcarbonyl, C₁-C₆-haloalkoxy-C₁-C₆-alkyl, C₁-C₆-hydroxyalkyl, C₂-C₆-hydroxyalkenyl, C₂-C₆-hydroxyalkynyl, C₂-C₆-alkenyloxy, C₂-C₆-haloalkenyloxy, C₂-C₆-alkynyloxy, C₁-C₆-alkylcarbonylalkoxy, C₁-C₆-alkylthio, C₁-C₆-haloalkylthio, C₃-C₈-cycloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkylsulfonyl, C₃-C₈-cycloalkylsulfinyl, C₁-C₆-alkylsulfonylamino, C₁-C₆-haloalkylsulfonylamino, C₁-C₆-alkylsulfonyloxy, C₆-C₁₀-arylsulfonyloxy, C₆-C₁₀-arylsulfonyl, C₆-C₁₀-arylsulfinyl, C₆-C₁₀-arylthio, C₁-C₆-cyanoalkyl, C₁-C₆-haloalkylamino, C₁-C₆-alkoxyamino, C₁-C₆-haloalkoxyamino, C₁-C₆-alkoxycarbonylamino, C₁-C₆-alkylcarbonyl-C₁-C₆-alkylamino, C₂-C₆-alkenylthio, di(C₁-C₆-haloalkyl)amino-C₁-C₆-alkyl, C₁-C₆-alkylaminocarbonylamino, di(C₁-C₆-haloalkyl)amino, sulfilimines, sulfoximines, or SF₅; wherein, R³ may be optionally substituted with halogen, cyano, amino, C₁-C₆-alkyl, C₁-C₆-alkoxy, C₁-C₆-alkylthio, and C₃-C₈-cycloalkyl;
R⁷ is selected from C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, aryloxy, heteroaryloxy, arylamino, heteroarylamino, arylthio, heteroarylthio, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, C₁-C₆-alkylamino-C₁-C₆-alkyl, C₁-C₆-haloalkoxycarbonyl, and amino-C₁-C₆-alkyl; or
R⁷ is phenyl, benzyl, a 5-membered aromatic ring, a 5- or 6-membered heteroaromatic ring; wherein heteroatom of the heteroaromatic ring is selected from N, O or S; or
R⁷ is a 3- to 7-membered nonaromatic carbocyclic ring, a 4-, 5-, 6- or 7-membered nonaromatic heterocyclic ring, wherein, the heteroatom of the nonaromatic heterocyclic ring is selected from N, O or S(O)₀₋₂, and C ring member of the nonaromatic carbocyclic or nonaromatic heterocyclic ring may be replaced with C(═O), C(═S), C(═CR²²R²³), or C(═NR²⁴) wherein, R⁷ may be further substituted with one or more R¹⁶ on C atom and with one or more R¹⁷ on N atom,
R⁴, R⁵, R⁸, R⁹, R¹⁶, R²⁰, R²¹, R²², and R²³ are independently selected from hydrogen, halogen, cyano, nitro, NR¹⁰R¹¹, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, C₃-C₆-halocycloalkyl, C₁-C₄-alkoxy, C₃-C₈-cycloalkoxy, or C₁-C₄-haloalkoxy,
R⁶, R¹⁰, R¹¹, R¹⁷, R¹⁸, R¹⁹, and R²⁴ are independently selected from the group of hydrogen, cyano, hydroxy, NR^{b}R^{c}, (C═O)—R^{d}, S(O)₀₋₂R^{e}, C₁-C₆-alkyl, C₁-C₆-haloalkyl, C₁-C₆-alkoxy, C₁-C₆-haloalkoxy, C₃-C₆-cycloalkyl, phenyl, aryl-C₁-C₆-alkyl, heteroaryl, heteroaryl-C₁-C₆-alkyl, C₁-C₆-alkylamino, di-C₁-C₆-alkylamino, tri-C₁-C₆-alkylamino, or C₃-C₈-cycloalkyl;
R^{b} and R^{C} represent hydrogen, hydroxyl, cyano, amino, C₁-C₄-alkyl, C₁-C₄-haloalkyl, C₁-C₄-alkoxy, C₃-C₈-cycloalkyl, or C₃-C₈-halocycloalkyl;
R^{d} represents hydrogen, hydroxy, halogen, NR^{b}R^{c}, C₁-C₆-alkyl, C₁-C₆-haloalkyl, C₁-C₆-alkoxy, C₁-C₆-haloalkoxy, C₃-C₈-cycloalkyl, or C₃-C₈-halocycloalkyl; and
R^{e} represents hydrogen, halogen, cyano, amino, C₁-C₆-alkyl, C₁-C₆-haloalkyl, C₁-C₆-alkoxy, C₁-C₆-haloalkoxy, C₃-C₈-cycloalkyl, or C₃-C₈-halocycloalkyl;
or N-oxides, metal complexes, isomers, polymorphs or the agriculturally acceptable salts thereof,
proviso that the following compounds are excluded from the definition of Formula I:
1-[4-[[5-[5-(trifluoromethyl)-1,2,4-oxadiazol-3-yl]-2-pyrimidinyl]amino]-1-piperidinyl]-ethanone, (2360451-15-4);
3-[2-chloro-4-[5-(trifluoromethyl)-1,2,4-oxadiazol-3-yl]phenoxy]-4-hydroxy-4-methyl-1,1-dimethylethyl ester-1-piperidinecarboxylic acid, (2127083-53-6);
3-hydroxy-3-methyl-4[[5-[5-(trifluoromethyl)-1,2,4-oxadiazol-3-yl]-2-pyridinyl]oxy]-1,1-dimethylethyl ester-1-piperidinecarboxylic acid, (2125466-28-4);
N-[1-[(1-methyl-1H-indol-3-yl)methyl]-4-piperidinyl]-5-[5-(trifluoromethyl)-1,2,4-oxadiazol-3-yl]-2-pyrimidinamine, (1434044-32-2);
N-[1-[(1-methyl-1H-indol-3-yl)methyl]-4-piperidinyl]-5-[5-(trifluoromethyl)-1,2,4-oxadiazol-3-yl]-2-pyridinamine, (1434044-31-1),
4[[5-[5-(trifluoromethyl)-1,2,4-oxadiazol-3-yl]-2-pyrimidinyl]amino]-1,1-dimethylethyl ester-1-piperidinecarboxylic acid, (1433958-20-3);
4[[5-[5-(trifluoromethyl)-1,2,4-oxadiazol-3-yl]-2-pyridinyl]amino]-1,1-dimethylethyl ester-1-piperidinecarboxylic acid, (1433958-19-0);
N-[1-[(1-methyl-1H-indol-3-yl)methyl]-4-piperidinyl]-5-[5-(trifluoromethyl)-1,2,4-oxadiazol-3-yl]-2-pyrimidinamine, hydrochloride, (1433958-05-4); and
N-[1-[(1-methyl-1H-indol-3-yl)methyl]-4-piperidinyl]-5-[5-(trifluoromethyl)-1,2,4-oxadiazol-3-yl]-2-pyridinamine, hydrochloride, (1433958-02-1).

2. The compound of claim 1,
wherein,
R¹ is difluoromethyl or trifluoromethyl;
A¹ is C;
A² is C or N;
A³ is C;
A⁴ is C or N; and
A⁵ is C or N; wherein no more than one of A², A⁴, and A⁵ are nitrogen,
wherein, A¹, A², A³, A⁴, and A⁵ are independently and optionally substituted with one or more R^{G} selected from the group consisting of hydrogen, halogen, cyano, C₁-C₆-alkoxy, C₃-C₆-cycloalkyl, and C₁-C₆-haloalkoxy;
L¹ is O, S(═O)₀₋₂, or NR⁶,
wherein, L¹ may be attached to either of A², A⁴, or A⁵;
A is a nitrogen containing 4-, 5- or 6-membered nonaromatic heterocyclic ring; wherein, ring A may be optionally substituted with one or more R^{A},
wherein, R^{A} is independently selected from halogen, cyano, C₁-C₆-alkyl, C₂-C₆-alkenyl, C₂-C₆-alkynyl, C₁-C₆-alkoxy, and C₃-C₈-cycloalkyl;
L² is C(═O), (CR⁸R⁹), S(═O)₂, or NR¹⁸;
wherein, R² is selected from hydrogen, C₁-C₆-alkyl, C₂-C₆-alkenyl, C₂-C₆-alkynyl, C₃-C₈-cycloalkyl, C₃-C₈-cycloalkylalkyl, C₁-C₆-haloalkyl, C₁-C₆-alkoxy-C₁-C₄-alkyl, C₁-C₆-hydroxyalkyl, C₂-C₆-haloalkenyl, C₂-C₆-haloalkynyl, C₃-C₈-halocycloalkyl, C₁-C₆-alkoxy, C₁-C₆-haloalkoxy, aryloxy, heteroaryloxy, C₄-C₈-heterocyclyloxy, C₃-C₈-cycloalkyloxy, C₁-C₆-haloalkoxycarbonyl, C₁-C₆-alkylthio, C₁-C₆-haloalkylthio, C₁-C₆-haloalkylsulfinyl, C₁-C₆-haloalkylsulfonyl, C₁-C₆-alkylsulfinyl, C₁-C₆-alkylsulfonyl, C₁-C₆-alkylamino, C₄-C₈-heterocyclylamino, heteroarylamino, and arylamino; R² may optionally further be substituted with one or more R⁷; or
R² is phenyl, benzyl, a 5- or 6-membered aromatic ring, a 5- or 6-membered heteroaromatic ring; wherein heteroatom of the heteroaromatic ring or ring system is N, and each aromatic or heteroaromatic ring or ring system may be optionally substituted with one or more substituents selected from R³; or
R² is a 3- to 7-membered nonaromatic carbocyclic ring, a 4-, 5-, 6- or 7-membered nonaromatic heterocyclic ring, the heteroatom of the nonaromatic heterocyclic ring or ring system is selected from N, O or S(O)₀₋₂, and each nonaromatic carbocyclic or nonaromatic heterocyclic ring or ring system may be optionally substituted with one or more substituents selected from R³,
wherein, R³ is independently selected from halogen, cyano, C₁-C₆-alkyl, C₁-C₆-haloalkyl, C₂-C₆-haloalkenyl, C₃-C₈-cycloalkyl, C₃-C₈-halocycloalkyl, C₁-C₆-alkoxy, and C₁-C₆-haloalkoxy;
R⁷ is selected from halogen, hydroxy, amino, C₁-C₆-alkyl, and C₃-C₈-cycloalkyl; or
R⁷ is phenyl, benzyl, a 5-membered aromatic ring, a 5- or 6-membered heteroaromatic ring; wherein heteroatom of the heteroaromatic ring is selected from N, O or S; or
R⁷ is a 3- to 7-membered nonaromatic carbocyclic ring, a 4-, 5-, 6- or 7-membered nonaromatic heterocyclic ring, wherein, the heteroatom of the nonaromatic heterocyclic ring is selected from N;
wherein, R⁷ may be further substituted with one or more R¹⁶ on C atom and with one or more R¹⁷ on N atom,
R⁴, R⁸, R⁹ and R¹⁶ are independently selected from hydrogen, halogen, cyano, C₁-C₄-alkyl, C₁-C₄-haloalkyl, C₃-C₆-cycloalkyl, C₃-C₆-halocycloalkyl, C₁-C₄-alkoxy, C₃-C₈-cycloalkoxy, and C₁-C₄-haloalkoxy,
R⁶, R¹⁷, and R¹⁶ are selected from the group of hydrogen, cyano, (C═O)—R^{d}, S(O)₀₋₂R^{e}, C₁-C₆-alkyl, C₁-C₆-haloalkyl, C₁-C₆-alkoxy, C₁-C₆-haloalkoxy, and C₃-C₆-cycloalkyl;
R^{d} represents hydrogen, hydroxy, halogen, NR^{b}R^{c}, C₁-C₆-alkyl, C₁-C₆-haloalkyl, C₁-C₆-alkoxy, or C₃-C₈-cycloalkyl;
R^{b} and R^{C} represent hydrogen, C₁-C₄-alkyl, C₁-C₄-haloalkyl, C₁-C₄-alkoxy, or C₃-C₈-cycloalkyl; and
R^{e} represents hydrogen, amino, C₁-C₆-alkyl, C₁-C₆-haloalkyl, C₁-C₆-alkoxy, or C₃-C₈-cycloalkyl;
or N-oxides, metal complexes, isomers, polymorphs or the agriculturally acceptable salts thereof.

3. The compound of claim 1,
wherein,
R¹ is trifluoromethyl;
A¹ is C;
A² is C or NI;
A³ is C;
A⁴ is C or N; and
A⁵ is C; wherein no more than one of A² & A⁴ are nitrogen,
wherein, A¹, A², A³, A⁴, and A⁵ are independently and optionally substituted with one or more R^{G} selected from the group consisting of hydrogen, halogen, methyl, ethyl, propyl, isopropyl, methoxy, trifluoro methoxy, trifluoro methyl, difluoro methyl, and cyclopropyl;
L¹ is O, S, S(═O)₂, N-methyl, N-ethyl, N-propyl, N-isopropyl and N-cyclopropyl, wherein, L¹ may be attached to either of A², A⁴ or A⁵;
A is a nitrogen containing 4-, 5- or 6-membered nonaromatic heterocyclic ring; wherein, ring A may be optionally substituted with one or more R^{A}, wherein, R^{A} is independently selected from fluorine, bromine, chlorine, iodine, cyano, methyl, ethyl, propyl, isopropyl, methoxy, ethoxy and cyclopropyl;
L² is C(═O), (CH₂), (CHCH₃), or S(═O)₂;
wherein, R² is selected from hydrogen, C₁-C₆-alkyl, C₃-C₈-cycloalkyl, C₁-C₆-haloalkyl, C₃-C₈-halocycloalkyl, C₁-C₆-alkoxy, C₁-C₆-haloalkoxy, heteroarylamino, arylamino; R² may optionally further be substituted with one or more R⁷; or
R² is phenyl, benzyl, a 5- or 6-membered heteroaromatic ring; wherein heteroatom of the heteroaromatic ring or ring system is N, and each aromatic or heteroaromatic ring or ring system may be optionally substituted with one or more substituents selected from R³; or
R² is a 3- to 6-membered nonaromatic carbocyclic ring, a 4-, 5-, 6- or 7-membered nonaromatic heterocyclic ring, the heteroatom of the nonaromatic heterocyclic ring or ring system is selected from N, O or S(O)₀₋₂, and each nonaromatic carbocyclic or nonaromatic heterocyclic ring or ring system may be optionally substituted with one or more substituents selected from R³;
wherein, R³ is independently selected from halogen, cyano, C₁-C₆-alkyl, C₁-C₆-haloalkyl, C₃-C₈-cycloalkyl, and C₁-C₆-alkoxy;
R⁷ is selected from halogen, hydroxy, amino, C₁-C₆-alkyl, and C₃-C₈-cycloalkyl; or
R⁷ is phenyl, benzyl, a 5-membered aromatic ring, a 5- or 6-membered heteroaromatic ring; wherein heteroatom of the heteroaromatic ring is selected from N, O or S; or
R⁷ is a 3- to 6-membered nonaromatic carbocyclic ring, a 4-, 5-, 6- or 7-membered nonaromatic heterocyclic ring, wherein, the heteroatom of the nonaromatic heterocyclic ring is selected from N;
wherein, R⁷ may be further substituted with one or more R¹⁶ on C atom and with one or more R¹⁷ on N atom;
R¹⁶ is independently selected from hydrogen, halogen, cyano, C₁-C₄-alkyl, C₁-C₄-haloalkyl, C₃-C₆-cycloalkyl, C₃-C₆-halocycloalkyl, C₁-C₄-alkoxy, C₃-C₈-cycloalkoxy, and C₁-C₄-haloalkoxy,
R¹⁷ is independently selected from the group of hydrogen, cyano, (C═O)—R^{d}, S(O)₀₋₂R^{e}, C₁-C₆-alkyl, C₁-C₆-haloalkyl, C₁-C₆-alkoxy, C₁-C₆-haloalkoxy, and C₃-C₆-cycloalkyl;
R^{d} represents hydrogen, hydroxy, halogen, NR^{b}R^{c}, C₁-C₆-alkyl, C₁-C₆-haloalkyl, C₁-C₆-alkoxy, or C₃-C₈-cycloalkyl;
R^{b} and R^{c} represent hydrogen, C₁-C₄-alkyl, C₁-C₄-haloalkyl, C₁-C₄-alkoxy, or C₃-C₈-cycloalkyl; and
R^{e} represents hydrogen, amino, C₁-C₆-alkyl, C₁-C₆-haloalkyl, C₁-C₆-alkoxy, or C₃-C₈-cycloalkyl;
or N-oxides, metal complexes, isomers, polymorphs or the agriculturally acceptable salts thereof.

4. The compound of claim 1, wherein the compound is selected from the group consisting of:
(S)-(3-methoxyphenyl)(3-(4-(5-(trifluoromethyl)-1,2,4-oxadiazol-3-yl)phenoxy)pyrrolidin-1-yl)methanone; (S)-1-(3-(4-(5-(trifluoromethyl)-1,2,4-oxadiazol-3-yl)phenoxy)pyrrolidin-1-yl)ethan-1-one; (S)-(3-bromophenyl)(3-(4-(5-(trifluoromethyl)-1,2,4-oxadiazol-3-yl)phenoxy)pyrrolidin-1-yl)methanone; (S)-4-(3-(4-(5-(trifluoromethyl)-1,2,4-oxadiazol-3-yl)phenoxy)pyrrolidine-1-carbonyl)benzonitrile; (S)-2-(3,4-dimethoxyphenyl)-1-(3-(4-(5-(trifluoromethyl)-1,2,4-oxadiazol-3-yl)phenoxy)pyrrolidin-1-yl)ethan-1-one; (S)-(2-fluorophenyl)(3-(4-(5-(trifluoromethyl)-1,2,4-oxadiazol-3-yl)phenoxy)pyrrolidin-1-yl)methanone; (S)-pyridin-2-yl(3-(4-(5-(trifluoromethyl)-1,2,4-oxadiazol-3-yl)phenoxy)pyrrolidin-1-yl)methanone; (S)-(4-(dimethylamino)phenyl)(3-(4-(5-(trifluoromethyl)-1,2,4-oxadiazol-3-yl)phenoxy)pyrrolidin-1-yl)methanone; (S)-cyclobutyl(3-(4-(5-(trifluoromethyl)-1,2,4-oxadiazol-3-yl)phenoxy)pyrrolidin-1-yl)methanone; (S)-(4-methoxyphenyl)(3-(4-(5-(trifluoromethyl)-1,2,4-oxadiazol-3-yl)phenoxy)pyrrolidin-1-yl)methanone; (S)-2-phenyl-1-(3-(4-(5-(trifluoromethyl)-1,2,4-oxadiazol-3-yl)phenoxy)pyrrolidin-1-yl)ethan-1-one; (S)-pyridin-3-yl(3-(4-(5-(trifluoromethyl)-1,2,4-oxadiazol-3-yl)phenoxy)pyrrolidin-1-yl)methanone; (S)-pyridin-4-yl(3-(4-(5-(trifluoromethyl)-1,2,4-oxadiazol-3-yl)phenoxy)pyrrolidin-1-yl)methanone; (S)-(4-fluorophenyl)(3-(4-(5-(trifluoromethyl)-1,2,4-oxadiazol-3-yl)phenoxy)pyrrolidin-1-yl)methanone; (S)-phenyl(3-(4-(5-(trifluoromethyl)-1,2,4-oxadiazol-3-yl)phenoxy)pyrrolidin-1-yl)methanone; (3-bromophenyl)(3-(4-(5-(trifluoromethyl)-1,2,4-oxadiazol-3-yl)phenoxy)azetidin-1-yl)methanone; (3-methoxyphenyl)(3-(4-(5-(trifluoromethyl)-1,2,4-oxadiazol-3-yl)phenoxy)azetidin-1-yl)methanone; (3-fluorophenyl)(3-(4-(5-(trifluoromethyl)-1,2,4-oxadiazol-3-yl)phenoxy)azetidin-1-yl)methanone; (3-(4-(5-(trifluoromethyl)-1,2,4-oxadiazol-3-yl)phenoxy)azetidin-1-yl)(4-(trifluoromethyl)phenyl)methanone; (2-fluorophenyl)(3-(4-(5-(trifluoromethyl)-1,2,4-oxadiazol-3-yl)phenoxy)azetidin-1-yl)methanone; phenyl(3-(4-(5-(trifluoromethyl)-1,2,4-oxadiazol-3-yl)phenoxy)azetidin-1-yl)methanone; 3-(4-((1-(benzylsulfonyl)azetidin-3-yl)oxy)phenyl)-5-(trifluoromethyl)-1,2,4-oxadiazole; tert-butyl (S)-3-(4-(5-(trifluoromethyl)-1,2,4-oxadiazol-3-yl)phenoxy)pyrrolidine-1-carboxylate; (S)-2-(3-methoxyphenyl)-1-(3-(4-(5-(trifluoromethyl)-1,2,4-oxadiazol-3-yl)phenoxy)pyrrolidin-1-yl)ethan-1-one; (S)-3-(4-((1-(phenylsulfonyl)pyrrolidin-3-yl)oxy)phenyl)-5-(trifluoromethyl)-1,2,4-oxadiazole; (S)-3-(4-((1-((3-fluorophenyl)sulfonyl)pyrrolidin-3-yl)oxy)phenyl)-5-(trifluoromethyl)-1,2,4-oxadiazole; tert-butyl (S)-3-((6-(5-(trifluoromethyl)-1,2,4-oxadiazol-3-yl)pyridin-3-yl)oxy)pyrrolidine-1-carboxylate; (S)-(2-fluorophenyl)(3-((6-(5-(trifluoromethyl)-1,2,4-oxadiazol-3-yl)pyridin-3-yl)oxy)pyrrolidin-1-yl)methanone; (S)-(4-methoxyphenyl)(3-((6-(5-(trifluoromethyl)-1,2,4-oxadiazol-3-yl)pyridin-3-yl)oxy)pyrrolidin-1-yl)methanone; (S)-(3-fluorophenyl)(3-((6-(5-(trifluoromethyl)-1,2,4-oxadiazol-3-yl)pyridin-3-yl)oxy)pyrrolidin-1-yl)methanone; (S)-pyridin-3-yl(3-((6-(5-(trifluoromethyl)-1,2,4-oxadiazol-3-yl)pyridin-3-yl)oxy)pyrrolidin-1-yl)methanone; (S)-pyridin-4-yl(3-((6-(5-(trifluoromethyl)-1,2,4-oxadiazol-3-yl)pyridin-3-yl)oxy)pyrrolidin-1-yl)methanone; (S)-3-(4-((1-((2-fluorophenyl)sulfonyl)pyrrolidin-3-yl)oxy)phenyl)-5-(trifluoromethyl)-1,2,4-oxadiazole; (S)-3-(4-((1-((4-methoxyphenyl)sulfonyl)pyrrolidin-3-yl)oxy)phenyl)-5-(trifluoromethyl)-1,2,4-oxadiazole; (S)-3-(4-((1-((4-fluorophenyl)sulfonyl)pyrrolidin-3-yl)oxy)phenyl)-5-(trifluoromethyl)-1,2,4-oxadiazole; (S)-(4-(trifluoromethoxy)phenyl)(3-(4-(5-(trifluoromethyl)-1,2,4-oxadiazol-3-yl)phenoxy)pyrrolidin-1-yl)methanone; (S)—N-(2-fluorophenyl)-3-(4-(5-(trifluoromethyl)-1,2,4-oxadiazol-3-yl)phenoxy)pyrrolidine-1-carboxamide; (S)—N-(4-fluorophenyl)-3-(4-(5-(trifluoromethyl)-1,2,4-oxadiazol-3-yl)phenoxy)pyrrolidine-1-carboxamide; (S)—N-(4-methoxyphenyl)-3-(4-(5-(trifluoromethyl)-1,2,4-oxadiazol-3-yl)phenoxy)pyrrolidine-1-carboxamide; (S)-3-(4-((1-(ethylsulfonyl)pyrrolidin-3-yl)oxy)phenyl)-5-(trifluoromethyl)-1,2,4-oxadiazole; (S)-3-(4-((1-(cyclopropylsulfonyl)pyrrolidin-3-yl)oxy)phenyl)-5-(trifluoromethyl)-1,2,4-oxadiazole; (S)-3-(4-((1-((2,4-difluorophenyl)sulfonyl)pyrrolidin-3-yl)oxy)phenyl)-5-(trifluoromethyl)-1,2,4-oxadiazole; (S)-cyclopropyl(3-(4-(5-(trifluoromethyl)-1,2,4-oxadiazol-3-yl)phenoxy)pyrrolidin-1-yl)methanone; (S)-(4-chlorophenyl)(3-(4-(5-(trifluoromethyl)-1,2,4-oxadiazol-3-yl)phenoxy)pyrrolidin-1-yl)methanone; (S)-(2-fluorophenyl)(3-((5-(5-(trifluoromethyl)-1,2,4-oxadiazol-3-yl)pyridin-2-yl)oxy)pyrrolidin-1-yl)methanone; (S)-(4-methoxyphenyl)(3-((5-(5-(trifluoromethyl)-1,2,4-oxadiazol-3-yl)pyridin-2-yl)oxy)pyrrolidin-1-yl)methanone; (S)-(3-fluorophenyl)(3-((5-(5-(trifluoromethyl)-1,2,4-oxadiazol-3-yl)pyridin-2-yl)oxy)pyrrolidin-1-yl)methanone; (S)-pyridin-3-yl(3-((5-(5-(trifluoromethyl)-1,2,4-oxadiazol-3-yl)pyridin-2-yl)oxy)pyrrolidin-1-yl)methanone; (S)-pyridin-4-yl(3-((5-(5-(trifluoromethyl)-1,2,4-oxadiazol-3-yl)pyridin-2-yl)oxy)pyrrolidin-1-yl)methanone; (S)-3-(6-((1-(ethylsulfonyl)pyrrolidin-3-yl)oxy)pyridin-3-yl)-5-(trifluoromethyl)-1,2,4-oxadiazole; (S)-1,1-dimethyl-3-((5-(5-(trifluoromethyl)-1,2,4-oxadiazol-3-yl)pyridin-2-yl)oxy)pyrrolidin-1-ium 2,2,2-trifluoroacetate; (S)-(3-(2-fluoro-4-(5-(trifluoromethyl)-1,2,4-oxadiazol-3-yl)phenoxy)pyrrolidin-1-yl)(2-fluorophenyl)methanone; (S)-(3-(2-fluoro-4-(5-(trifluoromethyl)-1,2,4-oxadiazol-3-yl)phenoxy)pyrrolidin-1-yl)(4-methoxyphenyl)methanone; (S)-(3-(2-fluoro-4-(5-(trifluoromethyl)-1,2,4-oxadiazol-3-yl)phenoxy)pyrrolidin-1-yl)(3-fluorophenyl)methanone; (S)-2-(pyridin-2-yl)-1-(3-(4-(5-(trifluoromethyl)-1,2,4-oxadiazol-3-yl)phenoxy)pyrrolidin-1-yl)ethan-1-one; (S)-(6-methoxypyridin-3-yl)(3-(4-(5-(trifluoromethyl)-1,2,4-oxadiazol-3-yl)phenoxy)pyrrolidin-1-yl)methanone; (S)-pyrimidin-5-yl(3-(4-(5-(trifluoromethyl)-1,2,4-oxadiazol-3-yl)phenoxy)pyrrolidin-1-yl)methanone; (S)-(3-(2-fluoro-4-(5-(trifluoromethyl)-1,2,4-oxadiazol-3-yl)phenoxy)pyrrolidin-1-yl)(pyridin-3-yl)methanone; (S)-(3-(2-fluoro-4-(5-(trifluoromethyl)-1,2,4-oxadiazol-3-yl)phenoxy)pyrrolidin-1-yl)(pyridin-4-yl)methanone; tert-butyl (S)-3-(2-fluoro-4-(5-(trifluoromethyl)-1,2,4-oxadiazol-3-yl)phenoxy)pyrrolidine-1-carboxylate; (S)-3-(4-((1-(ethylsulfonyl)pyrrolidin-3-yl)oxy)-3-fluorophenyl)-5-(trifluoromethyl)-1,2,4-oxadiazole; tert-butyl (S)-3-(3-fluoro-4-(5-(trifluoromethyl)-1,2,4-oxadiazol-3-yl)phenoxy)pyrrolidine-1-carboxylate; (S)-(3-(3-fluoro-4-(5-(trifluoromethyl)-1,2,4-oxadiazol-3-yl)phenoxy)pyrrolidin-1-yl)(2-fluorophenyl)methanone; (S)-(3-(3-fluoro-4-(5-(trifluoromethyl)-1,2,4-oxadiazol-3-yl)phenoxy)pyrrolidin-1-yl)(3-fluorophenyl)methanone; (S)-(3-(3-fluoro-4-(5-(trifluoromethyl)-1,2,4-oxadiazol-3-yl)phenoxy)pyrrolidin-1-yl)(pyridin-3-yl)methanone; (S)-(3-(3-fluoro-4-(5-(trifluoromethyl)-1,2,4-oxadiazol-3-yl)phenoxy)pyrrolidin-1-yl)(pyridin-4-yl)methanone; (S)-3-(4-((1-(ethylsulfonyl)pyrrolidin-3-yl)oxy)-2-fluorophenyl)-5-(trifluoromethyl)-1,2,4-oxadiazole; (S)-1-(3-(3-fluoro-4-(5-(trifluoromethyl)-1,2,4-oxadiazol-3-yl)phenoxy)pyrrolidin-1-yl)ethan-1-one; (S)-1-(3-(2-fluoro-4-(5-(trifluoromethyl)-1,2,4-oxadiazol-3-yl)phenoxy)pyrrolidin-1-yl)ethan-1-one; tert-butyl (R)-3-((5-(5-(trifluoromethyl)-1,2,4-oxadiazol-3-yl)pyridin-2-yl)oxy)pyrrolidine-1-carboxylate; (R)-3-(6-((1-(phenylsulfonyl)pyrrolidin-3-yl)oxy)pyridin-3-yl)-5-(trifluoromethyl)-1,2,4-oxadiazole; (R)-3-(6-((1-(ethylsulfonyl)pyrrolidin-3-yl)oxy)pyridin-3-yl)-5-(trifluoromethyl)-1,2,4-oxadiazole; (R)-3-(6-((1-((4-fluorophenyl)sulfonyl)pyrrolidin-3-yl)oxy)pyridin-3-yl)-5-(trifluoromethyl)-1,2,4-oxadiazole; (R)-(2-fluorophenyl)(3-((5-(5-(trifluoromethyl)-1,2,4-oxadiazol-3-yl)pyridin-2-yl)oxy)pyrrolidin-1-yl)methanone; (R)-(4-methoxyphenyl)(3-((5-(5-(trifluoromethyl)-1,2,4-oxadiazol-3-yl)pyridin-2-yl)oxy)pyrrolidin-1-yl)methanone; (R)-3-(6-((1-(cyclopropylsulfonyl)pyrrolidin-3-yl)oxy)pyridin-3-yl)-5-(trifluoromethyl)-1,2,4-oxadiazole; (R)-(3-fluorophenyl)(3-((5-(5-(trifluoromethyl)-1,2,4-oxadiazol-3-yl)pyridin-2-yl)oxy)pyrrolidin-1-yl)methanone; (R)-pyridin-4-yl(3-((5-(5-(trifluoromethyl)-1,2,4-oxadiazol-3-yl)pyridin-2-yl)oxy)pyrrolidin-1-yl)methanone; (R)-pyridin-3-yl(3-((5-(5-(trifluoromethyl)-1,2,4-oxadiazol-3-yl)pyridin-2-yl)oxy)pyrrolidin-1-yl)methanone; tert-butyl 3-(methyl(4-(5-(trifluoromethyl)-1,2,4-oxadiazol-3-yl)phenyl)amino)azetidine-1-carboxylate; phenyl(4-(4-(5-(trifluoromethyl)-1,2,4-oxadiazol-3-yl)phenoxy)piperidin-1-yl)methanone; tert-butyl 4-(4-(5-(trifluoromethyl)-1,2,4-oxadiazol-3-yl)phenoxy)piperidine-1-carboxylate; (3-(methyl(4-(5-(trifluoromethyl)-1,2,4-oxadiazol-3-yl)phenyl)amino)azetidin-1-yl)(phenyl)methanone; (3-chlorophenyl)(3-(methyl(4-(5-(trifluoromethyl)-1,2,4-oxadiazol-3-yl)phenyl)amino)azetidin-1-yl)methanone; 1-(3-(methyl(4-(5-(trifluoromethyl)-1,2,4-oxadiazol-3-yl)phenyl)amino)azetidin-1-yl)ethan-1-one; (2-fluorophenyl)(4-(4-(5-(trifluoromethyl)-1,2,4-oxadiazol-3-yl)phenoxy)piperidin-1-yl)methanone; tert-butyl 3-((4-(5-(trifluoromethyl)-1,2,4-oxadiazol-3-yl)phenyl)thio)pyrrolidine-1-carboxylate; 1-(4-(4-(5-(trifluoromethyl)-1,2,4-oxadiazol-3-yl)phenoxy)piperidin-1-yl)ethan-1-one; (3-fluorophenyl)(3-(methyl(4-(5-(trifluoromethyl)-1,2,4-oxadiazol-3-yl)phenyl)amino)azetidin-1-yl)methanone; (3-(methyl(4-(5-(trifluoromethyl)-1,2,4-oxadiazol-3-yl)phenyl)amino)azetidin-1-yl)(p-tolyl)methanone; (4-methoxyphenyl)(3-(methyl(4-(5-(trifluoromethyl)-1,2,4-oxadiazol-3-yl)phenyl)amino)azetidin-1-yl)methanone; 1-(3-(methyl(4-(5-(trifluoromethyl)-1,2,4-oxadiazol-3-yl)phenyl)amino)azetidin-1-yl)-2-phenylethan-1-one; 1-(3-(methyl(4-(5-(trifluoromethyl)-1,2,4-oxadiazol-3-yl)phenyl)amino)azetidin-1-yl)propan-1-one; (3-(methyl(4-(5-(trifluoromethyl)-1,2,4-oxadiazol-3-yl)phenyl)amino)azetidin-1-yl)(4-(trifluoromethyl)phenyl)methanone; (4-(4-(5-(trifluoromethyl)-1,2,4-oxadiazol-3-yl)phenoxy)piperidin-1-yl)(4-(trifluoromethyl)phenyl)methanone; p-tolyl(4-(4-(5-(trifluoromethyl)-1,2,4-oxadiazol-3-yl)phenoxy)piperidin-1-yl)methanone; (S)-3-(6-((1-(phenylsulfonyl)pyrrolidin-3-yl)oxy)pyridin-3-yl)-5-(trifluoromethyl)-1,2,4-oxadiazole; (S)-3-(6-((1-((4-methoxyphenyl)sulfonyl)pyrrolidin-3-yl)oxy)pyridin-3-yl)-5-(trifluoromethyl)-1,2,4-oxadiazole; (S)-3-(6-((1-(cyclopropylsulfonyl)pyrrolidin-3-yl)oxy)pyridin-3-yl)-5-(trifluoromethyl)-1,2,4-oxadiazole; (S)-3-(6-((1-tosylpyrrolidin-3-yl)oxy)pyridin-3-yl)-5-(trifluoromethyl)-1,2,4-oxadiazole; (S)-3-(6-((1-((3-chlorophenyl)sulfonyl)pyrrolidin-3-yl)oxy)pyridin-3-yl)-5-(trifluoromethyl)-1,2,4-oxadiazole; (S)-5-(trifluoromethyl)-3-(6-((1-((4-(trifluoromethyl)phenyl)sulfonyl)pyrrolidin-3-yl)oxy)pyridin-3-yl)-1,2,4-oxadiazole; (S)-3-(6-((1-(propylsulfonyl)pyrrolidin-3-yl)oxy)pyridin-3-yl)-5-(trifluoromethyl)-1,2,4-oxadiazole; (S)-3-(6-((1-(methylsulfonyl)pyrrolidin-3-yl)oxy)pyridin-3-yl)-5-(trifluoromethyl)-1,2,4-oxadiazole; (S)-3-(6-((1-(m-tolylsulfonyl)pyrrolidin-3-yl)oxy)pyridin-3-yl)-5-(trifluoromethyl)-1,2,4-oxadiazole; (S)-5-(trifluoromethyl)-3-(4-((1-((trifluoromethyl)sulfonyl)pyrrolidin-3-yl)oxy)phenyl)-1,2,4-oxadiazole; (S)-3-(4-((1-(propylsulfonyl)pyrrolidin-3-yl)oxy)phenyl)-5-(trifluoromethyl)-1,2,4-oxadiazole; (S)-3-(4-((1-((4-bromophenyl)sulfonyl)pyrrolidin-3-yl)oxy)phenyl)-5-(trifluoromethyl)-1,2,4-oxadiazole; (S)-3-(4-((1-(pyridin-3-ylsulfonyl)pyrrolidin-3-yl)oxy)phenyl)-5-(trifluoromethyl)-1,2,4-oxadiazole; (S)-5-(trifluoromethyl)-3-(4-((1-((4-(trifluoromethyl)phenyl)sulfonyl)pyrrolidin-3-yl)oxy)phenyl)-1,2,4-oxadiazole; (S)-3-(4-((1-tosylpyrrolidin-3-yl)oxy)phenyl)-5-(trifluoromethyl)-1,2,4-oxadiazole; (S)-3-(4-((1-((2,4-dichlorophenyl)sulfonyl)pyrrolidin-3-yl)oxy)phenyl)-5-(trifluoromethyl)-1,2,4-oxadiazole; (S)-4-((3-(4-(5-(trifluoromethyl)-1,2,4-oxadiazol-3-yl)phenoxy)pyrrolidin-1-yl)sulfonyl)benzonitrile; (S)-3-(4-((1-((3-chlorophenyl)sulfonyl)pyrrolidin-3-yl)oxy)phenyl)-5-(trifluoromethyl)-1,2,4-oxadiazole; (4-(dimethylamino)phenyl)(3-(methyl(4-(5-(trifluoromethyl)-1,2,4-oxadiazol-3-yl)phenyl)amino)azetidin-1-yl)methanone; (4-fluorophenyl)(3-(methyl(4-(5-(trifluoromethyl)-1,2,4-oxadiazol-3-yl)phenyl)amino)azetidin-1-yl)methanone; (2-fluorophenyl)(3-(methyl(4-(5-(trifluoromethyl)-1,2,4-oxadiazol-3-yl)phenyl)amino)azetidin-1-yl)methanone; (3-(methyl(4-(5-(trifluoromethyl)-1,2,4-oxadiazol-3-yl)phenyl)amino)azetidin-1-yl)(m-tolyl)methanone; tert-butyl 3-(3-(5-(trifluoromethyl)-1,2,4-oxadiazol-3-yl)phenoxy)pyrrolidine-1-carboxylate; 2,2-dimethyl-1-(3-(methyl(4-(5-(trifluoromethyl)-1,2,4-oxadiazol-3-yl)phenyl)amino)azetidin-1-yl)propan-1-one; 2,2-dimethyl-1-(3-(3-(5-(trifluoromethyl)-1,2,4-oxadiazol-3-yl)phenoxy)pyrrolidin-1-yl)propan-1-one; (3-chlorophenyl)(4-(4-(5-(trifluoromethyl)-1,2,4-oxadiazol-3-yl)phenoxy)piperidin-1-yl)methanone; (2-fluorophenyl)(3-(3-(5-(trifluoromethyl)-1,2,4-oxadiazol-3-yl)phenoxy)pyrrolidin-1-yl)methanone; (3-fluorophenyl)(3-(3-(5-(trifluoromethyl)-1,2,4-oxadiazol-3-yl)phenoxy)pyrrolidin-1-yl)methanone; (4-fluorophenyl)(3-(3-(5-(trifluoromethyl)-1,2,4-oxadiazol-3-yl)phenoxy)pyrrolidin-1-yl)methanone; p-tolyl(3-(3-(5-(trifluoromethyl)-1,2,4-oxadiazol-3-yl)phenoxy)pyrrolidin-1-yl)methanone; (S)-3-(4-((1-(isopropylsulfonyl)pyrrolidin-3-yl)oxy)phenyl)-5-(trifluoromethyl)-1,2,4-oxadiazole; (S)-3-(4-((1-benzylpyrrolidin-3-yl)oxy)phenyl)-5-(trifluoromethyl)-1,2,4-oxadiazole; 2-phenyl-1-(4-(4-(5-(trifluoromethyl)-1,2,4-oxadiazol-3-yl)phenoxy)piperidin-1-yl)ethan-1-one; 2,2-dimethyl-1-(4-(4-(5-(trifluoromethyl)-1,2,4-oxadiazol-3-yl)phenoxy)piperidin-1-yl)propan-1-one; (4-methoxyphenyl)(4-(4-(5-(trifluoromethyl)-1,2,4-oxadiazol-3-yl)phenoxy)piperidin-1-yl)methanone; m-tolyl(3-(3-(5-(trifluoromethyl)-1,2,4-oxadiazol-3-yl)phenoxy)pyrrolidin-1-yl)methanone; (4-fluorophenyl)(4-(4-(5-(trifluoromethyl)-1,2,4-oxadiazol-3-yl)phenoxy)piperidin-1-yl)methanone; (3-(3-(5-(trifluoromethyl)-1,2,4-oxadiazol-3-yl)phenoxy)pyrrolidin-1-yl)(4-(trifluoromethyl)phenyl)methanone; (3-chlorophenyl)(3-(3-(5-(trifluoromethyl)-1,2,4-oxadiazol-3-yl)phenoxy)pyrrolidin-1-yl)methanone; 2-(4-chlorophenyl)-1-(4-(4-(5-(trifluoromethyl)-1,2,4-oxadiazol-3-yl)phenoxy)piperidin-1-yl)ethan-1-one; (4-methoxyphenyl)(3-(3-(5-(trifluoromethyl)-1,2,4-oxadiazol-3-yl)phenoxy)pyrrolidin-1-yl)methanone; (S)-3-(4-((1-((3-methylthiophen-2-yl)sulfonyl)pyrrolidin-3-yl)oxy)phenyl)-5-(trifluoromethyl)-1,2,4-oxadiazole; (S)-3-(4-((1-((1-methyl-1H-imidazol-4-yl)sulfonyl)pyrrolidin-3-yl)oxy)phenyl)-5-(trifluoromethyl)-1,2,4-oxadiazole; (S)-3-(6-((1-((3-fluorophenyl)sulfonyl)pyrrolidin-3-yl)oxy)pyridin-3-yl)-5-(trifluoromethyl)-1,2,4-oxadiazole; (S)-4-((3-((5-(5-(trifluoromethyl)-1,2,4-oxadiazol-3-yl)pyridin-2-yl)oxy)pyrrolidin-1-yl)sulfonyl)benzonitrile; (S)-5-(trifluoromethyl)-3-(6-((1-((3-(trifluoromethyl)phenyl)sulfonyl)pyrrolidin-3-yl)oxy)pyridin-3-yl)-1,2,4-oxadiazole; (S)-3-(6-((1-((2-fluorophenyl)sulfonyl)pyrrolidin-3-yl)oxy)pyridin-3-yl)-5-(trifluoromethyl)-1,2,4-oxadiazole; (S)-3-(6-((1-(pyridin-3-ylsulfonyl)pyrrolidin-3-yl)oxy)pyridin-3-yl)-5-(trifluoromethyl)-1,2,4-oxadiazole; (S)-3-(6-((1-(benzylsulfonyl)pyrrolidin-3-yl)oxy)pyridin-3-yl)-5-(trifluoromethyl)-1,2,4-oxadiazole; (S)-3-(6-((1-(isopropylsulfonyl)pyrrolidin-3-yl)oxy)pyridin-3-yl)-5-(trifluoromethyl)-1,2,4-oxadiazole; tert-butyl (R)-3-((4-(5-(trifluoromethyl)-1,2,4-oxadiazol-3-yl)phenyl)thio)pyrrolidine-1-carboxylate; m-tolyl(4-(4-(5-(trifluoromethyl)-1,2,4-oxadiazol-3-yl)phenoxy)piperidin-1-yl)methanone; (S)-3-(6-((1-((2-chlorophenyl)sulfonyl)pyrrolidin-3-yl)oxy)pyridin-3-yl)-5-(trifluoromethyl)-1,2,4-oxadiazole; (S)-3-(6-((1-((4-chlorobenzyl)sulfonyl)pyrrolidin-3-yl)oxy)pyridin-3-yl)-5-(trifluoromethyl)-1,2,4-oxadiazole; (S)-3-(6-((1-((2-methoxyethyl)sulfonyl)pyrrolidin-3-yl)oxy)pyridin-3-yl)-5-(trifluoromethyl)-1,2,4-oxadiazole; (S)-3-(4-((1-(4-methylbenzyl)pyrrolidin-3-yl)oxy)phenyl)-5-(trifluoromethyl)-1,2,4-oxadiazole; N-methyl-1-(phenylsulfonyl)-N-(4-(5-(trifluoromethyl)-1,2,4-oxadiazol-3-yl)phenyl)azetidin-3-amine; (R)-1-(3-((4-(5-(trifluoromethyl)-1,2,4-oxadiazol-3-yl)phenyl)thio)pyrrolidin-1-yl)ethan-1-one; tert-butyl 3-(methyl(4-(5-(trifluoromethyl)-1,2,4-oxadiazol-3-yl)phenyl)amino)pyrrolidine-1-carboxylate; N-methyl-1-tosyl-N-(4-(5-(trifluoromethyl)-1,2,4-oxadiazol-3-yl)phenyl)azetidin-3-amine; 1-((2-fluorophenyl)sulfonyl)-N-methyl-N-(4-(5-(trifluoromethyl)-1,2,4-oxadiazol-3-yl)phenyl)azetidin-3-amine; 1-((4-methoxyphenyl)sulfonyl)-N-methyl-N-(4-(5-(trifluoromethyl)-1,2,4-oxadiazol-3-yl)phenyl)azetidin-3-amine; N-methyl-N-(4-(5-(trifluoromethyl)-1,2,4-oxadiazol-3-yl)phenyl)-1-((3-(trifluoromethyl)phenyl)sulfonyl)azetidin-3-amine; (R)-m-tolyl(3-((4-(5-(trifluoromethyl)-1,2,4-oxadiazol-3-yl)phenyl)thio)pyrrolidin-1-yl)methanone; N-methyl-N-(4-(5-(trifluoromethyl)-1,2,4-oxadiazol-3-yl)phenyl)-1-((4-(trifluoromethyl)phenyl)sulfonyl)azetidin-3-amine; 1-((3-chlorophenyl)sulfonyl)-N-methyl-N-(4-(5-(trifluoromethyl)-1,2,4-oxadiazol-3-yl)phenyl)azetidin-3-amine; (S)-3-(6-((1-(phenethylsulfonyl)pyrrolidin-3-yl)oxy)pyridin-3-yl)-5-(trifluoromethyl)-1,2,4-oxadiazole; (R)-(4-methoxyphenyl)(3-((4-(5-(trifluoromethyl)-1,2,4-oxadiazol-3-yl)phenyl)thio)pyrrolidin-1-yl)methanone; (R)-phenyl(3-((4-(5-(trifluoromethyl)-1,2,4-oxadiazol-3-yl)phenyl)thio)pyrrolidin-1-yl)methanone; (R)-2-phenyl-1-(3-((4-(5-(trifluoromethyl)-1,2,4-oxadiazol-3-yl)phenyl)thio)pyrrolidin-1-yl)ethan-1-one; pyridin-4-yl(4-(4-(5-(trifluoromethyl)-1,2,4-oxadiazol-3-yl)phenoxy)piperidin-1-yl)methanone; (S)-3-(4-((1-(4-chlorobenzyl)pyrrolidin-3-yl)oxy)phenyl)-5-(trifluoromethyl)-1,2,4-oxadiazole; (S)-3-(4-((1-isopropylpyrrolidin-3-yl)oxy)phenyl)-5-(trifluoromethyl)-1,2,4-oxadiazole; (R)-2-phenyl-1-(3-((4-(5-(trifluoromethyl)-1,2,4-oxadiazol-3-yl)phenyl)sulfonyl)pyrrolidin-1-yl)ethan-1-one; (R)-(2-fluorophenyl)(3-((4-(5-(trifluoromethyl)-1,2,4-oxadiazol-3-yl)phenyl)thio)pyrrolidin-1-yl)methanone; (S)-1-(3-((5-(5-(trifluoromethyl)-1,2,4-oxadiazol-3-yl)pyridin-2-yl)oxy)pyrrolidin-1-yl)ethan-1-one; (S)-1-(3-((5-(5-(trifluoromethyl)-1,2,4-oxadiazol-3-yl)pyridin-2-yl)oxy)pyrrolidin-1-yl)propan-1-one; (R)-pyridin-4-yl(3-((4-(5-(trifluoromethyl)-1,2,4-oxadiazol-3-yl)phenyl)thio)pyrrolidin-1-yl)methanone; (R)-2-(4-chlorophenyl)-1-(3-((4-(5-(trifluoromethyl)-1,2,4-oxadiazol-3-yl)phenyl)thio)pyrrolidin-1-yl)ethan-1-one; 2-(4-methoxyphenyl)-1-(4-(4-(5-(trifluoromethyl)-1,2,4-oxadiazol-3-yl)phenoxy)piperidin-1-yl)ethan-1-one; (R)-2-(4-chlorophenyl)-1-(3-((4-(5-(trifluoromethyl)-1,2,4-oxadiazol-3-yl)phenyl)sulfonyl)pyrrolidin-1-yl)ethan-1-one; (R)-2-(4-methoxyphenyl)-1-(3-((4-(5-(trifluoromethyl)-1,2,4-oxadiazol-3-yl)phenyl)sulfonyl)pyrrolidin-1-yl)ethan-1-one; (S)-(1-methyl-1H-pyrazol-3-yl)(3-((5-(5-(trifluoromethyl)-1,2,4-oxadiazol-3-yl)pyridin-2-yl)oxy)pyrrolidin-1-yl)methanone; (S)-isoxazol-3-yl(3-((5-(5-(trifluoromethyl)-1,2,4-oxadiazol-3-yl)pyridin-2-yl)oxy)pyrrolidin-1-yl)methanone; (4-(dimethylamino)phenyl)(4-(4-(5-(trifluoromethyl)-1,2,4-oxadiazol-3-yl)phenoxy)piperidin-1-yl)methanone; N-methyl-1-(propylsulfonyl)-N-(4-(5-(trifluoromethyl)-1,2,4-oxadiazol-3-yl)phenyl)azetidin-3-amine; N-methyl-N-(4-(5-(trifluoromethyl)-1,2,4-oxadiazol-3-yl)phenyl)-1-((trifluoromethyl)sulfonyl)azetidin-3-amine; (R)-(4-(dimethylamino)phenyl)(3-((4-(5-(trifluoromethyl)-1,2,4-oxadiazol-3-yl)phenyl)thio)pyrrolidin-1-yl)methanone; 1-((3-methoxyphenyl)sulfonyl)-N-methyl-N-(4-(5-(trifluoromethyl)-1,2,4-oxadiazol-3-yl)phenyl)azetidin-3-amine; (S)-oxazol-4-yl(3-((5-(5-(trifluoromethyl)-1,2,4-oxadiazol-3-yl)pyridin-2-yl)oxy)pyrrolidin-1-yl)methanone; (S)-thiazol-4-yl(3-((5-(5-(trifluoromethyl)-1,2,4-oxadiazol-3-yl)pyridin-2-yl)oxy)pyrrolidin-1-yl)methanone; 3-(4-((1-(phenylsulfonyl)azetidin-3-yl)oxy)phenyl)-5-(trifluoromethyl)-1,2,4-oxadiazole; 3-(4-((1-(methylsulfonyl)azetidin-3-yl)oxy)phenyl)-5-(trifluoromethyl)-1,2,4-oxadiazole; 2-(4-methoxyphenyl)-1-(3-((4-(5-(trifluoromethyl)-1,2,4-oxadiazol-3-yl)phenyl)thio)pyrrolidin-1-yl)ethan-1-one; (R)-(3-((4-(5-(trifluoromethyl)-1,2,4-oxadiazol-3-yl)phenyl)thio)pyrrolidin-1-yl)(4-(trifluoromethyl)phenyl)methanone; (4-chlorophenyl)(4-(4-(5-(trifluoromethyl)-1,2,4-oxadiazol-3-yl)phenoxy)piperidin-1-yl)methanone; (R)-(3-((4-(5-(trifluoromethyl)-1,2,4-oxadiazol-3-yl)phenyl)sulfonyl)pyrrolidin-1-yl)(4-(trifluoromethyl)phenyl)methanone; (3-(methyl(4-(5-(trifluoromethyl)-1,2,4-oxadiazol-3-yl)phenyl)amino)pyrrolidin-1-yl)(phenyl)methanone; pyridin-2-yl(3-(3-(5-(trifluoromethyl)-1,2,4-oxadiazol-3-yl)phenoxy)pyrrolidin-1-yl)methanone; pyridin-4-yl(3-(3-(5-(trifluoromethyl)-1,2,4-oxadiazol-3-yl)phenoxy)pyrrolidin-1-yl)methanone; pyridin-3-yl(3-(3-(5-(trifluoromethyl)-1,2,4-oxadiazol-3-yl)phenoxy)pyrrolidin-1-yl)methanone; (S)-2-methyl-1-(3-((5-(5-(trifluoromethyl)-1,2,4-oxadiazol-3-yl)pyridin-2-yl)oxy)pyrrolidin-1-yl)propan-1-one; (S)-cyclopropyl(3-((5-(5-(trifluoromethyl)-1,2,4-oxadiazol-3-yl)pyridin-2-yl)oxy)pyrrolidin-1-yl)methanone; (4-chloro-3-(trifluoromethyl)phenyl)(4-(4-(5-(trifluoromethyl)-1,2,4-oxadiazol-3-yl)phenoxy)piperidin-1-yl)methanone; (R)-(3-methoxyphenyl)(3-(4-(5-(trifluoromethyl)-1,2,4-oxadiazol-3-yl)phenoxy)pyrrolidin-1-yl)methanone; (R)-1-(3-(4-(5-(trifluoromethyl)-1,2,4-oxadiazol-3-yl)phenoxy)pyrrolidin-1-yl)ethan-1-one; (R)-(3-bromophenyl)(3-(4-(5-(trifluoromethyl)-1,2,4-oxadiazol-3-yl)phenoxy)pyrrolidin-1-yl)methanone; (R)-4-(3-(4-(5-(trifluoromethyl)-1,2,4-oxadiazol-3-yl)phenoxy)pyrrolidine-1-carbonyl)benzonitrile; (R)-2-(3,4-dimethoxyphenyl)-1-(3-(4-(5-(trifluoromethyl)-1,2,4-oxadiazol-3-yl)phenoxy)pyrrolidin-1-yl)ethan-1-one; (R)-(2-fluorophenyl)(3-(4-(5-(trifluoromethyl)-1,2,4-oxadiazol-3-yl)phenoxy)pyrrolidin-1-yl)methanone; (R)-pyridin-2-yl(3-(4-(5-(trifluoromethyl)-1,2,4-oxadiazol-3-yl)phenoxy)pyrrolidin-1-yl)methanone; (R)-(4-(dimethylamino)phenyl)(3-(4-(5-(trifluoromethyl)-1,2,4-oxadiazol-3-yl)phenoxy)pyrrolidin-1-yl)methanone; (R)-cyclobutyl(3-(4-(5-(trifluoromethyl)-1,2,4-oxadiazol-3-yl)phenoxy)pyrrolidin-1-yl)methanone; (R)-(4-methoxyphenyl)(3-(4-(5-(trifluoromethyl)-1,2,4-oxadiazol-3-yl)phenoxy)pyrrolidin-1-yl)methanone; (R)-2-phenyl-1-(3-(4-(5-(trifluoromethyl)-1,2,4-oxadiazol-3-yl)phenoxy)pyrrolidin-1-yl)ethan-1-one; (R)-pyridin-3-yl(3-(4-(5-(trifluoromethyl)-1,2,4-oxadiazol-3-yl)phenoxy)pyrrolidin-1-yl)methanone; (R)-pyridin-4-yl(3-(4-(5-(trifluoromethyl)-1,2,4-oxadiazol-3-yl)phenoxy)pyrrolidin-1-yl)methanone; (R)-(4-fluorophenyl)(3-(4-(5-(trifluoromethyl)-1,2,4-oxadiazol-3-yl)phenoxy)pyrrolidin-1-yl)methanone; (R)-phenyl(3-(4-(5-(trifluoromethyl)-1,2,4-oxadiazol-3-yl)phenoxy)pyrrolidin-1-yl)methanone; tert-butyl (R)-3-(4-(5-(trifluoromethyl)-1,2,4-oxadiazol-3-yl)phenoxy)pyrrolidine-1-carboxylate; (R)-2-(3-methoxyphenyl)-1-(3-(4-(5-(trifluoromethyl)-1,2,4-oxadiazol-3-yl)phenoxy)pyrrolidin-1-yl)ethan-1-one; (R)-3-(4-((1-(phenylsulfonyl)pyrrolidin-3-yl)oxy)phenyl)-5-(trifluoromethyl)-1,2,4-oxadiazole; (R)-3-(4-((1-((3-fluorophenyl)sulfonyl)pyrrolidin-3-yl)oxy)phenyl)-5-(trifluoromethyl)-1,2,4-oxadiazole; tert-butyl (R)-3-((6-(5-(trifluoromethyl)-1,2,4-oxadiazol-3-yl)pyridin-3-yl)oxy)pyrrolidine-1-carboxylate; (R)-(2-fluorophenyl)(3-((6-(5-(trifluoromethyl)-1,2,4-oxadiazol-3-yl)pyridin-3-yl)oxy)pyrrolidin-1-yl)methanone; (R)-(4-methoxyphenyl)(3-((6-(5-(trifluoromethyl)-1,2,4-oxadiazol-3-yl)pyridin-3-yl)oxy)pyrrolidin-1-yl)methanone; (R)-(3-fluorophenyl)(3-((6-(5-(trifluoromethyl)-1,2,4-oxadiazol-3-yl)pyridin-3-yl)oxy)pyrrolidin-1-yl)methanone; (R)-pyridin-3-yl(3-((6-(5-(trifluoromethyl)-1,2,4-oxadiazol-3-yl)pyridin-3-yl)oxy)pyrrolidin-1-yl)methanone; (R)-pyridin-4-yl(3-((6-(5-(trifluoromethyl)-1,2,4-oxadiazol-3-yl)pyridin-3-yl)oxy)pyrrolidin-1-yl)methanone; (R)-3-(4-((1-((2-fluorophenyl)sulfonyl)pyrrolidin-3-yl)oxy)phenyl)-5-(trifluoromethyl)-1,2,4-oxadiazole; (R)-3-(4-((1-((4-methoxyphenyl)sulfonyl)pyrrolidin-3-yl)oxy)phenyl)-5-(trifluoromethyl)-1,2,4-oxadiazole; (R)-3-(4-((1-((4-fluorophenyl)sulfonyl)pyrrolidin-3-yl)oxy)phenyl)-5-(trifluoromethyl)-1,2,4-oxadiazole; (R)-(4-(trifluoromethoxy)phenyl)(3-(4-(5-(trifluoromethyl)-1,2,4-oxadiazol-3-yl)phenoxy)pyrrolidin-1-yl)methanone; (R)—N-(2-fluorophenyl)-3-(4-(5-(trifluoromethyl)-1,2,4-oxadiazol-3-yl)phenoxy)pyrrolidine-1-carboxamide; (R)—N-(4-fluorophenyl)-3-(4-(5-(trifluoromethyl)-1,2,4-oxadiazol-3-yl)phenoxy)pyrrolidine-1-carboxamide; (R)—N-(4-methoxyphenyl)-3-(4-(5-(trifluoromethyl)-1,2,4-oxadiazol-3-yl)phenoxy)pyrrolidine-1-carboxamide; (R)-3-(4-((1-(ethylsulfonyl)pyrrolidin-3-yl)oxy)phenyl)-5-(trifluoromethyl)-1,2,4-oxadiazole; (R)-3-(4-((1-(cyclopropylsulfonyl)pyrrolidin-3-yl)oxy)phenyl)-5-(trifluoromethyl)-1,2,4-oxadiazole; (R)-3-(4-((1-((2,4-difluorophenyl)sulfonyl)pyrrolidin-3-yl)oxy)phenyl)-5-(trifluoromethyl)-1,2,4-oxadiazole; (R)-cyclopropyl(3-(4-(5-(trifluoromethyl)-1,2,4-oxadiazol-3-yl)phenoxy)pyrrolidin-1-yl)methanone; (R)-(4-chlorophenyl)(3-(4-(5-(trifluoromethyl)-1,2,4-oxadiazol-3-yl)phenoxy)pyrrolidin-1-yl)methanone; (R)-(2-fluorophenyl)(3-((5-(5-(trifluoromethyl)-1,2,4-oxadiazol-3-yl)pyridin-2-yl)oxy)pyrrolidin-1-yl)methanone; (R)-(4-methoxyphenyl)(3-((5-(5-(trifluoromethyl)-1,2,4-oxadiazol-3-yl)pyridin-2-yl)oxy)pyrrolidin-1-yl)methanone; (R)-(3-fluorophenyl)(3-((5-(5-(trifluoromethyl)-1,2,4-oxadiazol-3-yl)pyridin-2-yl)oxy)pyrrolidin-1-yl)methanone; (R)-pyridin-3-yl(3-((5-(5-(trifluoromethyl)-1,2,4-oxadiazol-3-yl)pyridin-2-yl)oxy)pyrrolidin-1-yl)methanone; (R)-pyridin-4-yl(3-((5-(5-(trifluoromethyl)-1,2,4-oxadiazol-3-yl)pyridin-2-yl)oxy)pyrrolidin-1-yl)methanone; (R)-3-(6-((1-(ethylsulfonyl)pyrrolidin-3-yl)oxy)pyridin-3-yl)-5-(trifluoromethyl)-1,2,4-oxadiazole; (R)-1,1-dimethyl-3-((5-(5-(trifluoromethyl)-1,2,4-oxadiazol-3-yl)pyridin-2-yl)oxy)pyrrolidin-1-ium 2,2,2-trifluoroacetate; (R)-(3-(2-fluoro-4-(5-(trifluoromethyl)-1,2,4-oxadiazol-3-yl)phenoxy)pyrrolidin-1-yl)(2-fluorophenyl)methanone; (R)-(3-(2-fluoro-4-(5-(trifluoromethyl)-1,2,4-oxadiazol-3-yl)phenoxy)pyrrolidin-1-yl)(4-methoxyphenyl)methanone; (R)-(3-(2-fluoro-4-(5-(trifluoromethyl)-1,2,4-oxadiazol-3-yl)phenoxy)pyrrolidin-1-yl)(3-fluorophenyl)methanone; (R)-2-(pyridin-2-yl)-1-(3-(4-(5-(trifluoromethyl)-1,2,4-oxadiazol-3-yl)phenoxy)pyrrolidin-1-yl)ethan-1-one; (R)-(6-methoxypyridin-3-yl)(3-(4-(5-(trifluoromethyl)-1,2,4-oxadiazol-3-yl)phenoxy)pyrrolidin-1-yl)methanone; (R)-pyrimidin-5-yl(3-(4-(5-(trifluoromethyl)-1,2,4-oxadiazol-3-yl)phenoxy)pyrrolidin-1-yl)methanone; (R)-(3-(2-fluoro-4-(5-(trifluoromethyl)-1,2,4-oxadiazol-3-yl)phenoxy)pyrrolidin-1-yl)(pyridin-3-yl)methanone; (R)-(3-(2-fluoro-4-(5-(trifluoromethyl)-1,2,4-oxadiazol-3-yl)phenoxy)pyrrolidin-1-yl)(pyridin-4-yl)methanone; tert-butyl (R)-3-(2-fluoro-4-(5-(trifluoromethyl)-1,2,4-oxadiazol-3-yl)phenoxy)pyrrolidine-1-carboxylate; (R)-3-(4-((1-(ethylsulfonyl)pyrrolidin-3-yl)oxy)-3-fluorophenyl)-5-(trifluoromethyl)-1,2,4-oxadiazole; tert-butyl (R)-3-(3-fluoro-4-(5-(trifluoromethyl)-1,2,4-oxadiazol-3-yl)phenoxy)pyrrolidine-1-carboxylate; (R)-(3-(3-fluoro-4-(5-(trifluoromethyl)-1,2,4-oxadiazol-3-yl)phenoxy)pyrrolidin-1-yl)(2-fluorophenyl)methanone; (R)-(3-(3-fluoro-4-(5-(trifluoromethyl)-1,2,4-oxadiazol-3-yl)phenoxy)pyrrolidin-1-yl)(3-fluorophenyl)methanone; (R)-(3-(3-fluoro-4-(5-(trifluoromethyl)-1,2,4-oxadiazol-3-yl)phenoxy)pyrrolidin-1-yl)(pyridin-3-yl)methanone; (R)-(3-(3-fluoro-4-(5-(trifluoromethyl)-1,2,4-oxadiazol-3-yl)phenoxy)pyrrolidin-1-yl)(pyridin-4-yl)methanone; (R)-3-(4-((1-(ethylsulfonyl)pyrrolidin-3-yl)oxy)-2-fluorophenyl)-5-(trifluoromethyl)-1,2,4-oxadiazole; (R)-1-(3-(3-fluoro-4-(5-(trifluoromethyl)-1,2,4-oxadiazol-3-yl)phenoxy)pyrrolidin-1-yl)ethan-1-one; (R)-1-(3-(2-fluoro-4-(5-(trifluoromethyl)-1,2,4-oxadiazol-3-yl)phenoxy)pyrrolidin-1-yl)ethan-1-one; tert-butyl (S)-3-((5-(5-(trifluoromethyl)-1,2,4-oxadiazol-3-yl)pyridin-2-yl)oxy)pyrrolidine-1-carboxylate; (S)-3-(6-((1-(phenylsulfonyl)pyrrolidin-3-yl)oxy)pyridin-3-yl)-5-(trifluoromethyl)-1,2,4-oxadiazole; (S)-3-(6-((1-(ethylsulfonyl)pyrrolidin-3-yl)oxy)pyridin-3-yl)-5-(trifluoromethyl)-1,2,4-oxadiazole; (S)-3-(6-((1-((4-fluorophenyl)sulfonyl)pyrrolidin-3-yl)oxy)pyridin-3-yl)-5-(trifluoromethyl)-1,2,4-oxadiazole; (S)-(2-fluorophenyl)(3-((5-(5-(trifluoromethyl)-1,2,4-oxadiazol-3-yl)pyridin-2-yl)oxy)pyrrolidin-1-yl)methanone; (S)-(4-methoxyphenyl)(3-((5-(5-(trifluoromethyl)-1,2,4-oxadiazol-3-yl)pyridin-2-yl)oxy)pyrrolidin-1-yl)methanone; (S)-3-(6-((1-(cyclopropylsulfonyl)pyrrolidin-3-yl)oxy)pyridin-3-yl)-5-(trifluoromethyl)-1,2,4-oxadiazole; (S)-(3-fluorophenyl)(3-((5-(5-(trifluoromethyl)-1,2,4-oxadiazol-3-yl)pyridin-2-yl)oxy)pyrrolidin-1-yl)methanone; (S)-pyridin-4-yl(3-((5-(5-(trifluoromethyl)-1,2,4-oxadiazol-3-yl)pyridin-2-yl)oxy)pyrrolidin-1-yl)methanone; (S)-pyridin-3-yl(3-((5-(5-(trifluoromethyl)-1,2,4-oxadiazol-3-yl)pyridin-2-yl)oxy)pyrrolidin-1-yl)methanone; (R)-3-(6-((1-(phenylsulfonyl)pyrrolidin-3-yl)oxy)pyridin-3-yl)-5-(trifluoromethyl)-1,2,4-oxadiazole; (R)-3-(6-((1-((4-methoxyphenyl)sulfonyl)pyrrolidin-3-yl)oxy)pyridin-3-yl)-5-(trifluoromethyl)-1,2,4-oxadiazole; (R)-3-(6-((1-(cyclopropylsulfonyl)pyrrolidin-3-yl)oxy)pyridin-3-yl)-5-(trifluoromethyl)-1,2,4-oxadiazole; (R)-3-(6-((1-tosylpyrrolidin-3-yl)oxy)pyridin-3-yl)-5-(trifluoromethyl)-1,2,4-oxadiazole; (R)-3-(6-((1-((3-chlorophenyl)sulfonyl)pyrrolidin-3-yl)oxy)pyridin-3-yl)-5-(trifluoromethyl)-1,2,4-oxadiazole; (R)-5-(trifluoromethyl)-3-(6-((1-((4-(trifluoromethyl)phenyl)sulfonyl)pyrrolidin-3-yl)oxy)pyridin-3-yl)-1,2,4-oxadiazole; (R)-3-(6-((1-(propylsulfonyl)pyrrolidin-3-yl)oxy)pyridin-3-yl)-5-(trifluoromethyl)-1,2,4-oxadiazole; (R)-3-(6-((1-(methylsulfonyl)pyrrolidin-3-yl)oxy)pyridin-3-yl)-5-(trifluoromethyl)-1,2,4-oxadiazole; (R)-3-(6-((1-(m-tolylsulfonyl)pyrrolidin-3-yl)oxy)pyridin-3-yl)-5-(trifluoromethyl)-1,2,4-oxadiazole; (R)-5-(trifluoromethyl)-3-(4-((1-((trifluoromethyl)sulfonyl)pyrrolidin-3-yl)oxy)phenyl)-1,2,4-oxadiazole; (R)-3-(4-((1-(propylsulfonyl)pyrrolidin-3-yl)oxy)phenyl)-5-(trifluoromethyl)-1,2,4-oxadiazole; (R)-3-(4-((1-((4-bromophenyl)sulfonyl)pyrrolidin-3-yl)oxy)phenyl)-5-(trifluoromethyl)-1,2,4-oxadiazole; (R)-3-(4-((1-(pyridin-3-ylsulfonyl)pyrrolidin-3-yl)oxy)phenyl)-5-(trifluoromethyl)-1,2,4-oxadiazole; (R)-5-(trifluoromethyl)-3-(4-((1-((4-(trifluoromethyl)phenyl)sulfonyl)pyrrolidin-3-yl)oxy)phenyl)-1,2,4-oxadiazole; (R)-3-(4-((1-tosylpyrrolidin-3-yl)oxy)phenyl)-5-(trifluoromethyl)-1,2,4-oxadiazole; (R)-3-(4-((1-((2,4-dichlorophenyl)sulfonyl)pyrrolidin-3-yl)oxy)phenyl)-5-(trifluoromethyl)-1,2,4-oxadiazole; (R)-4-((3-(4-(5-(trifluoromethyl)-1,2,4-oxadiazol-3-yl)phenoxy)pyrrolidin-1-yl)sulfonyl)benzonitrile; (R)-3-(4-((1-((3-chlorophenyl)sulfonyl)pyrrolidin-3-yl)oxy)phenyl)-5-(trifluoromethyl)-1,2,4-oxadiazole; (R)-3-(4-((1-(isopropylsulfonyl)pyrrolidin-3-yl)oxy)phenyl)-5-(trifluoromethyl)-1,2,4-oxadiazole; (R)-3-(4-((1-benzylpyrrolidin-3-yl)oxy)phenyl)-5-(trifluoromethyl)-1,2,4-oxadiazole; (R)-3-(4-((1-((3-methylthiophen-2-yl)sulfonyl)pyrrolidin-3-yl)oxy)phenyl)-5-(trifluoromethyl)-1,2,4-oxadiazole; (R)-3-(4-((1-((1-methyl-1H-imidazol-4-yl)sulfonyl)pyrrolidin-3-yl)oxy)phenyl)-5-(trifluoromethyl)-1,2,4-oxadiazole; (R)-3-(6-((1-((3-fluorophenyl)sulfonyl)pyrrolidin-3-yl)oxy)pyridin-3-yl)-5-(trifluoromethyl)-1,2,4-oxadiazole; (R)-4-((3-((5-(5-(trifluoromethyl)-1,2,4-oxadiazol-3-yl)pyridin-2-yl)oxy)pyrrolidin-1-yl)sulfonyl)benzonitrile; (R)-5-(trifluoromethyl)-3-(6-((1-((3-(trifluoromethyl)phenyl)sulfonyl)pyrrolidin-3-yl)oxy)pyridin-3-yl)-1,2,4-oxadiazole; (R)-3-(6-((1-((2-fluorophenyl)sulfonyl)pyrrolidin-3-yl)oxy)pyridin-3-yl)-5-(trifluoromethyl)-1,2,4-oxadiazole; (R)-3-(6-((1-(pyridin-3-ylsulfonyl)pyrrolidin-3-yl)oxy)pyridin-3-yl)-5-(trifluoromethyl)-1,2,4-oxadiazole; (R)-3-(6-((1-(benzylsulfonyl)pyrrolidin-3-yl)oxy)pyridin-3-yl)-5-(trifluoromethyl)-1,2,4-oxadiazole; (R)-3-(6-((1-(isopropylsulfonyl)pyrrolidin-3-yl)oxy)pyridin-3-yl)-5-(trifluoromethyl)-1,2,4-oxadiazole; tert-butyl (S)-3-((4-(5-(trifluoromethyl)-1,2,4-oxadiazol-3-yl)phenyl)thio)pyrrolidine-1-carboxylate; (R)-3-(6-((1-((2-chlorophenyl)sulfonyl)pyrrolidin-3-yl)oxy)pyridin-3-yl)-5-(trifluoromethyl)-1,2,4-oxadiazole; (R)-3-(6-((1-((4-chlorobenzyl)sulfonyl)pyrrolidin-3-yl)oxy)pyridin-3-yl)-5-(trifluoromethyl)-1,2,4-oxadiazole; (R)-3-(6-((1-((2-methoxyethyl)sulfonyl)pyrrolidin-3-yl)oxy)pyridin-3-yl)-5-(trifluoromethyl)-1,2,4-oxadiazole; (R)-3-(4-((1-(4-methylbenzyl)pyrrolidin-3-yl)oxy)phenyl)-5-(trifluoromethyl)-1,2,4-oxadiazole; (S)-1-(3-((4-(5-(trifluoromethyl)-1,2,4-oxadiazol-3-yl)phenyl)thio)pyrrolidin-1-yl)ethan-1-one; (S)-m-tolyl(3-((4-(5-(trifluoromethyl)-1,2,4-oxadiazol-3-yl)phenyl)thio)pyrrolidin-1-yl)methanone; (R)-3-(6-((1-(phenethylsulfonyl)pyrrolidin-3-yl)oxy)pyridin-3-yl)-5-(trifluoromethyl)-1,2,4-oxadiazole; (S)-(4-methoxyphenyl)(3-((4-(5-(trifluoromethyl)-1,2,4-oxadiazol-3-yl)phenyl)thio)pyrrolidin-1-yl)methanone; (S)-phenyl(3-((4-(5-(trifluoromethyl)-1,2,4-oxadiazol-3-yl)phenyl)thio)pyrrolidin-1-yl)methanone; (S)-2-phenyl-1-(3-((4-(5-(trifluoromethyl)-1,2,4-oxadiazol-3-yl)phenyl)thio)pyrrolidin-1-yl)ethan-1-one; (R)-3-(4-((1-(4-chlorobenzyl)pyrrolidin-3-yl)oxy)phenyl)-5-(trifluoromethyl)-1,2,4-oxadiazole; (R)-3-(4-((1-isopropylpyrrolidin-3-yl)oxy)phenyl)-5-(trifluoromethyl)-1,2,4-oxadiazole; (S)-2-phenyl-1-(3-((4-(5-(trifluoromethyl)-1,2,4-oxadiazol-3-yl)phenyl)sulfonyl)pyrrolidin-1-yl)ethan-1-one; (S)-(2-fluorophenyl)(3-((4-(5-(trifluoromethyl)-1,2,4-oxadiazol-3-yl)phenyl)thio)pyrrolidin-1-yl)methanone; (R)-1-(3-((5-(5-(trifluoromethyl)-1,2,4-oxadiazol-3-yl)pyridin-2-yl)oxy)pyrrolidin-1-yl)ethan-1-one; (R)-1-(3-((5-(5-(trifluoromethyl)-1,2,4-oxadiazol-3-yl)pyridin-2-yl)oxy)pyrrolidin-1-yl)propan-1-one; (S)-pyridin-4-yl(3-((4-(5-(trifluoromethyl)-1,2,4-oxadiazol-3-yl)phenyl)thio)pyrrolidin-1-yl)methanone; (S)-2-(4-chlorophenyl)-1-(3-((4-(5-(trifluoromethyl)-1,2,4-oxadiazol-3-yl)phenyl)thio)pyrrolidin-1-yl)ethan-1-one; (S)-2-(4-chlorophenyl)-1-(3-((4-(5-(trifluoromethyl)-1,2,4-oxadiazol-3-yl)phenyl)sulfonyl)pyrrolidin-1-yl)ethan-1-one; (S)-2-(4-methoxyphenyl)-1-(3-((4-(5-(trifluoromethyl)-1,2,4-oxadiazol-3-yl)phenyl)sulfonyl)pyrrolidin-1-yl)ethan-1-one; (R)-(1-methyl-1H-pyrazol-3-yl)(3-((5-(5-(trifluoromethyl)-1,2,4-oxadiazol-3-yl)pyridin-2-yl)oxy)pyrrolidin-1-yl)methanone; (R)-isoxazol-3-yl(3-((5-(5-(trifluoromethyl)-1,2,4-oxadiazol-3-yl)pyridin-2-yl)oxy)pyrrolidin-1-yl)methanone; (S)-(4-(dimethylamino)phenyl)(3-((4-(5-(trifluoromethyl)-1,2,4-oxadiazol-3-yl)phenyl)thio)pyrrolidin-1-yl)methanone; (R)-oxazol-4-yl(3-((5-(5-(trifluoromethyl)-1,2,4-oxadiazol-3-yl)pyridin-2-yl)oxy)pyrrolidin-1-yl)methanone; (R)-thiazol-4-yl(3-((5-(5-(trifluoromethyl)-1,2,4-oxadiazol-3-yl)pyridin-2-yl)oxy)pyrrolidin-1-yl)methanone; (S)-(3-((4-(5-(trifluoromethyl)-1,2,4-oxadiazol-3-yl)phenyl)thio)pyrrolidin-1-yl)(4-(trifluoromethyl)phenyl)methanone; (S)-(3-((4-(5-(trifluoromethyl)-1,2,4-oxadiazol-3-yl)phenyl)sulfonyl)pyrrolidin-1-yl)(4-(trifluoromethyl)phenyl)methanone; (R)-2-methyl-1-(3-((5-(5-(trifluoromethyl)-1,2,4-oxadiazol-3-yl)pyridin-2-yl)oxy)pyrrolidin-1-yl)propan-1-one; (R)-cyclopropyl(3-((5-(5-(trifluoromethyl)-1,2,4-oxadiazol-3-yl)pyridin-2-yl)oxy)pyrrolidin-1-yl)methanone; (3-methoxyphenyl)(3-(4-(5-(trifluoromethyl)-1,2,4-oxadiazol-3-yl)phenoxy)pyrrolidin-1-yl)methanone; 1-(3-(4-(5-(trifluoromethyl)-1,2,4-oxadiazol-3-yl)phenoxy)pyrrolidin-1-yl)ethan-1-one; (3-bromophenyl)(3-(4-(5-(trifluoromethyl)-1,2,4-oxadiazol-3-yl)phenoxy)pyrrolidin-1-yl)methanone; 4-(3-(4-(5-(trifluoromethyl)-1,2,4-oxadiazol-3-yl)phenoxy)pyrrolidine-1-carbonyl)benzonitrile; 2-(3,4-dimethoxyphenyl)-1-(3-(4-(5-(trifluoromethyl)-1,2,4-oxadiazol-3-yl)phenoxy)pyrrolidin-1-yl)ethan-1-one; (2-fluorophenyl)(3-(4-(5-(trifluoromethyl)-1,2,4-oxadiazol-3-yl)phenoxy)pyrrolidin-1-yl)methanone; pyridin-2-yl(3-(4-(5-(trifluoromethyl)-1,2,4-oxadiazol-3-yl)phenoxy)pyrrolidin-1-yl)methanone; (4-(dimethylamino)phenyl)(3-(4-(5-(trifluoromethyl)-1,2,4-oxadiazol-3-yl)phenoxy)pyrrolidin-1-yl)methanone; cyclobutyl(3-(4-(5-(trifluoromethyl)-1,2,4-oxadiazol-3-yl)phenoxy)pyrrolidin-1-yl)methanone; (4-methoxyphenyl)(3-(4-(5-(trifluoromethyl)-1,2,4-oxadiazol-3-yl)phenoxy)pyrrolidin-1-yl)methanone; 2-phenyl-1-(3-(4-(5-(trifluoromethyl)-1,2,4-oxadiazol-3-yl)phenoxy)pyrrolidin-1-yl)ethan-1-one; pyridin-3-yl(3-(4-(5-(trifluoromethyl)-1,2,4-oxadiazol-3-yl)phenoxy)pyrrolidin-1-yl)methanone; pyridin-4-yl(3-(4-(5-(trifluoromethyl)-1,2,4-oxadiazol-3-yl)phenoxy)pyrrolidin-1-yl)methanone; (4-fluorophenyl)(3-(4-(5-(trifluoromethyl)-1,2,4-oxadiazol-3-yl)phenoxy)pyrrolidin-1-yl)methanone; phenyl(3-(4-(5-(trifluoromethyl)-1,2,4-oxadiazol-3-yl)phenoxy)pyrrolidin-1-yl)methanone; tert-butyl-3-(4-(5-(trifluoromethyl)-1,2,4-oxadiazol-3-yl)phenoxy)pyrrolidine-1-carboxylate; 2-(3-methoxyphenyl)-1-(3-(4-(5-(trifluoromethyl)-1,2,4-oxadiazol-3-yl)phenoxy)pyrrolidin-1-yl)ethan-1-one; 3-(4-((1-(phenylsulfonyl)pyrrolidin-3-yl)oxy)phenyl)-5-(trifluoromethyl)-1,2,4-oxadiazole; 3-(4-((1-((3-fluorophenyl)sulfonyl)pyrrolidin-3-yl)oxy)phenyl)-5-(trifluoromethyl)-1,2,4-oxadiazole; tert-butyl-3-((6-(5-(trifluoromethyl)-1,2,4-oxadiazol-3-yl)pyridin-3-yl)oxy)pyrrolidine-1-carboxylate; (2-fluorophenyl)(3-((6-(5-(trifluoromethyl)-1,2,4-oxadiazol-3-yl)pyridin-3-yl)oxy)pyrrolidin-1-yl)methanone; (4-methoxyphenyl)(3-((6-(5-(trifluoromethyl)-1,2,4-oxadiazol-3-yl)pyridin-3-yl)oxy)pyrrolidin-1-yl)methanone; (3-fluorophenyl)(3-((6-(5-(trifluoromethyl)-1,2,4-oxadiazol-3-yl)pyridin-3-yl)oxy)pyrrolidin-1-yl)methanone; pyridin-3-yl(3-((6-(5-(trifluoromethyl)-1,2,4-oxadiazol-3-yl)pyridin-3-yl)oxy)pyrrolidin-1-yl)methanone; pyridin-4-yl(3-((6-(5-(trifluoromethyl)-1,2,4-oxadiazol-3-yl)pyridin-3-yl)oxy)pyrrolidin-1-yl)methanone; 3-(4-((1-((2-fluorophenyl)sulfonyl)pyrrolidin-3-yl)oxy)phenyl)-5-(trifluoromethyl)-1,2,4-oxadiazole; 3-(4-((1-((4-methoxyphenyl)sulfonyl)pyrrolidin-3-yl)oxy)phenyl)-5-(trifluoromethyl)-1,2,4-oxadiazole; 3-(4-((1-((4-fluorophenyl)sulfonyl)pyrrolidin-3-yl)oxy)phenyl)-5-(trifluoromethyl)-1,2,4-oxadiazole; (4-(trifluoromethoxy)phenyl)(3-(4-(5-(trifluoromethyl)-1,2,4-oxadiazol-3-yl)phenoxy)pyrrolidin-1-yl)methanone; N-(2-fluorophenyl)-3-(4-(5-(trifluoromethyl)-1,2,4-oxadiazol-3-yl)phenoxy)pyrrolidine-1-carboxamide; N-(4-fluorophenyl)-3-(4-(5-(trifluoromethyl)-1,2,4-oxadiazol-3-yl)phenoxy)pyrrolidine-1-carboxamide; N-(4-methoxyphenyl)-3-(4-(5-(trifluoromethyl)-1,2,4-oxadiazol-3-yl)phenoxy)pyrrolidine-1-carboxamide; 3-(4-((1-(ethylsulfonyl)pyrrolidin-3-yl)oxy)phenyl)-5-(trifluoromethyl)-1,2,4-oxadiazole; 3-(4-((1-(cyclopropylsulfonyl)pyrrolidin-3-yl)oxy)phenyl)-5-(trifluoromethyl)-1,2,4-oxadiazole; 3-(4-((1-((2,4-difluorophenyl)sulfonyl)pyrrolidin-3-yl)oxy)phenyl)-5-(trifluoromethyl)-1,2,4-oxadiazole; cyclopropyl(3-(4-(5-(trifluoromethyl)-1,2,4-oxadiazol-3-yl)phenoxy)pyrrolidin-1-yl)methanone; (4-chlorophenyl)(3-(4-(5-(trifluoromethyl)-1,2,4-oxadiazol-3-yl)phenoxy)pyrrolidin-1-yl)methanone; (2-fluorophenyl)(3-((5-(5-(trifluoromethyl)-1,2,4-oxadiazol-3-yl)pyridin-2-yl)oxy)pyrrolidin-1-yl)methanone; (4-methoxyphenyl)(3-((5-(5-(trifluoromethyl)-1,2,4-oxadiazol-3-yl)pyridin-2-yl)oxy)pyrrolidin-1-yl)methanone; (3-fluorophenyl)(3-((5-(5-(trifluoromethyl)-1,2,4-oxadiazol-3-yl)pyridin-2-yl)oxy)pyrrolidin-1-yl)methanone; pyridin-3-yl(3-((5-(5-(trifluoromethyl)-1,2,4-oxadiazol-3-yl)pyridin-2-yl)oxy)pyrrolidin-1-yl)methanone; pyridin-4-yl(3-((5-(5-(trifluoromethyl)-1,2,4-oxadiazol-3-yl)pyridin-2-yl)oxy)pyrrolidin-1-yl)methanone; 3-(6-((1-(ethylsulfonyl)pyrrolidin-3-yl)oxy)pyridin-3-yl)-5-(trifluoromethyl)-1,2,4-oxadiazole; 1,1-dimethyl-3-((5-(5-(trifluoromethyl)-1,2,4-oxadiazol-3-yl)pyridin-2-yl)oxy)pyrrolidin-1-ium 2,2,2-trifluoroacetate; (3-(2-fluoro-4-(5-(trifluoromethyl)-1,2,4-oxadiazol-3-yl)phenoxy)pyrrolidin-1-yl)(2-fluorophenyl)methanone; (3-(2-fluoro-4-(5-(trifluoromethyl)-1,2,4-oxadiazol-3-yl)phenoxy)pyrrolidin-1-yl)(4-methoxyphenyl)methanone; (3-(2-fluoro-4-(5-(trifluoromethyl)-1,2,4-oxadiazol-3-yl)phenoxy)pyrrolidin-1-yl)(3-fluorophenyl)methanone; 2-(pyridin-2-yl)-1-(3-(4-(5-(trifluoromethyl)-1,2,4-oxadiazol-3-yl)phenoxy)pyrrolidin-1-yl)ethan-1-one; (6-methoxypyridin-3-yl)(3-(4-(5-(trifluoromethyl)-1,2,4-oxadiazol-3-yl)phenoxy)pyrrolidin-1-yl)methanone; pyrimidin-5-yl(3-(4-(5-(trifluoromethyl)-1,2,4-oxadiazol-3-yl)phenoxy)pyrrolidin-1-yl)methanone; (3-(2-fluoro-4-(5-(trifluoromethyl)-1,2,4-oxadiazol-3-yl)phenoxy)pyrrolidin-1-yl)(pyridin-3-yl)methanone; (3-(2-fluoro-4-(5-(trifluoromethyl)-1,2,4-oxadiazol-3-yl)phenoxy)pyrrolidin-1-yl)(pyridin-4-yl)methanone; tert-butyl-3-(2-fluoro-4-(5-(trifluoromethyl)-1,2,4-oxadiazol-3-yl)phenoxy)pyrrolidine-1-carboxylate; 3-(4-((1-(ethylsulfonyl)pyrrolidin-3-yl)oxy)-3-fluorophenyl)-5-(trifluoromethyl)-1,2,4-oxadiazole; tert-butyl-3-(3-fluoro-4-(5-(trifluoromethyl)-1,2,4-oxadiazol-3-yl)phenoxy)pyrrolidine-1-carboxylate; (3-(3-fluoro-4-(5-(trifluoromethyl)-1,2,4-oxadiazol-3-yl)phenoxy)pyrrolidin-1-yl)(2-fluorophenyl)methanone; (3-(3-fluoro-4-(5-(trifluoromethyl)-1,2,4-oxadiazol-3-yl)phenoxy)pyrrolidin-1-yl)(3-fluorophenyl)methanone; (3-(3-fluoro-4-(5-(trifluoromethyl)-1,2,4-oxadiazol-3-yl)phenoxy)pyrrolidin-1-yl)(pyridin-3-yl)methanone; (3-(3-fluoro-4-(5-(trifluoromethyl)-1,2,4-oxadiazol-3-yl)phenoxy)pyrrolidin-1-yl)(pyridin-4-yl)methanone; 3-(4-((1-(ethylsulfonyl)pyrrolidin-3-yl)oxy)-2-fluorophenyl)-5-(trifluoromethyl)-1,2,4-oxadiazole; 1-(3-(3-fluoro-4-(5-(trifluoromethyl)-1,2,4-oxadiazol-3-yl)phenoxy)pyrrolidin-1-yl)ethan-1-one; 1-(3-(2-fluoro-4-(5-(trifluoromethyl)-1,2,4-oxadiazol-3-yl)phenoxy)pyrrolidin-1-yl)ethan-1-one; tert-butyl-3-((5-(5-(trifluoromethyl)-1,2,4-oxadiazol-3-yl)pyridin-2-yl)oxy)pyrrolidine-1-carboxylate; 3-(6-((1-(phenylsulfonyl)pyrrolidin-3-yl)oxy)pyridin-3-yl)-5-(trifluoromethyl)-1,2,4-oxadiazole; 3-(6-((1-(ethylsulfonyl)pyrrolidin-3-yl)oxy)pyridin-3-yl)-5-(trifluoromethyl)-1,2,4-oxadiazole; 3-(6-((1-((4-fluorophenyl)sulfonyl)pyrrolidin-3-yl)oxy)pyridin-3-yl)-5-(trifluoromethyl)-1,2,4-oxadiazole; (2-fluorophenyl)(3-((5-(5-(trifluoromethyl)-1,2,4-oxadiazol-3-yl)pyridin-2-yl)oxy)pyrrolidin-1-yl)methanone; (4-methoxyphenyl)(3-((5-(5-(trifluoromethyl)-1,2,4-oxadiazol-3-yl)pyridin-2-yl)oxy)pyrrolidin-1-yl)methanone; 3-(6-((1-(cyclopropylsulfonyl)pyrrolidin-3-yl)oxy)pyridin-3-yl)-5-(trifluoromethyl)-1,2,4-oxadiazole; (3-fluorophenyl)(3-((5-(5-(trifluoromethyl)-1,2,4-oxadiazol-3-yl)pyridin-2-yl)oxy)pyrrolidin-1-yl)methanone; pyridin-4-yl(3-((5-(5-(trifluoromethyl)-1,2,4-oxadiazol-3-yl)pyridin-2-yl)oxy)pyrrolidin-1-yl)methanone; pyridin-3-yl(3-((5-(5-(trifluoromethyl)-1,2,4-oxadiazol-3-yl)pyridin-2-yl)oxy)pyrrolidin-1-yl)methanone; 3-(6-((1-(phenylsulfonyl)pyrrolidin-3-yl)oxy)pyridin-3-yl)-5-(trifluoromethyl)-1,2,4-oxadiazole; 3-(6-((1-((4-methoxyphenyl)sulfonyl)pyrrolidin-3-yl)oxy)pyridin-3-yl)-5-(trifluoromethyl)-1,2,4-oxadiazole; 3-(6-((1-(cyclopropylsulfonyl)pyrrolidin-3-yl)oxy)pyridin-3-yl)-5-(trifluoromethyl)-1,2,4-oxadiazole; 3-(6-((1-tosylpyrrolidin-3-yl)oxy)pyridin-3-yl)-5-(trifluoromethyl)-1,2,4-oxadiazole; 3-(6-((1-((3-chlorophenyl)sulfonyl)pyrrolidin-3-yl)oxy)pyridin-3-yl)-5-(trifluoromethyl)-1,2,4-oxadiazole; 5-(trifluoromethyl)-3-(6-((1-((4-(trifluoromethyl)phenyl)sulfonyl)pyrrolidin-3-yl)oxy)pyridin-3-yl)-1,2,4-oxadiazole; 3-(6-((1-(propylsulfonyl)pyrrolidin-3-yl)oxy)pyridin-3-yl)-5-(trifluoromethyl)-1,2,4-oxadiazole; 3-(6-((1-(methylsulfonyl)pyrrolidin-3-yl)oxy)pyridin-3-yl)-5-(trifluoromethyl)-1,2,4-oxadiazole; 3-(6-((1-(m-tolylsulfonyl)pyrrolidin-3-yl)oxy)pyridin-3-yl)-5-(trifluoromethyl)-1,2,4-oxadiazole; 5-(trifluoromethyl)-3-(4-((1-((trifluoromethyl)sulfonyl)pyrrolidin-3-yl)oxy)phenyl)-1,2,4-oxadiazole; 3-(4-((1-(propylsulfonyl)pyrrolidin-3-yl)oxy)phenyl)-5-(trifluoromethyl)-1,2,4-oxadiazole; 3-(4-((1-((4-bromophenyl)sulfonyl)pyrrolidin-3-yl)oxy)phenyl)-5-(trifluoromethyl)-1,2,4-oxadiazole; 3-(4-((1-(pyridin-3-ylsulfonyl)pyrrolidin-3-yl)oxy)phenyl)-5-(trifluoromethyl)-1,2,4-oxadiazole; 5-(trifluoromethyl)-3-(4-((1-((4-(trifluoromethyl)phenyl)sulfonyl)pyrrolidin-3-yl)oxy)phenyl)-1,2,4-oxadiazole; 3-(4-((1-tosylpyrrolidin-3-yl)oxy)phenyl)-5-(trifluoromethyl)-1,2,4-oxadiazole; 3-(4-((1-((2,4-dichlorophenyl)sulfonyl)pyrrolidin-3-yl)oxy)phenyl)-5-(trifluoromethyl)-1,2,4-oxadiazole; 4-((3-(4-(5-(trifluoromethyl)-1,2,4-oxadiazol-3-yl)phenoxy)pyrrolidin-1-yl)sulfonyl)benzonitrile; 3-(4-((1-((3-chlorophenyl)sulfonyl)pyrrolidin-3-yl)oxy)phenyl)-5-(trifluoromethyl)-1,2,4-oxadiazole; 3-(4-((1-(isopropylsulfonyl)pyrrolidin-3-yl)oxy)phenyl)-5-(trifluoromethyl)-1,2,4-oxadiazole; 3-(4-((1-benzylpyrrolidin-3-yl)oxy)phenyl)-5-(trifluoromethyl)-1,2,4-oxadiazole; 3-(4-((1-((3-methylthiophen-2-yl)sulfonyl)pyrrolidin-3-yl)oxy)phenyl)-5-(trifluoromethyl)-1,2,4-oxadiazole; 3-(4-((1-((1-methyl-1H-imidazol-4-yl)sulfonyl)pyrrolidin-3-yl)oxy)phenyl)-5-(trifluoromethyl)-1,2,4-oxadiazole; 3-(6-((1-((3-fluorophenyl)sulfonyl)pyrrolidin-3-yl)oxy)pyridin-3-yl)-5-(trifluoromethyl)-1,2,4-oxadiazole; 4-((3-((5-(5-(trifluoromethyl)-1,2,4-oxadiazol-3-yl)pyridin-2-yl)oxy)pyrrolidin-1-yl)sulfonyl)benzonitrile; 5-(trifluoromethyl)-3-(6-((1-((3-(trifluoromethyl)phenyl)sulfonyl)pyrrolidin-3-yl)oxy)pyridin-3-yl)-1,2,4-oxadiazole; 3-(6-((1-((2-fluorophenyl)sulfonyl)pyrrolidin-3-yl)oxy)pyridin-3-yl)-5-(trifluoromethyl)-1,2,4-oxadiazole; 3-(6-((1-(pyridin-3-ylsulfonyl)pyrrolidin-3-yl)oxy)pyridin-3-yl)-5-(trifluoromethyl)-1,2,4-oxadiazole; 3-(6-((1-(benzylsulfonyl)pyrrolidin-3-yl)oxy)pyridin-3-yl)-5-(trifluoromethyl)-1,2,4-oxadiazole; 3-(6-((1-(isopropylsulfonyl)pyrrolidin-3-yl)oxy)pyridin-3-yl)-5-(trifluoromethyl)-1,2,4-oxadiazole; tert-butyl 3-((4-(5-(trifluoromethyl)-1,2,4-oxadiazol-3-yl)phenyl)thio)pyrrolidine-1-carboxylate; 3-(6-((1-((2-chlorophenyl)sulfonyl)pyrrolidin-3-yl)oxy)pyridin-3-yl)-5-(trifluoromethyl)-1,2,4-oxadiazole; 3-(6-((1-((4-chlorobenzyl)sulfonyl)pyrrolidin-3-yl)oxy)pyridin-3-yl)-5-(trifluoromethyl)-1,2,4-oxadiazole; 3-(6-((1-((2-methoxyethyl)sulfonyl)pyrrolidin-3-yl)oxy)pyridin-3-yl)-5-(trifluoromethyl)-1,2,4-oxadiazole; 3-(4-((1-(4-methylbenzyl)pyrrolidin-3-yl)oxy)phenyl)-5-(trifluoromethyl)-1,2,4-oxadiazole; 1-(3-((4-(5-(trifluoromethyl)-1,2,4-oxadiazol-3-yl)phenyl)thio)pyrrolidin-1-yl)ethan-1-one; m-tolyl(3-((4-(5-(trifluoromethyl)-1,2,4-oxadiazol-3-yl)phenyl)thio)pyrrolidin-1-yl)methanone; 3-(6-((1-(phenethylsulfonyl)pyrrolidin-3-yl)oxy)pyridin-3-yl)-5-(trifluoromethyl)-1,2,4-oxadiazole; (4-methoxyphenyl)(3-((4-(5-(trifluoromethyl)-1,2,4-oxadiazol-3-yl)phenyl)thio)pyrrolidin-1-yl)methanone; phenyl(3-((4-(5-(trifluoromethyl)-1,2,4-oxadiazol-3-yl)phenyl)thio)pyrrolidin-1-yl)methanone; 2-phenyl-1-(3-((4-(5-(trifluoromethyl)-1,2,4-oxadiazol-3-yl)phenyl)thio)pyrrolidin-1-yl)ethan-1-one; 3-(4-((1-(4-chlorobenzyl)pyrrolidin-3-yl)oxy)phenyl)-5-(trifluoromethyl)-1,2,4-oxadiazole; 3-(4-((1-isopropylpyrrolidin-3-yl)oxy)phenyl)-5-(trifluoromethyl)-1,2,4-oxadiazole; 2-phenyl-1-(3-((4-(5-(trifluoromethyl)-1,2,4-oxadiazol-3-yl)phenyl)sulfonyl)pyrrolidin-1-yl)ethan-1-one; (2-fluorophenyl)(3-((4-(5-(trifluoromethyl)-1,2,4-oxadiazol-3-yl)phenyl)thio)pyrrolidin-1-yl)methanone; 1-(3-((5-(5-(trifluoromethyl)-1,2,4-oxadiazol-3-yl)pyridin-2-yl)oxy)pyrrolidin-1-yl)ethan-1-one; 1-(3-((5-(5-(trifluoromethyl)-1,2,4-oxadiazol-3-yl)pyridin-2-yl)oxy)pyrrolidin-1-yl)propan-1-one; pyridin-4-yl(3-((4-(5-(trifluoromethyl)-1,2,4-oxadiazol-3-yl)phenyl)thio)pyrrolidin-1-yl)methanone; 2-(4-chlorophenyl)-1-(3-((4-(5-(trifluoromethyl)-1,2,4-oxadiazol-3-yl)phenyl)thio)pyrrolidin-1-yl)ethan-1-one; 2-(4-chlorophenyl)-1-(3-((4-(5-(trifluoromethyl)-1,2,4-oxadiazol-3-yl)phenyl)sulfonyl)pyrrolidin-1-yl)ethan-1-one; 2-(4-methoxyphenyl)-1-(3-((4-(5-(trifluoromethyl)-1,2,4-oxadiazol-3-yl)phenyl)sulfonyl)pyrrolidin-1-yl)ethan-1-one; (1-methyl-1H-pyrazol-3-yl)(3-((5-(5-(trifluoromethyl)-1,2,4-oxadiazol-3-yl)pyridin-2-yl)oxy)pyrrolidin-1-yl)methanone; isoxazol-3-yl(3-((5-(5-(trifluoromethyl)-1,2,4-oxadiazol-3-yl)pyridin-2-yl)oxy)pyrrolidin-1-yl)methanone; (4-(dimethylamino)phenyl)(3-((4-(5-(trifluoromethyl)-1,2,4-oxadiazol-3-yl)phenyl)thio)pyrrolidin-1-yl)methanone; oxazol-4-yl(3-((5-(5-(trifluoromethyl)-1,2,4-oxadiazol-3-yl)pyridin-2-yl)oxy)pyrrolidin-1-yl)methanone; thiazol-4-yl(3-((5-(5-(trifluoromethyl)-1,2,4-oxadiazol-3-yl)pyridin-2-yl)oxy)pyrrolidin-1-yl)methanone; (3-((4-(5-(trifluoromethyl)-1,2,4-oxadiazol-3-yl)phenyl)thio)pyrrolidin-1-yl)(4-(trifluoromethyl)phenyl)methanone; (3-((4-(5-(trifluoromethyl)-1,2,4-oxadiazol-3-yl)phenyl)sulfonyl)pyrrolidin-1-yl)(4-(trifluoromethyl)phenyl)methanone; 2-methyl-1-(3-((5-(5-(trifluoromethyl)-1,2,4-oxadiazol-3-yl)pyridin-2-yl)oxy)pyrrolidin-1-yl)propan-1-one; cyclopropyl(3-((5-(5-(trifluoromethyl)-1,2,4-oxadiazol-3-yl)pyridin-2-yl)oxy)pyrrolidin-1-yl)methanone; tert-butyl (R)-3-((4-(5-(trifluoromethyl)-1,2,4-oxadiazol-3-yl)phenyl)thio)pyrrolidine-1-carboxylate; tert-butyl (R)-3-(3-(5-(trifluoromethyl)-1,2,4-oxadiazol-3-yl)phenoxy)pyrrolidine-1-carboxylate; (R)-2,2-dimethyl-1-(3-(3-(5-(trifluoromethyl)-1,2,4-oxadiazol-3-yl)phenoxy)pyrrolidin-1-yl)propan-1-one; (R)-(2-fluorophenyl)(3-(3-(5-(trifluoromethyl)-1,2,4-oxadiazol-3-yl)phenoxy)pyrrolidin-1-yl)methanone; (R)-(3-fluorophenyl)(3-(3-(5-(trifluoromethyl)-1,2,4-oxadiazol-3-yl)phenoxy)pyrrolidin-1-yl)methanone; (R)-(4-fluorophenyl)(3-(3-(5-(trifluoromethyl)-1,2,4-oxadiazol-3-yl)phenoxy)pyrrolidin-1-yl)methanone; (R)-p-tolyl(3-(3-(5-(trifluoromethyl)-1,2,4-oxadiazol-3-yl)phenoxy)pyrrolidin-1-yl)methanone; (R)-m-tolyl(3-(3-(5-(trifluoromethyl)-1,2,4-oxadiazol-3-yl)phenoxy)pyrrolidin-1-yl)methanone; (R)-(3-(3-(5-(trifluoromethyl)-1,2,4-oxadiazol-3-yl)phenoxy)pyrrolidin-1-yl)(4-(trifluoromethyl)phenyl)methanone; (R)-(3-chlorophenyl)(3-(3-(5-(trifluoromethyl)-1,2,4-oxadiazol-3-yl)phenoxy)pyrrolidin-1-yl)methanone; (R)-(4-methoxyphenyl)(3-(3-(5-(trifluoromethyl)-1,2,4-oxadiazol-3-yl)phenoxy)pyrrolidin-1-yl)methanone; tert-butyl (R)-3-(methyl(4-(5-(trifluoromethyl)-1,2,4-oxadiazol-3-yl)phenyl)amino)pyrrolidine-1-carboxylate; (R)-2-(4-methoxyphenyl)-1-(3-((4-(5-(trifluoromethyl)-1,2,4-oxadiazol-3-yl)phenyl)thio)pyrrolidin-1-yl)ethan-1-one; (R)-(3-(methyl(4-(5-(trifluoromethyl)-1,2,4-oxadiazol-3-yl)phenyl)amino)pyrrolidin-1-yl)(phenyl)methanone; (R)-pyridin-2-yl(3-(3-(5-(trifluoromethyl)-1,2,4-oxadiazol-3-yl)phenoxy)pyrrolidin-1-yl)methanone; (R)-pyridin-4-yl(3-(3-(5-(trifluoromethyl)-1,2,4-oxadiazol-3-yl)phenoxy)pyrrolidin-1-yl)methanone; (R)-pyridin-3-yl(3-(3-(5-(trifluoromethyl)-1,2,4-oxadiazol-3-yl)phenoxy)pyrrolidin-1-yl)methanone; tert-butyl (S)-3-((4-(5-(trifluoromethyl)-1,2,4-oxadiazol-3-yl)phenyl)thio)pyrrolidine-1-carboxylate; tert-butyl (S)-3-(3-(5-(trifluoromethyl)-1,2,4-oxadiazol-3-yl)phenoxy)pyrrolidine-1-carboxylate; (S)-2,2-dimethyl-1-(3-(3-(5-(trifluoromethyl)-1,2,4-oxadiazol-3-yl)phenoxy)pyrrolidin-1-yl)propan-1-one; (S)-(2-fluorophenyl)(3-(3-(5-(trifluoromethyl)-1,2,4-oxadiazol-3-yl)phenoxy)pyrrolidin-1-yl)methanone; (S)-(3-fluorophenyl)(3-(3-(5-(trifluoromethyl)-1,2,4-oxadiazol-3-yl)phenoxy)pyrrolidin-1-yl)methanone; (S)-(4-fluorophenyl)(3-(3-(5-(trifluoromethyl)-1,2,4-oxadiazol-3-yl)phenoxy)pyrrolidin-1-yl)methanone; (S)-p-tolyl(3-(3-(5-(trifluoromethyl)-1,2,4-oxadiazol-3-yl)phenoxy)pyrrolidin-1-yl)methanone; (S)-m-tolyl(3-(3-(5-(trifluoromethyl)-1,2,4-oxadiazol-3-yl)phenoxy)pyrrolidin-1-yl)methanone; (S)-(3-(3-(5-(trifluoromethyl)-1,2,4-oxadiazol-3-yl)phenoxy)pyrrolidin-1-yl)(4-(trifluoromethyl)phenyl)methanone; (S)-(3-chlorophenyl)(3-(3-(5-(trifluoromethyl)-1,2,4-oxadiazol-3-yl)phenoxy)pyrrolidin-1-yl)methanone; (S)-(4-methoxyphenyl)(3-(3-(5-(trifluoromethyl)-1,2,4-oxadiazol-3-yl)phenoxy)pyrrolidin-1-yl)methanone; tert-butyl (S)-3-(methyl(4-(5-(trifluoromethyl)-1,2,4-oxadiazol-3-yl)phenyl)amino)pyrrolidine-1-carboxylate; (S)-2-(4-methoxyphenyl)-1-(3-((4-(5-(trifluoromethyl)-1,2,4-oxadiazol-3-yl)phenyl)thio)pyrrolidin-1-yl)ethan-1-one; (S)-(3-(methyl(4-(5-(trifluoromethyl)-1,2,4-oxadiazol-3-yl)phenyl)amino)pyrrolidin-1-yl)(phenyl)methanone; (S)-pyridin-2-yl(3-(3-(5-(trifluoromethyl)-1,2,4-oxadiazol-3-yl)phenoxy)pyrrolidin-1-yl)methanone; (S)-pyridin-4-yl(3-(3-(5-(trifluoromethyl)-1,2,4-oxadiazol-3-yl)phenoxy)pyrrolidin-1-yl)methanone; and (S)-pyridin-3-yl(3-(3-(5-(trifluoromethyl)-1,2,4-oxadiazol-3-yl)phenoxy)pyrrolidin-1-yl)methanone.

5. A composition comprising the compound of claim 1 in a combination with at least one further pesticidally active substance selected from the group consisting of fungicides, insecticides, nematicides, acaricides, biopesticides, herbicides, safeners, plant growth regulators, antibiotics, fertilizers and nutrients.

6. A composition comprising the compound of claim 1 and at least one agrochemically acceptable auxiliary.

7. The composition of claim 6, further comprising at least one additional active ingredient.

8. A composition comprising the compound of claim 1 applied to seed, wherein the amount of the compound of the formula I is from 0.1 g ai to 10 kg ai per 100 kg of seeds.

9. A method for controlling or preventing phytopathogenic fungi, wherein the method comprises: treating the fungi or the materials, plants, plant parts, locus thereof, soil or seeds to be protected against fungal attack, with an effective amount of the compound of claim 1.

10. A method for controlling or preventing infestation of plants by phytopathogenic micro-organisms in agricultural crops and or horticultural crops wherein an effective amount of at least one compound of claim 1 is applied to the seeds of plants.

11. The compound of claim 1, for controlling or preventing plant diseases.

12. The compound of claim 1, used as a fungicides.

13. The method of claim 9, wherein the plant diseases are rust pathogens selected from the group consisting of Puccinia spp. (rusts), comprising P. triticina (brown or leaf rust), P. striiformis (stripe or yellow rust), P. hordei (dwarf rust), P. graminis (stem or black rust) and P. recondita (brown or leaf rust) on cereals viz., wheat, barley or rye, P. melanocephala on sugarcane; and Phakopsora spp. comprising Phakopsora pachyrhizi and P. meibomiae on soybeans, Hemileia vastatrix (Coffee rust), Uromyces spp., comprising U fabae (rust of beans).

14. A process for preparing the compound of claim 1, said process comprising the steps of:
a. coupling a compound of Formula VIII and a compound of Formula II to obtain a compound of Formula III, 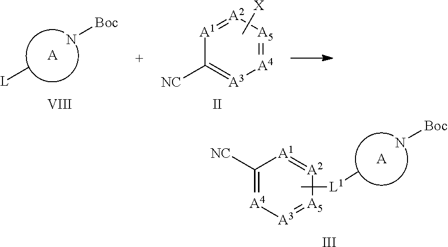
wherein, L is OH, SH or NHR⁶: L¹ is O, S, or NR⁶; X is F, C₁, Br, I, OH, SH or NHR⁶;
b. converting the compound of Formula III into a compound of Formula IV, 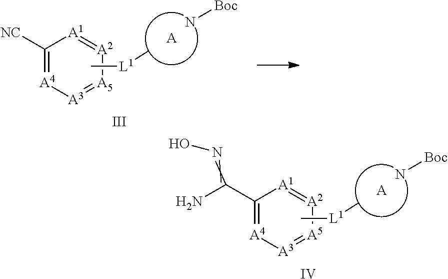
wherein, L¹ is O, S, or NR⁶;
c. cyclizing the compound of Formula IV with a compound of Formula V-a or V-b to obtain a compound of Formula Ia, 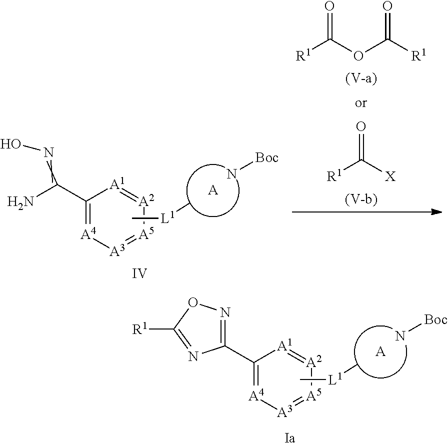
wherein, L¹ is O, S, or NR⁶;
d. converting the compound of Formula Ia into a compound of Formula VI using a suitable acid, 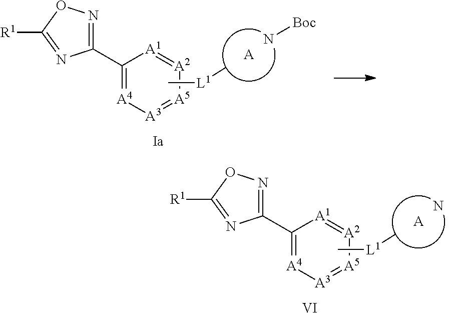
wherein, L¹ is O, S, or NR⁶;
e. reacting the compound of Formula VI with a suitable reactant selected from the group consisting of acid, acyl chloride, sulphonyl chloride, alkyl halide, benzyl halide or amine using suitable base or coupling reagent and a suitable solvent to obtain the compound of Formula I, 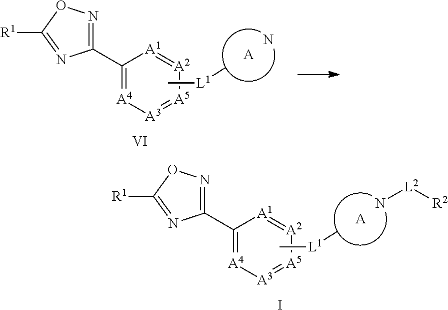
wherein, L¹ is O, S, or NR⁶; L² is (C═O), S(═O)₂ and (CR⁸R⁹)₁₋₃;
f. oxidizing a compound of Formula Ib using a suitable oxidizing agent to obtain the compound of Formula I, 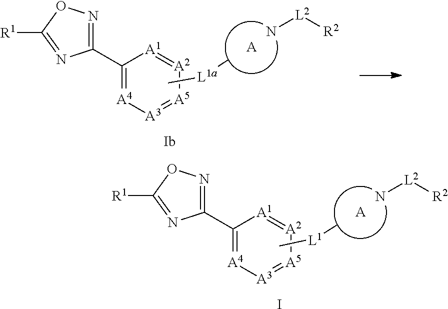
wherein, L^{1a} is S; L¹ is S(═O) or S(═O)₂; and
g. converting the compound of Formula Ib into the compound of Formula I using a suitable oxidizing agent and an amine source, 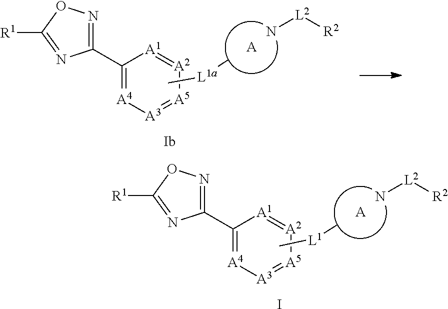
wherein, L^{1a} is [chemical structure]
